(19) United States
(12) Patent Application Publication
(10) Pub. No.: US 2025/0034122 A1
(43) Pub. Date: Jan. 30, 2025

(54) SUBSTITUTED N-CYANOPYRROLIDINES WITH ACTIVITY AS USP30 INHIBITORS

(71) Applicant: MISSION THERAPEUTICS LIMITED, Cambridge (GB)

(72) Inventors: Mark Ian KEMP, Cambridge (GB); Christopher Andrew LUCKHURST, Cambridge (GB)

(21) Appl. No.: 18/712,785

(22) PCT Filed: Nov. 30, 2022
(86) PCT No.: PCT/EP2022/083842

§ 371 Date: May 23, 2024

(30) Foreign Application Priority Data
Dec. 1, 2021 (GB) 2117366.1
Apr. 27, 2022 (GB) 2206136.0

(51) Int. Cl. C07D 413/14; A61K 31/4155; A61K 31/416; A61K 31/4192; A61K 31/422; A61K 31/4245; A61K 31/4439; C07D 401/14; C07D 403/14

(52) U.S. Cl. CPC C07D 413/14; A61K 31/4155; A61K 31/416; A61K 31/4192; A61K 31/422; A61K 31/4245; A61K 31/4439; C07D 401/14; C07D 403/14

(57) ABSTRACT

The present invention relates to a class of substituted N-cyanopyrrolidines with activity as inhibitors of the deubiquitylating enzyme USP30, having utility in a variety of therapeutic areas, including conditions involving mitochondrial dysfunction, cancer and fibrosis: Formula (I).

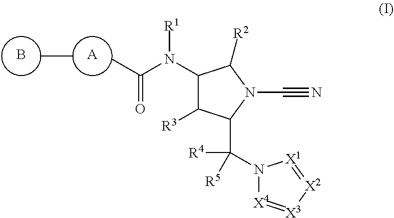

### FIELD OF THE INVENTION

The present invention relates to a class of N-cyanopyrrolidines with activity as inhibitors of the deubiquitylating enzyme ubiquitin C-terminal hydrolase 30, also known as ubiquitin specific peptidase 30 (USP30), uses thereof, processes for the preparation thereof and composition containing said inhibitors. These inhibitors have utility in a variety of therapeutic areas, including conditions involving mitochondrial dysfunction, cancer and fibrosis.

All documents cited or relied upon below are expressly incorporated herein by reference.

### BACKGROUND OF THE INVENTION

Ubiquitin is a small protein consisting of 76 amino acids that is important for the regulation of protein function in the cell. Ubiquitylation and deubiquitylation are enzymatically mediated processes by which ubiquitin is covalently bound or cleaved from a target protein by deubiquitylating enzymes (DUBs), of which there are approximately 100 DUBs in human cells, divided into sub-families based on sequence homology. The USP family are characterised by their common Cys and His boxes which contain Cys and His residues critical for their DUB activities. The ubiquitylation and deubiquitylation processes have been implicated in the regulation of many cellular functions including cell cycle progression, apoptosis, modification of cell surface receptors, regulation of DNA transcription and DNA repair. Thus, the ubiquitin system has been implicated in the pathogenesis of numerous disease states including inflammation, viral infection, metabolic dysfunction, CNS disorders, and oncogenesis.

Ubiquitin is a master regulator of mitochondrial dynamics. Mitochondria are dynamic organelles whose biogenesis, fusion and fission events are regulated by the post-translational regulation via ubiquitylation of many key factors such as mitofusins. In humans, USP30 is a 517 amino acid protein which is found in the mitochondrial outer membrane (Nakamura et al, 2008, Mol Biol 19:1903-11). It is the sole deubiquitylating enzyme bearing a mitochondrial addressing signal and has been shown to deubiquitylate a number of mitochondrial proteins. It has been demonstrated that USP30 opposes parkin-mediated mitophagy and that reduction of USP30 activity can rescue parkin-mediated defects in mitophagy (Bingol et al, 2015, Nature 510:370-5; Gersch et al, 2017, Nat Struct Mol Biol 24(11): 920-930; Cunningham et al, 2015, Nat Cell Biol 17(2): 160-169). USP30 inactivation can also increase mitochondrial protein import, potentially through ubiquitylation of TOM proteins (Jacoupy et al, 2019, Sci Rep 9(1): 11829). A small proportion of USP30 has been localized to peroxisomes, which are generated through fusion of mitochondrial and ER vesicles, with USP30 potentially antagonizing the Pex2/pexophagy pathway (Riccio et al, 2019, J Cell Biol 218(3): 798-807). The E3 Ub ligase March5 and the deubiquitinase USP30 associate with the translocase and regulate mitochondrial import, and while March5 opposes mitochondrial import and directs degradation of substrates, USP30 deubiquitinates substrates to promote their import (Phu et al, 2020, Molecular Cell 77, 1107-1123).

Mitochondrial dysfunction can be defined as diminished mitochondrial content (mitophagy or mitochondrial biogenesis), as a decrease in mitochondrial activity and oxidative phosphorylation, but also as modulation of reactive oxygen species (ROS) generation. Hence a role for mitochondrial dysfunctions in a very large number of aging processes and pathologies.

For example, Parkinson's disease affects around 10 million people worldwide (Parkinson's Disease Foundation) and is characterised by the loss of dopaminergic neurons in the substantia nigra. The exact mechanisms underlying PD are unclear; however mitochondrial dysfunction is increasingly appreciated as a key determinant of dopaminergic neuronal susceptibility in PD and is a feature of both familial and sporadic disease, as well as in toxin-induced Parkinsonism. Parkin is one of a number of proteins that have been implicated with early onset PD. While most PD cases are linked to defects in alpha-synuclein, 10% of Parkinson's cases are linked to specific genetic defects, one of which is in the ubiquitin E3 ligase parkin. Parkin and the protein kinase PTEN-induced putative kinase 1 (PINK1) collaborate to ubiquitylate mitochondrial membrane proteins of damaged mitochondria resulting in mitophagy. Dysregulation of mitophagy results in increased oxidative stress, which has been described as a characteristic of PD. Inhibition of USP30 could therefore be a potential strategy for the treatment of PD. For example, PD patients with parkin mutations leading to reduced activity could be therapeutically compensated by inhibition of USP30.

It has been reported that depletion of USP30 enhances mitophagic clearance of mitochondria and also enhances parkin-induced cell death. USP30 has also been shown to regulate BAX/BAK-dependent apoptosis independently of parkin overexpression. Depletion of USP30 sensitises cancer cells to BH-3 mimetics such as ABT-737, without the need for parkin overexpression. Thus, an anti-apoptotic role has been demonstrated for USP30 and USP30 is therefore a potential target for anti-cancer therapy.

The ubiquitin-proteasome system has gained interest as a target for the treatment of cancer following the approval ofthe proteasome inhibitor bortezomib (Velcade®) for the treatment of multiple myeloma. Extended treatment with bortezomib is limited by its associated toxicity and drug resistance. However, therapeutic strategies that target specific aspects of the ubiquitin-proteasome pathway upstream of the proteasome, such as DUBs, are predicted to be better tolerated (Bedford et al, 2011, Nature Rev 10:29-46).

Fibrotic diseases, including renal, hepatic and pulmonary fibrosis, are a leading cause of morbidity and mortality and can affect all tissues and organ systems. Fibrosis is considered to be the result of acute or chronic stress on the tissue or organ, characterized by extracellular matrix deposition, reduction of vascular/tubule/duct/airway patency and impairment of function ultimately resulting in organ failure. Many fibrotic conditions are promoted by lifestyle or environmental factors; however, a proportion of fibrotic conditions can be initiated through genetic triggers or indeed are considered idiopathic (i.e., without a known cause). Certain fibrotic disease, such as idiopathic pulmonary fibrosis (IPF), can be treated with non-specific kinase inhibitor (nintedanib) or drugs without a well-characterized mechanism of action (pirfenidone). Other treatments for organ fibrosis, such as kidney or liver fibrosis, alleviate pressure on the organ itself (e.g., beta blockers for cirrhosis, angiotensin receptor blockers for chronic kidney disease). Attention to lifestyle factors, such as glucose and diet control, may also influence the course and severity of disease.

Mitochondrial dysfunction has been implicated in a number of fibrotic diseases, with oxidative stress downstream of dysfunction being the key pathogenic mediator, alongside decreased ATP production.

In preclinical models, disruption of the mitophagy pathway (through mutation or knockout of either parkin or PINK1) exacerbates lung fibrosis and kidney fibrosis, with evidence of increased oxidative stress.

Kurita et al, 2017, Respiratory Research 18:114, discloses that accumulation of profibrotic myofibroblasts is a crucial process for fibrotic remodelling in IPF. Recent findings are said to show participation of autophagy/mitophagy, part of the lysosomal degradation machinery, in IPF pathogenesis, and that mitophagy has been implicated in myofibroblast differentiation through regulating mitochondrial reactive oxygen species (ROS)-mediated platelet-derived growth factor receptor (PDGFR) activation. Kurita's results suggested that pirfenidone induces PARK2-mediated mitophagy and also inhibits lung fibrosis development in the setting of insufficient mitophagy, which may at least partly explain the anti-fibrotic mechanisms for IPF treatment.

Williams et al, 2015, Pharmacol Res. December; 102: 264-269, discuss the role of PINK1-Parkin-mediated autophagy in protecting against alcohol and acetaminophen-induced liver injury by removing damaged mitochondria via mitophagy. It is suggested that pharmacological stabilization of USP8 or inactivation of USP15 and USP30 may be potential therapeutic targets for upregulating Parkin-induced mitophagy and in turn protect against drug-induced liver injury. However, it is noted that the DUBs are regulated both transcriptionally and post-translationally, which may make drug development for targeting these specific enzymes challenging, and in addition, phosphorylated ubiquitin was shown to be resistant to DUBs. The authors conclude that upregulating PINK1 stabilization or kinase activity may be a more effective target than inhibiting DUBs.

Williams et al, 2015, Biomolecules 5, 2619-2642, and Williams et al, 2015, Am J Physiol Gastrointest Liver Physiol 309: G324-G340, review mechanisms involved in regulation of mitochondrial homeostasis in the liver and how these mechanisms may protect against alcohol-induced liver disease.

Luciani et al, 2020, Nat. Commun. 11, 970, reports deregulation of mitochondrial network in terminally differentiated cells contributes to a broad spectrum of disorders, including methylmalonic acidemia (MMA). MMA is one of the most common inherited metabolic disorders, due to deficiency of the mitochondrial methylmalonyl-coenzyme A mutase (MMUT). MMUT deficiency induces metabolic and mitochondrial alterations that are exacerbated by anomalies in PINK1/Parkin-mediated mitophagy, causing the accumulation of dysfunctional mitochondria that trigger epithelial stress and ultimately cell damage. A link is suggested between primary MMUT deficiency, diseased mitochondria, mitophagy dysfunction and epithelial stress, and potential therapeutic perspectives for MMA is provided.

Kluge et al, Bioorganic & Medicinal Chemistry Letters, 2018, 28 2655-2659, reports that selective inhibitors of USP30 accelerate mitophagy.

Series of derivatives of N-cyano-substituted heterocycles are disclosed as deubiquitylating enzyme inhibitors in PCT applications WO 2016/046530 (U.S. Ser. No. 15/513,125, U.S. Ser. No. 15/894,025, U.S. Ser. No. 16/448,066), WO 2016/156816 (U.S. Ser. No. 15/558,632, U.S. Ser. No. 16/297,937, U.S. Ser. No. 16/419,558, U.S. Ser. No. 16/419,747, U.S. Ser. No. 16/788,446), WO 2017/009650 (U.S. Ser. No. 15/738,900), WO 2017/093718 (U.S. Ser. No. 15/776,149), WO 2017/103614 (U.S. Ser. No. 15/781,615), WO 2017/149313 (U.S. Ser. No. 16/078,518), WO 2017/109488 (U.S. Ser. No. 16/060,299), WO 2017/141036 (U.S. Ser. No. 16/070,936), WO 2017/163078 (U.S. Ser. No. 16/087,515), WO 2017/158381 (U.S. Ser. No. 16/080,229), WO 2017/158388 (U.S. Ser. No. 16/080,506), WO 2018/065768 (U.S. Ser. No. 16/336,685), WO 2018/060742 (U.S. Ser. No. 16/336,202), WO 2018/060689 (U.S. Ser. No. 16/334,836), WO 2018/060691 (U.S. Ser. No. 16/336,363), WO 2018/220355 (U.S. Ser. No. 16/615,040), WO 2018/234755 (U.S. Ser. No. 16/615,709), WO 2020/212350, WO 2020/212351, WO 2021/043870, WO 2021/204856, WO 2021/239863, WO 2021/245186, WO 2021/249909 and WO 2022/084479, each of which are expressly incorporated herein by reference. PCT application WO 2019/171042 (U.S. Ser. No. 16/977,019), which is expressly incorporated herein by reference, discloses the use of N-cyanopyrrolidines as inhibitors of USP30 for the treatment of fibrotic diseases.

Falgueyret et al, 2001, J. Med. Chem. 44, 94-104, and PCT application WO 01/77073 refer to cyanopyrrolidines as inhibitors of Cathepsins K and L, with potential utility in treating osteoporosis and other bone-resorption related conditions. PCT application WO 2015/179190 refers to N-acylethanolamine hydrolysing acid amidase inhibitors, with potential utility in treating ulcerative colitis and Crohn's disease. PCT application WO 2013/030218 refers to quinazolin-4-one compounds as inhibitors of ubiquitin specific proteases, such as USP7, with potential utility in treating cancer, neurodegenerative diseases, inflammatory disorders and viral infections. PCT applications WO 2015/017502 and WO 2016/019237 refer to inhibitors of Bruton's tyrosine kinase with potential utility in treating disease such as autoimmune disease, inflammatory disease and cancer. PCT applications WO 2009/026197, WO 2009/129365, WO 2009/129370, and WO 2009/129371, refer to cyanopyrrolidines as inhibitors of Cathepsin C with potential utility in treating COPD. United States patent application US 2008/0300268 refers to polyaromatic compounds as inhibitors of tyrosine kinase receptor PDGFR. PCT applications WO 2019/222468, WO 2019/071073, WO 2020/036940 and WO 2020/072964, Rusilowicz-Jones et al, 2020, bioRxiv 2020.04.16.044206 (20 Apr. 2020), and Tsefou et al, bioRxiv 2021.02.02.429344 (2 Feb. 2021), refer to cyanamide-containing compounds as USP30 inhibitors. Yue et al, 2014, Cell Research, 24, 482-496, refers to a diterpenoid derivative 15-oxospiramilactone as a USP30 inhibitor that induced mitochondrial fusion. PCT application WO 2015/183987 refers to pharmaceutical compositions comprising deubiquitinase inhibitors and human serum albumin in methods of treating cancer, fibrosis, an autoimmune disease or condition, an inflammatory disease or condition, a neurodegenerative disease or condition or an infection. It is noted that deubiquitinases, including UCHL5/UCH37, USP4, USP9X, USP11 and USP15, are said to have been implicated in the regulation of the TGF-beta signalling pathway, the disruption of which gives rise to neurodegenerative and fibrotic diseases, autoimmune dysfunction and cancer.

PCT application WO 2006/067165 refers to a method for treating fibrotic diseases using indolinone kinase inhibitors. PCT application WO 2007/119214 refers to a method for treating early stage pulmonary fibrosis using an endothelin receptor antagonist. PCT application WO 2012/170290 refers to a method for treating fibrotic diseases using THC acids. PCT application WO 2018/213150 refers to sulfonamide USP30 inhibitors with potential utility in the treatment of conditions involving mitochondrial defects. Larson-Casey et al, 2016, Immunity 44, 582-596, concerns macrophage Akt1 kinase-mediated mitophagy, apoptosis resistance and pulmonary fibrosis. Tang et al, 2015, Kidney Diseases 1, 71-79, reviews the potential role of mitophagy in renal pathophysiology.

There exists a need for safe, alternative, and/or improved methods and compositions for the treatment or prevention of conditions involving mitochondrial dysfunction, cancer and fibrosis, and the various symptoms and conditions associated therewith. While not wishing to be bound by any particular theory or mechanism, it is believed that the compounds of the present invention act to inhibit the enzyme USP30, which in turn upregulates Parkin-induced mitophagy.

Acute Kidney Injury (AKI) is defined as an abrupt decrease in kidney function occurring over 7 days or less, with severity of injury staged based on increased serum creatinine (SCr) and decreased urine output as described in the Kidney Disease Improving Global Outcomes (KDIGO) guidelines. AKI occurs in about 13.3 million people per year, 85% of whom live in the developing world and it is thought to contribute to about 1.7 million deaths every year (Mehta et al, 2015, Lancet 385(9987): 2616-2643).

AKI more than likely results in permanent kidney damage (i.e., chronic kidney disease; CKD) and may also result in damage to non-renal organs. AKI is a significant public health concern particularly when considering the absolute number of patients developing incident CKD, progressive CKD, end-stage renal disease and cardiovascular events. AKI has been found to be prevalent in patients hospitalised by COVID-19 and is strongly associated with hospital mortality, with mitochondrial damage and dysfunction reported as a potential pathophysiological mechanism and therapeutic target (Kellum et al, Nephrol Dial Transplant (2020) 35: 1652-1662).

AKI and CKD are viewed as a continuum on the same disease spectrum (Chawla et al, 2017, Nat Rev Nephrol 13(4): 241-257). Patients undergoing coronary artery bypass graft (CABG) are at high risk for kidney injury. There is an obvious unmet medical need in the development of medicinal products for the treatment and/or prevention of AKI.

The kidney is a site of high metabolic demand, with high mitophagy rates demonstrated in vivo (McWilliams et al, 2018, Cell Metab 27(2): 439-449 e435). Renal Proximal Tubule Epithelial Cells (RPTECs), a cell type with significant ATP requirement for solute/ion exchange, are rich in mitochondria and are the primary effector cells of Acute Kidney Injury (AKI) in the kidney.

Mitochondrial dysfunction has been implicated in AKI/CKD mechanisms, both through multiple lines of evidence from preclinical AKI and CKD models and also through data demonstrating abnormal mitochondrial phenotypes in patient biopsies (Emma et al, 2016, Nat Rev Nephrol 12(5): 267-280; Eirin et al, 2017, Handb Exp Pharmacol 240: 229-250). Furthermore, Primary mitochondrial disease often manifest in renal symptoms, such as focal segmental glomerulosclerosis (Kawakami et al, 2015, J Am Soc Nephrol 26(5): 1040-1052) in patients with MELAS/MIDD, and also primary tubular pathologies in patients with Coenzyme Q deficiencies. Mutations in mtDNA can cause maternally inherited tubulointerstitial disease (Connor et al, 2017, PLoS Genet 13(3): e1006620).

Regarding mitochondrial quality control in renal injury (Tang et al, 2018, Autophagy 14(5): 880-897) demonstrated that renal injury was exacerbated following ischemic AKI in both PINK1 KO and PARK2 KO mice, suggesting that PINK1/PARKIN-mediated mitophagy plays a protective role following IRI in the kidney. In addition, parkin/PINK1 mitophagy protects against cisplatin induced kidney injury (Wang et al, 2018, Cell Death Dis 9(11): 1113). Limited models of CKD are available for mitophagy investigation, supportive evidence for mitochondrial quality control in fibrosis comes from studies on fibrotic lung conditions such as COPD and IPF. Parkin knockout animals show exacerbated lung fibrosis in response to bleomycin (Kobayashi et al, 2016, J Immunol, 197:504-516). Similarly, airway epithelial cells from parkin knockout (KO) animals show exacerbated fibrotic and senescent responses to cigarette smoke (Araya et al, 2019, Autophagy 15(3): 510-526).

Preclinical models are available to study potential novel therapeutics, through their ability to model fibrosis pathology (e.g., collagen deposition) consistent with the human condition. Preclinical models can be toxin-mediated (e.g., bleomycin for lung and skin fibrosis), surgical (e.g., ischemia/reperfusion injury model and unilateral ureter obstruction model for acute tubulointerstitial fibrosis), and genetic (e.g., diabetic (db/db) mice for diabetic nephropathy). For example, both examples previously given for indicated IPF treatments (nintedanib and pirfenidone) show efficacy in the bleomycin lung fibrosis model.

Leigh syndrome is a rare inherited neurometabolic disorder that affects the central nervous system. This progressive disorder begins in infants between the ages of three months and two years. Rarely, it occurs in teenagers and adults. Leigh syndrome can be caused by mutations in nuclear DNA encoding for mitochondrial proteins, mutations in mitochondrial DNA (maternally inherited Leigh syndrome—MILS), or by deficiencies of an enzyme called pyruvate dehydrogenase located on the short arm of the X Chromosome (X-linked Leigh syndrome). Symptoms of Leigh syndrome usually progress rapidly. The earliest signs may be poor sucking ability, and the loss of head control and motor skills. These symptoms may be accompanied by loss of appetite, vomiting, irritability, continuous crying, and seizures. As the disorder progresses, symptoms may also include generalized weakness, lack of muscle tone, and episodes of lactic acidosis, which can lead to impairment of respiratory and kidney function.

In maternally inherited Leigh syndrome (MILS), genetic mutations in mitochondrial DNA (at a high proportion of >90%) interfere with the energy sources that run cells in an area of the brain that plays a role in motor movements. Genetic mutations in mitochondrial DNA result in a chronic lack of energy in these cells, which in turn affects the central nervous system and causes progressive degeneration of motor functions. When the genetic mutations in mitochondrial DNA that causes MILS are less abundant (less than 90%), the condition is known as neuropathy ataxia and retinitis pigmentosa (NARP). There is also a form of Leigh's disease (called X-linked Leigh's disease) which is the result of mutations in a gene that produces another group of substances that are important for cell metabolism. A further variant of Leigh syndrome exists which is called French Canadian variant, characterized by mutations in a gene called LRPPRC. Similar neurological symptoms are expressed as those for Leigh syndrome, although Liver Steatosis is commonly also observed in the French Canadian variant.

Muscular dystrophies are characterized by specific abnormalities (e.g., variation of muscle fiber size, muscle fiber necrosis, scar tissue formation and inflammation) in muscle biopsy from the patients. Approximately thirty different genetic conditions make up the muscular dystrophies. Duchenne Muscular Dystrophy (DMD) is classified as a dystrophinopathy, one of a spectrum of muscle diseases, each caused by alterations in the dystrophin gene. The clinical hallmarks of DMD include weakness and wasting of various voluntary muscles of the body. In most advanced stages of the disease, the heart and gut muscles will be affected, with cardiomyopathy and subsequent heart failure being a primary cause of mortality.

Reid, et al, “The Interplay of Mitophagy and Inflammation in Duchenne Muscular Dystrophy.” Life 2021, 11, 648, reports that mitochondrial dysfunction is one of the first characteristics that can be seen in dystrophic muscle before the overt breakdown of muscle, suggesting that it could be a significant contributor to the pathology of the disease rather than a later consequence of muscle necrosis. In healthy tissue, these damaged mitochondria are marked for mitophagy, however, it has been shown that mitophagy is significantly impaired in DMD.

Vila et al, Cell Death and Differentiation (2017) 24, 330-342, identified that active mitochondria are required for the repair of sarcolemmal injury in healthy myofibers, and that proteins facilitating mitochondrial function, calcium homeostasis, and sarcolemmal stability are significantly altered at disease onset in dystrophin-deficient mdx mice. It was hypothesised that increased calcium overload in dystrophic myofibers and mitochondria causes mitochondrial dysfunction, which in turn diminishes the repair ability of the dystrophic myofibers and results in their death and, therefore, enhanced autophagy may aid to clear these mitochondria.

Luan et al, Science Translational Medicine (2021) 13, 1-12, demonstrated that animal DMD models and DMD patient tissue contain reduced expressions of genes involved in mitophagy which contributes to mitochondrial dysfunction. Administration of Urolithin A, a natural compound able to stimulate mitophagy, increased muscle metabolism and stem cell regenerative ability, resulting in muscle function recovery in the mdx mouse model of DMD. Urolithin A also reduced CD45 positive inflammatory cells in TA muscles, suggesting potential anti-inflammatory mechanisms may contribute to benefit in the mdx mouse model.

Accordingly, there is a need for compounds that are inhibitors of USP30 for the treatment or prevention of conditions where inhibition of USP30 is indicated. In particular, there exists a need for USP30 inhibitors that have suitable and/or improved properties in order to maximise efficacy against the target disease.

### SUMMARY OF THE INVENTION

The present invention is directed to compounds of formula (I):

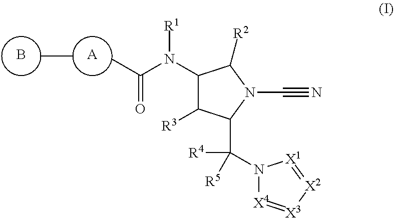

a tautomer thereof, or a pharmaceutically acceptable salt of said compound or tautomer, wherein either:
   (a) X¹ is N; and
      X², X³ and X⁴ are CR⁶; or
   (b) X¹ is N;
      one of X², X³ and X⁴ are N; and
      two of X², X³ and X⁴ are CR⁶; or
   (c) X¹ and X⁴ are CR⁶; and
      X² and X³ are N;
ring A is selected from:
   (i) a 5-membered monocyclic heteroaryl ring comprising 1 to 3 heteroatoms, each independently selected from N and 0;
   (ii) a 6-membered monocyclic heteroaryl ring comprising 1 to 3 heteroatoms, each independently selected from N, O and S;
   (iii) a 4 to 6-membered saturated or partially saturated monocyclic heterocyclyl ring comprising 1 to 3 heteroatoms, each independently selected from N, O and S; and
   (iv) phenyl or naphthyl;
ring A is either unsubstituted or substituted by 1 or 2 R⁷ substituents;
ring B is selected from:
   (i) phenyl or naphthyl;
   (ii) a 5 to 6-membered monocyclic heteroaryl ring comprising 1 to 3 heteroatoms, each independently selected from N, O and S; and
   (iii) a 9 to 10-membered bicyclic heteroaryl ring comprising 1 to 4 heteroatoms, each independently selected from N, O and S;
ring B is either unsubstituted or substituted by 1 to 5 substituents, each independently selected from halo, CN, hydroxy, oxo, (C₁-C₆)alkyl, (C₁-C₆)alkoxy, halo(C₁-C₆)alkyl, halo(C₁-C₆)alkoxy, (C₃-C₆)cycloalkyl, O(C₃-C₆)cycloalkyl, (C₁-C₆)alkoxy(C₁-C₆)alkyl, oxetanyloxy, azetidinyl, pyrrolidinyl, piperidinyl, NH(C₁-C₆)alkyl, N((C₁-C₆)alkyl)₂, C(O)NH(C₁-C₆)alkyl, C(O)N((C₁-C₆)alkyl)₂, NHC(O)(C₁-C₆)alkyl, N(C₁-C₆)alkyl)C(O)(C₁-C₆)alkyl), C(O)(C₁-C₆)alkyl, C(O)O(C₁-C₆)alkyl, CO₂H, CONH₂, SO₂NH(C₁-C₆)alkyl and SO₂N((C₁-C₆)alkyl)₂;
R¹ is selected from hydrogen, (C₁-C₆)alkyl and (C₃-C₆)cycloalkyl;
R² and R³ are each independently selected from hydrogen, halo, (C₁-C₄)alkyl and (C₁-C₄)alkoxy;
R⁴ and R are each independently selected from hydrogen and (C₁-C₄)alkyl;
R⁶ is hydrogen or (C₁-C₄)alkyl; and
each R⁷ is independently selected from halo, (C₁-C₆)alkyl and (C₁-C₆)alkoxy;
with the proviso that when ring A is unsubstituted oxazolyl or oxadiazolyl, and ring B is phenyl, and R¹, R², R³, R⁴ and R are each hydrogen, and two of X¹, X², X³ and X⁴ are N, then ring B is not substituted by CF₃ in the position meta to ring A.

The present invention is also directed to uses of the compounds of formula (I), particularly in the treatment of conditions involving mitochondrial dysfunction, cancer and fibrosis, and also processes for the preparation thereof and pharmaceutical compositions containing said compounds.

### DETAILED DESCRIPTION OF THE INVENTION

The present invention is directed to USP30 inhibitors that have suitable and/or improved properties in order to maximise efficacy against the target disease. Such properties include, for example, potency, selectivity, physicochemical properties, ADME (absorption, distribution, metabolism and excretion) properties, including PK (pharmacokinetic) profile, and safety profile.

It is generally desirable to maximise the potency of a drug molecule against the target enzyme in relevant assays in order to lower the effective/efficacious dosage that is to be administered to patients.

Compounds of the invention may be tested for USP30 affinity using the in vitro biochemical fluorescence polarization (FP) assay described herein.

USP30 is a transmembrane protein located in the outer membrane of mitochondria, which are energy-producing organelles present inside cells. Therefore, being able to demonstrate cellular activity in vitro is advantageous, as this is one of a number of components that may indicate a greater ability to engage the target in its physiological setting, i.e., where the USP30 inhibitor compound is able to penetrate cells. The USP30 cellular western blot (WB) assay aims to test the activity of compounds against USP30 in cells using an irreversible activity probe to monitor USP30 activity. Analogously to the cellular western blot assay, target engagement assessment (ex vivo) may be carried out in either brain or kidney tissue samples from compound-dosed animals.

To extend target binding knowledge to downstream pharmacodynamics, assessment of TOM20 (an outer mitochondrial membrane protein) ubiquitylation may be made.

In general, it is important for a drug to be as selective as possible for its desired target enzyme; additional activities give rise to the possibility of side effects. The exact physiological role of many DUBs has yet to be fully determined, however, irrespective of whatever role these DUBs may or may not play, it is a sound medicinal chemistry precept to ensure that any drug has selectivity over related mechanistic targets of unknown physiological function. Representative examples of DUB enzymes for which the compounds of the present invention may be screened against are UCHL1, UCHL3, UCHL5, YOD1, SENP2, SENP6, TRABID, BAP1, Cezanne, MINDY2/FAM63B, OTUD1, OTUD3, OTUD5, OTUD6A, OTUD6B, OTUB1/UBCH5B, OTUB2, CYLD, VCPIP, AMSH-LP, JOSD1, JOSD2, USP1/UAF1, USP2, USP4, USP5, USP6, USP7, USP8, USP9x, USP10, USP11, USP12/UAF1, USP13, USP14, USP15, USP16, USP19, USP20, USP21, USP22, USP24, USP25, USP28, USP32, USP34, USP35, USP36, USP45, USP46/UAF1, USP47 and USP48. Preferably, compounds of the invention have good selectivity for USP30 over one or more of these DUB enzymes.

Aside from selectivity over other DUB enzymes, it is important for a drug to have low affinity for other targets, and pharmacological profiling may be performed against panels of targets to assess the potential for, and to minimise, potential off-target effects. Examples of targets for which the compounds of the present invention may be screened against are those of the industry standard Eurofins-Cerep SafetyScreen44 panel, which includes 44 targets as a representative selection of GPCR receptors, transporters, ion channels, nuclear receptors, and kinase and non-kinase enzymes. Preferably, compounds of the invention have insignificant affinity against targets of this screening panel. Further examples of targets for which the compounds of the present invention may be screened against are kinases of the Thermo Fisher SelectScreen kinase profiling panel, which includes 39 targets as a representative selection of kinase enzymes. Preferably, compounds of the invention have insignificant affinity against targets of this screening panel. Additionally, examples of a particular enzyme class for which the compounds of the present invention may be screened against are the cathepsins (e.g., cathepsins A, B, C, H, K, L, S, V and Z). Preferably, compounds of the invention have good selectivity for USP30 over one or more of these enzymes.

There is also a need for compounds that have favourable pharmacokinetic properties such that they are suitable for oral administration. An orally administered drug should have good bioavailability; that is an ability to readily cross the gastrointestinal (GI) tract and not be subject to extensive metabolism as it passes from the GI tract into the systemic circulation. Once a drug is in the systemic circulation the rate of metabolism is also important in determining the time of residence of the drug in the body.

Thus, it is clearly favourable for drug molecules to have the properties of being readily able to cross the GI tract and being only slowly metabolised in the body. The Caco-2 assay is a widely accepted model for predicting the ability of a given molecule to cross the GI tract. The majority of metabolism of drug molecules generally occurs in the liver, and in vitro assays using whole cell hepatocytes (animal or human) are widely accepted methods for measuring the susceptibility of a given molecule towards metabolism in the liver. Such assays aim to predict in vivo clearance from the hepatocyte calculated clearance value.

Compounds which have good Caco-2 flux and are stable towards hepatocytes are predicted to have good oral bioavailability (good absorption across the GI tract and minimal extraction of compound as it passes through the liver) and a long residence time in the body that is sufficient for the drug to be efficacious.

The solubility of a compound is an important factor in achieving a desired concentration of drug in systemic circulation for the anticipated pharmacological response. Low aqueous solubility is a problem encountered with formulation development of new chemical entities and to be absorbed a drug must be present in the form of solution at the site of absorption. The kinetic solubility of a compound may be measured using a turbidimetric solubility assay, the data from which may also be used in conjunction with Caco-2 permeability data to predict dose dependent human intestinal absorption.

Other parameters that may be measured using standard assays that are indicative of a compound's exposure profile include, for example plasma stability (half-life measurement), blood AUC, Cₘₐₓ, Cₘᵢₙ and Tₘₐₓ values.

The treatment of CNS disorders, including Alzheimer's disease, Parkinson's disease, and other disorders described herein, requires drug molecules to target the brain, which requires adequate penetration of the blood brain barrier. There is, therefore, a need for USP30 inhibitors that possess effective blood brain penetration properties and provide suitable residence time in the brain to be efficacious. The probability that a compound can cross the blood brain barrier may be measured by an in vitro flux assay utilizing a MDR1-MDCK cell monolayer (Madin-Darby Canine Kidney cells transfected with MDR-1 resulting in overexpression of the human efflux transporter P-glycoprotein). Additionally, exposure may also be measured directly in brain and plasma using in vivo animal models.

There is also a need for compounds that have a favourable safety profile, which may be measured by a variety of standard in vitro and in vivo methods. A cell toxicity counter-screen may be used to assay the anti-proliferative/cytotoxic effect in a particular cell line (e.g., HCT116) by fluorometric detection of rezasurin (alamarBlue™) to resofurin in response to mitochondrial activity.

Toxicology and safety studies may also be conducted to identify potential target organs for adverse effects and define the Therapeutic Index to set the initial starting doses in clinical trials. Regulatory requirements generally require studies to be conducted in at least two laboratory animal species, one rodent (rat or mouse) and one nonrodent (rabbit, dog, non-human primate, or other suitable species).

The bacterial reverse mutation assay (Ames Test) may be used to evaluate the mutagenic properties of compounds of the invention, commonly by using the bacterial strain Salmonella typhimurium, which is mutant for the biosynthesis of the amino acid histidine.

The micronucleus assay may be used to determine if a compound is genotoxic by evaluating the presence of micronuclei. Micronuclei may contain chromosome fragments produced from DNA breakage (clastogens) or whole chromosomes produced by disruption of the mitotic apparatus (aneugens).

The hERG predictor assay provides valuable information about the possible binding of test compounds to the potassium channel and potential QT prolongation on echocardiogram. Inhibition of the hERG current causes QT interval prolongation resulting in potentially fatal ventricular tachyarrhythmia (Torsades de Pointes). Typically, assay data may be generated from an automated patch-clamp assay platform.

According to a first aspect, the present invention provides a compound of formula (I):

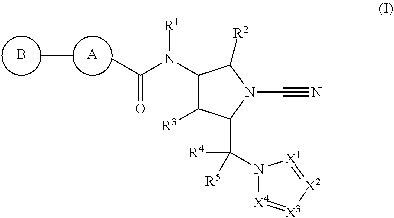

a tautomer thereof, or a pharmaceutically acceptable salt of said compound or tautomer, wherein either:
   (a) X¹ is N; and
      X², X³ and X⁴ are CR⁶; or
   (b) X¹ is N;
      one of X², X³ and X⁴ are N; and
      two of X², X³ and X⁴ are CR⁶; or
   (c) X¹ and X⁴ are CR⁶; and
      X² and X³ are N;
ring A is selected from:
   (i) a 5-membered monocyclic heteroaryl ring comprising 1 to 3 heteroatoms, each independently selected from N and 0;
   (ii) a 6-membered monocyclic heteroaryl ring comprising 1 to 3 heteroatoms, each independently selected from N, O and S;
   (iii) a 4 to 6-membered saturated or partially saturated monocyclic heterocyclyl ring comprising 1 to 3 heteroatoms, each independently selected from N, O and S; and
   (iv) phenyl or naphthyl;
ring A is either unsubstituted or substituted by 1 or 2 R⁷ substituents;
ring B is selected from:
   (i) phenyl or naphthyl;
   (ii) a 5 to 6-membered monocyclic heteroaryl ring comprising 1 to 3 heteroatoms, each independently selected from N, O and S; and
   (iii) a 9 to 10-membered bicyclic heteroaryl ring comprising 1 to 4 heteroatoms, each independently selected from N, O and S;
ring B is either unsubstituted or substituted by 1 to 5 substituents, each independently selected from halo, CN, hydroxy, oxo, (C₁-C₆)alkyl, (C₁-C₆)alkoxy, halo(C₁-C₆)alkyl, halo(C₁-C₆)alkoxy, (C₃-C₆)cycloalkyl, O(C₃-C₆)cycloalkyl, (C₁-C₆)alkoxy(C₁-C₆)alkyl, oxetanyloxy, azetidinyl, pyrrolidinyl, piperidinyl, NH(C₁-C₆)alkyl, N((C₁-C₆)alkyl)₂, C(O)NH(C₁-C₆)alkyl, C(O)N((C₁-C₆)alkyl)₂, NHC(O)(C₁-C₆)alkyl, N(C₁-C₆)alkyl)C(O)(C₁-C₆)alkyl), C(O)(C₁-C₆)alkyl, C(O)O(C₁-C₆)alkyl, CO₂H, CONH₂, SO₂NH(C₁-C₆)alkyl and SO₂N((C₁-C₆)alkyl)₂;
R¹ is selected from hydrogen, (C₁-C₆)alkyl and (C₃-C₆)cycloalkyl;
R² and R³ are each independently selected from hydrogen, halo, (C₁-C₄)alkyl and (C₁-C₄)alkoxy;
R⁴ and R⁵ are each independently selected from hydrogen and (C₁-C₄)alkyl;
R⁶ is hydrogen or (C₁-C₄)alkyl; and
each R⁷ is independently selected from halo, (C₁-C₆)alkyl and (C₁-C₆)alkoxy;
with the proviso that when ring A is unsubstituted oxazolyl or oxadiazolyl, and ring B is phenyl, and R¹, R², R³, R⁴ and R are each hydrogen, and two of X¹, X², X³ and X⁴ are N, then ring B is not substituted by CF₃ in the position meta to ring A.

The disclaimer relates to WO 2021/239863, which includes Example 4, which is an N-cyanopyrrolidine substituted by CH₂-triazolyl.

Unless otherwise indicated, alkyl and alkoxy groups may be straight or branched and contain 1 to 6 carbon atoms, and more typically, 1 to 4 carbon atoms. Examples of alkyl include methyl, ethyl, n-propyl, isopropyl, n-butyl, isobutyl, sec-butyl, pentyl and hexyl. Examples of alkoxy include methoxy, ethoxy, n-propoxy, isobutoxy and n-butoxy. Examples of alkoxyalkyl include methoxymethyl, methoxyethoxy and ethoxymethoxy.

Unless otherwise indicated, cycloalkyl and cycloalkoxy (O-cycloalkyl) groups contain 3 to 6 carbon atoms, and more typically, 3 to 4 carbon atoms. Examples of cycloalkyl include cyclopropyl, cyclobutyl, cyclopentyl and cyclohexyl. Examples of cycloalkoxy include cyclopropoxy and cyclobutoxy.

Halo means fluoro, chloro, bromo or iodo, and is in particular, fluoro or chloro. Haloalkyl and haloalkoxy groups may contain one or more halo substituents. Examples are fluoromethyl, difluoromethyl, trifluoromethyl and trifluoromethoxy.

Heterocyclyl rings may be monocyclic or fused bicyclic rings, which are either saturated or partially saturated, and comprise 1 to 4 heteroatoms, preferably 1, 2 or 3 heteroatoms, each independently selected from nitrogen, oxygen and sulfur. Examples of heterocyclyl groups include azetidinyl, oxetanyl, pyrrolidinyl, tetrahydrofuranyl, tetrahydrothiophenyl, pyrazolidinyl, pyrrolinyl, dihydrofuranyl, dihydrothiophenyl, dihydrooxazolyl, thiazolinyl, dioxolanyl, oxathiolanyl, dithiolanyl, imidazolidinyl, imidazolinyl, pyrazolinyl, thiazolidinyl, pyranyl, piperidinyl, dioxanyl, morpholino, dithianyl, thiomorpholino, piperazinyl and tetrahydropyrazolopyrazinyl.

Heteroaryl rings may be monocyclic or bicyclic and comprise 1 to 4 heteroatoms, preferably 1, 2 or 3 heteroatoms, each independently selected from nitrogen, oxygen and sulfur, except in respect of ring A, where each heteroatom is independently selected (solely) from nitrogen and oxygen. Monocyclic heteroaryl rings are aromatic, and bicyclic heteroaryl rings are fused rings where either both rings are aromatic or one of the rings is aromatic and the other ring is saturated or partially saturated. Examples of heteroaryl groups include furanyl, thiophenyl, pyrrolyl, oxazolyl, isoxazolyl, oxadiazolyl (in particular, 1,3,4-oxadiazolyl, 1,2,3-oxadiazolyl, 1,2,4-oxadiazolyl and 1,2,5-oxadiazolyl), thiazolyl, isothiazolyl, thiadiazolyl (in particular, 1,3,4-thiadiazolyl, 1,2,3-thiadiazolyl, 1,2,4-thiadiazolyl and 1,2,5-thiadiazolyl), imidazolyl, pyrazolyl, triazolyl (in particular, 1,2,3-triazolyl and 1,2,4-triazolyl), tetrazolyl, pyridinyl, pyrimidinyl, pyridazinyl, pyrazinyl, triazinyl (in particular, 1,2,3-triazinyl, 1,2,4-triazinyl and 1,3,4-triazinyl), indazolyl, indolyl, indolinyl, indolizinyl, isoindolyl, isoindolinyl, purinyl, benzofuranyl, isobenzofuranyl, benzothiophenyl, isobenzothiophenyl, benzimidazolyl, benzothiazolyl, benzoxazolyl, benzotriazolyl, imidazopyridinyl, pyrazolopyridinyl, thiazolopyridinyl, dihydropyrrolopyridinyl, oxazolopyridinyl, naphthyridinyl, pteridinyl, quinolinyl, isoquinolinyl, cinnolinyl, phthalazinyl, quinazolinyl, quinoxalinyl, benzothiazinyl, dihydrobenzoxazinyl, dihydroquinazolinyl, tetrahydroquinolinyl and tetrahydroisoquinolinyl.

Unless otherwise indicated, the term substituted means substituted by one or more defined groups. In the case where groups may be selected from more than one alternative, the selected groups may be the same or different. The term ‘independently’ means that where more than one substituent is selected from more than one possible substituent, those substituents may be the same or different.

Preferred aspects and embodiments of the compound of formula (I) are defined below.

In one preferred aspect of the invention, (a), X¹ is N, and X², X³ and X⁴ are CR⁶.

In another preferred aspect of the invention, (b), X¹ is N, one of X², X³ and X⁴ are N, and two of X², X³ and X⁴ are CR⁶.

In another preferred aspect of the invention, (c), X¹ and X⁴ are CR⁶; and X² and X³ are N.

In a preferred embodiment of the invention, ring B is selected from:
(i) phenyl or naphthyl;
(ii) a 5 to 6-membered monocyclic heteroaryl ring selected from furanyl, thiophenyl, pyrrolyl, oxazolyl, isoxazolyl, oxadiazolyl (in particular, 1,3,4-oxadiazolyl, 1,2,3-oxadiazolyl, 1,2,4-oxadiazolyl and 1,2,5-oxadiazolyl), thiazolyl, isothiazolyl, thiadiazolyl (in particular, 1,3,4-thiadiazolyl, 1,2,3-thiadiazolyl, 1,2,4-thiadiazolyl and 1,2,5-thiadiazolyl), imidazolyl, pyrazolyl, triazolyl (in particular, 1,2,3-triazolyl and 1,2,4-triazolyl), pyridinyl, pyrimidinyl, pyridazinyl, pyrazinyl and triazinyl; and
(iii) a 9 to 10-membered bicyclic heteroaryl ring selected from indazolyl, indolyl, indolinyl, indolizinyl, isoindolyl, isoindolinyl, purinyl, benzofuranyl, isobenzofuranyl, benzothiophenyl, isobenzothiophenyl, benzimidazolyl, benzothiazolyl, benzoxazolyl, benzotriazolyl, imidazopyridinyl, pyrazolopyridinyl, thiazolopyridinyl, dihydropyrrolopyridinyl, oxazolopyridinyl, naphthyridinyl, pteridinyl, quinolinyl, isoquinolinyl, cinnolinyl, phthalazinyl, quinazolinyl, quinoxalinyl, benzothiazinyl, dihydrobenzoxazinyl, dihydroquinazolinyl, tetrahydroquinolinyl and tetrahydroisoquinolinyl.

More preferably, ring B is selected from phenyl, imidazolyl, pyrazolyl, pyridinyl, pyrimidinyl, pyridazinyl, pyrazinyl, indazolyl, indolyl, isoindolyl, benzofuranyl, isobenzofuranyl, benzothiophenyl, isobenzothiophenyl, benzimidazolyl, benzothiazolyl, benzoxazolyl, quinolinyl, isoquinolinyl, cinnolinyl, phthalazinyl, quinazolinyl, quinoxalinyl and dihydroquinazolinyl.

Yet more preferably, ring B is selected from phenyl, pyridinyl, pyrimidinyl, pyridazinyl, pyrazinyl, indazolyl, indolyl and isoindolyl.

Most preferably, ring B is selected from phenyl, pyridinyl and indazolyl.

In a preferred embodiment of the invention, ring B is either unsubstituted or substituted by 1 to 5 substituents, more preferably 1 to 3 substituents, and most preferably 1 to 2 substituents, each independently selected from halo, CN, hydroxy, oxo, (C₁-C₆)alkyl, (C₁-C₆)alkoxy, halo(C₁-C₆)alkyl, halo(C₁-C₆)alkoxy, (C₃-C₆)cycloalkyl, O(C₃-C₆)cycloalkyl, (C₁-C₆)alkoxy(C₁-C₆)alkyl, oxetanyloxy, azetidinyl, pyrrolidinyl and piperidinyl.

More preferably, ring B is either unsubstituted or substituted by 1 to 5 substituents, preferably 1 to 3 substituents, and most preferably 1 to 2 substituents, each independently selected from halo, CN, hydroxy, oxo, (C₁-C₃)alkyl, (C₁-C₃)alkoxy, halo(C₁-C₃)alkyl, halo(C₁-C₃)alkoxy, (C₃-C₄)cycloalkyl, O(C₃-C₄)cycloalkyl, (C₁-C₃)alkoxy(C₁-C₃)alkyl, oxetanyloxy, azetidinyl, pyrrolidinyl and piperidinyl.

Yet more preferably, ring B is either unsubstituted or substituted by 1 to 5 substituents, preferably 1 to 3 substituents, and most preferably 1 to 2 substituents, each independently selected from halo, CN, (C₁-C₃)alkyl, (C₁-C₃)alkoxy, cyclopropyl, cyclopropoxy, CF₃, OCF₃ and oxetanyloxy.

Even more preferably, ring B is either unsubstituted or substituted by 1 to 5 substituents, preferably 1 to 3 substituents, and most preferably 1 to 2 substituents, each independently selected from chloro, fluoro, CN, methyl, ethyl, propyl, methoxy, ethoxy, propoxy, cyclopropyl, cyclopropoxy, CF₃, OCF₃ and oxetan-3-yloxy.

Most preferably, ring B is either unsubstituted or substituted by 1 to 5 substituents, preferably 1 to 3 substituents, and most preferably 1 to 2 substituents, each independently selected from chloro, fluoro, CN, methyl, ethyl, methoxy, cyclopropyl, cyclopropoxy, CF₃, OCF₃ and oxetan-3-yloxy.

In one preferred aspect, the present invention is directed to a compound of formula (I) having the formula (I)(A):

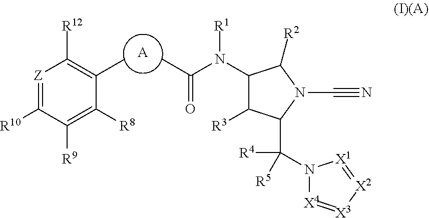

a tautomer thereof, or a pharmaceutically acceptable salt of said compound or tautomer, wherein:
Z is N or CR¹¹;
R⁸, R¹¹ and R¹² are each independently selected from hydrogen, halo, CN, hydroxy, oxo, (C₁-C₆)alkyl, (C₁-C₆)alkoxy, halo(C₁-C₆)alkyl, halo(C₁-C₆)alkoxy, (C₃-C₆)cycloalkyl, O(C₃-C₆)cycloalkyl, (C₁-C₆)alkoxy(C₁-C₆)alkyl, oxetanyloxy, azetidinyl, pyrrolidinyl and piperidinyl; R⁹ and R¹⁰ are each independently selected from hydrogen, halo, CN, hydroxy, oxo, (C₁-C₆)alkyl, (C₁-C₆)alkoxy, halo(C₁-C₆)alkyl, halo(C₁-C₆)alkoxy, (C₃-C₆)cycloalkyl, O(C₃-C₆)cycloalkyl, (C₁-C₆)alkoxy(C₁-C₆)alkyl, oxetanyloxy, azetidinyl, pyrrolidinyl and piperidinyl;
or R⁹ and R¹⁰ together form a 5 to 6-membered saturated, partially saturated, or aromatic ring comprising 1 to 2 heteroatoms, each independently selected from N, O and S, wherein the ring is either unsubstituted or substituted with 1 to 2 substituents, each independently selected from halo, CN, hydroxy, oxo, (C₁-C₆)alkyl, (C₁-C₆)alkoxy, halo(C₁-C₆)alkyl and halo(C₁-C₆)alkoxy;
with the proviso that when ring A is unsubstituted oxazolyl or oxadiazolyl, and R¹, R², R³, R⁴ and R⁵ are each hydrogen, and two of X¹, X², X³ and X⁴ are N, and Z is CR¹¹, and R¹¹ is hydrogen or halo, then R⁹ is not CF₃.

In one embodiment, Z is CR¹¹, which provides a compound of formula (I) having the formula (I)(B):

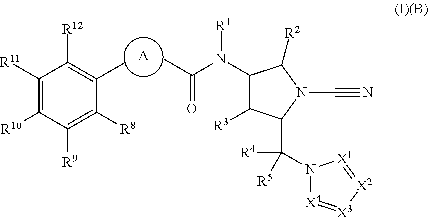

a tautomer thereof, or a pharmaceutically acceptable salt of said compound or tautomer. This corresponds to the compound of formula (I), wherein ring B is either phenyl or a 9 to 10-membered bicyclic heteroaryl ring (R⁹ and R¹⁰ together form a heterocyclyl or heteroaryl ring).

In another embodiment, Z is N, which provides a compound of formula (I) having the formula (I)(C):

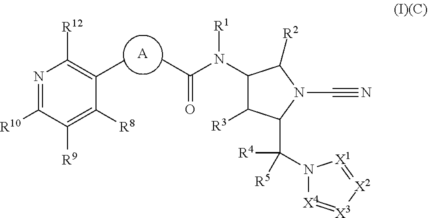

a tautomer thereof, or a pharmaceutically acceptable salt of said compound or tautomer. This corresponds to the compound of formula (I), wherein ring B is pyridinyl (6-membered monocyclic heteroaryl ring).

In another preferred aspect of the invention, ring A is a 5-membered monocyclic heteroaryl ring comprising 1 to 3 heteroatoms, each independently selected from N and O. More preferably, ring A is a heteroaryl ring selected from furanyl, pyrrolyl, oxazolyl, isoxazolyl, oxadiazolyl (in particular, 1,3,4-oxadiazolyl, 1,2,3-oxadiazolyl, 1,2,4-oxadiazolyl and 1,2,5-oxadiazolyl), imidazolyl, pyrazolyl and triazolyl (in particular, 1,2,3-triazolyl and 1,2,4-triazolyl). Yet more preferably, ring A is selected from oxazolyl and oxadiazolyl. Most preferably, ring A is selected from oxazolyl and 1,3,4-oxadiazolyl.

In another preferred aspect, when ring A is selected from oxazolyl and oxadiazolyl, the present invention is directed to a compound of formula (I) having the formula (I)(D):

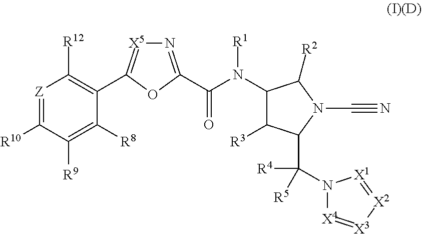

a tautomer thereof, or a pharmaceutically acceptable salt of said compound or tautomer, wherein X⁵ is selected from N, CH and CR⁷. In one preferred embodiment, X⁵ is selected from CH and CR⁷, and Z is CR¹¹. In another preferred embodiment, X⁵ is N, and Z is CR¹¹. In another preferred embodiment, X⁵ is selected from CH and CR⁷, and Z is N. In another preferred embodiment, X⁵ is N, and Z is N. Most preferably, in each embodiment, X⁵ is CH.

In another preferred aspect of the invention, ring A is a 6-membered monocyclic heteroaryl ring comprising 1 to 3 heteroatoms, each independently selected from N, O and S. More preferably, ring A is a heteroaryl ring selected from pyridinyl, pyrimidinyl, pyridazinyl, pyrazinyl and triazinyl. Most preferably, ring A is pyridinyl.

In another preferred aspect, when ring A is pyridinyl, the present invention is directed to a compound of formula (I) having the formula (I)(E):

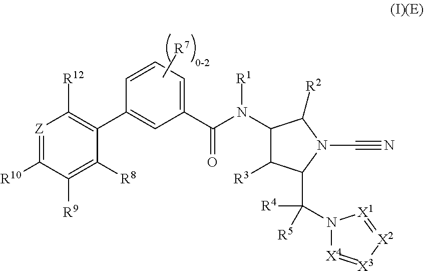

a tautomer thereof, or a pharmaceutically acceptable salt of said compound or tautomer. In one preferred embodiment, Z is CR¹¹. In another preferred embodiment, Z is N. The pyridinyl ring is either unsubstituted or substituted by 1 or 2 R⁷ substituents. Preferably, the pyridinyl ring is unsubstituted.

In another preferred aspect of the invention, ring A is a 4 to 6-membered saturated or partially saturated monocyclic heterocyclyl ring comprising 1 to 3 heteroatoms, each independently selected from N, O and S. More preferably, ring A is a heterocyclyl ring selected from azetidinyl, pyrrolidinyl, tetrahydrofuranyl, tetrahydrothiophenyl, pyrazolidinyl, pyrrolinyl, dihydrofuranyl, dihydrothiophenyl, dihydrooxazolyl, thiazolinyl, dioxolanyl, oxathiolanyl, dithiolanyl, imidazolidinyl, imidazolinyl, pyrazolinyl, thiazolidinyl, pyranyl, piperidinyl, dioxanyl, morpholino, dithianyl, thiomorpholino and piperazinyl. Most preferably, ring A is pyrrolidinyl.

In another preferred aspect, when ring A is pyrrolidinyl, the present invention is directed to a compound of formula (I) having the formula (I)(F):

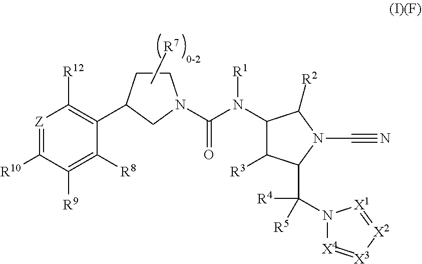

a tautomer thereof, or a pharmaceutically acceptable salt of said compound or tautomer. In one preferred embodiment, Z is CR¹¹. In another preferred embodiment, Z is N. The pyrrolidinyl ring is either unsubstituted or substituted by 1 or 2 R⁷ substituents. Preferably, the pyrrolidinyl ring is unsubstituted.

In another preferred aspect of the invention, ring A is phenyl.

In another preferred aspect, when ring A is phenyl, the present invention is directed to a compound of formula (I) having the formula (I)(G):

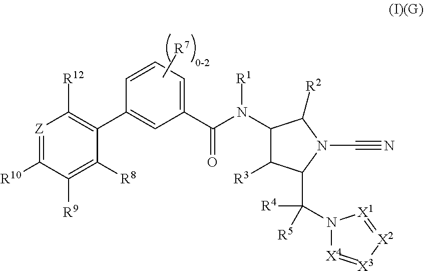

a tautomer thereof, or a pharmaceutically acceptable salt of said compound or tautomer. In one preferred embodiment, Z is CR¹¹. In another preferred embodiment, Z is N. The phenyl ring is either unsubstituted or substituted by 1 or 2 R⁷ substituents. Preferably, the phenyl ring is unsubstituted or substituted by fluoro.

In a preferred embodiment of all aspects of the invention, R¹ is selected from hydrogen, methyl and cyclopropyl. Most preferably, R¹ is selected from hydrogen and cyclopropyl.

In a preferred embodiment of all aspects of the invention, R², R³, R⁴ and R⁵ are each hydrogen.

In a preferred embodiment of all aspects of the invention, R⁶ is hydrogen or methyl. Most preferably, R⁶ is hydrogen.

In one preferred embodiment of all aspects of the invention, ring A is unsubstituted.

In another preferred embodiment of all aspects of the invention, ring A is substituted by 1 or 2 R⁷ groups, which are independently selected from fluoro, chloro, methyl and methoxy. Preferably, ring A is substituted by 1 R⁷ group, which is selected from fluoro, chloro, methyl and methoxy.

In a preferred embodiment of all aspects of the invention, R⁸ and R¹¹ are each independently selected from hydrogen, halo, CN, methyl, ethyl, isopropyl, methoxy, ethoxy, isopropoxy, cyclopropyl, cyclopropoxy, CF₃ and OCF₃. More preferably, R⁸ and R¹¹ are each independently selected from hydrogen, halo and CN. Yet more preferably, R⁸ and R¹¹ are each independently selected from hydrogen, fluoro and CN. Most preferably, R⁸ and R¹¹ are each hydrogen.

In a preferred embodiment of all aspects of the invention, R⁹ is selected from hydrogen, halo, CN, hydroxy, oxo, (C₁-C₆)alkyl, (C₁-C₆)alkoxy, halo(C₁-C₃)alkyl, halo(C₁-C₃)alkoxy, (C₃-C₄)cycloalkyl, O(C₃-C₄)cycloalkyl and oxetanyloxy. More preferably, R⁹ is selected from hydrogen, halo, CN, (C₁-C₃)alkyl, (C₁-C₃)alkoxy, cyclopropyl, cyclopropoxy, CF₃, OCF₃ and oxetan-3-yloxy. Yet more preferably, R⁹ is selected from hydrogen, fluoro, chloro, CN, methyl, ethyl, isopropyl, methoxy, ethoxy, isopropoxy, cyclopropyl, cyclopropoxy, CF₃ and OCF₃. Most preferably, R⁹ is selected from hydrogen, chloro, CN, ethyl, cyclopropyl, CF₃ and OCF₃.

In a preferred embodiment of all aspects of the invention, R¹⁰ is selected from hydrogen, halo, CN, methyl, ethyl, isopropyl, methoxy, ethoxy, isopropoxy, cyclopropyl, cyclopropoxy, CF₃ and OCF₃.

More preferably, R¹⁰ is selected from hydrogen, fluoro, chloro and CN. Most preferably, R¹⁰ is hydrogen.

Alternatively, in a preferred embodiment of all aspects of the invention, R⁹ and R¹⁰ together form a 5 to 6-membered partially saturated or aromatic ring comprising 1 to 2 heteroatoms, each independently selected from N, O and S. More preferably, R⁹ and R¹⁰ together form a 5 to 6-membered partially saturated or aromatic ring comprising 1 to 2 nitrogen atoms. Most preferably, R⁹ and R¹⁰ together form a 5-membered partially saturated or aromatic ring comprising 1 to 2 nitrogen atoms. In a particularly preferred embodiment of all aspects of the invention, R⁹ and R¹⁰, together with the phenyl ring to which they are attached, form an indazolyl ring.

Preferably, the ring formed by R⁹ and R¹⁰ is either unsubstituted or substituted with 1 to 2 substituents, each independently selected from halo, CN, methyl, ethyl, isopropyl, methoxy, ethoxy, isopropoxy, cyclopropyl, cyclopropoxy, CF₃ and OCF₃. More preferably, the ring formed by R⁹ and R¹⁰ is either unsubstituted or substituted with 1 to 2 substituents, each independently selected from fluoro, chloro, methyl and methoxy. Most preferably, the ring formed by R⁹ and R¹⁰ is either unsubstituted or substituted with 1 substituent. Where R⁹ and R¹⁰, together with the phenyl ring to which they are attached, form an indazolyl ring, the ring is preferably 1-methyl-1H-indazolyl.

In a preferred embodiment of all aspects of the invention, R¹² is selected from hydrogen, halo, CN, hydroxy, oxo, (C₁-C₃)alkyl, (C₁-C₃)alkoxy, halo(C₁-C₃)alkyl, halo(C₁-C₃)alkoxy, (C₃-C₄)cycloalkyl, O(C₃-C₄)cycloalkyl and oxetanyloxy. More preferably, R¹² is selected from hydrogen, halo, CN, (C₁-C₃)alkyl, (C₁-C₃)alkoxy, cyclopropyl, cyclopropoxy, CF₃, OCF₃ and oxetan-3-yloxy. Yet more preferably, R¹² is selected from hydrogen, fluoro, chloro, CN, methyl, ethyl, isopropyl, methoxy, ethoxy, isopropoxy, cyclopropyl, cyclopropoxy, CF₃, OCF₃ and oxetan-3-yloxy. Most preferably, R¹² is selected from hydrogen, methoxy, cyclopropyl, cyclopropoxy, OCF₃ and oxetan-3-yloxy.

In one preferred aspect, where the compound of formula (I) is the compound of formula (I)(D):
R⁸ is selected from hydrogen, halo, CN, methyl, ethyl, isopropyl, methoxy, ethoxy, isopropoxy, cyclopropyl, cyclopropoxy, CF₃ and OCF₃;
R⁹ is selected from hydrogen, halo, CN, (C₁-C₃)alkyl, (C₁-C₃)alkoxy, cyclopropyl, cyclopropoxy, OCF₃ and oxetan-3-yloxy;
R¹⁰ is selected from hydrogen, halo, CN, methyl, ethyl, isopropyl, methoxy, ethoxy, isopropoxy, cyclopropyl, cyclopropoxy, CF₃ and OCF₃;
R¹¹ is selected from hydrogen, halo, CN, methyl, ethyl, isopropyl, methoxy, ethoxy, isopropoxy, cyclopropyl, cyclopropoxy and OCF₃; and
R¹² is selected from hydrogen, halo, CN, (C₁-C₃)alkyl, (C₁-C₃)alkoxy, cyclopropyl, cyclopropoxy, CF₃, OCF₃ and oxetan-3-yloxy.

In a preferred embodiment of all aspects of the invention, 2 of the R⁸, R⁹, R¹⁰, R¹¹ and R¹² substituents are hydrogen and the remaining 3 substituents are as defined in the embodiments herein. Most preferably, 3 of the R⁸, R⁹, R¹⁰, R¹¹ and R¹² substituents are hydrogen and the remaining 2 substituents are as defined in the embodiments herein.

In a preferred embodiment of all aspects of the invention, R⁸ and R¹¹ are hydrogen, and R⁹, R¹⁰ and R¹² are as defined in the embodiments herein. More preferably, R⁸ and R¹¹ are hydrogen, R¹⁰ is selected from hydrogen, fluoro and CN, and R⁹ and R¹² are as defined in the embodiments herein. Most preferably, R⁸, R¹⁰ and R¹¹ are hydrogen, and R⁹ and R¹² are as defined in the embodiments herein.

For example, in one preferred embodiment of all aspects of the invention: R⁸, R¹⁰ and R¹¹ are hydrogen; and R⁹ and R¹² are each independently selected from hydrogen, halo, CN, (C₁-C₃)alkyl, (C₁-C₃)alkoxy, cyclopropyl, cyclopropoxy, CF₃, OCF₃ and oxetan-3-yloxy.

Compounds of formula (I) contain two or more asymmetric carbon atoms (chiral centres) and can exist as four or more stereoisomers.

The compounds of formula (I) contain two chiral centres at the carbon atoms of the pyrrolidine ring that are substituted by the NR¹C(O) and CR⁴R⁵-ring groups and said stereocentres could exist in either the (R) or (S) configuration. The designation of the absolute configuration (R) and (S) for stereoisomers in accordance with IUPAC nomenclature is dependent on the nature of the substituents and application of the sequence-rule procedure. The compounds of formula (I) may contain other chiral centres, for example where R² or R³ are other than hydrogen or where R⁴ and R are different. Included within the scope of the present invention are all stereoisomers of the compounds of formula (I) and combinations thereof.

In respect of the chiral centres of the NR¹C(O) and CR⁴R⁵-ring groups, the compounds of formula (I) may thus exist in any of the following configurations:

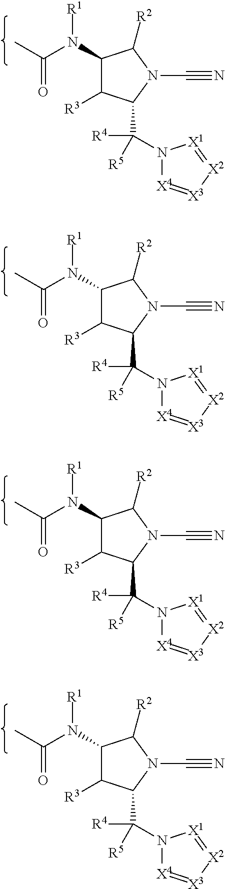

The compounds of formula (I) of the present invention, and all aspects and preferred embodiments thereof, preferably exist as a single stereoisomer having the absolute configuration:

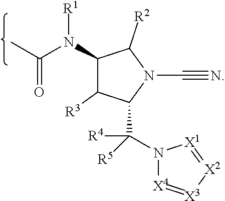

Where the groups R², R³, R⁴ and R are all hydrogen, the compounds of formula (I) of the present invention, and all aspects and preferred embodiments thereof, preferably exist as a single stereoisomer having the absolute configuration (3R,5S):

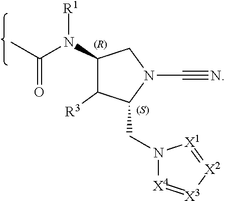

Where the compound of formula (I) is a single stereoisomer, it preferably exists with a stereoisomeric excess of at least 60%, more preferably at least 80%, yet more preferably at least 90%, and most preferably at least 95%, for example 96%, 97%, 98%, 99%, or 100%.

Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC). Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of propan-2-ol, typically from 2% to 20%, and from 0 to 5% by volume of an alkylamine, typically 0.1% diethylamine. Concentration of the eluate affords the enriched mixture. The present invention includes all crystal forms of the compounds of formula (I) including racemates and racemic mixtures (conglomerates) thereof. Stereoisomeric conglomerates may be separated by conventional techniques known to those skilled in the art—see, for example, “Stereochemistry of Organic Compounds” by E. L. Eliel and S. H. Wilen (Wiley, New York, 1994).

Where the present invention is directed to a compound of formula (I), preferably the compound has the absolute stereochemical configuration of formula (I)(i):

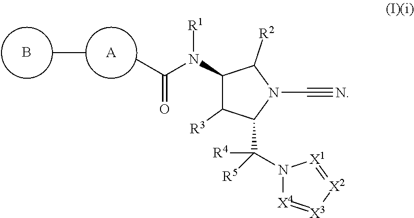

Where the present invention is directed to a compound of formula (I)(A), preferably the compound has the absolute stereochemical configuration of formula (I)(A)(i):

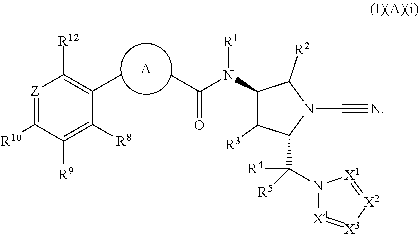

Where the present invention is directed to a compound of formula (I)(B), preferably the compound has the absolute stereochemical configuration of formula (I)(B)(i):

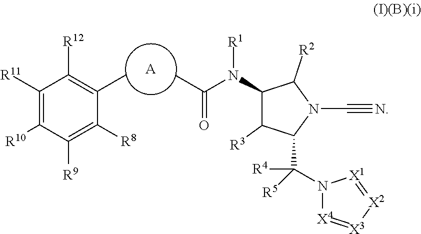

Where the present invention is directed to a compound of formula (I)(C), preferably the compound has the absolute stereochemical configuration of formula (I)(C)(i):

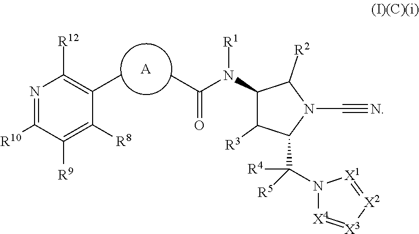

Where the present invention is directed to a compound of formula (I)(D), preferably the compound has the absolute stereochemical configuration of formula (I)(D)(i):

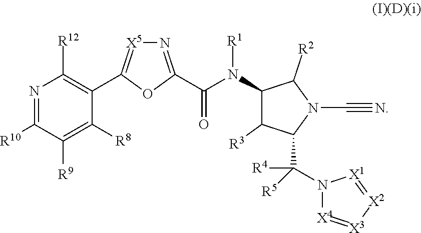

Where the present invention is directed to a compound of formula (I)(E), preferably the compound has the absolute stereochemical configuration of formula (I)(E)(i):

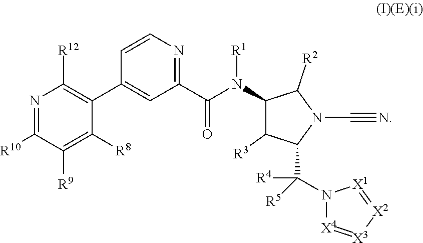

Where the present invention is directed to a compound of formula (I)(F), preferably the compound has the absolute stereochemical configuration of formula (I)(F)(i):

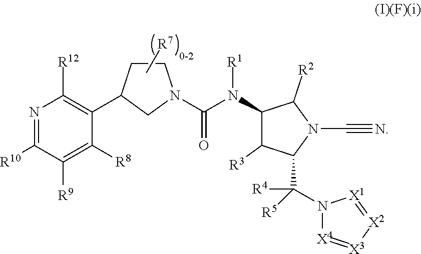

Where the present invention is directed to a compound of formula (I)(G), preferably the compound has the absolute stereochemical configuration of formula (I)(G)(i):

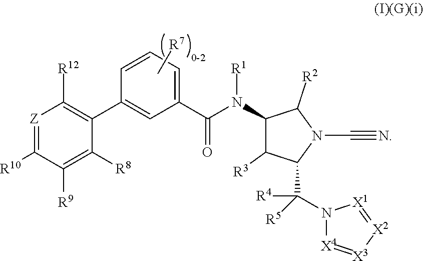

In one particularly preferred aspect of each the compounds of formula (I) and formula (I)(i) of the present invention:
ring B is selected from phenyl, imidazolyl, pyrazolyl, pyridinyl, pyrimidinyl, pyridazinyl, pyrazinyl, indazolyl, indolyl, isoindolyl, benzofuranyl, isobenzofuranyl, benzothiophenyl, isobenzothiophenyl, benzimidazolyl, benzothiazolyl, benzoxazolyl, quinolinyl, isoquinolinyl, cinnolinyl, phthalazinyl, quinazolinyl, quinoxalinyl and dihydroquinazolinyl, and is preferably selected from phenyl, pyridinyl and indazolyl;
ring B is either unsubstituted or substituted by 1 to 2 substituents, each independently selected from halo, CN, (C₁-C₃)alkyl, (C₁-C₃)alkoxy, cyclopropyl, cyclopropoxy, CF₃, OCF₃ and oxetanyloxy;
ring A is selected from furanyl, pyrrolyl, oxazolyl, isoxazolyl, oxadiazolyl, imidazolyl, pyrazolyl, triazolyl, pyridinyl, pyrimidinyl, pyridazinyl, pyrazinyl, triazinyl, pyrrolidinyl and phenyl, and is preferably selected from oxazolyl, 1,3,4-oxadiazolyl, pyridinyl, pyrrolidinyl and phenyl;
ring A is either unsubstituted or substituted by 1 R⁷ group, which is selected from fluoro, chloro, methyl and methoxy;
R¹ is selected from hydrogen, methyl and cyclopropyl;
R², R³, R⁴ and R⁵ are each hydrogen; and
R⁶ is hydrogen or methyl.

In a more particularly preferred aspect of each the compounds of formula (I) and formula (I)(i) of the present invention:
ring B is selected from phenyl, pyridinyl and indazolyl;
ring B is either unsubstituted or substituted by 1 to 2 substituents, each independently selected from halo, CN, (C₁-C₃)alkyl, (C₁-C₃)alkoxy, cyclopropyl, cyclopropoxy, CF₃, OCF₃ and oxetanyloxy;
ring A is selected from oxazolyl, 1,3,4-oxadiazolyl, pyridinyl, pyrrolidinyl and phenyl;
ring A is either unsubstituted or substituted by methyl;
R¹ is selected from hydrogen and cyclopropyl;
R², R³, R⁴ and R⁵ are each hydrogen; and
R⁶ is hydrogen or methyl.

In one particularly preferred aspect of each of the compounds of formulae (I)(A), (I)(B), (I)(C), (I)(D), (I)(E), (I)(F), (I)(G), (I)(A)(i), (I)(B)(i), (I)(C)(i), (I)(D)(i), (I)(E)(i), (I)(F)(i) and (I)(G)(i) of the present invention:
R¹ is selected from hydrogen, methyl and cyclopropyl;
R², R³, R⁴ and R are each hydrogen;
R⁶ is hydrogen or methyl;
ring A is either unsubstituted or substituted by 1 or 2 R⁷ groups, which are independently selected from fluoro, chloro, methyl and methoxy;
R⁸ and R¹¹ are each hydrogen;
R⁹ is selected from hydrogen, halo, CN, (C₁-C₃)alkyl, (C₁-C₃)alkoxy, cyclopropyl, cyclopropoxy, CF₃, OCF₃ and oxetan-3-yloxy;
R¹⁰ is selected from hydrogen, fluoro and CN, and is preferably hydrogen; and
R¹² is selected from hydrogen, halo, CN, (C₁-C₃)alkyl, (C₁-C₃)alkoxy, cyclopropyl, cyclopropoxy, CF₃, OCF₃ and oxetan-3-yloxy.

In a more particularly preferred aspect of each of the compounds of formulae (I)(A), (I)(B), (I)(C), (I)(D), (I)(E), (I)(F), (I)(G), (I)(A)(i), (I)(B)(i), (I)(C)(i), (I)(D)(i), (I)(E)(i), (I)(F)(i) and (I)(G)(i) of the present invention:
R¹ is selected from hydrogen and cyclopropyl;
R², R³, R⁴, R⁵, R⁸, R¹⁰ and R¹¹ are each hydrogen;
R⁶ is hydrogen or methyl;
ring A is either unsubstituted or substituted by methyl;
R⁹ is selected from hydrogen, halo, CN, (C₁-C₃)alkyl, (C₁-C₃)alkoxy, cyclopropyl, cyclopropoxy, CF₃, OCF₃ and oxetan-3-yloxy; and
R¹² is selected from hydrogen, halo, CN, (C₁-C₃)alkyl, (C₁-C₃)alkoxy, cyclopropyl, cyclopropoxy, CF₃, OCF₃ and oxetan-3-yloxy.

In another particularly preferred aspect of each of the compounds of formulae (I)(A), (I)(B), (I)(C), (I)(D), (I)(E), (I)(F), (I)(G), (I)(A)(i), (I)(B)(i), (I)(C)(i), (I)(D)(i), (I)(E)(i), (I)(F)(i) and (I)(G)(i) of the present invention:
R¹ is selected from hydrogen, methyl and cyclopropyl;
R², R³, R⁴, R⁵, R⁸ and R¹¹ are each hydrogen;
R⁶ is hydrogen or methyl;
ring A is either unsubstituted or substituted by 1 or 2 R⁷ groups, which are independently selected from fluoro, chloro, methyl and methoxy;
R⁹ and R¹⁰ together form a 5 to 6-membered partially saturated or aromatic ring comprising 1 to 2 nitrogen atoms, which is either unsubstituted or substituted with 1 to 2 substituents, each independently selected from fluoro, chloro, methyl and methoxy; and
R¹² is selected from hydrogen, halo, CN, (C₁-C₃)alkyl, (C₁-C₃)alkoxy, cyclopropyl, cyclopropoxy, CF₃, OCF₃ and oxetan-3-yloxy, and is preferably hydrogen.

In a more particularly preferred aspect of each of the compounds of formulae (I)(A), (I)(B), (I)(C), (I)(D), (I)(E), (I)(F), (I)(G), (I)(A)(i), (I)(B)(i), (I)(C)(i), (I)(D)(i), (I)(E)(i), (I)(F)(i) and (I)(G)(i) of the present invention:
R¹ is selected from hydrogen and cyclopropyl;
R², R³, R⁴, R⁵, R⁸, R¹¹ and R¹² are each hydrogen; R⁶ is hydrogen or methyl;
ring A is either unsubstituted or substituted by methyl; and
R⁹ and R¹⁰, together with the phenyl ring to which they are attached, form an indazole ring, which is either unsubstituted or substituted with 1 to 2 substituents, each independently selected from fluoro and methyl, and is preferably substituted by methyl.

Preferred compounds of formula (I) for use in the present invention are selected from:
N-((3R,5S)-5-((1H-1,2,3-triazol-1-yl)methyl)-1-cyanopyrrolidin-3-yl)-5-(5-cyano-2-cyclopropylphenyl)-N-cyclopropyloxazole-2-carboxamide;
N-((3R,5S)-5-((1H-1,2,3-triazol-1-yl)methyl)-1-cyanopyrrolidin-3-yl)-5-(5-cyano-2-cyclopropylphenyl)-N-cyclopropyloxazole-2-carboxamide;
N-((3R,5S)-5-((1H-1,2,3-triazol-1-yl)methyl)-1-cyanopyrrolidin-3-yl)-5-(3-cyanophenyl)oxazole-2-carboxamide;
N-((3R,5S)-5-((1H-1,2,3-triazol-1-yl)methyl)-1-cyanopyrrolidin-3-yl)-5-(3-(trifluoromethoxy)phenyl)oxazole-2-carboxamide;
N-((3R,5S)-5-((1H-1,2,3-triazol-1-yl)methyl)-1-cyanopyrrolidin-3-yl)-5-(3-chlorophenyl)oxazole-2-carboxamide;
N-((3R,5S)-5-((1H-1,2,3-triazol-1-yl)methyl)-1-cyanopyrrolidin-3-yl)-5-(2-methoxyphenyl)oxazole-2-carboxamide;
N-((3R,5S)-5-((1H-1,2,3-triazol-1-yl)methyl)-1-cyanopyrrolidin-3-yl)-5-(2-cyclopropylphenyl)-1,3,4-oxadiazole-2-carboxamide;
N-((3R,5S)-5-((1H-1,2,3-triazol-1-yl)methyl)-1-cyanopyrrolidin-3-yl)-5-(2-cyclopropylphenyl)oxazole-2-carboxamide;
N-((3R,5S)-5-((1H-1,2,3-triazol-1-yl)methyl)-1-cyanopyrrolidin-3-yl)-5-(5-cyano-2-cyclopropylphenyl)oxazole-2-carboxamide;
N-((3R,5S)-5-((1H-1,2,3-triazol-1-yl)methyl)-1-cyanopyrrolidin-3-yl)-5-(2-(trifluoromethoxy)phenyl)oxazole-2-carboxamide;
N-((3R,5S)-5-((1H-1,2,3-triazol-1-yl)methyl)-1-cyanopyrrolidin-3-yl)-5-(3-ethylphenyl)oxazole-2-carboxamide;
N-((3R,5S)-5-((1H-1,2,3-triazol-1-yl)methyl)-1-cyanopyrrolidin-3-yl)-5-(3-cyclopropylphenyl)oxazole2-carboxamide;
N-((3R,5S)-5-((1H-1,2,3-triazol-1-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-5-(5-cyano-2-cyclopropylphenyl)-1,3,4-oxadiazole-2-carboxamide;
N-((3R,5S)-5-((1H-1,2,3-triazol-1-yl)methyl)-1-cyanopyrrolidin-3-yl)-5-(2-cyclopropoxy-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-5-(5-cyano-2-(oxetan-3-yloxy)phenyl)-N-cyclopropyloxazole-2-carboxamide;
N-((3R,5S)-5-((2H-1,2,3-triazol-2-yl)methyl)-1-cyanopyrrolidin-3-yl)-5-(3-cyanophenyl)oxazole-2-carboxamide;
N-((3R,5S)-5-((1H-1,2,4-triazol-1-yl)methyl)-1-cyanopyrrolidin-3-yl)-5-(3-cyanophenyl)oxazole-2-carboxamide;
N-((3R,5S)-5-((4H-1,2,4-triazol-4-yl)methyl)-1-cyanopyrrolidin-3-yl)-5-(3-cyanophenyl)oxazole-2-carboxamide;
N-((3R,5S)-5-((1H-1,2,3-triazol-1-yl)methyl)-1-cyanopyrrolidin-3-yl)-4-(3-(trifluoromethyl)phenyl)picolinamide;
N-((3R,5S)-5-((1H-1,2,3-triazol-1-yl)methyl)-1-cyanopyrrolidin-3-yl)-4-(3-(trifluoromethoxy)phenyl)picolinamide;
N-((3R,5S)-5-((1H-pyrazol-1-yl)methyl)-1-cyanopyrrolidin-3-yl)-5-(3-cyanophenyl)oxazole-2-carboxamide;
N-((3R,5S)-5-((1H-pyrazol-1-yl)methyl)-1-cyanopyrrolidin-3-yl)-5-(3-(trifluoromethoxy)phenyl)oxazole-2-carboxamide;
N-((3R,5S)-1-cyano-5-((3-methyl-1H-pyrazol-1-yl)methyl)pyrrolidin-3-yl)-5-(3-(trifluoromethoxy)phenyl)oxazole-2-carboxamide;
N-((3R,5S)-1-cyano-5-((5-methyl-1H-pyrazol-1-yl)methyl)pyrrolidin-3-yl)-5-(3-(trifluoromethoxy)phenyl)oxazole-2-carboxamide;
N-((3R,5S)-5-((1H-pyrazol-1-yl)methyl)-1-cyanopyrrolidin-3-yl)(3(trifluoromethyl)phenyl)oxazole-2-carboxamide;
N-((3R,5S)-5-((1H-pyrazol-1-yl)methyl)-1-cyanopyrrolidin-3-yl)-2-fluoro-4-(1-methyl-1H-indazol-5-yl)benzamide;
N-((3R,5S)-5-((1H-1,2,3-triazol-1-yl)methyl)-1-cyanopyrrolidin-3-yl)-2-fluoro-4-(1-methyl-1H-indazol-5-yl)benzamide;
N-((3R,5S)-5-((1H-pyrazol-1-yl)methyl)-1-cyanopyrrolidin-3-yl)-3-(3-(trifluoromethoxy)phenyl)pyrrolidine-1-carboxamide;
(S)—N-((3R,5S)-5-((1H-pyrazol-1-yl)methyl)-1-cyanopyrrolidin-3-yl)-3-(3-(trifluoromethoxy)phenyl)pyrrolidine-1-carboxamide;
(R)—N-((3R,5S)-5-((1H-pyrazol-1-yl)methyl)-1-cyanopyrrolidin-3-yl)-3-(3-(trifluoromethoxy)phenyl)pyrrolidine-1-carboxamide;
N-((3R,5S)-5-((1H-1,2,3-triazol-1-yl)methyl)-1-cyanopyrrolidin-3-yl)-5-(4-fluoro-3-(trifluoromethoxy)phenyl)oxazole-2-carboxamide;
N-((3R,5S)-5-((1H-1,2,3-triazol-1-yl)methyl)-1-cyanopyrrolidin-3-yl)-5-(2-(azetidin-1-yl)-5-cyanopyridin-3-yl)-1,3,4-oxadiazole-2-carboxamide;
N-((3R,5S)-5-((1H-1,2,3-triazol-1-yl)methyl)-1-cyanopyrrolidin-3-yl)-5-(5-cyano-2-(pyrrolidin-1-yl)pyridin-3-yl)-1,3,4-oxadiazole-2-carboxamide;
N-((3R,5S)-5-((1H-1,2,3-triazol-1-yl)methyl)-1-cyanopyrrolidin-3-yl)-5-(2-chloro-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-5-(3-(trifluoromethoxy)phenyl)-1,3,4-oxadiazole-2-carboxamide;
N-((3R,5S)-5-((1H-pyrazol-1-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((2H-1,2,3-triazol-2-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((1H-pyrazol-1-yl)methyl)-1-cyanopyrrolidin-3-yl)-5-(5-cyano-2-cyclopropylphenyl)-1,3,4-oxadiazole-2-carboxamide;
N-((3R,5S)-5-((1H-1,2,4-triazol-1-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-5-(2-propyl-5-(trifluoromethoxy)phenyl)-1,3,4-oxadiazole-2-carboxamide;
N-((3R,5S)-5-((4H-1,2,4-triazol-4-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-3-(2-cyclopropyl-5-(trifluoromethoxy)phenyl)-1,2,4-oxadiazole-5-carboxamide;
N-((3R,5S)-5-((1H-1,2,3-triazol-1-yl)methyl)-1-cyanopyrrolidin-3-yl)-5-(2-cyclopropyl-5-(trifluoromethoxy)phenyl)-1,2,4-oxadiazole-3-carboxamide; and
N-((3R,5S)-5-((1H-pyrazol-1-yl)methyl)-1-cyanopyrrolidin-3-yl)-6-(1H-indazol-4-yl)nicotinamide;

a tautomer thereof, or a pharmaceutically acceptable salt of said compound or tautomer.

Pharmaceutical acceptable salts of the compounds of formula (I) include the acid addition and base salts (including di-salts) thereof.

Suitable acid addition salts are formed from acids which form non-toxic salts. Examples include the acetate, aspartate, benzoate, besylate, bicarbonate/carbonate, bisulfate, camsylate, citrate, edisylate, esylate, fumarate, gluceptate, gluconate, glucuronate, hibenzate, hydrochloride/chloride, hydrobromide/bromide, hydroiodide/iodide, hydrogen phosphate, isethionate, D- and L-lactate, malate, maleate, malonate, mesylate, methylsulfate, 2-napsylate, nicotinate, nitrate, orotate, palmate, phosphate, saccharate, stearate, succinate sulfate, D- and L-tartrate, and tosylate salts.

Suitable base salts are formed from bases which form non-toxic salts. Examples include the aluminium, ammonium, arginine, benzathine, calcium, choline, diethylamine, diolamine, glycine, lysine, magnesium, meglumine, olamine, potassium, sodium, tromethamine and zinc salts.

For a review on suitable salts, see Stahl and Wermuth, Handbook of Pharmaceutical Salts: Properties, Selection, and Use, Wiley-VCH, Weinheim, Germany (2002).

A pharmaceutical acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent.

Pharmaceutical acceptable solvates in accordance with the invention include hydrates and solvates wherein the solvent of crystallization may be isotopically substituted, e.g., D₂O, acetone-d₆, DMSO-d₆.

Also, within the scope of the invention are clathrates, drug-host inclusion complexes wherein, in contrast to the aforementioned solvates, the drug and host are present in non-stoichiometric amounts.

For a review of such complexes, see J. Pharm Sci, 64 (8), 1269-1288 by Haleblian (August 1975).

Hereinafter all references to compounds of formula (I) include references to salts thereof and to solvates and clathrates of compounds of formula (I) and salts thereof.

The invention includes all polymorphs of the compounds of formula (I) as hereinbefore defined.

Also, within the scope of the invention are so-called “prodrugs” of the compounds of formula (I). Thus, certain derivatives of compounds of formula (I) which have little or no pharmacological activity themselves can, when metabolised upon administration into or onto the body, give rise to compounds of formula (I), having the desired activity. Such derivatives are referred to as “prodrugs”.

Prodrugs in accordance with the invention can, for example, be produced by replacing appropriate functionalities present in the compounds of formula (I) with certain moieties known to those skilled in the art as “pro-moieties” as described, for example, in “Design of Prodrugs” by H Bundgaard (Elsevier, 1985). Finally, certain compounds of formula (I) may themselves act as prodrugs of other compounds of formula (I).

Also, within the scope of the invention are metabolites of the compounds of formula (I), that is, compounds formed in vivo upon administration of the compound of formula (I). Such metabolites may themselves be a compound of formula (I), which are particularly included with the scope of the present invention.

Included within the scope of the present invention are all tautomeric forms of the compounds of formula (I).

Certain derivatives of compounds of formula (I) which contain a nitrogen atom may also form the corresponding N-oxide, and such compounds are also within the scope of the present invention.

The present invention also includes all pharmaceutically acceptable isotopic variations of a compound of formula (I). An isotopic variation is defined as one in which at least one atom is replaced by an atom having the same atomic number, but an atomic mass different from the atomic mass usually found in nature.

Examples of isotopes suitable for inclusion in the compounds of the invention include isotopes of hydrogen, such as ²H and ³H, carbon, such as ¹³C and ¹⁴C, nitrogen, such as ¹⁵N, oxygen, such as ¹⁷O and ¹⁸O, phosphorus, such as ³²P, sulfur, such as ³⁵S, fluorine, such as ¹⁸F, and chlorine, such as ³⁶Cl.

Substitution of the compounds of the invention with isotopes such as deuterium may afford certain therapeutic advantages resulting from greater metabolic stability, for example, increased in vivo half-life or reduced dosage requirements, and hence may be preferred in some circumstances.

Certain isotopic variations of the compounds of formula (I), for example, those incorporating a radioactive isotope, are useful in drug and/or substrate tissue distribution studies. The radioactive isotopes tritium, and ¹⁴C, are particularly useful for this purpose in view of their ease of incorporation and ready means of detection.

Isotopic variations of the compounds of formula (I) can generally be prepared by conventional techniques known to those skilled in the art or by processes analogous to those described in the accompanying examples and preparations using appropriate isotopic variations of suitable reagents.

The compounds of formula (I) are inhibitors of the deubiquitylating enzyme USP30. According to a further aspect, the present invention provides a compound of formula (I) as defined herein, a tautomer thereof, or a pharmaceutically acceptable salt of said compound or tautomer for use in inhibiting USP30, either in vitro or in vivo.

According to a further aspect, the present invention provides a compound of formula (I) as defined herein, a tautomer thereof, or a pharmaceutically acceptable salt of said compound or tautomer for use as a medicament.

According to a further aspect, the present invention provides a method of treatment or prevention of a disorder or condition where inhibition of USP30 is known, or can be shown, to produce a beneficial effect, in a mammal, comprising administering to said mammal a therapeutically effective amount of a compound of formula (I) as defined herein, a tautomer thereof, or a pharmaceutically acceptable salt of said compound or tautomer. In one preferred embodiment of all aspects of the invention, the disorder or condition is a CNS indication. In a further preferred embodiment of all aspects of the invention, the disorder or condition is a peripheral indication.

According to a further aspect, the present invention provides the use of a compound of formula (I) as defined herein, a tautomer thereof, or a pharmaceutically acceptable salt of said compound or tautomer, in the manufacture of a medicament for the treatment or prevention of a disorder or condition where inhibition of USP30 is known, or can be shown, to produce a beneficial effect. The manufacture of a medicament may include, inter alia, the chemical synthesis of the compound of formula (I) or a salt thereof, or the preparation of a composition or formulation comprising the compound or salt, or the packaging of any medicament comprising the compound. In one preferred embodiment of all aspects of the invention, the disorder or condition is a CNS indication. In a further preferred embodiment of all aspects of the invention, the disorder or condition is a peripheral indication.

According to a further aspect, the present invention provides a method of inhibition of USP30 in a patient comprising administering to the patient a therapeutically effective amount of a compound of formula (I) as defined herein, a tautomer thereof, or a pharmaceutically acceptable salt of said compound or tautomer.

The disorder or condition benefiting from USP30 activity may be selected from a condition involving mitochondrial dysfunction, cancer and fibrosis.

In one preferred embodiment of all aspects of the invention, the disorder or condition benefiting from USP30 activity is a condition involving mitochondrial dysfunction. The condition involving mitochondrial dysfunction may be a CNS indication or a peripheral indication.

Mitochondrial dysfunctions result from defects of the mitochondria, which are specialized compartments present in every cell of the body except red blood cells. When mitochondria fail, less and less energy is generated within the cell and cell injury or even cell death will follow. If this process is repeated throughout the body the life of the subject in whom this is happening is severely compromised. Diseases of the mitochondria appear most often in organs that are very energy demanding such as the brain, heart, liver, skeletal muscles, kidney and the endocrine and respiratory system.

The condition involving mitochondrial dysfunction may be selected from a condition involving a mitophagy defect, a condition involving a mutation in mitochondrial DNA, a condition involving mitochondrial oxidative stress, a condition involving a defect in mitochondrial membrane potential, mitochondrial biogenesis, a condition involving a defect in mitochondrial shape or morphology, and a condition involving a lysosomal storage defect.

In particular, the condition involving mitochondrial dysfunction may be selected from: a neurodegenerative disease; multiple sclerosis (MS); mitochondrial encephalopathy, lactic acidosis and stroke-like episodes (MELAS) syndrome; materially-inherited diabetes and deafness (MIDD); Leber's hereditary optic neuropathy (LHON); cancer (including, for example, breast, ovarian, prostate, lung, kidney, gastric, colon, testicular, head and neck, pancreas, brain, melanoma, bone or other cancers of tissue organs and cancers of the blood cells, such as lymphoma and leukaemia, multiple myeloma, metastatic carcinoma, osteosarcoma, chondosarcoma, Ewing's sarcoma, nasopharyngeal carcinoma, colorectal cancer, and non-small cell lung carcinoma); neuropathy, ataxia, retinitis pigmentosa, maternally inherited Leigh syndrome (NARP-MILS); Danon disease; diabetes; diabetic nephropathy; metabolic disorders; heart failure; ischemic heart disease leading to myocardial infarction; psychiatric diseases, for example schizophrenia; multiple sulfatase deficiency (MSD); mucolipidosis II (ML II); mucolipidosis III (ML III); mucolipidosis IV (ML IV); GM1-gangliosidosis (GM1); neuronal ceroid-lipofuscinoses (NCL1); Alpers disease; Barth syndrome; beta-oxidation defects; carnitine-acyl-carnitine deficiency; carnitine deficiency; creatine deficiency syndromes; co-enzyme Q10 deficiency; complex I deficiency; complex II deficiency; complex III deficiency; complex IV deficiency; complex V deficiency; COX deficiency; chronic progressive external ophthalmoplegia syndrome (CPEO); CPT I deficiency; CPT II deficiency; glutaric aciduria type II; Kearns-Sayre syndrome; lactic acidosis; long-chain acyl-CoA dehydrogenase deficiency (LCHAD); Leigh disease or syndrome; Leigh syndrome French Canadian (LSFC) variant; lethal infantile cardiomyopathy (LIC); Luft disease; medium-chain acyl-CoA dehydrogenase deficiency (MCAD); myoclonic epilepsy and ragged-red fiber (MERRF) syndrome; mitochondrial cytopathy; mitochondrial recessive ataxia syndrome; mitochondrial DNA depletion syndrome; myoneurogastointestinal disorder and encephalopathy; Pearson syndrome; pyruvate dehydrogenase deficiency; pyruvate carboxylase deficiency; POLG mutations; medium/short-chain 3-hydroxyacyl-CoA dehydrogenase (M/SCHAD) deficiency; very long-chain acyl-CoA dehydrogenase (VLCAD) deficiency; peroxisomal disorders; methylmalonic acidemia; mevalonate kinase deficiency; age-dependent decline in cognitive function and muscle strength; muscle structure disorders; and cognitive impairment associated with neurodegenerative and neuropsychiatric disorders.

The condition involving mitochondrial dysfunction may be a CNS disorder, for example a neurodegenerative disease.

Neurodegenerative diseases include, but are not limited to, Parkinson's disease, Alzheimer's disease, amyotrophic lateral sclerosis (ALS), Huntington's disease, ischemia, stroke, dementia with Lewy bodies, multiple system atrophy (MSA), progressive supranuclear palsy (PSP), corticobasal degeneration (CBD), and frontotemporal dementia.

In particular, the compounds of the invention may be useful in the treatment or prevention of Parkinson's disease, including, but not limited to, PD related to mutations in α-synuclein, parkin, PINK1, GBA, and LRRK2, and autosomal recessive juvenile Parkinson's disease (AR-JP), or early onset Parkinson's disease (EOPD), where parkin or PINK1 is mutated, truncated, or deleted.

In particular, the compounds of the invention may be useful in treatment of cognitive impairment associated with neurodegenerative and neuropsychiatric disorders, including, for example, cognitive impairment associated with Alzheimer's disease and Parkinson's disease, preclinical or prodromal forms of AD and PD, Huntington's disease, dementia with lewy body disease, cognitive impairment associated with schizophrenia, mood disorders, bipolar and major depressive disorders.

In a preferred embodiment, the present invention is directed to the treatment or prevention of Leigh syndrome or disease, including for example, X-linked Leigh's disease, Leigh syndrome French Canadian variant, and/or the symptoms associated with Leigh's disease. In particular, the compounds of the invention may be useful in the treatment of a muscle structure disorder selected from muscular dystrophy, Duchenne muscular dystrophy, Becker muscular dystrophy, limb-girdle muscular dystrophy, congenital muscular dystrophy, facioscapulohumeral muscular dystrophy, myotonic dystrophy, oculopharyngeal muscular dystrophy, distal muscular dystrophy, Emery-Dreifuss muscular dystrophy, Bethlem myopathy, central core disease, congenital fiber type disproportion, hyaline body myopathy, muscle sodium channel disorders, myotonic chondrodystrophy, myotubular myopathy, nemaline body disease, and stress urinary incontinence.

The compounds of the invention or pharmaceutical compositions thereof as described herein may be combined with one or more additional agents when used for the treatment or prevention of conditions involving mitochondrial dysfunction. The compounds may be combined with one or more additional agents selected from levodopa, a dopamine agonist, a monoamino oxygenase (MAO) B inhibitor, a catechol O-methyltransferase (COMT) inhibitor, an anticholinergic, riluzole, amantadine, a cholinesterase inhibitor, memantine, tetrabenazine, an antipsychotic, diazepam, clonazepam, an antidepressant, and an anti-convulsant. The compounds may be combined with agents which reduce/remove pathogenic protein aggregates in neurodegenerative diseases, such as agents which reduce/remove alpha-synuclein in Parkinson's disease, multiple system atrophy or dementia with Lewy bodies; agents which reduce/remove Tau in Alzheimer's disease or progressive supranuclear palsy; agents which reduce/remove TDP-43 in ALS or frontotemporal dementia.

The compounds may be combined with novel agents which may be used as treatments for mitochondrial disease, including, but not limited to, nicotinamide riboside.

The compounds may be combined with agents which are used as treatments for muscular dystrophies such as DMD, including corticosteroids (e.g., prednisone and deflazacort), ataluren, eteplirsen, golodirsen, casimersen, viltepso, and other exon-skipping/nonsense readthrough/gene therapies, givinostat, pamrevlumab and vamorolone, and also heart medications, such as angiotensin-converting enzyme inhibitors and beta blockers.

In another preferred embodiment of all aspects of the invention, the disorder or condition benefiting from USP30 activity is cancer. The cancer may be linked to mitochondrial dysfunction. Preferred cancers include, for example, breast, ovarian, prostate, lung, kidney, gastric, colon, testicular, head and neck, pancreas, brain, melanoma, bone or other cancers of tissue organs and cancers of the blood cells, such as lymphoma and leukaemia, multiple myeloma, metastatic carcinoma, osteosarcoma, chondosarcoma, Ewing's sarcoma, nasopharyngeal carcinoma, colorectal cancer, colorectal cancer, and non-small cell lung carcinoma.

In particular, the compounds of the invention may be useful in the treatment or prevention of cancer where apoptotic pathways are dysregulated and more particularly where proteins of the BCL-2 family are mutated, or over or under expressed.

Fibrosis refers to the accumulation of extracellular matrix constituents that occurs following trauma, inflammation, tissue repair, immunological reactions, cellular hyperplasia, and neoplasia. Fibrotic disorders that may be treated by the compounds and compositions ofthe present invention include, inter alia, fibrosis/fibrotic disorders associated with major organ diseases, for example, interstitial lung disease (ILD), liver cirrhosis, non-alcoholic fatty liver disease (NAFLD) and non-alcoholic steatohepatitis (NASH) (hepatic fibrosis), kidney disease (renal fibrosis), acute kidney injury (AKI), acute kidney disease (AKD), chronic kidney disease (CKD), delayed kidney graft function, heart or vascular disease (cardiac fibrosis, hypertrophic cardiomyopathy (HCM)), and diseases of the eye; fibroproliferative disorders, for example, systemic and local scleroderma, keloids and hypertrophic scars, atherosclerosis, restenosis, and Dupuytren's contracture; scarring associated with trauma, for example, surgical complications, chemotherapeutics drug-induced fibrosis (e.g., bleomycin-induced fibrosis), radiation-induced fibrosis, accidental injury and burns); retroperitoneal fibrosis (Ormond's disease); and peritoneal fibrosis/peritoneal scarring in patients receiving peritoneal dialysis, usually following renal transplantation. See, for example, Wynn et al, 2004, Nat Rev Immunol. August; 4(8): 583-594. The present invention therefore relates to methods of treatment or prevention, and compounds and compositions used in said methods, of fibrosis/fibrotic disorders of and/or associated with the major organs, including for example, the lung, liver, kidney, heart, skin, eye, gastrointestinal tract, peritoneum and bone marrow, and other diseases/disorders herein described.

The compounds may be combined with agents which are used as treatments for kidney disease, including anti-diabetic agents, cardiovascular disease agents, and novel agents targeting disease relevant pathways such as oxidative stress (including, but not limited to, the nrf2/keap-1 pathway) and anti-apoptotic pathways (including, but not limited to, anti p53 agents).

Interstitial lung disease (ILD) includes disorders in which pulmonary inflammation and fibrosis are the final common pathways of pathology, for example, sarcoidosis, silicosis, drug reactions, infections, and collagen vascular diseases, such as rheumatoid arthritis and systemic sclerosis (scleroderma). The fibrotic disorder of the lung includes, for example, pulmonary fibrosis, idiopathic pulmonary fibrosis (IPF), usual interstitial pneumonitis (UIP), interstitial lung disease, cryptogenic fibrosing alveolitis (CFA), bronchiolitis obliterans, and bronchiectasis.

Idiopathic pulmonary fibrosis (IPF) is the most common type of ILD and has no known cause.

The compounds may be combined with agents which are treatments for IPF and potentially for ILD, including nintedanib and pirfenidone.

Liver cirrhosis has similar causes to ILD and includes, for example, cirrhosis associated with viral hepatitis, schistosomiasis and chronic alcoholism.

Kidney disease may be associated with diabetes, which can damage and scar the kidneys leading to a progressive loss of function, and also hypertensive diseases. Kidney fibrosis may occur at any stage of kidney disease, from acute kidney disease (AKD) post injury and chronic kidney disease (CKD), such as incident CKD and progressive CKD, through to end-stage renal disease (ESRD). Kidney fibrosis can develop as a result of cardiovascular disease such as hypertension or diabetes, both of which place immense strain on kidney function which promotes a fibrotic response. However, kidney fibrosis can also be idiopathic (without a known cause), and certain genetic mitochondrial diseases also present kidney fibrosis manifestations and associated symptoms.

Heart disease may result in scar tissue that can impair the ability of the heart to pump.

Diseases of the eye include, for example, macular degeneration and retinal and vitreal retinopathy, which can impair vision.

In a preferred embodiment, the present invention is directed to the treatment or prevention of idiopathic pulmonary fibrosis (IPF).

In another preferred embodiment, the present invention is directed to the treatment or prevention of kidney fibrosis.

In another preferred embodiment, the present invention is directed to the treatment or prevention of acute kidney injury (AKI), especially in high risk patients. Examples include post-surgical AKI, for example organ transplantation, such as due to ischemia reperfusion injury, delayed graft function; oncology, such as AKI due to chemotherapy; contrast medium-induced nephropathy, such as direct-tubular cytotoxicity, hemodynamic ischemia and osmotic effects; acute interstitial nephritis, such as due to drugs or infection; AKI due to obstruction, such as kidney stones; and COVID-19-induced AKI.

A particular high risk patient sub-group are those undergoing cardiac surgery, for example, coronary artery bypass graft and/or valve surgery. There are established static risk factors for AKI such as age 65 years or over, insulin dependent diabetes, CKD (adults with an estimated glomerular filtration rate [eGFR] less than 60 ml/min/1.73 m2 are at particular risk), heart failure, liver disease, history of AKI.

In another preferred embodiment, the present invention is directed to the treatment or prevention of acute kidney disease (AKD) or chronic kidney disease (CKD) stemming from such AKI, including for example, tubulointerstitial fibrosis and diabetic nephropathy.

In another preferred embodiment, the present invention is directed to the treatment or prevention of liver diseases, including, for example, NAFLD, NASH, liver cirrhosis, portal hypertension, acute liver failure, and hepatocellular carcinoma. Liver disease such as NAFLD and NASH may be associated with various metabolic conditions such as metabolic syndrome and Type II diabetes, which also would increase risk for various diabetes associated pathologies, including diabetic retinopathy and peripiheral neuropathies.

The compounds of the invention or pharmaceutical compositions thereof as described herein may be combined with one or more additional agents when used for the treatment or prevention of conditions involving liver disease and metabolic dysfunction, including metformin, sulfonylureas, DPP-4 inhibitors, GLP-1 agonists, PPAR agonists, SGLT2 inhibitors, angiotensin-converting enzyme (ACE) inhibitors and angiotensin II receptor blockers (ARBs).

References to ‘treatment’ includes means to ameliorate, alleviate symptoms, eliminate the causation of the symptoms either on a temporary or permanent basis. The compounds of the invention are useful in the treatment of the diseases disclosed herein in humans and other mammals.

In another embodiment, the invention encompasses prophylactic therapy of the diseases disclosed herein and includes means to prevent or slow the appearance of symptoms of the named disorder or condition. The compounds of the invention are useful in the prevention of the diseases disclosed herein in humans and other mammals.

A patient in need of treatment or prevention may, for example, be a human or other mammal suffering from the condition or at risk of suffering from the condition.

According to a further aspect, the present invention provides a pharmaceutical composition comprising a compound of formula (I) as defined herein, or a pharmaceutically acceptable salt of said compound or tautomer, together with a pharmaceutically acceptable diluent or carrier.

Pharmaceutical compositions of the invention comprise any of the compounds of the invention combined with any pharmaceutically acceptable carrier, adjuvant or vehicle. Examples of pharmaceutically acceptable carriers are known to those skilled in the art and include, but are not limited to, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may be in the form of, for example, tablets, capsules, powders, granules, elixirs, lozenges, suppositories, syrups, and liquid preparations including suspensions and solutions. The term “pharmaceutical composition” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms.

The compounds of the invention or pharmaceutical compositions thereof, as described herein, may be used alone or combined with one or more additional pharmaceutical agents. The compounds may be combined with an additional anti-tumour therapeutic agent, for example, chemotherapeutic drugs or inhibitors of other regulatory proteins. In one embodiment, the additional anti-tumour therapeutic agent is a BH-3 mimetic. In a further embodiment, BH-3 mimetics may be selected from but not limited to one or more of ABT-737, ABT-199, ABT-263, and Obatoclax. In a further embodiment, the additional anti-tumour agent is a chemotherapeutic agent. Chemotherapeutic agents may be selected from but not limited to, olaparib, mitomycin C, cisplatin, carboplatin, oxaliplatin, ionizing radiation (IR), camptothecin, irinotecan, topotecan, temozolomide, taxanes, 5-fluoropyrimidines, gemcitabine, and doxorubicin.

For the treatment or prevention of fibrotic disorders, for example, the compounds of the invention or pharmaceutical compositions thereof, as described herein, may be used alone or combined with one or more additional pharmaceutical agents selected from the group consisting of anticholinergic agents, beta-2 mimetics, steroids, PDE-IV inhibitors, p38 MAP kinase inhibitors, NK1 antagonists, LTD4 antagonists, EGFR inhibitors and endothelin antagonists.

In particular, the compounds of the invention or pharmaceutical compositions thereof, as described herein, may be used alone or combined with one or more additional pharmaceutical agents selected from the group consisting of general immunosuppressive drugs, such as a corticosteroid, immunosuppressive or cytotoxic agents, or antifibrotics, such as pirfenidone or a non-specific kinase inhibitor (e.g., nintedanib).

The pharmaceutical compositions of the invention may be administered in any suitably effective manner, such as oral, parenteral, topical, inhaled, intranasal, rectal, intravaginal, ocular and aural.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulfate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25 (2), 1-14 (2001). Other modified release formulations are described in U.S. Pat. No. 6,106,864.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle (including microneedle) injectors, needle-free injectors, and infusion techniques.

Parenteral formulations are typically aqueous solutions which may contain excipients such as salts, carbohydrates, and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by suitable processing, for example, the use of high energy spray-dried dispersions and/or using appropriate formulation techniques, such as the use of solubility-enhancing agents.

Formulations for parenteral administration may be formulated to be immediate and/or modified release. Modified release formulations include delayed, sustained, pulsed, controlled dual, targeted, and programmed release.

Pharmaceutical compositions of the present invention also include compositions and methods known in the art for bypassing the blood brain barrier or can be injected directly into the brain. Suitable areas for injection include the cerebral cortex, cerebellum, midbrain, brainstem, hypothalamus, spinal cord and ventricular tissue, and areas of the peripheral nervous system including the carotid body and the adrenal medulla.

### Dosage

The magnitude of an effective dose of a compound will, of course, vary with the nature of the severity of the condition to be treated and the route of administration. The selection of appropriate dosages is within the remit of the physician. The daily dose range is about 10 μg to about 100 mg per kg body weight of a human and non-human animal and in general may be around 10 μg to 30 mg per kg body weight per dose. The above dose may be given from one to three times per day.

For example, oral administration may require a total daily dose of from 5 mg to 1000 mg, such as from 5 to 500 mg, while an intravenous dose may only require from 0.01 to 30 mg/kg body weight, such as from 0.1 to 10 mg/kg, more preferably from 0.1 to 1 mg/kg body weight. The total daily dose may be administered in single or divided doses. The skilled person will also appreciate that, in the treatment or prevention of certain conditions, compounds of the invention may be taken as a single dose on an “as required” basis (i.e. as needed or desired).

### Synthetic Methodologies

Compounds of formula (I) may be prepared using methods as described below in the general reaction schemes and the representative examples. Where appropriate, the individual transformations within a scheme may be completed in a different order. The invention is illustrated by the following non-limiting examples in which the following abbreviations and definitions are used. Compounds were characterised by liquid chromatography-mass spectrometry (LCMS) or ¹H NMR or both.

According to a further aspect, the present invention provides a process for the preparation of a compound of formula (I) comprising reacting a compound of formula (IV), where Y is OH with an amine of formula (V), where PG is a protecting group, such as BOC or Cbz, to give an amide of formula (III) (Scheme 1). The amide-coupling reaction can be performed using standard methodology, for example by reaction using a coupling reagent such as DCC, HATU, HBTU, EDC or via a mixed anhydride. Alternatively, the acid (IV), where Y is OH, or the carboxylate salt thereof, such as the lithium salt, can be converted into the acid chloride (IV), where Y is Cl, using SOCl₂, POCl₃, PCl₃ or PCl₅, which can then be reacted with the amine (V), preferably in a suitable solvent in the presence of a suitable base. Alternatively, the compound (IV), where Y forms the ester, can be reacted directly with the amine (V), preferably in a suitable solvent.

Compounds of formula (IV) may be prepared according to the processes disclosed in WO 2016/156816, WO 2017/103614, WO 2018/234775, WO 2020/212350, WO 2020/212351, WO 2021/043870, WO 2021/204856, WO 2021/239863, WO 2021/245186, WO 2021/249909 and WO 2022/084479.

The compound of formula (III) may be deprotected using standard methods to give amine (II) which may then be reacted with cyanogen bromide to give the corresponding compound of formula (I).

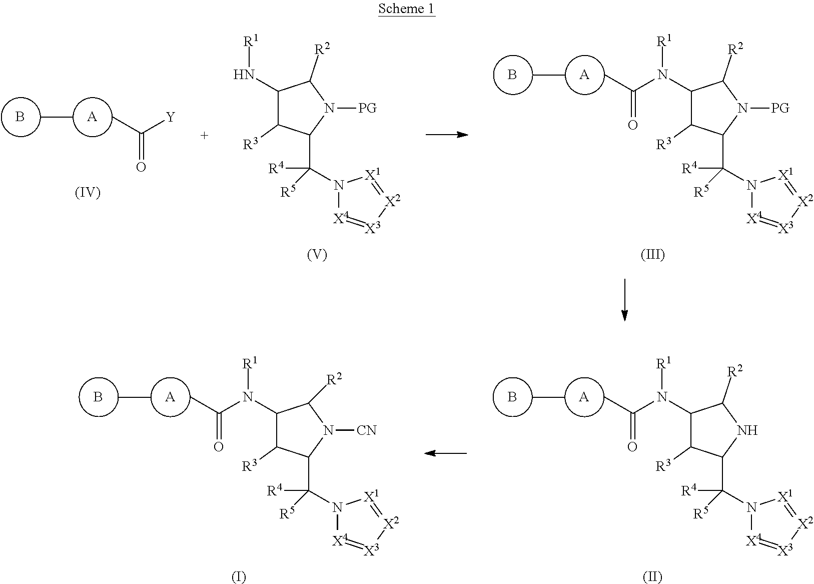

In a further aspect, the present invention provides a compound selected from formulae (II) and (III):

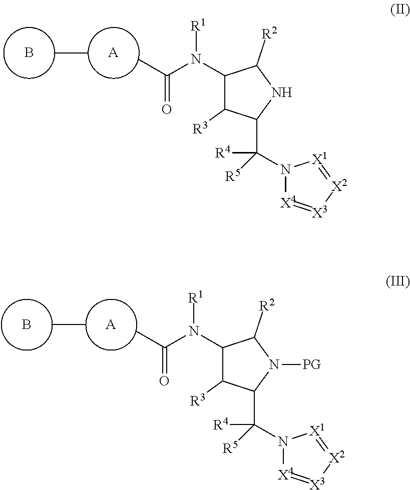

wherein PG is a protecting group and ring A, ring B, X¹, X², X³, X⁴, R¹, R², R³, R⁴ and R are as defined herein for the compound of formula (I) and all preferred aspects and embodiments thereof, a tautomer thereof, or a salt of said compound or tautomer.

For the intermediates of the synthesis of each of the compounds of formulae (I), (I)(A), (I)(B), (I)(C), (I)(D), (I)(E), (I)(F) and (I)(G) of the present invention, the corresponding compounds of formulae (II) and (III) [(II)(i) to (II)(G)(i) and (III)(i) to (III)(G)(i)] preferably exist as single stereoisomers having the absolute configuration of formulae (II)(i) and (III)(i):

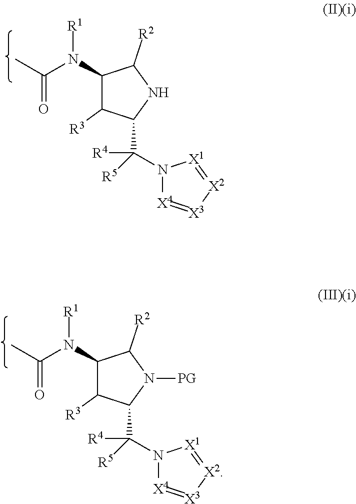

Where the compounds of formulae (II) and (III) exist as single stereoisomers, they preferably exist with a stereoisomeric excess of at least 60%, more preferably at least 80%, yet more preferably at least 90%, and most preferably at least 95%, for example 96%, 97%, 98%, 99%, or 100%.

Protecting groups are preferably selected from tert-butyloxycarbonyl (BOC), benzyloxycarbonyl (Cbz), p-methoxybenzyl carbonyl (MeOZ), 9-fluorenylmethyloxycarbonyl (Fmoc), acetyl (Ac), benzoyl (Bz), benzyl (Bn), carbamate, p-methoxybenzyl (PMB), 3,4-dimethoxybenzyl (DMPM), p-methoxyphenyl (PMP), tosyl (Ts), trichloroethoxycarbonyl (Troc), 4-nitrobenzenesulfonyl (Nosyl) and 2-nitrophenylsulfenyl (Nps). Most preferred are BOC and Cbz.

### Abbreviations

| | | | |
|---|---|---|---|
| br s | broad singlet (NMR signal) | MeOH | methanol |
| d | doublet (NMR signal) | min | minute(s) |
| DCM | dichloromethane | NMP | N-methyl-2-pyrrolidone |
| DEA | diethylamine | NMR | nuclear magnetic resonance |
| DMF | N,N-dimethylformamide | nOe | nuclear Overhauser effect |
| DMSO | dimethylsulfoxide | PDA | Photodiode-Array |
| ES | electrospray | rac | racemic |
| EtOAc | ethyl acetate | rt | room temperature |
| h | hour(s) | S | singlet (NMR signal) |
| HPLC | High performance liquid | SFC | Supercritical fluid chromatography |
| | chromatography | TBD | 1,5,7-triazabicyclo[4.4.0]dec-5-ene |
| IPA | isopropyl alcohol | TEA | triethylamine |
| LCMS | Liquid chromatography-mass | TFA | trifluoroacetic acid |
| | spectrometry | THF | tetrahydrofuran |
| m | multiplet (NMR signal) | vol | volumes |
| Ms | mesyl/methanesulfonyl | | |
| MeCN | acetonitrile | | |

### LCMS Methods

### Method C

| Mobile | (A) | 2 mM Ammonium acetate & 0.1% formic acid in water | |
|---|---|---|---|
| phase | (B) | 0.1% Formic acid in acetonitrile | |
| Instrument | | Shimadzu Nexera UFLC with 2020 single quadrupole mass detector | |
| Column | | Waters X-Bridge C18 (50 × 4.6 mm), 3.5 μm | |
| Column oven temperature | | Ambient | |
| Flow rate | | 0.550 | mL/min |
| Run time | | 3.0 | min |
| Gradient | | | |
| TIME | Flow Rate | | |
| (min) | (mL/min) | % A | % B |
| 0.01 | 0.55 | 98 | 2 |
| 0.30 | 0.55 | 98 | 2 |
| 0.60 | 0.55 | 50 | 50 |
| 1.10 | 0.55 | 25 | 75 |
| 2.00 | 0.60 | 0 | 100 |
| 2.70 | 0.60 | 0 | 100 |
| 2.71 | 0.55 | 98 | 2 |
| 3.00 | 0.55 | 98 | 2 |

| Mobile | (A) | 2 mM Ammonium acetate & 0.1% formic acid in water | |
|---|---|---|---|
| phase | (B) | 0.1% Formic acid in acetonitrile | |
| Instrument | | Waters ACQUITY UPLC H Class with PDA and SQ detector | |
| Column | | BEH C18 (50 × 2.1 mm), 1.7 μm | |
| Column oven temperature | | Ambient | |
| Flow rate | | 0.550 | mL/min |
| Run time | | 2.0 | min |
| Gradient | | | |
| TIME | Flow Rate | | |
| (min) | (mL/min) | % A | % B |
| 0.00 | 0.55 | 95 | 5 |
| 0.60 | 0.60 | 30 | 70 |
| 0.80 | 0.65 | 10 | 90 |
| 1.10 | 0.65 | 0 | 100 |
| 1.70 | 0.65 | 0 | 100 |
| 1.71 | 0.55 | 95 | 5 |
| 2.00 | 0.55 | 95 | 5 |

### Method F

| Mobile | (A) | 10 mM Ammonium acetate in water | |
|---|---|---|---|
| phase | (B) | 100% Acetonitrile | |
| Instrument | | Agilent 1290 Infinity RRLC attached with Agilent 6120 | |
| | | mass detector and diode array detector | |
| Column | | YMC TRAIT, C18 (150 mm × 4.6 mm), 5 μm | |
| Column oven temperature | | Ambient | |
| Flow rate | | 1.0 | mL/min |
| Run time | | 12.0 | min |
| Gradient | | | |
| TIME | Flow Rate | | |
| (min) | (mL/min) | % A | % B |
| 0.01 | 1.0 | 90 | 10 |
| 5.00 | 1.0 | 10 | 90 |
| 7.00 | 1.0 | 0 | 100 |
| 11.00 | 1.0 | 0 | 100 |
| 11.01 | 1.0 | 90 | 10 |
| 12.00 | 1.0 | 90 | 10 |

### Method H

| Mobile | (A) | 5 mM Ammonium bicarbonate in water | |
|---|---|---|---|
| phase | (B) | 100% Acetonitrile | |
| Instrument | | Shimadzu Nexera UFLC with 2020 single quadrupole mass detector | |
| Column | | Waters X-Bridge C18 (50 × 4.6 mm), 3.5 μm | |
| Column oven temperature | | Ambient | |
| Flow rate | | 1.0 | mL/min |
| Run time | | 8.0 | min |
| Gradient | | | |
| TIME | Flow Rate | | |
| (min) | (mL/min) | % A | % B |
| 0.01 | 1.0 | 95 | 5 |
| 3.50 | 1.0 | 10 | 90 |
| 4.50 | 1.0 | 5 | 95 |
| 6.00 | 1.0 | 5 | 95 |
| 6.01 | 1.0 | 95 | 5 |
| 8.00 | 1.0 | 95 | 5 |

### Methd H1

| Mobile | (A) | 5 mM Ammonium bicarbonate in water | |
|---|---|---|---|
| phase | (B) | 100% Acetonitrile | |
| Instrument | | Shimadzu Nexera UFLC with 2020 single | |
| | | quadrupole mass detector | |
| Column | | Waters X-Bridge C18 (50 × 4.6 mm), 3.5 μm | |
| Column oven | | Ambient | |
| temperature | | | |
| Flow rate | | 1.0 mL/min | |
| Run time | | 6.0 min | |
| Gradient | | | |
| TIME | Flow Rate | | |
| (min) | (mL/min) | % A | % B |
| 0.01 | 1.0 | 95 | 5 |
| 2.80 | 1.0 | 15 | 85 |
| 3.50 | 1.0 | 5 | 95 |
| 5.00 | 1.0 | 5 | 95 |
| 5.01 | 1.0 | 95 | 5 |
| 6.00 | 1.0 | 95 | 5 |

### Method J

| Mobile | (A) | 2 mM Ammonium acetate & 0.1% formic | |
|---|---|---|---|
| phase | | acid in water | |
| | (B) | 0.1% Formic acid in acetonitrile | |
| Instrument | | Waters ACQUITY H Class with PDA and SQ detector | |
| Column | | BEH C18 (50 × 2.1 mm), 1.7 μm | |
| Column oven | | Ambient | |
| temperature | | | |
| Flow rate | | 0.450 mL/min | |
| Run time | | 8.0 min | |
| Gradient | | | |
| TIME | Flow Rate | | |
| (min) | (mL/min) | % A | % B |
| 0.01 | 0.45 | 98 | 2 |
| 0.50 | 0.45 | 98 | 2 |
| 5.00 | 0.45 | 10 | 90 |
| 6.00 | 0.45 | 5 | 95 |
| 7.00 | 0.45 | 5 | 95 |
| 7.01 | 0.45 | 98 | 2 |
| 8.00 | 0.45 | 98 | 2 |

### Method J1

| Mobile | (A) | 2 mM Ammonium acetate & 0.1% formic acid in water | |
|---|---|---|---|
| phase | (B) | 0.1% Formic acid in acetonitrile | |
| Instrument | | Agilent 1290 Infinity RRLC attached with Agilent | |
| | | 6120 mass detector and diode array detector | |
| Column | | BEH C18 (50 × 2.1 mm), 1.7 μm | |
| Column oven | | Ambient | |
| temperature | | | |
| Flow rate | | 0.450 mL/min | |
| Run time | | 6.0 min | |
| Gradient | | | |
| TIME | Flow Rate | | |
| (min) | (mL/min) | % A | % B |
| 0.01 | 0.450 | 98 | 2 |
| 0.50 | 0.450 | 98 | 2 |
| 3.00 | 0.450 | 30 | 70 |
| 4.00 | 0.500 | 5 | 95 |
| 5.50 | 0.500 | 5 | 95 |
| 5.51 | 0.450 | 98 | 2 |
| 6.00 | 0.450 | 98 | 2 |

### Method N

| Mobile | (A) | 10 mM Ammonium acetate & 0.1% formic acid | |
|---|---|---|---|
| phase | | in water | |
| | (B) | 0.1% Formic acid in acetonitrile | |
| Instrument | | Agilent 1290 Infinity RRLC attached with Agilent | |
| | | 6120 mass detector and PDA detector | |
| Column | | YMC-Pack ODS-AQ (250 × 4.6 mm), 5 μm | |
| Column oven | | Ambient | |
| temperature | | | |
| Flow rate | | 1.0 mL/min | |
| Run time | | 35.0 min | |
| Gradient | | | |
| TIME | Flow Rate | | |
| (min) | (mL/min) | % A | % B |
| 0.00 | 1.0 | 90 | 10 |
| 10.00 | 1.0 | 60 | 40 |
| 20.00 | 1.0 | 30 | 70 |
| 30.00 | 1.0 | 0 | 100 |
| 33.00 | 1.0 | 0 | 100 |
| 33.01 | 1.0 | 90 | 10 |
| 35.00 | 1.0 | 90 | 10 |

### Method X1 Method Used for Reverse Phase Preparative HPLC

| Mobile | (A) | 5 mM Ammonium bicarbonate & 0.1% | |
|---|---|---|---|
| Phase | | ammonia in water | |
| | (B) | 100% Acetonitrile | |
| Instrument | | Shimadzu LC-20AP and UV detector | |
| Column | | X-Bridge C8 (250 × 19 mm), 5 μm | |
| Flow rate | | 15.0 mL/min | |
| Run time | | 30.0 min | |
| Gradient | | | |
| TIME | Flow Rate | | |
| (min) | (mL/min) | % A | % B |
| 0.01 | 15.0 | 80 | 20 |
| 24.00 | 15.0 | 55 | 45 |
| 24.01 | 15.0 | 0 | 100 |
| 26.00 | 15.0 | 0 | 100 |
| 26.01 | 15.0 | 80 | 20 |
| 30.00 | 15.0 | 80 | 20 |

### Method X3 Method Used for Reverse Phase Preparative HPLC

| Mobile | (A) | 5 mM Ammonium bicarbonate & 0.1% | |
|---|---|---|---|
| Phase | | ammonia in water | |
| | (B) | 100% Acetonitrile | |
| Instrument | | Shimadzu LC-20AP and UV detector | |
| Column | | X-Bridge C8 (250 × 19 mm), 5 μm | |
| Flow rate | | 12.0 mL/min | |
| Run time | | 34.0 min | |
| Gradient | | | |
| TIME | Flow Rate | | |
| (min) | (mL/min) | % A | % B |
| 0.01 | 12.0 | 80 | 20 |
| 22.00 | 12.0 | 50 | 50 |
| 25.00 | 12.0 | 50 | 50 |
| 25.01 | 12.0 | 0 | 100 |
| 27.00 | 12.0 | 0 | 100 |
| 27.01 | 12.0 | 80 | 20 |
| 34.00 | 12.0 | 80 | 20 |

### Method X4 Method Used for Reverse Phase Preparative HPLC

| Mobile | (A) | 5 mM Ammonium bicarbonate & 0.1% | |
|---|---|---|---|
| Phase | | ammonia in water | |
| | (B) | 100% Acetontrile | |
| Instrument | | Shimadzu LC-20AP and UV detector | |
| Column | | X-select phenyl-hexyl (250 × 19 mm), 5 μm | |
| Flow rate | | 15.0 mL/min | |
| Run time | | 31.0 min | |
| Gradient | | | |
| TIME | Flow Rate | | |
| (min) | (mL/min) | % A | % B |
| 0.01 | 15.0 | 80 | 20 |
| 24.00 | 15.0 | 50 | 50 |
| 24.01 | 15.0 | 0 | 100 |
| 26.00 | 15.0 | 0 | 100 |
| 26.01 | 15.0 | 80 | 20 |
| 31.00 | 15.0 | 80 | 20 |

### Method X8 Method Used for Reverse Phase Preparative HPLC

| Mobile | (A) | 5 mM Ammonium bicarbonate & 0.1% | |
|---|---|---|---|
| Phase | | ammonia in water | |
| | (B) | 100% Acetonitrile | |
| Instrument | | Shimadzu LC-20AP and UV detector | |
| Column | | X-Bridge C18 (250 × 19 mm), 5 μm | |
| Flow rate | | 15.0 mL/min | |
| Run time | | 30.0 min | |
| Gradient | | | |
| TIME | Flow Rate | | |
| (min) | (mL/min) | % A | % B |
| 0.01 | 15.0 | 65 | 35 |
| 22.00 | 15.0 | 45 | 55 |
| 22.01 | 15.0 | 0 | 100 |
| 25.00 | 15.0 | 0 | 100 |
| 25.01 | 15.0 | 65 | 35 |
| 30.00 | 15.0 | 65 | 35 |

### Method X9 Method Used for Reverse Phase Preparative HPLC

| Mobile | (A) | 0.1% Ammonia in water | |
|---|---|---|---|
| Phase | (B) | 100% Acetonitrile | |
| Instrument | | Shimadzu LC-20AP and UV detector | |
| Column | | X-Bridge C18 (250 × 19 mm), 5 μm | |
| Flow rate | | 15.0 mL/min | |
| Run time | | 33.0 min | |
| Gradient | | | |
| TIME | Flow Rate | | |
| (min) | (mL/min) | % A | % B |
| 0.01 | 15.0 | 90 | 10 |
| 25.00 | 15.0 | 55 | 45 |
| 25.01 | 15.0 | 0 | 100 |
| 27.00 | 15.0 | 0 | 100 |
| 27.01 | 15.0 | 90 | 10 |
| 33.00 | 15.0 | 90 | 10 |

### Method X10 Method Used for Reverse Phase Preparative HPLC

| Mobile | (A) | 5 mM Ammonium bicarbonate & 0.1% | |
|---|---|---|---|
| Phase | | ammonia in water | |
| | (B) | 100% Acetonitrile | |
| Instrument | | Shimadzu LC-20AP and UV detector | |
| Column | | X-Bridge C18 (250 × 19 mm), 5 μm | |
| Flow rate | | 13.0 mL/min | |
| Run time | | 25.0 min | |
| Gradient | | | |
| TIME | Flow Rate | | |
| (min) | (mL/min) | % A | % B |
| 0.01 | 13.0 | 65 | 35 |
| 18.00 | 13.0 | 58 | 42 |
| 18.01 | 13.0 | 0 | 100 |
| 20.00 | 13.0 | 0 | 100 |
| 20.01 | 13.0 | 65 | 35 |
| 25.00 | 13.0 | 65 | 35 |

### Method X11 Method Used for Reverse Phase Preparative HPLC

| Mobile | (A) | 0.1% Ammonia in water | |
|---|---|---|---|
| Phase | (B) | 100% Acetonitrile | |
| Instrument | | Shimadzu LC-20AP and UV detector | |
| Column | | X-Bridge C18 (250 × 19 mm), 5 μm | |
| Flow rate | | 12.0 mL/min | |
| Run time | | 27.0 min | |
| Gradient | | | |
| TIME | Flow Rate | | |
| (min) | (mL/min) | % A start | % B end |
| 0.01 | 12.0 | 90 | 10 |
| 20.00 | 12.0 | 55 | 45 |
| 20.01 | 12.0 | 0 | 100 |
| 22.00 | 12.0 | 0 | 100 |
| 22.01 | 12.0 | 90 | 10 |
| 27.00 | 12.0 | 90 | 10 |

### Method X14 Method Used for Reverse Phase Preparative HPLC

| Mobile | (A) | 5 mM Ammonium bicarbonate & 0.1% | |
|---|---|---|---|
| Phase | | ammonia in water | |
| | (B) | 100% Acetonitrile | |
| Instrument | | Shimadzu LC-20AP and UV detector | |
| Column | | YMC Actus Triart (250 × 20 mm), 5 μm | |
| Flow rate | | 14.0 mL/min | |
| Run time | | 35.0 min | |
| Gradient | | | |
| TIME | Flow Rate | | |
| (min) | (mL/min) | % A start | % B end |
| 0.01 | 14.0 | 70 | 30 |
| 25.00 | 14.0 | 50 | 50 |
| 27.00 | 14.0 | 50 | 50 |
| 27.01 | 14.0 | 0 | 100 |
| 29.00 | 14.0 | 0 | 100 |
| 29.01 | 14.0 | 70 | 30 |
| 35.00 | 14.0 | 70 | 30 |

### Method X15 Method Used for Reverse Phase Preparative HPLC

| Mobile | (A) | 5 mM Ammonium bicarbonate & 0.1% | |
|---|---|---|---|
| Phase | | ammonia in water | |
| | (B) | 100% Acetonitrile | |
| Instrument | | Shimadzu LC-20AP and UV detector | |
| Column | | Phenomenex Gemini C6 phenyl (250 × 21.2 mm), 5 μm | |
| Flow rate | | 14.0 mL/min | |
| Run time | | 43.0 min | |
| Gradient | | | |
| TIME | Flow Rate | | |
| (min) | (mL/min) | % A start | % B end |
| 0.01 | 14.0 | 70 | 30 |
| 26.00 | 14.0 | 55 | 45 |
| 36.00 | 14.0 | 55 | 45 |
| 36.01 | 14.0 | 0 | 100 |
| 38.00 | 14.0 | 0 | 100 |
| 38.01 | 14.0 | 70 | 30 |
| 43.00 | 14.0 | 70 | 30 |

### Method X16 Method Used for Reverse Phase Preparative HPLC

| Mobile | (A) | 0.1% Ammonia in water | |
|---|---|---|---|
| Phase | (B) | 100% Acetonitrile | |
| Instrument | | Shimadzu LC-20AP and UV detector | |
| Column | | Waters X-Bridge (250 × 19 mm), 5 μm | |
| Flow rate | | 11.0 mL/min | |
| Run time | | 44.0 min | |
| Gradient | | | |
| TIME | Flow Rate | | |
| (min) | (mL/min) | % A start | % B end |
| 0.01 | 11.0 | 80 | 20 |
| 30.00 | 11.0 | 40 | 60 |
| 36.00 | 11.0 | 40 | 60 |
| 36.01 | 11.0 | 0 | 100 |
| 38.00 | 11.0 | 0 | 100 |
| 38.01 | 11.0 | 80 | 20 |
| 44.00 | 11.0 | 80 | 20 |

### Method X17 Method Used for Reverse Phase Preparative HPLC

| Mobile | (A) | 0.1% Ammonia in water | |
|---|---|---|---|
| Phase | (B) | 100% Acetonitrile | |
| Instrument | | Shimadzu LC-20AP and UV detector | |
| Column | | Waters X-Bridge (250 × 19 mm), 5 μm | |
| Flow rate | 15.0 mL/min | Run time | 35.0 min |
| Gradient | | | |
| TIME | Flow Rate | | |
| (min) | (mL/min) | % A start | % B end |
| 0.01 | 15.0 | 90 | 10 |
| 28.00 | 15.0 | 30 | 70 |
| 28.01 | 15.0 | 0 | 100 |
| 30.00 | 15.0 | 0 | 100 |
| 30.01 | 15.0 | 90 | 10 |
| 35.00 | 15.0 | 90 | 10 |

### Method Y4 Method Used for Analytical Chiral SFC

| | | |
|---|---|---|
| Mobile | (A) | Liquid carbon dioxide |
| Phase | (B) | 0.1% Diethylamine in propan-2-ol: acetonitrile (50:50) |

| Instrument | Waters SFC Investigator and PDA detector | | |
|---|---|---|---|
| Column | Chiralpak IH (250 × 4.6 mm), 5 μm | | |
| Flow rate | 4.0 mL/min | Run time | 10.0 min |
| Gradient | | | |
| TIME | Flow Rate | | |
| (min) | (mL/min) | % B start | % B end |
| 0 to 5 | 4.0 | 5 | 50 |
| 5 to 10 | 4.0 | 50 | 50 |

### Method Y8 Method Used for Analytical Chiral SFC

| | | |
|---|---|---|
| Mobile | (A) | Liquid carbon dioxide |
| Phase | (B) | 0.1% Diethylamine in propan-2-ol: acetonitrile (50:50) |

| Instrument | Waters SFC Investigator and PDA detector | | |
|---|---|---|---|
| Column | Chiralpak IC (250 × 4.6 mm), 5 μm | | |
| Flow rate | 4.0 mL/min | Run time | 10.0 min |
| Gradient | | | |
| TIME | Flow Rate | | |
| (min) | (mL/min) | % B start | % B end |
| 0 to 5 | 4.0 | 5 | 50 |
| 5 to 10 | 4.0 | 50 | 50 |

### Method Y9 Method Used for Analytical Chiral HPLC

| Mobile | (A) | Liquid carbon dioxide | |
|---|---|---|---|
| Phase | (B) | 0.1% Diethylamine in propan-2-ol: acetonitrile (50:50) | |
| Instrument | | Waters SFC Investigator and PDA detector | |
| Column | | Chiralpak IB-N (250 × 4.6 mm), 5 μm | |
| Flow rate | | 4.0 mL/min | |
| Run time | | 10.0 min | |
| Gradient | | | |
| TIME | Flow Rate | | |
| (min) | (mL/min) | % B start | % B end |
| 0 to 5 | 4.0 | 5 | 50 |
| 5 to 10 | 4.0 | 50 | 50 |

### Method Y10 Method Used for Analytical Chiral HPLC

| Mobile | (A) | 0.1% Diethylamine in n-hexane | |
|---|---|---|---|
| Phase | (B) | 0.1% Diethylamine in propan-2-ol: acetonitrile | |
| | | (70:30) | |
| Instrument | | Agilent 1260 Series HPLC and PDA detector | |
| Column | | Chiralpak IH (250 × 4.6 mm), 5 μm | |
| Flow rate | 1.0 mL/min | Run time | 25.0 min |
| Gradient | | | |
| TIME | Flow Rate | | |
| (min) | (mL/min) | % A | % B |
| 0.01 | 1.0 | 80 | 20 |
| 5.00 | 1.0 | 50 | 50 |
| 10.00 | 1.0 | 30 | 70 |
| 20.00 | 1.0 | 30 | 70 |
| 20.01 | 1.0 | 80 | 20 |
| 25.00 | 1.0 | 80 | 20 |

### Method Y12A Method Used for Analytical Chiral SFC

| Mobile | (A) | Liquid carbon dioxide | |
|---|---|---|---|
| Phase | (B) | 0.1% Diethylamine in propan-2-ol: acetonitrile (50:50) | |
| Instrument | | Waters SFC Investigator and PDA detector | |
| Column | | Chiralcel OJ-H (250 × 4.6 mm), 5 μm | |
| Flow rate | | 4.0 mL/min | |
| Run time | | 8.0 min | |
| Gradient | | | |
| TIME | Flow Rate | | |
| (min) | (mL/min) | % B start | % B end |
| 0 to 5 | 4.0 | 5 | 50 |
| 5 to 8 | 4.0 | 50 | 50 |

### Method Y14 Method Used for Analytical Chiral SFC

| Mobile | (A) | Liquid carbon dioxide | |
|---|---|---|---|
| Phase | (B) | 0.1% Diethylamine in propan-2-ol: acetonitrile (50:50) | |
| Instrument | | Waters SFC Investigator and PDA detector | |
| Column | | Chiralpak IH (250 × 4.6 mm), 5 μm | |
| Flow rate | | 4.0 mL/min | |
| Run time | | 8.0 min | |
| Gradient | | | |
| TIME | Flow Rate | | |
| (min) | (mL/min) | % B start | % B end |
| 0 to 5 | 4.0 | 5 | 50 |
| 5 to 8 | 4.0 | 50 | 50 |

### Method Y20 Method Used for Analytical Chiral SFC

| Mobile | (A) | Liquid carbon dioxide | |
|---|---|---|---|
| Phase | (B) | 0.1% Diethylamine in propan-2-ol: acetonitrile (50:50) | |
| Instrument | | Waters SFC Investigator and PDA detector | |
| Column | | Chiralpak IH (250 × 4.6 mm), 5 μm | |
| Flow rate | | 4.0 mL/min | |
| Run time | | 9.0 min | |
| Gradient | | | |
| TIME | Flow Rate | | |
| (min) | (mL/min) | % B start | % B end |
| 0 to 5 | 4.0 | 5 | 50 |
| 5 to 9 | 4.0 | 50 | 50 |

### Method Y21 Method Used for Analytical Chiral HPLC

| Mobile | (A) | 0.1% Diethylamine in n-hexane | |
|---|---|---|---|
| Phase | (B) | 0.1% Diethylamine in propan-2-ol: acetonitrile (70:30) | |
| Instrument | | Agilent 1260 series HPLC and PDA detector | |
| Column | | Chiralpak IB-N (250 × 4.6 mm), 5 μm | |
| Flow rate | | 1.0 mL/min | |
| Run time | | 25.0 min | |
| Gradient | | | |
| TIME | Flow Rate | | |
| (min) | (mL/min) | % A | % B |
| 0.01 | 1.0 | 80 | 20 |
| 5.00 | 1.0 | 45 | 55 |
| 10.00 | 1.0 | 30 | 70 |
| 15.00 | 1.0 | 30 | 70 |
| 20.00 | 1.0 | 80 | 20 |
| 25.00 | 1.0 | 80 | 20 |

### Method Y28 Method Used for Analytical Chiral HPLC

| Mobile | (A) | 0.1% Diethylamine in n-hexane | |
|---|---|---|---|
| Phase | (B) | 0.1% Diethylamine in propan-2-ol: acetonitrile (70:30) | |
| Instrument | | Agilent 1100 series HPLC with PDA detector | |
| Column | | Chiralcel OX-H (250 × 4.6 mm), 5 μm | |
| Flow rate | | 1.0 mL/min | |
| Run time | | 25.0 min | |
| Gradient | | | |
| TIME | Flow Rate | | |
| (min) | (mL/min) | % A | % B |
| 0.01 | 1.0 | 80 | 20 |
| 5.00 | 1.0 | 45 | 55 |
| 10.00 | 1.0 | 30 | 70 |
| 15.00 | 1.0 | 30 | 70 |
| 20.00 | 1.0 | 80 | 20 |
| 25.00 | 1.0 | 80 | 20 |

### Method Y30 Method Used for Analytical Chiral HPLC

| Mobile | (A) | 0.1% Diethylamine in n-hexanes | |
|---|---|---|---|
| Phase | (B) | 0.1% Diethylamine in propan-2-ol: acetonitrile (70:30) | |
| Instrument | | Agilent HPLC 1100 series with PDA Detector | |
| Column | | Chiralpak IB-N (250 × 4.6 mm), 5 μm | |
| Flow rate | | 1.0 mL/min | |
| Run time | | 15 min | |
| Isocratic | | | |
| TIME | Flow Rate | | |
| (min) | (mL/min) | % A | % B |
| 0 to 15 | 1.0 | 70 | 30 |

### Method Y31 Method Used for Analytical Chiral HPLC

| Mobile | (A) | 0.1% Diethylamine in n-hexanes | |
|---|---|---|---|
| Phase | (B) | 0.1% Diethylamine in propan-2-ol: acetonitrile (70:30) | |
| Instrument | | Agilent 1260 Infinity series with PDA detector | |
| Column | | Chiralpak IH (250 × 4.6 mm), 5 μm | |
| Flow rate | | 1.0 mL/min | |
| Run time | | 22 min | |
| Isocratic | | | |
| TIME | Flow Rate | | |
| (min) | (mL/min) | % A | % B |
| 0 to 22 | 1.0 | 85 | 15 |

### Method Y32 Method Used for Analytical Chiral HPLC

| Mobile | (A) | Liquid carbon dioxide | |
|---|---|---|---|
| Phase | (B) | 0.1% Diethylamine in propan-2-ol: acetonitrile (50:50) | |
| Instrument | | Waters SFC Investigator with PDA detector | |
| Column | | Chiralpak IH (250 × 4.6 mm), 5 μm | |
| Flow rate | | 4.0 mL/min | |
| Run time | | 12 min | |
| Gradient | | | |
| TIME | Flow Rate | | |
| (min) | (mL/min) | % B start | % B end |
| 0 to 5 | 4.0 | 5 | 50 |
| 5 to 12 | 4.0 | 50 | 50 |

### Method Z1 Method Used for Preparative Chiral HPLC

| Mobile | (A) | 0.1% Diethylamine in n-hexanes | |
|---|---|---|---|
| Phase | (B) | 0.1% Diethylamine in propan-2-ol: acetonitrile (70:30) | |
| Instrument | | Shimadzu LC-20AP with PDA detector | |
| Column | | Chiralpak IB-N (250 × 21 mm), 5 μm | |
| Flow rate | | 20.0 mL/min | |
| Run time | | 23 min | |
| Isocratic | | | |
| TIME | Flow Rate | | |
| (min) | (mL/min) | % A | % B |
| 0 to 23 | 20.0 | 80 | 20 |

### Method Z2 Method Used for Preparative Chiral HPLC

| Mobile | (A) | 0.1% Diethylamine in n-hexanes | |
|---|---|---|---|
| Phase | (B) | 0.1% Diethylamine in propan-2-ol: acetonitrile (70:30) | |
| Instrument | | Shimadzu LC-20AP with PDA detector | |
| Column | | Chiralpak IH (250 × 21 mm), 5 μm | |
| Flow rate | | 20.0 mL/ min | |
| Run time | | 25 min | |
| Isocratic | | | |
| TIME | Flow Rate | | |
| (min) | (mL/min) | % A | % B |
| 0 to 25 | 20.0 | 85 | 15 |

### Synthesis of Intermediates

### Intermediate A

### tert-Butyl (2S,4R)-2-((H-1, 2, 3-triazol-1-yl)methyl)-4-aminopyrrolidine-1-carboxylate

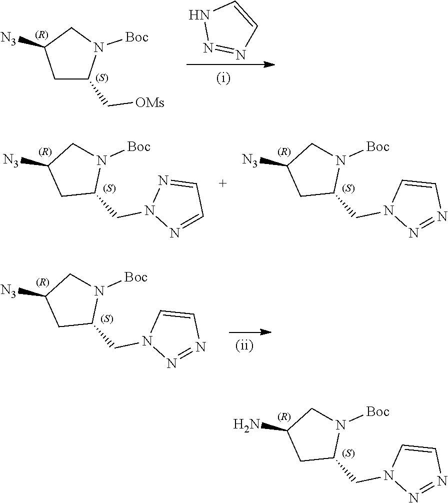

### Step i)

### tert-Butyl (2S,4R)-2-((1H-1, 2, 3-triazol-1-yl)methyl)-4-azidopyrrolidine-1-carboxylate

To a stirred solution of 1H-1,2,3-triazole (CAS 288-36-8, from Spectrochem, 0.76 g, 11.24 mmol) in DMF (30 mL) was added K₂CO₃ (3.88 g, 28.11 mmol) at 0° C. tert-Butyl (2S,4R)-4-azido-2-(((methylsulfonyl)oxy)methyl)pyrrolidine-1-carboxylate (3.0 g, 9.37 mmol) was added at 0° C. The mixture was allowed to warm to rt and stirred for 48 h, then poured into ice-cold water (700 mL) and extracted with EtOAc (5×100 mL). The combined organic phases were washed with ice-cold water (2×100 mL), dried over anhydrous Na₂SO₄ and concentrated under reduced pressure. The residue was purified by column chromatography (silica gel, 15-20% EtOAc in n-hexanes) to yield tert-butyl (2S,4R)-2-((2H-1,2,3-triazol-2-yl)methyl)-4-azidopyrrolidine-1-carboxylate (upper spot, 1.0 g, 3.41 mmol, 36% yield) and subsequently (40 to 50% EtOAc in n-hexanes) tert-butyl (2S,4R)-2-((1H-1,2,3-triazol-1-yl)methyl)-4-azidopyrrolidine-1-carboxylate (lower spot, 1.0 g, 3.41 mmol, 36% yield).

For tert-butyl (2S,4R)-2-((2H-1,2,3-triazol-2-yl)methyl)-4-azidopyrrolidine-1-carboxylate (upper spot, less polar): LCMS: Method C, 1.67 min, MS: ES+ 294.0; ¹H NMR (400 MHz, DMSO-d₆) δ ppm: 7.83 (s, 2H), 4.63-4.65 (m, 2H), 4.02-4.19 (m, 2H), 3.41-3.44 (m, 1H), 3.04-3.14 (m, 1H), 2.00-2.17 (m, 2H), 1.43 (s, 9H).

For tert-butyl (2S,4R)-2-((1H-1,2,3-triazol-1-yl)methyl)-4-azidopyrrolidine-1-carboxylate (lower spot, more polar): LCMS: Method C, 1.52 min, MS: ES+ 294.2; ¹H NMR (400 MHz, DMSO-d₆) δ ppm: 7.96-8.02 (m, 1H), 7.77 (s, 1H), 4.56-4.63 (m, 2H), 4.01-4.21 (m, 2H), 3.41-3.49 (m, 1H), 3.07-3.15 (m, 1H), 1.95-2.08 (m, 2H), 1.39 (s, 9H).

### Step (ii)

### tert-Butyl (2S,4R)-2-((1H-1,2,3-triazol-1-yl)methyl)-4-aminopyrrolidine-1-carboxylate

To a stirred solution of tert-butyl (2S,4R)-2-((1H-1,2,3-triazol-1-yl)methyl)-4-azidopyrrolidine-1-carboxylate (lower spot, 1.0 g, 3.41 mmol) in MeOH (20 mL) was added 10% Pd/C (50% moisture) (0.50 g, 0.5 w/w). The mixture was purged with H₂ gas for 1 h, then filtered through Celite Hyflow® and the filtrate was concentrated under reduced pressure to yield tert-butyl (2S,4R)-2-((1H-1,2,3-triazol-1-yl)methyl)-4-aminopyrrolidine-1-carboxylate (0.85 g, 3.18 mmol, 93% yield).

LCMS: Method C, 1.26 min, MS: ES+ 268.5.

### Intermediate B

### tert-Butyl (2S,4R)-2-((1H-1,2,3-triazol-1-yl)methyl)-4-(cyclopropylamino)pyrrolidine-1-carboxylate

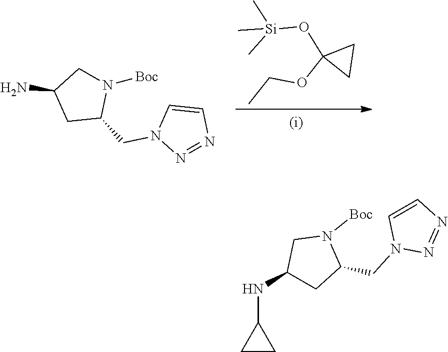

### Step (i)

### tert-Butyl (2S,4R)-2-((1H-1,2,3-triazol-1-yl)methyl)-4-(cyclopropylamino)pyrrolidine-1-carboxylate

To a stirred solution of tert-butyl (2S,4R)-2-((1H-1,2,3-triazol-1-yl)methyl)-4-aminopyrrolidine-1-carboxylate (1.0 g, 3.74 mmol) and (1-ethoxycyclopropoxy)trimethylsilane (0.78 g, 4.49 mmol) in MeOH (5 mL) was added AcOH (0.5 mL) dropwise at 0° C. After 5 min, NaBH₃CN (0.47 g, 7.48 mmol) was added in portions at 0° C. and mixture was heated at 60° C. for 6 h. The mixture was mixed with one more identical batch and was concentrated under reduced pressure. The crude product was then poured into water (100 mL) and extracted with EtOAc (2×100 mL). The combined organic phases were dried over anhydrous Na₂SO₄ and concentrated under reduced pressure. The residue was purified by flash column chromatography (silica gel, 4-5% MeOH in DCM) to yield tert-butyl (2S,4R)-2-((1H-1,2,3-triazol-1-yl)methyl)-4-(cyclopropylamino)pyrrolidine-1-carboxylate (0.38 g, 1.23 mmol, 16% yield). LCMS: Method H1, 2.34 min, MS: ES+ 308.2.

### Intermediate C

### tert-Butyl (2S,4R)-2-((2H-1,2,3-triazol-2-yl)methyl)-4-aminopyrrolidine-1-carboxylate

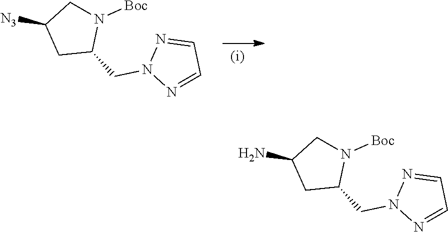 [SMILES: CC(C)(C)OC(=O)N1C[C@H](N=[N+]=[N-])C[C@H]1Cn1nccn1 ; CC(C)(C)OC(=O)N1C[C@H](N)C[C@H]1Cn1nccn1]

### Step (i)

### tert-Butyl (2S,4R)-2-((2H-1,2,3-triazol-2-yl)methyl)-4-aminopyrrolidine-1-carboxylate

To a stirred solution of tert-butyl (2S,4R)-2-((2H-1,2,3-triazol-2-yl)methyl)-4-azidopyrrolidine-1-carboxylate (upper spot, 1.0 g, 3.41 mmol) in MeOH (20 mL) was added 10% Pd/C (50% moisture) (0.50 g, 0.5 w/w). The mixture was purged with H₂ gas for 1 h, then filtered through Celite Hyflow® and the filtrate was concentrated under reduced pressure to yield tert-butyl (2S,4R)-2-((2H-1,2,3-triazol-2-yl)methyl)-4-aminopyrrolidine-1-carboxylate (0.98 g, 3.67 mmol, quantitative yield).

LCMS: Method C, 1.31 min, MS: ES+ 268.5.

### Intermediate D

### tert-Butyl (2S,4R)-2-((1H-1,2,4-triazol-1-yl)methyl)-4-aminopyrrolidine-1-carboxylate

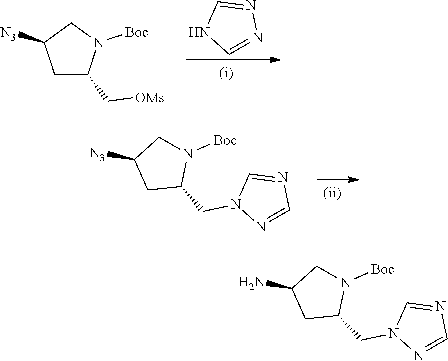

### Step (i)

### tert-Butyl (2S,4R)-2-((1H-1,2,4-triazol-1-yl)methyl)-4-azidopyrrolidine-1-carboxylate

To a stirred solution of 4H-1,2,4-triazole (CAS 288-88-0, from Spectrochem, 0.17 g, 2.40 mmol) in DMF (14 mL) was added K₂CO₃ (0.90 g, 6.56 mmol) at 0° C. tert-Butyl (2S,4R)-4-azido-2-(((methylsulfonyl)oxy)methyl)pyrrolidine-1-carboxylate (0.70 g, 2.18 mmol) was added at 0° C. The mixture was allowed to warm to rt and stirred for 48 h, then poured into ice-cold water (100 mL) and extracted with EtOAc (4×100 mL). The combined organic phases were dried over anhydrous Na₂SO₄ and concentrated under reduced pressure. The residue was purified by column chromatography (silica gel, 30-35% EtOAc in n-hexanes) to yield tert-butyl (2S,4R)-2-((1H-1,2,4-triazol-1-yl)methyl)-4-azidopyrrolidine-1-carboxylate (0.22 g, 0.75 mmol, 34% yield).

LCMS: Method C, 1.53 min, MS: ES+ 294.6; ¹H NMR (400 MHz, DMSO-d₆) δ ppm: 8.43 (m, 1H), 8.01 (s, 1H), 4.34-4.45 (m, 2H), 4.08-4.17 (m, 2H), 3.40-3.49 (m, 1H), 3.15-3.18 (m, 1H), 1.95-2.01 (m, 2H), 1.39 (s, 9H).

### Step (ii)

### tert-Butyl (2S,4R)-2-((1H-1,2,4-triazol-1-yl)methyl)-4-aminopyrrolidine-1-carboxylate

To a stirred solution of tert-butyl (2S,4R)-2-((1H-1,2,4-triazol-1-yl)methyl)-4-azidopyrrolidine-1-carboxylate (0.22 g, 0.75 mmol) in MeOH (2.2 mL) was added 10% Pd/C (50% moisture) (0.11 g, 0.5 w/w). The mixture was purged with H₂ gas for 2 h, then filtered through Celite Hyflow® and the filtrate was concentrated under reduced pressure to yield tert-butyl (2S,4R)-2-((1H-1,2,4-triazol-1-yl)methyl)-4-aminopyrrolidine-1-carboxylate (0.16 g, 0.59 mmol, 80% yield).

LCMS: Method C, 1.23 min, MS: ES+ 268.2.

### Intermediate E

### tert-Butyl (2S,4R)-2-((4H-1,2,4-triazol-4-yl)methyl)-4-aminopyrrolidine-1-carboxylate

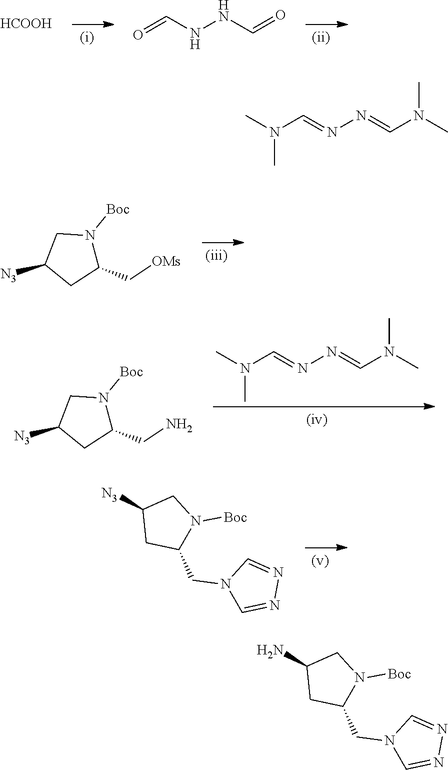

### Step (i)

### N′-Formylformohydrazide

To hydrazine hydrate (99%) (5.00 g, 99.88 mmol) was added formic acid (8.29 mL, 219.73 mmol) dropwise at 0° C. and stirred at 0° C. for 15 min. The mixture was allowed to warm to rt and heated at 120° C. for 6 h. The mixture was cooled to rt and concentrated under reduced pressure. The residue was purified by trituration using diethyl ether (3×50 mL) and dried under reduced pressure to yield N-formylformohydrazide (3.0 g, 34.06 mmol, 34% yield).

¹H NMR (400 MHz, DMSO-d₆) δ ppm: 9.84 (br s, 2H), 8.01 (s, 2H).

### Step (ii)

### (E)-N′—((E)-(Dimethylamino)methylene)-N,N-dimethylformohydrazonamide

To a stirred solution of N′-formylformohydrazide (3.0 g, 34.06 mmol) in DMF (30 mL) was added SOCl₂ (5.46 mL, 74.93 mmol) dropwise at 0° C. The mixture was allowed to warm to rt and stirred at rt for 48 h. The solid was filtered through a Buchner funnel, washed with diethyl ether (3×50 mL) and filtrate was concentrated under reduced pressure. The residue was purified by trituration using diethyl ether (2×50 mL), n-hexanes (2×50 mL) and dried under reduced pressure to yield (E)-N—((E)-(dimethylamino)methylene)-N,N-dimethylformohydrazonamide (4.0 g, 28.12 mmol, 82% yield).

¹H NMR (400 MHz, DMSO-d₆) δ ppm: 8.30 (s, 2H), 2.99 (s, 12H).

Step (iii)

### tert-Butyl (2S,4R)-2-(aminomethyl)-4-azidopyrrolidine-1-carboxylate

An autoclave, charged with a solution of tert-butyl (2S,4R)-4-azido-2-(((methylsulfonyl)oxy)methyl)pyrrolidine-1-carboxylate (4.0 g, 12.48 mmol) in MeOH (40 mL) was purged with NH₃ gas at −78° C. and then mixture was heated at 70° C. for 15 h. The resulting mixture was concentrated under reduced pressure to yield tert-butyl (2S,4R)-2-(aminomethyl)-4-azidopyrrolidine-1-carboxylate (1.80 g, 7.45 mmol, 59% yield).

LCMS: Method C, 1.29 min, MS: ES+ 186.0 (M-56).

### Step (iv)

### tert-Butyl (2S,4R)-2-((4H-1,2,4-triazol-4-yl)methyl)-4-azidopyrrolidine-1-carboxylate

To a stirred solution of tert-butyl (2S,4R)-2-(aminomethyl)-4-azidopyrrolidine-1-carboxylate (1.0 g, 4.14 mmol) and (E)-N—((E)-(dimethylamino)methylene)-N,N-dimethylformohydrazonamide (1.17 g, 4.14 mmol) in toluene (10 mL) were added p-TSA (0.08 g, 0.41 mmol) and 4 Å molecular sieves (0.5 g, 0.5 w/w) at rt, and the mixture was heated at 110° C. for 15 h. The mixture was poured into ice-cold water (200 mL) and extracted with EtOAc (3×200 mL). The combined organic phases were dried over anhydrous Na₂SO₄ and concentrated under reduced pressure. The residue was purified by column chromatography (silica gel, 5% MeOH in DCM) to yield tert-butyl (2S,4R)-2-((4H-1,2,4-triazol-4-yl)methyl)-4-azidopyrrolidine-1-carboxylate (0.49 g, 1.67 mmol, 40% yield).

¹H NMR (400 MHz, DMSO-d₆) δ ppm: 8.42 (s, 2H), 4.11-4.31 (m, 4H), 3.12-3.18 (m, 1H), 1.85-2.12 (m, 3H), 1.42 (s, 9H).

### Step (v)

### tert-Butyl (2S,4R)-2-((4H-1,2,4-triazol-4-yl)methyl)-4-aminopyrrolidine-1-carboxylate

To a stirred solution of tert-butyl (2S,4R)-2-((4H-1,2,4-triazol-4-yl)methyl)-4-azidopyrrolidine-1-carboxylate (0.49 g, 1.67 mmol) in MeOH (5 mL) was added 10% Pd/C (50% moisture) (0.24 g, 0.5 w/w). The mixture was purged with H₂ gas for 2 h, then filtered through Celite Hyflow® and the filtrate was concentrated under reduced pressure to yield tert-butyl (2S,4R)-2-((4H-1,2,4-triazol-4-yl)methyl)-4-aminopyrrolidine-1-carboxylate (0.25 g, 0.93 mmol, 56% yield).

¹H NMR (400 MHz, DMSO-d₆) δ ppm: 8.39 (s, 2H), 4.06-4.21 (m, 3H), 3.12-3.28 (m, 2H), 2.89-3.02 (m, 1H), 1.61-1.81 (m, 4H), 1.43 (s, 9H).

### Intermediate F

### tert-Butyl (2S,4R)-2-((1H-pyrazol-1-yl)methyl)-4-aminopyrrolidine-1-carboxylate

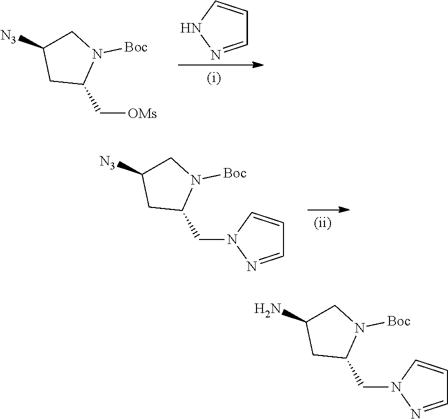 [SMILES: CC(C)(C)OC(=O)N1C[C@H](N=[N+]=[N-])C[C@H]1COS(C)(=O)=O ; c1cn[nH]c1 ; CC(C)(C)OC(=O)N1C[C@H](N=[N+]=[N-])C[C@H]1Cn1cccn1 ; CC(C)(C)OC(=O)N1C[C@H](N)C[C@H]1Cn1cccn1]

### Step (i)

### tert-Butyl (2S,4R)-2-((1H-pyrazol-1-yl)methyl)-4-azidopyrrolidine-1-carboxylate

To a stirred solution of 1H-pyrazole (CAS 288-13-1, from Combi-Blocks, 1.05 g, 15.46 mmol) in DMF (45 mL) was added K₂CO₃ (5.82 g, 42.18 mmol) at 0° C. tert-Butyl (2S,4R)-4-azido-2-(((methylsulfonyl)oxy)methyl)pyrrolidine-1-carboxylate (4.50 g, 14.06 mmol) was added at 0° C. The mixture was allowed to warm to rt and stirred for 48 h, then poured into ice-cold water (100 mL) and extracted with EtOAc (2×150 mL). The combined organic phases were dried over anhydrous Na₂SO₄ and concentrated under reduced pressure. The residue was purified by column chromatography (silica gel, 15% EtOAc in n-hexanes) to yield tert-butyl (2S,4R)-2-((1H-pyrazol-1-yl)methyl)-4-azidopyrrolidine-1-carboxylate (2.42 g, 8.25 mmol, 58% yield).

LCMS: Method J1, 3.92 min, MS: ES+ 293.0.

### Step (ii)

### tert-Butyl (2S,4R)-2-((1H-pyrazol-1-yl)methyl)-4-aminopyrrolidine-1-carboxylate

To a stirred solution of tert-butyl (2S,4R)-2-((1H-pyrazol-1-yl)methyl)-4-azidopyrrolidine-1-carboxylate (0.42 g, 1.43 mmol) in MeOH (4.5 mL) was added 10% Pd/C (50% moisture) (0.21 g, 0.5 w/w). The mixture was purged with H₂ gas for 2 h, then filtered through Celite Hyflow® and the filtrate was concentrated under reduced pressure to yield tert-butyl (2S,4R)-2-((1H-pyrazol-1-yl)methyl)-4-aminopyrrolidine-1-carboxylate (0.35 g, 1.31 mmol, 91% yield).

LCMS: Method J1, 2.64 min, MS: ES+ 267.0.

### Intermediate G

### tert-Butyl (2S,4R)-4-amino-2-((3-methyl-1H-pyrazol-1-yl)methyl)pyrrolidine-1-carboxylate & tert-Butyl (2S,4R)-4-amino-2-((5-methyl-1H-pyrazol-1-yl)methyl)pyrrolidine-1-carboxylate

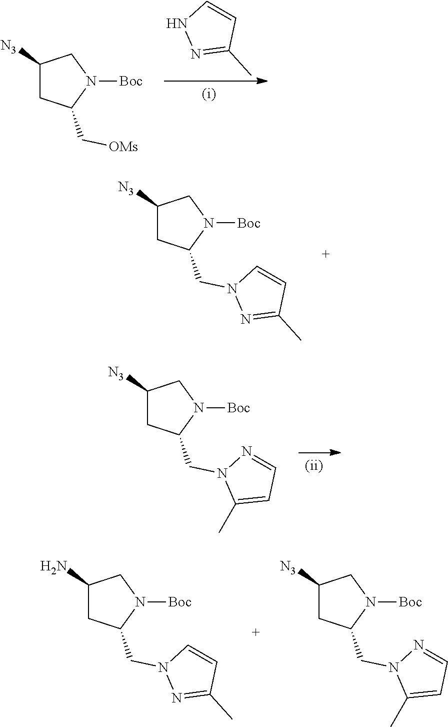

### Step (i)

### tert-Butyl (2S,4R)-4-azido-2-((3-methyl-1H-pyrazol-1-yl)methyl)pyrrolidine-1-carboxylate & tert-Butyl (2S,4R)-4-azido-2-((5-methyl-1H-pyrazol-1-yl)methyl)pyrrolidine-1-carboxylate

To a stirred solution of 3-methyl-1H-pyrazole (CAS 1453-58-3, from Combi-Blocks, 0.77 g, 9.4 mmol) in DMF (30 mL) was added NaH (60% in oil) (0.47 g, 9.8 mmol) in portions at 0° C. tert-Butyl (2S,4R)-4-azido-2-(((methylsulfonyl)oxy)methyl)pyrrolidine-1-carboxylate (1.37 g, 4.27 mmol) was added at 0° C. The mixture was allowed to warm to rt and stirred for 18 h, then poured into ice-cold water (200 mL) and extracted with EtOAc (3×100 mL). The combined organic phases were washed with ice-cold water (2×100 mL), dried over anhydrous Na₂SO₄, filtered and concentrated under reduced pressure. The residue was purified by column chromatography (silica gel, 15-20% EtOAc in n-hexanes) to yield tert-butyl (2S,4R)-4-azido-2-((3-methyl-1H-pyrazol-1-yl)methyl)pyrrolidine-1-carboxylate & tert-butyl (2S,4R)-4-azido-2-((5-methyl-1H-pyrazol-1-yl)methyl)pyrrolidine-1-carboxylate as a mixture (0.97 g, 3.18 mmol, 74% yield).

LCMS: Method H1, 3.17 min, MS: ES+ 307.0.

### Step (ii)

### tert-Butyl (2S,4R)-4-amino-2-((3-methyl-1H-pyrazol-1-yl)methyl)pyrrolidine-1-carboxylate &

### tert-Butyl (2S,4R)-4-amino-2-((5-methyl-1H-pyrazol-1-yl)methyl)pyrrolidine-1-carboxylate

To a stirred solution of tert-butyl (2S,4R)-4-azido-2-((3-methyl-1H-pyrazol-1-yl)methyl)pyrrolidine-1-carboxylate & tert-butyl (2S,4R)-4-azido-2-((5-methyl-1H-pyrazol-1-yl)methyl)pyrrolidine-1-carboxylate as a mixture (0.97 g, 3.18 mmol) in MeOH (20 mL) was added 10% Pd/C (50% moisture) (0.47 g, 0.5 w/w). The mixture was purged with H₂ gas for 2 h, then filtered through Celite Hyflow® and the filtrate was concentrated under reduced pressure to yield tert-butyl (2S,4R)-4-amino-2-((3-methyl-1H-pyrazol-1-yl)methyl)pyrrolidine-1-carboxylate & tert-butyl (2S,4R)-4-amino-2-((5-methyl-1H-pyrazol-1-yl)methyl)pyrrolidine-1-carboxylate as a mixture (0.73 g, quantitative yield).

LCMS: Method H, 2.21 min, MS: ES+ 281.0.

### Intermediate H

### Lithium 5-(5-cyano-2-cyclopropylphenyl)oxazole-2-carboxylate

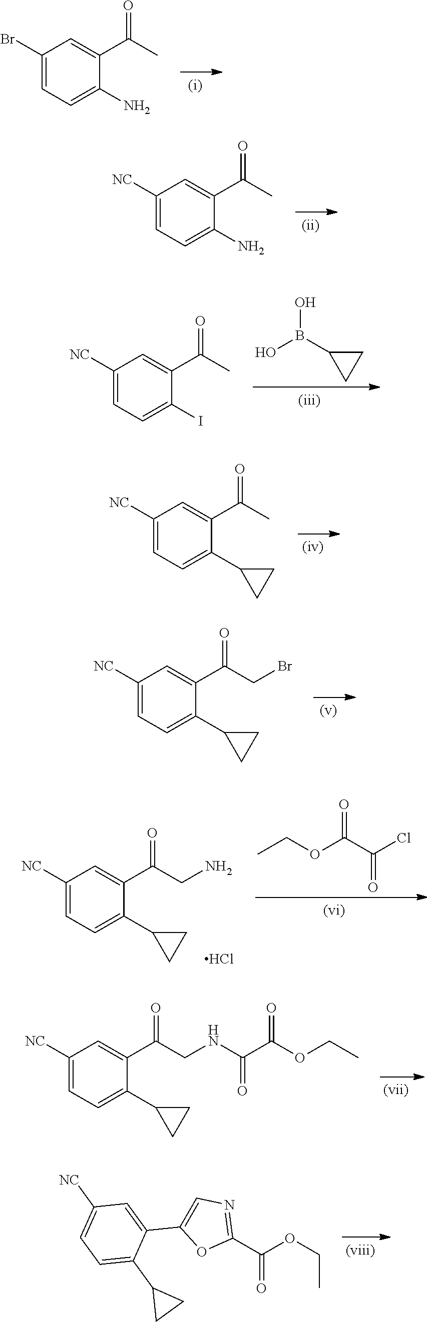

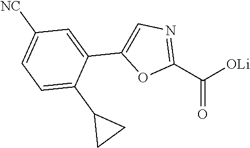

### Step (i)

### 3-Acetyl-4-aminobenzonitrile

A stirred solution of 1-(2-amino-5-bromophenyl)ethan-1-one (CAS 29124-56-9, from Combi-Blocks, 24.0 g, 112.68 mmol) in DMF (250 ml) was purged with N₂ gas for 15 min, followed by addition of CuCN (11.09 g, 123.94 mmol) and heated at 170° C. for 16 h. The mixture was poured into water (500 mL) and extracted with EtOAc (2×500 mL). The combined organic phases were dried over anhydrous Na₂SO₄ and concentrated under reduced pressure. The residue was purified by column chromatography (silica gel, 12% EtOAc in n-hexanes) to yield 3-acetyl-4-aminobenzonitrile (10.5 g, 65.62 mmol, 58% yield). LCMS: Method H1, 2.39 min, MS: ES+ 161.0.

### Step (ii)

### 3-Acetyl-4-iodobenzonitrile

To a stirred solution of 3-acetyl-4-aminobenzonitrile (9.5 g, 59.37 mmol) in conc. HCl (100 mL) was added NaNO₂ (8.19 g, 118.75 mmol) in portions at 0° C. and stirred for 10 min. A solution of KI (24.63 g, 148.42 mmol) in water (100 mL) was added dropwise at 0° C. and stirred for 0.5 h. The mixture was poured into water (200 mL) and extracted with EtOAc (2×100 mL). The combined organic phases were washed with 10% sodium thiosulphate solution (2×100 mL), dried over anhydrous Na₂SO₄ and concentrated under reduced pressure. The residue was purified by column chromatography (silica gel, 10% EtOAc in n-hexanes) to yield 3-acetyl-4-iodobenzonitrile (12.5 g, 46.13 mmol, 77% yield).

¹H NMR (400 MHz, DMSO-d₆) δ ppm: 8.18-8.21 (m, 2H), 7.67 (dd, J=8.0, 2.0 Hz, 1H), 2.61 (s, 3H).

Step (iii)

### 3-Acetyl-4-cyclopropylbenzonitrile

To a stirred solution of 3-acetyl-4-iodobenzonitrile (12.5 g, 46.13 mmol) and cyclopropylboronic acid (CAS 411235-57-9, from Combi-Blocks, 7.92 g, 92.26 mmol) in toluene: water (130 mL, 8:2) was added K₃PO₄ (19.55 g, 92.26 mmol). The mixture was purged with N₂ gas for 10 min, followed by addition of [1,1′-bis(diphenylphosphino)ferrocene]dichloropalladium(II) (3.37 g, 4.61 mmol) and heated at 100° C. for 5 h. The mixture was poured into water (400 mL) and extracted with EtOAc (2×400 mL). The combined organic phases were dried over anhydrous Na₂SO₄ and concentrated under reduced pressure. The residue was purified by column chromatography (silica gel, 10% EtOAc in n-hexanes) to yield 3-acetyl-4-cyclopropylbenzonitrile (4.8 g, 25.94 mmol, 56% yield).

LCMS: Method H1, 3.01 min, MS: ES+ 186.0.

### Step (iv)

### 3-(2-Bromoacetyl)-4-cyclopropylbenzonitrile

To a stirred solution of 3-acetyl-4-cyclopropylbenzonitrile (4.8 g, 25.94 mmol) in THF (50 mL) was added phenyl trimethylammonium tribromide (10.72 g, 28.53 mmol) in portions at rt and stirred for 16 h. The mixture was poured into water (200 mL) and extracted with EtOAc (2×200 mL). The combined organic phases were dried over Na₂SO₄ and concentrated under reduced pressure. The residue was purified by column chromatography (silica gel, 5% EtOAc in n-hexanes) to yield 3-(2-bromoacetyl)-4-cyclopropylbenzonitrile (4.5 g, 17.11 mmol, 65% yield).

LCMS: Method H1, 3.25 min, MS: ES− 262.0, 264.0.

### Step (v)

### 4-Cyclopropyl-3-glycylbenzonitrile HCl Salt

To a stirred solution of 3-(2-bromoacetyl)-4-cyclopropylbenzonitrile (4.5 g, 17.11 mmol) in MeCN (50 mL) was added sodium diformylamide (1.97 g, 20.53 mmol) and heated at 80° C. for 1 h. The mixture was cooled to rt and concentrated under reduced pressure. The residue was diluted with MeOH (50 mL) and conc. HCl (4.5 mL). The mixture was further heated at 80° C. for 3 h, then allowed to cool to rt. The mixture was concentrated under reduced pressure, and the residue was stirred with diethyl ether (40 mL) to form a precipitate. The solid was collected by filtration under reduced pressure to yield 4-cyclopropyl-3-glycylbenzonitrile HCl salt (5.5 g, quantitative yield).

LCMS: Method H1, 2.31 min, MS: ES+ 201.0.

### Step (vi)

### Ethyl 2-((2-(5-cyano-2-cyclopropylphenyl)-2-oxoethyl)amino)-2-oxoacetate

To a stirred solution of 4-cyclopropyl-3-glycylbenzonitrile HCl salt (5.5 g, 23.25 mmol) in DCM (60 mL) at 0° C. was added K₂CO₃ (12.83 g, 93.0 mmol). Ethyl oxalyl chloride (6.32 g, 5.17 mL, 46.5 mmol) was added dropwise at 0° C. The mixture was allowed to warm to rt, stirred for 1 h, then poured into water (200 mL) and extracted with EtOAc (2×200 mL). The combined organic phases were dried over Na₂SO₄ and concentrated under reduced pressure to yield ethyl 2-((2-(5-cyano-2-cyclopropylphenyl)-2-oxoethyl)amino)-2-oxoacetate (3.3 g, 11.0 mmol, 64% yield over two steps).

LCMS: Method H1, 2.84 min, MS: ES+ 301.0.

Step (vii)

### Ethyl 5-(5-cyano-2-cyclopropylphenyl)oxazole-2-carboxylate

A stirred solution of ethyl 2-((2-(5-cyano-2-cyclopropylphenyl)-2-oxoethyl)amino)-2-oxoacetate (3.3 g, 11.0 mmol) in POCl₃ (33 mL, 10 vol) was heated at 100° C. for 5 h. The mixture was cooled to rt, slowly poured into crushed ice and extracted with EtOAc (2×100 mL). The combined organic phases were dried over Na₂SO₄ and concentrated under reduced pressure. The residue was purified by flash column chromatography (silica gel, 15% EtOAc in n-hexanes) to yield ethyl 5-(5-cyano-2-cyclopropylphenyl)oxazole-2-carboxylate (1.2 g, 4.25 mmol, 39% yield).

LCMS: Method H1, 3.38 min, MS: ES+ 283.0.

Step (viii)

### Lithium 5-(5-cyano-2-cyclopropylphenyl)oxazole-2-carboxylate

To a stirred solution of ethyl 5-(5-cyano-2-cyclopropylphenyl)oxazole-2-carboxylate (0.5 g, 1.77 mmol) in THF (6 mL) was added a solution of LiOH·H₂O (0.15 g, 3.54 mmol) in water (1 mL) dropwise at 0° C. and stirred at rt for 3 h. The reaction mixture was concentrated under reduced pressure to yield lithium 5-(5-cyano-2-cyclopropylphenyl)oxazole-2-carboxylate (0.50 g, quantitative yield).

LCMS: Method H1, 2.14 min, MS: ES+ 255.0.

### Intermediate I

### Lithium 5-(5-cyano-2-cyclopropylphenyl)-1,3,4-oxadiazole-2-carboxylate

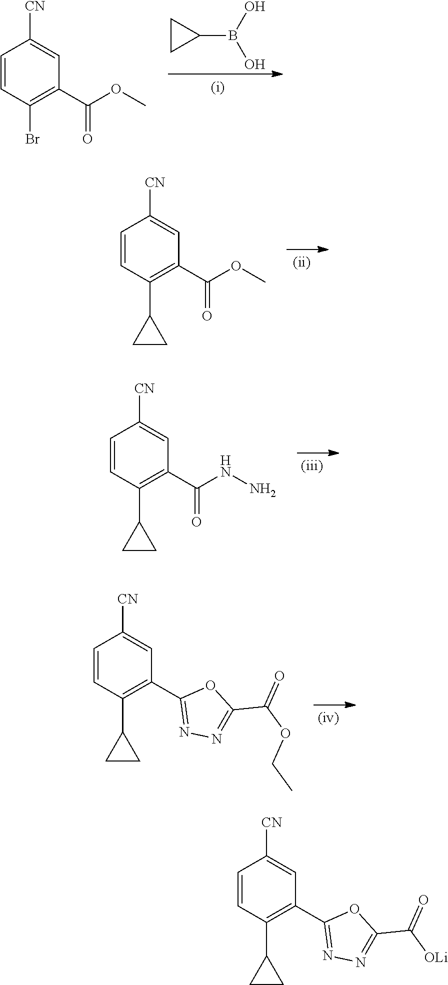

### Step (i)

### Methyl 5-cyano-2-cyclopropylbenzoate

To a stirred solution of methyl 2-bromo-5-cyanobenzoate (CAS 1031927-03-3, from Reddy N Reddy, 1.5 g, 6.27 mmol) and cyclopropylboronic acid (CAS 411235-57-9, from Combi-Blocks, 0.59 g, 6.90 mmol) in toluene:water (15 mL, 9:1) was added K₃PO₄ (2.65 g, 12.54 mmol). The mixture was purged with N₂ gas for 15 min, followed by addition of [1,1′-bis(diphenylphosphino)ferrocene]dichloropalladium(II) (0.45 g, 0.627 mmol) and heated at 100° C. for 3 h. The mixture was poured into water (30 mL) and extracted with EtOAc (2×50 mL). The combined organic phases were dried over anhydrous Na₂SO₄, filtered and concentrated under reduced pressure. The residue was purified by flash column chromatography (silica gel, 5% EtOAc in n-hexanes) to yield methyl 5-cyano-2-cyclopropylbenzoate (1.0 g, 4.97 mmol, 79% yield).

LCMS: Method H1, 3.19 min, MS: ES+ 219.0 (M+18).

### Step (ii)

### 5-Cyano-2-cyclopropylbenzohydrazide

To a stirred solution of methyl 5-cyano-2-cyclopropylbenzoate (1.0 g, 4.97 mmol) in EtOH (10 mL) was added hydrazine hydrate (99%) (5 mL, 5 vol) at 0° C. The mixture was allowed to warm to rt and stirred for 3 h. The mixture was concentrated under reduced pressure to yield 5-cyano-2-cyclopropylbenzohydrazide (0.7 g, 3.48 mmol, 70% yield).

LCMS: Method H1, 1.96 min, MS: ES+ 202.0.

Step (iii)

### Ethyl 5-(5-cyano-2-cyclopropylphenyl)-1,3,4-oxadiazole-2-carboxylate

To a stirred solution of 5-cyano-2-cyclopropylbenzohydrazide (0.7 g, 3.48 mmol) in DCM (7 mL) were added TEA (2.11 g, 2.90 mL, 20.89 mmol) and ethyl oxalyl chloride (0.1 g, 0.85 mL, 7.66 mmol) dropwise at 0° C. The mixture was allowed to warm to rt and stirred for 1 h. TsCl (0.79 g, 4.17 mmol) was added in portions at 0° C. The mixture was allowed to warm to rt and stirred at rt for 2 h, then poured into water (30 mL) and extracted with DCM (3×100 mL). The combined organic phases were dried over anhydrous Na₂SO₄, filtered and concentrated under reduced pressure. The residue was purified using flash column chromatography (silica gel, 40% EtOAc in n-hexanes) to yield ethyl 5-(5-cyano-2-cyclopropylphenyl)-1,3,4-oxadiazole-2-carboxylate (0.3 g, 1.05 mmol, 30% yield).

LCMS: Method H1, 3.20 min, MS: ES+ 284.0.

### Step (vi)

### Lithium 5-(5-cyano-2-cyclopropylphenyl)-1,3,4-oxadiazole-2-carboxylate

To a stirred solution of ethyl 5-(5-cyano-2-cyclopropylphenyl)-1,3,4-oxadiazole-2-carboxylate (0.24 g, 0.85 mmol) in THF (4 mL) at 0° C. was added a solution of lithium hydroxide monohydrate (0.03 g, 0.85 mmol) in water (1 mL) dropwise and stirred at rt for 2 h. The mixture was concentrated under reduced pressure to yield lithium 5-(5-cyano-2-cyclopropylphenyl)-1,3,4-oxadiazole-2-carboxylate (0.25 g, quantitative yield).

LCMS: Method H1, 1.94 min, MS: ES+ 256.0.

Intermediate J

### 5-(3-Chlorophenyl)oxazole-2-carboxylic acid

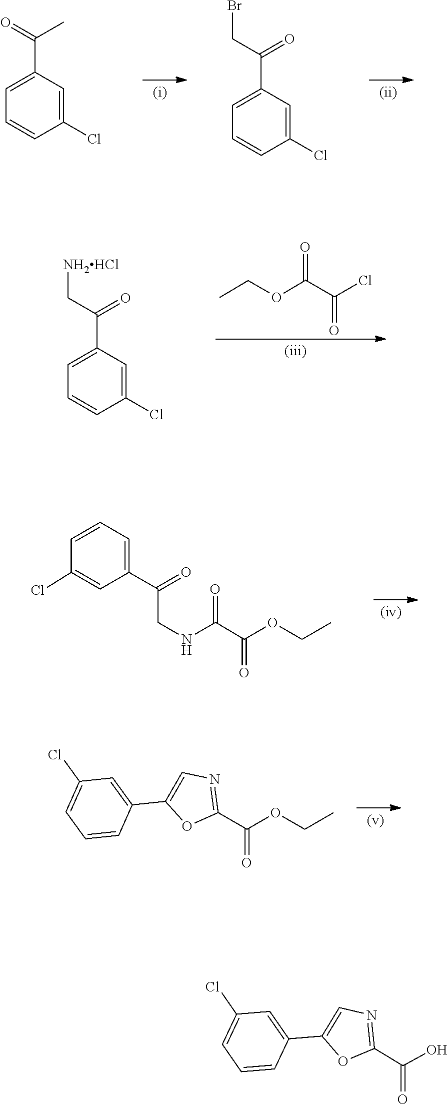

### Step (i)

### 2-Bromo-1-(3-chlorophenyl)ethan-1-one

To a stirred solution of 1-(3-chlorophenyl)ethan-1-one (CAS 99-02-5, from Sigma-Aldrich, 2.0 g, 12.98 mmol) in DCM (40 mL) was added phenyl trimethylammonium tribromide (4.88 g, 12.98 mmol) at 0° C. The mixture was allowed to warm to rt and stirred for 2 h, then poured into water (50 mL) and extracted with DCM (2×50 mL). The combined organic phases were dried over Na₂SO₄ and concentrated under reduced pressure to yield 2-bromo-1-(3-chlorophenyl)ethan-1-one (1.1 g, 4.74 mmol, 36% yield). This crude material was used directly in the next step.

### Step (ii)

### 2-Amino-1-(3-chlorophenyl)ethan-1-one HCl salt

To a stirred solution of 2-bromo-1-(3-chlorophenyl)ethan-1-one (1.0 g, 4.31 mmol) in MeCN (25 mL) was added sodium diformylamide (0.62 g, 6.46 mmol) and heated at 80° C. for 12 h. The reaction mixture was cooled to rt and concentrated under reduced pressure. The residue was diluted with MeOH (5 mL) and conc. HCl (2 mL). The mixture was further heated at 60° C. for 2 h, then allowed to cool to rt. The mixture was concentrated under reduced pressure, and the residue was stirred with n-hexanes (25 mL) to form a precipitate. The solid was collected by filtration under reduced pressure to yield 2-amino-1-(3-chlorophenyl)ethan-1-one HCl salt (0.8 g, 3.89 mmol, 90% yield).

LCMS: Method C1, 0.89 min, MS: ES+: 170.2.

Step (iii)

### Ethyl 2-((2-(3-chlorophenyl)-2-oxoethyl)amino)-2-oxoacetate

To a stirred solution of 2-amino-1-(3-chlorophenyl)ethan-1-one HCl salt (0.8 g, 3.89 mmol) in DCM (20 mL) was added K₂CO₃ (1.61 g, 11.67 mmol) at 0° C. Ethyl oxalyl chloride (0.79 g, 0.65 mL, 5.83 mmol) was added dropwise at 0° C. The mixture was allowed to warm to rt, stirred for 2 h, then poured into ice-cold water (50 mL) and extracted with EtOAc (2×50 mL). The combined organic phases were dried over Na₂SO₄ and concentrated under reduced pressure. The residue was purified by flash column chromatography (silica gel, 20% EtOAc in n-hexanes) to yield ethyl 2-((2-(3-chlorophenyl)-2-oxoethyl)amino)-2-oxoacetate (0.7 g, 2.60 mmol, 66% yield).

LCMS: Method C1, 1.22 min, MS: ES+ 270.3.

### Step (iv)

### Ethyl 5-(3-chlorophenyl)oxazole-2-carboxylate

A stirred solution of ethyl 2-((2-(3-chlorophenyl)-2-oxoethyl)amino)-2-oxoacetate (0.7 g, 2.60 mmol) in POCl₃ (15 mL, 20 vol) was heated at 100° C. for 16 h. The mixture was cooled to rt, poured into ice-cold water (100 mL) and extracted with EtOAc (2×200 mL). The combined organic phases were dried over Na₂SO₄ and concentrated under reduced pressure. The residue was suspended in MeOH (10 mL) and stirred at −78° C. for 15 min. The solid was collected by filtration under reduced pressure to afford ethyl 5-(3-chlorophenyl)oxazole-2-carboxylate (0.4 g, 1.59 mmol, 61% yield).

LCMS: Method C1, 1.35 min, MS: ES+: 252.2.

### Step (v)

### 5-(3-Chlorophenyl)oxazole-2-carboxylic acid

To a stirred solution of ethyl 5-(3-chlorophenyl)oxazole-2-carboxylate (0.2 g, 0.79 mmol) in THF water (3:1, 4 mL) was added lithium hydroxide monohydrate (0.17 g, 3.98 mmol) in portions at 0° C. The mixture was stirred at rt for 2 h, then poured into water (20 mL), acidified with 1N HCl to pH ˜2 to for a precipitate. The solid was collected by filtration under reduced pressure to yield 5-(3-chlorophenyl)oxazole-2-carboxylic acid (0.15 g, 0.67 mmol, 84% yield).

LCMS: Method H1, 1.93 min, MS: ES+ 224.0.

### Intermediate K

### 5-(2-Methoxyphenyl)oxazole-2-carboxylic acid

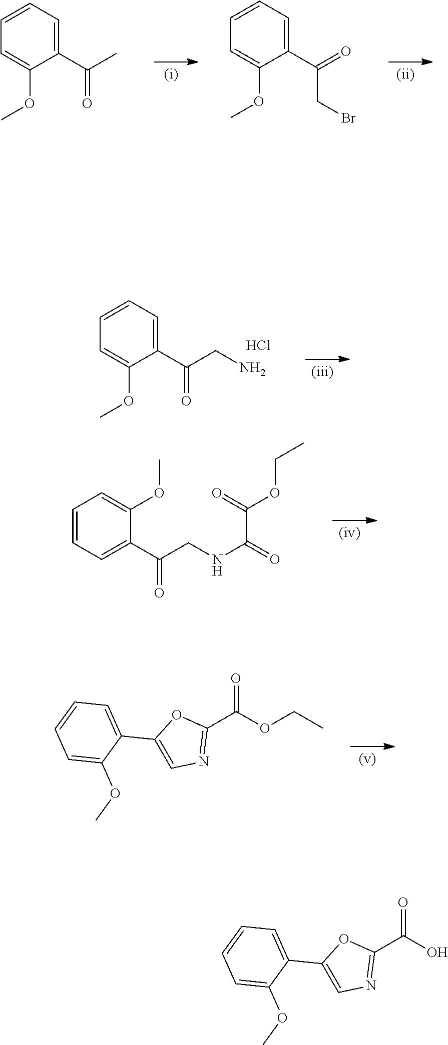

### Step (i)

### 2-Bromo-1-(2-methoxyphenyl) ethan-1-one

To a stirred solution of 1-(2-methoxyphenyl) ethan-1-one (CAS 579-74-8, from Spectrochem, 2.0 g, 13.31 mmol) in THF (40 mL) was added phenyl trimethylammonium tribromide (5.0 g, 13.31 mmol) in portions at 0° C. The mixture was allowed to warm to rt and stirred for 16 h, then poured into water (50 mL) and extracted with DCM (2×50 mL). The combined organic phases were dried over Na₂SO₄, filtered and concentrated under reduced pressure to yield 2-bromo-1-(2-methoxyphenyl) ethan-1-one (2 g, 8.73 mmol, 65% yield).

LCMS: Method F, 6.41 min, MS: ES+: 229.0, 231.0.

### Step (ii)

### 2-Amino-1-(2-methoxyphenyl)ethan-1-one HCl salt

To a stirred solution of 2-bromo-1-(2-methoxyphenyl)ethan-1-one (2.0 g, 8.73 mmol) in MeCN (50 mL) was added sodium diformylamide (1.25 g, 13.09 mmol) and heated at 80° C. for 12 h. The reaction mixture was cooled to rt and concentrated under reduced pressure. The residue was diluted with MeOH (20 mL) and conc. HCl (10 mL). The mixture was further heated at 80° C. for 3 h, then allowed to cool to rt. The mixture was concentrated under reduced pressure, and the residue was stirred with n-hexanes (50 mL) to form a precipitate. The solid was collected by filtration under reduced pressure to yield 2-amino-1-(2-methoxyphenyl)ethan-1-one HCl salt (1.2 g, 7.26 mmol, 83% yield).

LCMS: Method H1, 1.80 min, MS: ES+: 166.0.

Step (iii)

### Ethyl 2-((2-(2-methoxyphenyl)-2-oxoethyl)amino)-2-oxoacetate

To a stirred solution of 2-amino-1-(2-methoxyphenyl)ethan-1-one HCl salt (1.2 g, 7.26 mmol) in DCM (30 mL) was added K₂CO₃ (3.0 g, 21.78 mmol) at 0° C. Ethyl oxalyl chloride (1.48 g, 1.20 mL, 10.89 mmol) was added dropwise at 0° C. The mixture was allowed to warm to rt, stirred for 3 h, then poured into ice-cold water (50 mL) and extracted with EtOAc (2×50 mL). The combined organic phases were dried over Na₂SO₄ and concentrated under reduced pressure to yield ethyl 2-((2-(2-methoxyphenyl)-2-oxoethyl)amino)-2-oxoacetate (1.0 g, 3.76 mmol, 51% yield).

LCMS: Method H1, 2.64 min, MS: ES+ 266.0.

### Step (iv)

### Ethyl 5-(2-methoxyphenyl)oxazole-2-carboxylate

To a stirred solution of ethyl 2-((2-(2-methoxyphenyl)-2-oxoethyl)amino)-2-oxoacetate (1.0 g, 3.76 mmol) in POCl₃ (20 mL, 20 vol) was heated at 100° C. for 3 h. The mixture was cooled to rt, poured into ice-cold water (100 mL) and extracted with EtOAc (2×200 mL). The combined organic phases were dried over Na₂SO₄ and concentrated under reduced pressure to yield ethyl 5-(2-methoxyphenyl)oxazole-2-carboxylate (0.8 g, 3.23 mmol, 85% yield).

LCMS: Method H1, 3.19 min, MS: ES+: 248.0.

### Step (v)

### 5-(2-Methoxyphenyl)oxazole-2-carboxylic acid

To a stirred solution of ethyl 5-(2-methoxyphenyl)oxazole-2-carboxylate (0.8 g, 3.23 mmol) in THF: water (6 mL, 2:1) was added LiOH·H₂O (0.81 g, 19.42 mmol) in portions at 0° C. The mixture was stirred at rt for 12 h, then poured into water (20 mL), acidified with 1N HCl to pH ˜2 to form a precipitate. The solid was collected by filtration under reduced pressure to yield 5-(2-methoxyphenyl)oxazole-2-carboxylic acid (0.6 g, 2.73 mmol, 84% yield).

LCMS: Method C1, 1.00 min, MS: ES+ 220.1.

### Intermediate L

### Ethyl 5-(2-cyclopropylphenyl)-1,3,4-oxadiazole-2-carboxylate

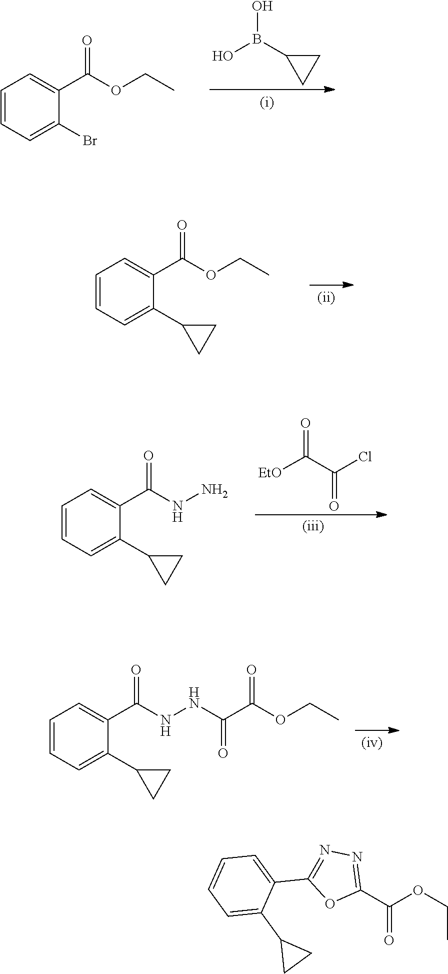

### Step (i)

### Ethyl 2-cyclopropylbenzoate

To a stirred solution of ethyl 2-bromobenzoate (CAS 6091-64-1, from Combi-Blocks, 8.0 g, 35.09 mmol) and cyclopropylboronic acid (CAS 411235-57-9, from Combi-Blocks, 3.01 g, 35.09 mmol) in toluene:water (80 mL, 9:1) was added K₃PO₄ (14.89 g, 70.18 mmol). The mixture was purged with N₂ gas for 20 min, followed by addition of [1,1′-bis(diphenylphosphino)ferrocene]-dichloropalladium(II) (2.56 g, 3.50 mmol) and heated at 120° C. for 3 h. The mixture was poured into water (50 mL) and extracted with EtOAc (2×40 mL). The combined organic phases were dried over anhydrous Na₂SO₄, filtered and concentrated under reduced pressure. The residue was purified by flash column chromatography (silica gel, 6% EtOAc in n-hexanes) to yield ethyl 2-cyclopropylbenzoate (5.7 g, 29.98 mmol, 85% yield).

LCMS: Method C1, 1.14 min, MS: ES+ 191.0.

### Step (ii)

### 2-Cyclopropylbenzohydrazide

To a stirred solution of ethyl 2-cyclopropylbenzoate (5.56 g, 29.24 mmol) in EtOH (28 mL) was added hydrazine hydrate (99%) (16.68 mL, 3 vol) at 0° C. and the mixture was heated at 80° C. for 48 h. The mixture was concentrated under reduced pressure to yield 2-cyclopropylbenzohydrazide (4.5 g, 25.55 mmol, 87% yield).

LCMS: Method C1, 0.97 min, MS: ES+ 177.2.

Step (iii)

### Ethyl 2-(2-(2-cyclopropylbenzoyl)hydrazineyl)-2-oxoacetate

To a stirred solution of 2-cyclopropylbenzohydrazide (4.4 g, 24.98 mmol) in DCM (44 mL) were added TEA (7.57 g, 10.42 mL, 74.94 mmol) and ethyl oxalyl chloride (6.82 g, 5.59 mL, 49.96 mmol) dropwise at 0° C. The mixture was allowed to warm to rt and stirred for 3 h, then poured into water (120 mL) and extracted with DCM (3×50 mL). The combined organic phases were dried over Na₂SO₄, filtered and concentrated under reduced pressure to yield ethyl 2-(2-(2-cyclopropylbenzoyl)hydrazineyl)-2-oxoacetate (13.0 g, quantitative yield).

LCMS: Method C1, 1.09 min, MS: ES+ 277.2.

### Step (iv)

### Ethyl 5-(2-cyclopropylphenyl)-1,3,4-oxadiazole-2-carboxylate

To a stirred solution of ethyl 2-(2-(2-cyclopropylbenzoyl)hydrazineyl)-2-oxoacetate (13 g, 47.08 mmol) in DCM (130 mL) was added TEA (14.26 g, 19.64 mL, 141.24 mmol) and TsCl (10.72 g, 56.49 mmol) in portions at 0° C. The mixture was allowed to warm at rt and stirred for 3 h, then poured into water (100 mL) and extracted with DCM (3×100 mL). The combined organic phases were dried over anhydrous Na₂SO₄, filtered and concentrated under reduced pressure. The residue was purified using flash column chromatography (silica gel, 20% EtOAc in n-hexanes) to yield ethyl 5-(2-cyclopropylphenyl)-1,3,4-oxadiazole-2-carboxylate (2.97 g, 11.49 mmol, 46% yield over two steps).

LCMS: Method C1, 1.36 min, MS: ES+ 259.16.

Intermediate M Lithium 5-(2-cyclopropylphenyl)oxazole-2-carboxylate

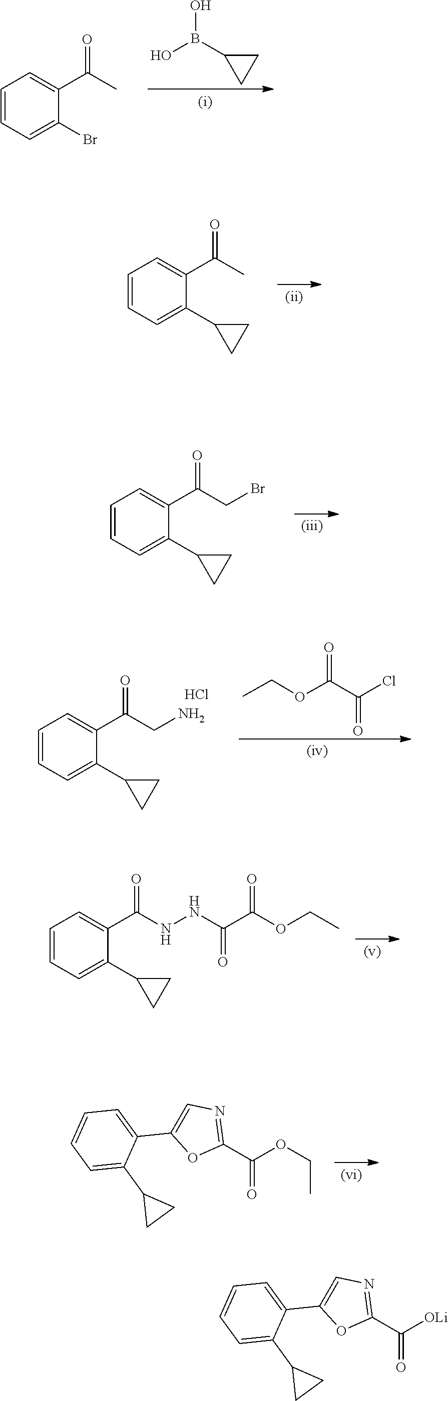

### Step (i)

### 1-(2-Cyclopropylphenyl)ethan-1-one

To a stirred solution of 1-(2-bromophenyl)ethan-1-one (CAS 2142-69-0, from Combi-Blocks, 10.0 g, 50.51 mmol) and cyclopropylboronic acid (CAS 411235-57-9, from Combi-Blocks, 8.67 g, 101.02 mmol) intoluene:water (100 mL, 8:2) was added K₃PO₄ (21.41 g, 101.02 mmol). The mixture was purged with N₂ gas for 10 min, followed by addition of [1,1′-bis(diphenylphosphino)ferrocene]-dichloropalladium(II) (3.69 g, 5.05 mmol) and heated at 100° C. for 5 h. The mixture was poured into water (300 mL) and extracted with EtOAc (2×300 mL). The combined organic phases were dried over anhydrous Na₂SO₄ and concentrated under reduced pressure. The residue was purified by column chromatography (silica gel, 1 to 2% EtOAc in n-hexanes) to yield 1-(2-cyclopropylphenyl)ethan-1-one (6.0 g, 37.5 mmol, 74% yield).

LCMS: Method H1, 3.15 min, MS: ES+ 161.0.

### Step (ii)

### 2-Bromo-1-(2-cyclopropylphenyl)ethan-1-one

To a stirred solution of 1-(2-cyclopropylphenyl)ethan-1-one (6.0 g, 37.5 mmol) in THF (70 mL) was added phenyl trimethylammonium tribromide (15.50 g, 41.25 mmol) in portions at 0° C. The mixture was allowed to warm to rt and stirred for 16 h, then poured into water (200 mL) and extracted with EtOAc (2×200 mL). The combined organic phases were dried over Na₂SO₄ and concentrated under reduced pressure. The residue was purified by column chromatography (silica gel, 1% EtOAc in n-hexanes) to yield 2-bromo-1-(2-cyclopropylphenyl)ethan-1-one (6.2 g, 26.05 mmol, 69% yield).

LCMS: Method H1, 3.38 min, MS: ES+ 239.0, 240.9.

Step (iii)

### 2-Amino-1-(2-cyclopropylphenyl)ethan-1-one HCl salt

To a stirred solution of 2-bromo-1-(2-cyclopropylphenyl)ethan-1-one (6.2 g, 26.05 mmol) in MeCN (60 mL) was added sodium diformylamide (3.0 g, 31.26 mmol) and heated at 80° C. for 3 h. The reaction mixture was cooled to rt and concentrated under reduced pressure. The residue was diluted with MeOH (60 mL) and conc. HCl (6.5 mL). The mixture was further heated at 80° C. for 16 h, then allowed to cool to rt. The mixture was concentrated under reduced pressure, and the residue was stirred with diethyl ether (50 mL) to form a precipitate. The solid was collected by filtration under reduced pressure to yield 2-amino-1-(2-cyclopropylphenyl)ethan-1-one HCl salt (8.2 g, quantitative yield).

LCMS: Method H1, 2.36 min, MS: ES+ 176.0.

### Step (iv)

### Ethyl 2-((2-(2-cyclopropylphenyl)-2-oxoethyl)amino)-2-oxoacetate

To a stirred solution of 2-amino-1-(2-cyclopropylphenyl)ethan-1-one HCl salt (8.2 g, 38.77 mmol) in DCM (85 mL) was added K₂CO₃ (21.40 g, 155.08 mmol) at 0° C. Ethyl oxalyl chloride (10.54 g, 8.63 mL, 77.54 mmol) was added dropwise at 0° C. The mixture was allowed to warm to rt, stirred for 1 h, then poured into water (200 mL) and extracted with EtOAc (2×200 mL). The combined organic phases were dried over Na₂SO₄ and concentrated under reduced pressure to yield ethyl 2-((2-(2-cyclopropylphenyl)-2-oxoethyl)amino)-2-oxoacetate (4.1 g, 14.90 mmol, 57% yield over two steps).

LCMS: Method H1, 3.09 min, MS: ES+ 276.0.

### Step (v)

### Ethyl 5-(2-cyclopropylphenyl)oxazole-2-carboxylate

A stirred solution of ethyl 2-((2-(2-cyclopropylphenyl)-2-oxoethyl)amino)-2-oxoacetate (4.1 g, 14.90 mmol) in POCl₃ (41 mL, 10 vol) was heated at 100° C. for 5 h. The mixture was cooled to rt, slowly poured into crushed ice and extracted with EtOAc (2×200 mL). The combined organic phases were dried over Na₂SO₄ and concentrated under reduced pressure. The residue was purified by flash column chromatography (silica gel, 20% EtOAc in n-hexanes) to yield ethyl 5-(2-cyclopropylphenyl)oxazole-2-carboxylate (1.5 g, 5.83 mmol, 39% yield).

LCMS: Method H1, 3.58 min, MS: ES+: 258.0.

### Step (vi)

### Lithium 5-(2-cyclopropylphenyl)oxazole-2-carboxylate

To a stirred solution of ethyl 5-(2-cyclopropylphenyl)oxazole-2-carboxylate (0.5 g, 1.94 mmol) in THF (4 mL) was added a solution of lithium hydroxide monohydrate (0.16 g, 3.88 mmol) in water (1 mL) dropwise at 0° C. and stirred at rt for 2 h. The reaction mixture was concentrated under reduced pressure to yield lithium 5-(2-cyclopropylphenyl)oxazole-2-carboxylate (0.51 g, quantitative yield).

LCMS: Method H1, 2.07 min, MS: ES+ 230.0.

### Intermediate O

### Lithium 5-(2-(trifluoromethoxy)phenyl)oxazole-2-carboxylate

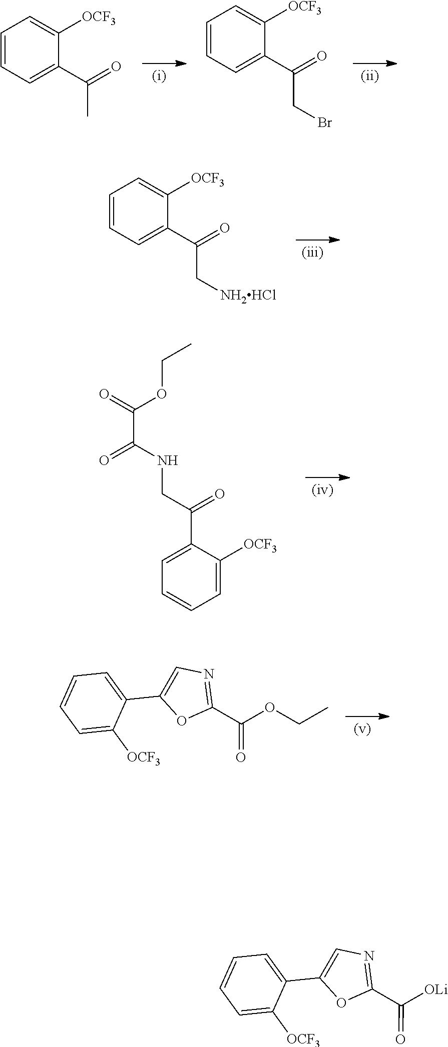

### Step (i)

### 2-Bromo-1-(2-(trifluoromethoxy)phenyl)ethan-1-one

To a stirred solution of 1-(2-(trifluoromethoxy)phenyl)ethan-1-one (CAS 220227-93-0, from Sigma-Aldrich, 1.0 g, 4.89 mmol) in THF (40 mL) was added phenyl trimethylammonium tribromide (1.84 g, 4.89 mmol) at 0° C. The mixture was allowed to warm to rt and stirred for 16 h, then poured into water (50 mL) and extracted with DCM (3×50 mL). The combined organic phases were dried over Na₂SO₄, filtered and concentrated under reduced pressure to yield 2-bromo-1-(2-(trifluoromethoxy)phenyl)ethan-1-one (1.2 g, 4.25 mmol, 86% yield). This crude material was used directly in the next step.

### Step (ii)

### 2-Amino-1-(2-(trifluoromethoxy)phenyl)ethan-1-one HCl salt

To a stirred solution of 2-bromo-1-(2-(trifluoromethoxy)phenyl)ethan-1-one (1.2 g, 4.23 mmol) in MeCN (30 mL) was added sodium diformylamide (0.61 g, 6.35 mmol) and heated at 80° C. for 16 h.

The reaction mixture was cooled to rt and concentrated under reduced pressure. The residue was diluted with MeOH (5 mL) and conc. HCl (6 mL). The mixture was further heated at 80° C. for 3 h, then allowed to cool to rt. The mixture was concentrated under reduced pressure, and the residue was stirred with n-hexanes (25 mL) to form a precipitate. The solid was collected by filtration under reduced pressure to yield 2-amino-1-(2-(trifluoromethoxy)phenyl)ethan-1-one HCl salt (1.2 g, quantitative yield).

LCMS: Method C1, 0.92 min, MS: ES+: 220.1.

Step (iii)

### Ethyl 2-oxo-2-((2-oxo-2-(2-(trifluoromethoxy)phenyl)ethyl)amino)acetate

To a stirred solution of 2-amino-1-(2-(trifluoromethoxy)phenyl)ethan-1-one HCl salt (1.2 g, 5.47 mmol) in DCM (30 mL) was added K₂CO₃ (2.26 g, 16.37 mmol) at 0° C. Ethyl oxalyl chloride (1.11 g, 0.90 mL, 8.21 mmol) was added dropwise at 0° C. The mixture was allowed to warm to rt and stirred for 3 h at rt, then poured into ice-cold water (50 mL) and extracted with EtOAc (3×50 mL). The combined organic phases were dried over Na₂SO₄, filtered and concentrated under reduced pressure.

The residue was purified by flash column chromatography (silica gel, 30% EtOAc in n-hexanes) to yield ethyl 2-oxo-2-((2-oxo-2-(2-(trifluoromethoxy)phenyl)ethyl)amino)acetate (0.65 g, 2.30 mmol, 48% yield over two steps).

LCMS: Method C1, 1.30 min, MS: ES+ 320.2.

### Step (iv)

### Ethyl 5-(2-(trifluoromethoxy)phenyl)oxazole-2-carboxylate

A stirred solution of ethyl 2-oxo-2-((2-oxo-2-(2-(trifluoromethoxy)phenyl)ethyl)amino)acetate (0.6 g, 2.03 mmol) in POCl₃ (10 mL, 15 vol) was heated at 110° C. for 3 h. The mixture was cooled to rt, poured into ice-cold water (100 mL) and extracted with EtOAc (3×100 mL). The combined organic phases were dried over Na₂SO₄, filtered and concentrated under reduced pressure. The residue was purified by flash column chromatography (silica gel, 30% EtOAc in n-hexanes) to yield ethyl 5-(2-(trifluoromethoxy)phenyl)oxazole-2-carboxylate (0.32 g, 1.06 mmol, 52% yield).

LCMS: Method C1, 1.47 min, MS: ES+: 302.2.

### Step (v)

Lithium 5-(2-(trifluoromethoxy)phenyl)oxazole-2-carboxylate

To a stirred solution of ethyl 5-(2-(trifluoromethoxy)phenyl)oxazole-2-carboxylate (0.3 g, 0.99 mmol) in THF:MeOH:water (1:1:1, 9 mL) was added LiOH·H₂O (0.12 g, 2.98 mmol) in portions at 0° C. The mixture was stirred at rt for 3 h, and the mixture was concentrated under reduced pressure. The residue was stirred with pentane (10 mL) to form a precipitate. The solid was collected by filtration under reduced pressure to yield lithium 5-(2-(trifluoromethoxy)phenyl)oxazole-2-carboxylate (0.3 g, quantitative yield).

LCMS: Method C1, 1.13 min, MS: ES+ 274.0.

### Intermediate P

### Lithium 5-(3-bromophenyl)oxazole-2-carboxylate

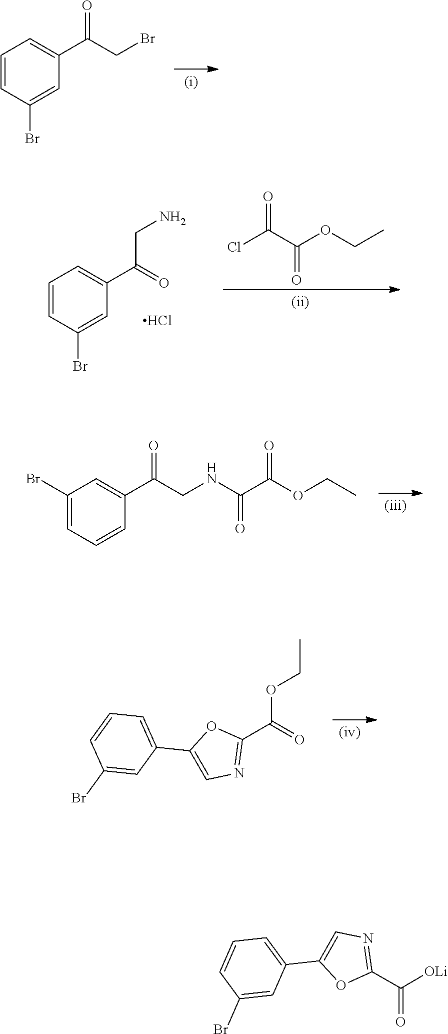

### Step (i)

### 2-Amino-1-(3-bromophenyl)ethan-1-one HCl salt

To a stirred solution of 2-bromo-1-(3-bromophenyl)ethan-1-one (CAS 18523-22-3, from Combi-Blocks, 6.0 g, 21.74 mmol) in MeCN (150 mL) was added sodium diformylamide (3.1 g, 32.62 mmol) and heated at 70° C. for 24 h. The mixture was cooled to rt and concentrated under reduced pressure. The residue was diluted with MeOH (60 mL) and conc. HCl (30 mL). The mixture was further heated at 70° C. for 4 h, then allowed to cool to rt. The mixture was concentrated under reduced pressure, and the residue was stirred with IPA (25 mL) to form a precipitate. The solid was collected by filtration under reduced pressure to afford 2-amino-1-(3-bromophenyl)ethan-1-one HCl salt (7.2 g, quantitative yield).

LCMS: Method C1, 0.99 min, MS: ES+: 214.1, 216.1.

### Step (ii)

### Ethyl 2-((2-(3-bromophenyl)-2-oxoethyl)amino)-2-oxoacetate

To a stirred solution of 2-amino-1-(3-bromophenyl)ethan-1-one HCl salt (7.0 g, 28.11 mmol) in DCM (100 mL) was added K₂CO₃ (11.65 g, 84.34 mmol) at 0° C. Ethyl oxalyl chloride (5.75 g, 4.71 mL, 42.17 mmol) was added dropwise at 0° C. The mixture was allowed to warm to rt, stirred for 3 h, then poured into water (100 mL) and extracted with DCM (2×100 mL). The combined organic phases were dried over Na₂SO₄ and concentrated under reduced pressure to afford ethyl 2-((2-(3-bromophenyl)-2-oxoethyl)amino)-2-oxoacetate (3.0 g, 9.58 mmol, 44% yield over two steps).

LCMS: Method C1, 1.19 min, MS: ES+ 314.0, 316.1.

Step (iii)

### Ethyl 5-(3-bromophenyl)oxazole-2-carboxylate

A stirred solution of ethyl 2-((2-(3-bromophenyl)-2-oxoethyl)amino)-2-oxoacetate (3.0 g, 9.58 mmol) in POCl₃ (9.0 mL, 3 vol) was heated at 100° C. for 3 h. The mixture was cooled to rt, poured into ice-cold water (100 mL) and extracted with EtOAc (3×100 mL). The combined organic phases were dried over Na₂SO₄, filtered and and concentrated under reduced pressure. The residue was purified by flash column chromatography (silica gel, 30% EtOAc in n-hexanes) to yield ethyl 5-(3-bromophenyl)oxazole-2-carboxylate (1.2 g, 4.06 mmol, 42% yield).

LCMS: Method C1, 1.36 min, MS: ES+: 295.9, 297.9.

### Step (iv)

### Lithium 5-(3-bromophenyl)oxazole-2-carboxylate

To a stirred solution of ethyl 5-(3-bromophenyl)oxazole-2-carboxylate (1.1 g, 3.72 mmol) in THF MeOH:water (10 mL, 1:1:1) was added LiOH·H₂O (0.45 g, 11.18 mmol) in portions at 0° C. The mixture was stirred at 0° C. for 3 h, then concentrated under reduced pressure to yield lithium 5-(3-bromophenyl)oxazole-2-carboxylate (1.1 g, quantitative yield).

LCMS: Method C1, 1.04 min, MS: ES+ 268.1, 269.9.

### Intermediate Q

### Ethyl 5-(2-cyclopropyl-5-(trifluoromethoxy)phenyl)-1,3,4-oxadiazole-2-carboxylate

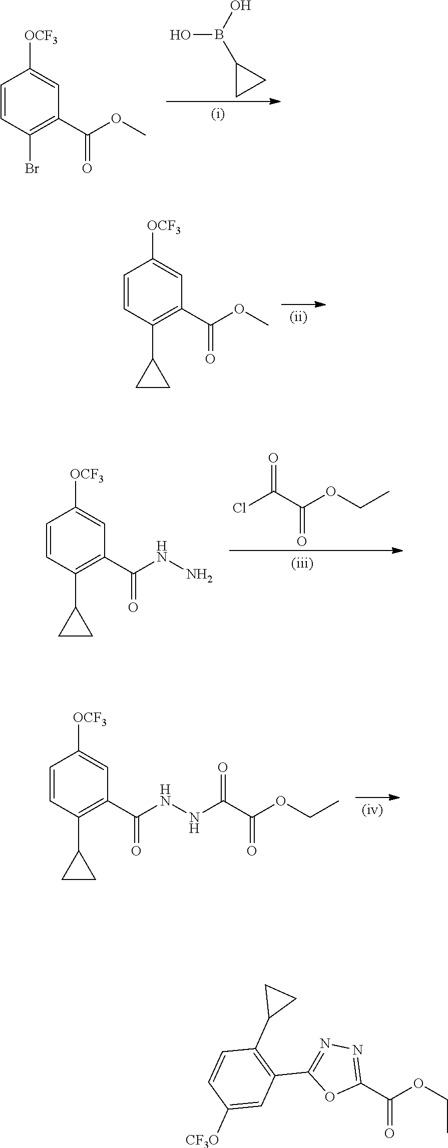

### Step (i)

### Methyl 2-cyclopropyl-5-(trifluoromethoxy)benzoate

To a stirred solution of methyl 2-bromo-5-(trifluoromethoxy)benzoate (CAS 1150114-81-0, from Combi-Blocks, 1.30 g, 4.36 mmol) and cyclopropylboronic acid (CAS 411235-57-9, from Angene, 0.37 g, 4.36 mmol) in toluene:water (13 mL, 7:3) was added K₃PO₄ (1.84 g, 8.69 mmol). The mixture was purged with N₂ gas for 10 min, followed by addition of [1,1′-bis(diphenylphosphino)ferrocene]-dichloropalladium(II) (0.31 g, 0.43 mmol) and heated at 100° C. for 3 h. The mixture was poured into water (15 mL) and extracted with EtOAc (3×15 mL). The combined organic phases were dried over anhydrous Na₂SO₄, filtered and concentrated under reduced pressure. The residue was purified by flash column chromatography (silica gel, 2% EtOAc in n-hexanes) to yield methyl 2-cyclopropyl-5-(trifluoromethoxy)benzoate (0.85 g, 3.26 mmol, 75% yield).

¹H NMR (400 MHz, CDCl₃) δ 7.68 (s, 1H), 7.26 (d, J=11.6 Hz, 1H), 7.05 (d, J=8.8 Hz, 1H), 3.95 (s, 3H), 2.65-2.69 (m, 1H), 1.02-1.05 (m, 2H), 0.68-0.70 (m, 2H).

### Step (ii)

### 2-Cyclopropyl-5-(trifluoromethoxy)benzohydrazide

To a stirred solution of methyl 2-cyclopropyl-5-(trifluoromethoxy)benzoate (1.1 g, 4.22 mmol) in MeOH (28 mL) was added hydrazine hydrate (99%) (3.3 mL, 3 vol) dropwise at 0° C. The mixture was allowed to warm to rt and stirred for 2 h. The mixture was concentrated under reduced pressure to yield 2-cyclopropyl-5-(trifluoromethoxy)benzohydrazide (1.07 g, 4.13 mmol, 97% yield).

LCMS: Method C1, 1.11 min, MS: ES+ 261.5.

Step (iii)

### Ethyl 2-(2-(2-cyclopropyl-5-(trifluoromethoxy)benzoyl)hydrazineyl)-2-oxoacetate

To a stirred solution of 2-cyclopropyl-5-(trifluoromethoxy)benzohydrazide (1.0 g, 3.84 mmol) in DCM (10 mL) were added TEA (1.16 g, 1.6 mL, 11.52 mmol) and ethyl oxalyl chloride (1.04 g, 0.85 mL, 7.68 mmol) dropwise at 0° C. The mixture was allowed to warm to rt and stirred for 2 h, then poured into water (15 mL) and extracted with EtOAc (3×15 mL). The combined organic phases were dried over Na₂SO₄, filtered and concentrated under reduced pressure to yield ethyl 2-(2-(2-cyclopropyl-5-(trifluoromethoxy)benzoyl)hydrazineyl)-2-oxoacetate (1.64 g, quantitative yield).

LCMS: Method C1, 1.20 min, MS: ES+ 361.1.

### Step (iv)

### Ethyl 5-(2-cyclopropyl-5-(trifluoromethoxy)phenyl)-1,3,4-oxadiazole-2-carboxylate

To a stirred solution of ethyl 2-(2-(2-cyclopropyl-5-(trifluoromethoxy)benzoyl)hydrazineyl)-2-oxoacetate (1.6 g, 4.44 mmol) in DCM (16 mL) were added TEA (1.34 g, 1.85 mL, 13.32 mmol) and TsCl (1.01 g, 5.32 mmol) in portions at 0° C. The mixture was allowed to warm at rt and stirred for 2 h, then poured into water (20 mL) and extracted with EtOAc (3×20 mL). The combined organic phases were dried over anhydrous Na₂SO₄, filtered and concentrated under reduced pressure. The residue was purified using flash column chromatography (silica gel, 20% EtOAc in n-hexanes) to yield ethyl 5-(2-cyclopropyl-5-(trifluoromethoxy)phenyl)-1,3,4-oxadiazole-2-carboxylate (0.6 g, 1.75 mmol, 39% yield over two steps). LCMS: Method C1, 1.43 min, MS: ES+ 343.1.

### Intermediate R

### Ethyl 5-(2-fluoro-5-(trifluoromethoxy)phenyl)-1,3,4-oxadiazole-2-carboxylate

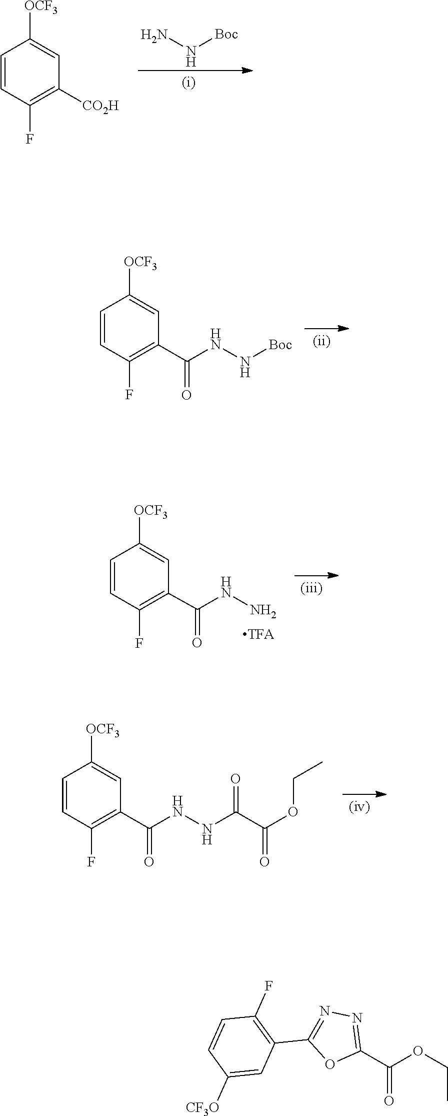

### Step (i)

### tert-Butyl 2-(2-fluoro-5-(trifluoromethoxy)benzoyl)hydrazine-1-carboxylate

To a stirred solution of 2-fluoro-5-(trifluoromethoxy)benzoic acid (CAS 886497-85-4, from Combi-Blocks, 7.0 g, 31.23 mmol) in THF (70 mL) were added DIPEA (12.08 g, 16 mL, 105.25 mmol) and HATU (23.7 g, 62.46 mmol) in portions at 0° C. After 30 min, tert-butyl hydrazinecarboxylate (4.95 g, 37.48 mmol) was added at 0° C. The mixture was slowly warmed to rt and stirred at rt for 16 h, then poured into ice-cold water (50 mL) and extracted with EtOAc (2×200 mL). The combined organic phases were dried over Na₂SO₄, filtered and concentrated under reduced pressure. The residue was purified by column chromatography (silica gel, 25% EtOAc in n-hexanes) to yield tert-butyl 2-(2-fluoro-5-(trifluoromethoxy)benzoyl)hydrazine-1-carboxylate (9.0 g, 26.62 mmol, 85% yield).

LCMS: Method J1, 3.43 min, MS: ES+ 283.1 (M-56).

### Step (ii)

### 2-Fluoro-5-(trifluoromethoxy)benzohydrazide TFA salt

To a stirred solution of tert-butyl 2-(2-fluoro-5-(trifluoromethoxy)benzoyl)hydrazine-1-carboxylate (5.0 g, 14.78 mmol) in DCM (50 mL) was added TFA (25 mL, 5 vol) dropwise at 0° C. The mixture was allowed to warm to rt and stirred for 1 h, then concentrated under reduced pressure to yield 2-fluoro-5-(trifluoromethoxy)benzohydrazide TFA salt (5.0 g, quantitative yield).

LCMS: Method C1, 1.02 min, MS: ES+ 239.2.

Step (iii)

### Ethyl 2-(2-(2-fluoro-5-(trifluoromethoxy)benzoyl)hydrazineyl)-2-oxoacetate

To a stirred solution of 2-fluoro-5-(trifluoromethoxy)benzohydrazide TFA salt (10.0 g, 20.99 mmol) in DCM (100 mL) was added K₂CO₃ (17.38 g, 125.97 mmol) at 0° C. Ethyl oxalyl chloride (8.59 g, 6.97 mL, 62.98 mmol) was added dropwise at 0° C. The mixture was allowed to warm to rt and stirred for 4 h. The mixture was poured into water (100 mL) to form a precipitate. The solid was collected by filtration under reduced pressure to afford ethyl 2-(2-(2-fluoro-5-(trifluoromethoxy)benzoyl)-hydrazineyl)-2-oxoacetate (10.0 g, 29.56 mmol, 88% yield over two steps).

LCMS: Method C1, 1.13 min, MS: ES+ 339.2.

### Step (iv)

### Ethyl 5-(2-fluoro-5-(trifluoromethoxy)phenyl)-1,3,4-oxadiazole-2-carboxylate

A stirred solution of ethyl 2-(2-(2-fluoro-5-(trifluoromethoxy)benzoyl)hydrazineyl)-2-oxoacetate (10 g, 29.56 mmol) in DCM (100 mL) were added TEA (12.2 mL, 8.85 g, 88.68 mmol) and TsCl (6.76 g, 35.48 mmol) in portions at 0° C. The mixture was allowed to warm to rt and stirred for 4 h.

The mixture was poured into water (100 mL) and extracted with DCM (2×250 mL). The combined organic phases were dried over Na₂SO₄, filtered and concentrated under reduced pressure. The residue was purified by flash column chromatography (silica gel, 10% EtOAc in n-hexanes) to afford ethyl 5-(2-fluoro-5-(trifluoromethoxy)phenyl)-1,3,4-oxadiazole-2-carboxylate (2.6 g, 8.12 mmol, 27% yield).

LCMS: Method J1, 4.02 min, MS: ES+: 320.8.

### Intermediate S

### Lithium 5-(5-cyano-2-fluorophenyl)oxazole-2-carboxylate

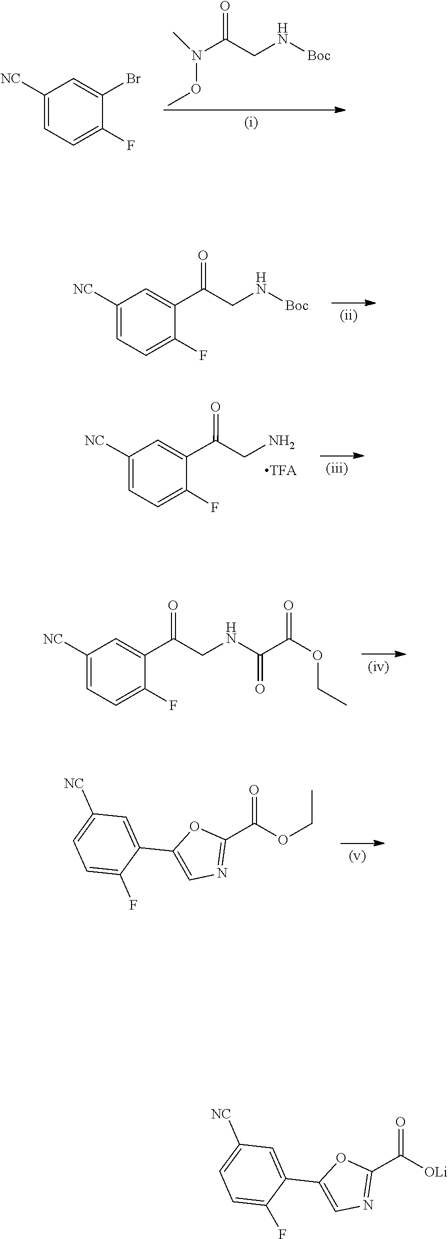

### Step (i)

### tert-Butyl (2-(5-cyano-2-fluorophenyl)-2-oxoethyl)carbamate

To a stirred solution of 3-bromo-4-fluorobenzonitrile (CAS 79630-23-2, from Combi-Blocks) (20.0 g, 100.53 mmol) in dry THF (150 mL) was added isopropylmagnesium chloride (2 M in THF) (50.26 mL, 100.53 mmol) dropwise at rt under N₂ atmosphere. The resulting dark solution was stirred at rt for 1.5 h (during which time a precipitate formed). Meanwhile, to a stirred suspension of tert-butyl (2-(methoxy(methyl)amino)-2-oxoethyl)carbamate (21.92 g, 100.53 mmol) in THF (50 mL) was added isopropylmagnesium chloride (2 M in THF) (50.26 mL, 100.53 mmol) dropwise at 0° C. The resulting solution was stirred for 15 minutes before being added dropwise at 0° C. to the aryl Grignard initially generated. The mixture was stirred at rt for 16 h. The resulting reaction mixture was poured into water (100 mL) and extracted with EtOAc (2×200 mL). The organic phase was combined, dried over Na₂SO₄, filtered and concentrated under reduced pressure. This crude material was purified by column chromatography (25% EtOAc in n-hexanes) to yield tert-butyl (2-(5-cyano-2-fluorophenyl)-2-oxoethyl)carbamate (3.0 g, 10.78 mmol, 10% yield).

LCMS: Method H1, 3.01 min, MS: ES+ 179.0 (M-100).

### Step (ii)

### 4-Fluoro-3-glycylbenzonitrile TFA Salt

To a stirred solution of tert-butyl (2-(5-cyano-2-fluorophenyl)-2-oxoethyl)carbamate (2.2 g, 7.9 mmol) in DCM (22 mL) was added TFA (6.6 mL, 3 vol) dropwise at 0° C. The mixture was allowed to warm to rt and stirred for 3 h, then concentrated under reduced pressure to yield 4-fluoro-3-glycylbenzonitrile TFA salt (4.0 g, quantitative yield).

LCMS: Method C1, 0.37 min, MS: ES+ 178.9.

Step (iii)

### Ethyl 2-((2-(5-cyano-2-fluorophenyl)-2-oxoethyl)amino)-2-oxoacetate

To a stirred solution of 4-fluoro-3-glycylbenzonitrile TFA salt (4.0 g, 13.6 mmol) in DCM (20 mL) was added TEA (4.1 g, 5.7 mL, 41.0 mmol) at 0° C. Ethyl oxalyl chloride (3.73 g, 3.0 mL, 27.3 mmol) was added dropwise at 0° C. The mixture was allowed to warm to rt and stirred for 2 h, then poured into ice-cold water (150 mL) and extracted with EtOAc (2×100 mL). The combined organic phases were dried over Na₂SO₄, filtered and concentrated under reduced pressure yield ethyl 2-((2-(5-cyano-2-fluorophenyl)-2-oxoethyl)amino)-2-oxoacetate (2.8 g, 10.07 mmol, 44% yield over two steps).

LCMS: Method C1, 1.08 min, MS: ES+ 279.1.

### Step (iv)

### Ethyl 5-(5-cyano-2-fluorophenyl)oxazole-2-carboxylate

A stirred solution of ethyl 2-((2-(5-cyano-2-fluorophenyl)-2-oxoethyl)amino)-2-oxoacetate (2.8 g, 10.07 mmol) in POCl₃ (14 mL, 5 vol) was heated at 100° C. for 3 h. The mixture was cooled to rt, poured into ice-cold water (100 mL) and extracted with EtOAc (2×200 mL). The combined organic phases were dried over Na₂SO₄, filtered and concentrated under reduced pressure. The residue was purified by flash column chromatography (silica gel, 30% EtOAc in n-hexanes) to yield ethyl 5-(5-cyano-2-fluorophenyl)oxazole-2-carboxylate (0.8 g, 3.07 mmol, 38% yield over two steps).

LCMS: Method C1, 1.19 min, MS: ES+ 261.1.

### Step (v)

### Lithium 5-(5-cyano-2-fluorophenyl)oxazole-2-carboxylate

To a stirred solution of ethyl 5-(5-cyano-2-fluorophenyl)oxazole-2-carboxylate (0.8 g, 3.07 mmol) in THF (8 mL) was added a solution of LiOH·H₂O (0.15 g, 3.69 mmol) in water (2 mL) dropwise at 0° C. and stirred at rt for 1 h. The reaction mixture was concentrated under reduced pressure to yield lithium 5-(5-cyano-2-fluorophenyl)oxazole-2-carboxylate (0.9 g, quantitative yield).

LCMS: Method C1, 0.92 min, MS: ES+ 232.9.

### Intermediate Y

### 2-Fluoro-4-(1-methyl-1H-indazol-5-yl)benzoic acid

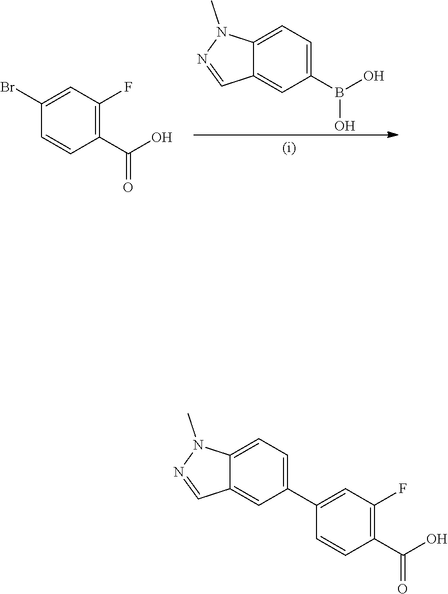 [SMILES: O=C(O)c1ccc(Br)cc1F ; Cn1ncc2cc(B(O)O)ccc21 ; Cn1ncc2cc(-c3ccc(C(=O)O)c(F)c3)ccc21]

To a stirred solution of 4-bromo-2-fluorobenzoic acid (CAS 112704-79-7, from Ark-Pharma, 2.5 g, 11.47 mmol) and (1-methyl-1H-indazol-5-yl)boronic acid (CAS 590418-08-9, from BLD-Pharma, 2.22 g, 12.61 mmol) in 1,4-dioxane:water (25 mL, 9:1) was added K₃PO₄ (4.86 g, 22.94 mmol). The mixture was purged with N₂ gas for 15 min, followed by addition of [1,1′-bis(diphenylphosphino)ferrocene]dichloropalladium(II) (0.83 g, 1.14 mmol) and heated at 100° C. for 3 h. The mixture was poured into water (100 mL) and extracted with EtOAc (2×150 mL). The combined organic phases were dried over anhydrous Na₂SO₄, filtered and concentrated under reduced pressure. The residue was purified by flash column chromatography (silica gel, 70% EtOAc in n-hexanes) to yield 2-fluoro-4-(1-methyl-1H-indazol-5-yl)benzoic acid (1.7 g, 6.29 mmol, 54% yield).

LCMS: Method J1, 3.08 min, MS: ES+ 271.1.

### Intermediate Z

### 3-(3-(Trifluoromethoxy)phenyl)pyrrolidine TFA salt

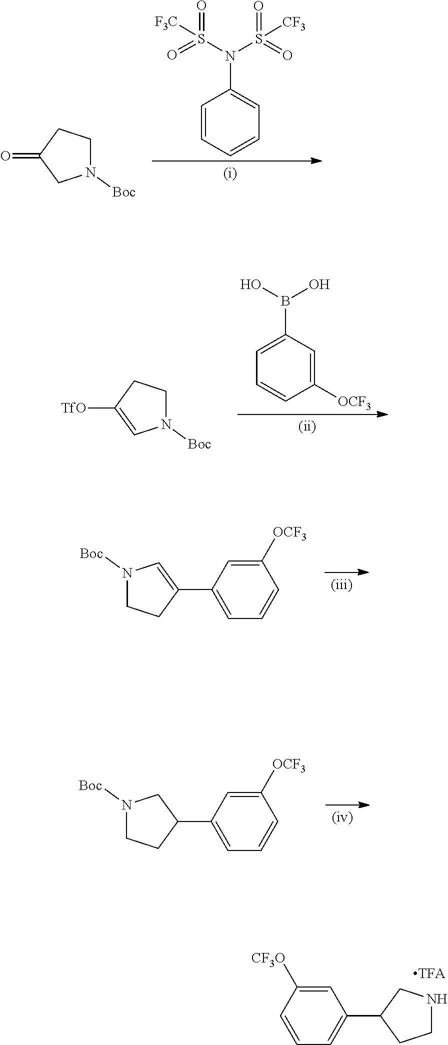

### Step (i)

### tert-Butyl 4-(((trifluoromethyl)sulfonyl)oxy)-2,3-dihydro-1H-pyrrole-1-carboxylate

To a stirred solution of NaHMDS (1M in THF) (5.39 mL, 5.39 mmol) in THF (4 mL) was added tert-butyl 3-oxopyrrolidine-1-carboxylate (CAS 101385-93-7, from Combi-Blocks, 0.50 g, 2.69 mmol) in portions at −78° C. and stirred at −78° C. for 15 min. A solution of 1,1,1-trifluoro-N-phenyl-N-((trifluoromethyl)sulfonyl)methanesulfonamide (CAS 37595-74-7, from TCI, 0.90 g, 2.69 mmol) in THF (4 mL) was added dropwise and stirred at −78° C. for 2 h. Three more identical batches were carried out in similar manner and all four reaction mixtures were mixed and poured into water (100 mL) and extracted with EtOAc (3×100 mL). The combined organic phases were dried over anhydrous Na₂SO₄, filtered and concentrated under reduced pressure to yield tert-butyl 4-(((trifluoromethyl)sulfonyl)oxy)-2,3-dihydro-1H-pyrrole-1-carboxylate (3.3 g, 10.4 mmol, 96% yield). The crude was carried to the next step.

### Step (ii)

### tert-Butyl 4-(3-(trifluoromethoxy)phenyl)-2,3-dihydro-1H-pyrrole-1-carboxylate

To a stirred solution of tert-butyl 4-(((trifluoromethyl)sulfonyl)oxy)-2,3-dihydro-1H-pyrrole-1-carboxylate (2.50 g, 7.88 mmol) and (3-(trifluoromethoxy)phenyl)boronic acid (CAS 179113-90-7, from Combi-Blocks, 1.61 g, 7.88 mmol) in 1,4-dioxane:water (10 mL, 8:2) was added K₃PO₄ (5.0 g, 2.36 mmol). The mixture was purged with N₂ gas for 20 min, followed by addition of [1,1′-bis(diphenylphosphino)ferrocene]dichloropalladium(II) (0.50 g, 0.78 mmol) and heated at 80° C. for 2 h. The mixture was poured into water (100 mL) and extracted with EtOAc (3×100 mL). The combined organic phases were dried over anhydrous Na₂SO₄, filtered and concentrated under reduced pressure. The residue was purified by flash column chromatography (silica gel, 4% EtOAc in n-hexanes) to yield tert-butyl 4-(3-(trifluoromethoxy)phenyl)-2,3-dihydro-1H-pyrrole-1-carboxylate (1.1 g, 3.34 mmol, 42% yield). MS: ES+ 273.97 (M-56).

Step (iii)

### tert-Butyl 3-(3-(trifluoromethoxy)phenyl)pyrrolidine-1-carboxylate

To a stirred solution of tert-butyl 4-(3-(trifluoromethoxy)phenyl)-2,3-dihydro-1H-pyrrole-1-carboxylate (1.0 g, 3.03 mmol) in MeOH (5 mL) was added 10% Pd/C (50% moisture) (0.5 g, 0.5 w/w). The mixture was purged with H₂ gas for 2 h, then filtered through Celite Hyflow® and the filtrate was concentrated under reduced pressure to yield tert-butyl 3-(3-(trifluoromethoxy)phenyl)pyrrolidine-1-carboxylate (0.5 g, 1.51 mmol, 49% yield).

LCMS: Method C1, 1.47 min, MS: ES+ 275.9 (M-56).

### Step (iv)

### 3-(3-(Trifluoromethoxy)phenyl)pyrrolidine TFA salt

To a stirred solution of tert-butyl 3-(3-(trifluoromethoxy)phenyl)pyrrolidine-1-carboxylate (0.45 g, 1.45 mmol) in DCM (4 mL) was added TFA (2.4 mL, 5 vol) dropwise at 0° C. The mixture was allowed to warm to rt and stirred for 1 h, then concentrated under reduced pressure to yield 3-(3-(trifluoromethoxy)phenyl) pyrrolidine TFA salt (0.65 g, quantitative yield).

LCMS: Method C1, 1.14 min, MS: ES+ 232.0.

### Intermediate Z1

### Ethyl 5-(4-fluoro-3-(trifluoromethoxy)phenyl)oxazole-2-carboxylate

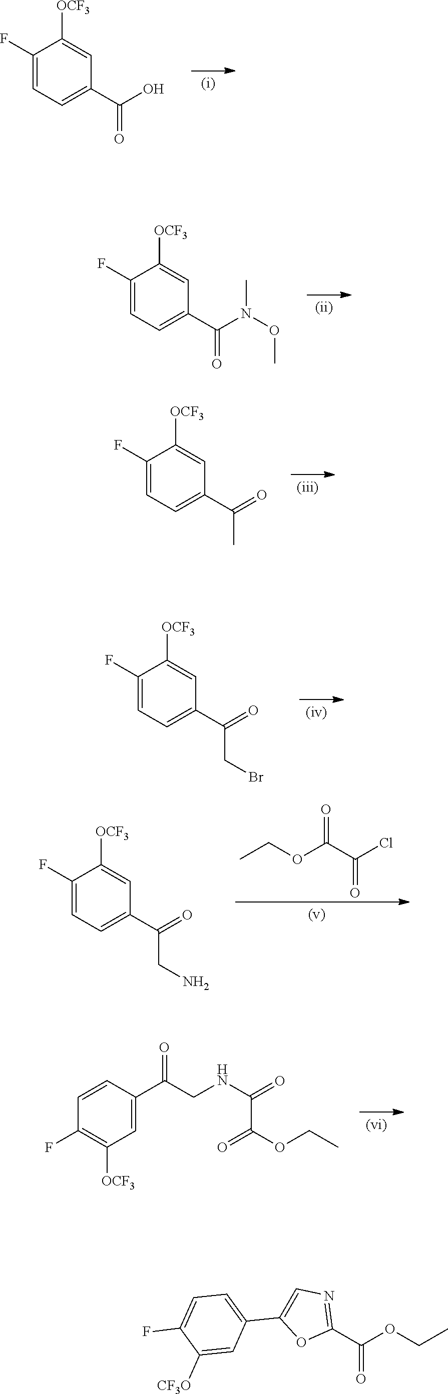

### Step (i)

### 4-Fluoro-N-methoxy-N-methyl-3-(trifluoromethoxy)benzamide

To a stirred solution of 4-fluoro-3-(trifluoromethoxy)benzoic acid (CAS 886496-49-7, from Combi-Blocks, 5.0 g, 22.32 mmol) in THF (50 mL) were added DIPEA (8.63 g, 11.44 mL, 66.96 mmol) and HATU (12.72 g, 33.48 mmol) in portions at 0° C. After 30 min, N,O-dimethylhydroxylamine HCl (2.39 g, 24.55 mmol) was added at 0° C. The mixture was allowed to warm to rt and stirred at rt for 16 h, then poured into ice-cold water (100 mL) and extracted with EtOAc (2×150 mL). The combined organic phases were dried over Na₂SO₄, filtered and concentrated under reduced pressure. The residue was purified by flash column chromatography (silica gel, 16% EtOAc in n-hexanes) to yield 4-fluoro-N-methoxy-N-methyl-3-(trifluoromethoxy)benzamide (4.80 g, 17.97 mmol, 81% yield).

LCMS: Method J1, 3.78 min, MS: ES+ 267.8.

### Step (ii)

### 1-(4-Fluoro-3-(trifluoromethoxy)phenyl)ethan-1-one

To a stirred solution of 4-fluoro-N-methoxy-N-methyl-3-(trifluoromethoxy)benzamide (4.80 g, 17.97 mmol) in THF (48 mL) was added methylmagnesium bromide (3 M in diethyl ether) (17.97 mL, 53.91 mmol) dropwise at 0° C. The mixture was allowed to warm to rt and stirred at rt for 16 h, then poured into water (150 mL), 1N HCl (10 mL) was added and extracted with EtOAc (2×150 mL). The combined organic phases were dried over Na₂SO₄ and concentrated under reduced pressure. The residue was purified by flash column chromatography (silica gel, 12% EtOAc in n-hexanes) to yield 1-(4-fluoro-3-(trifluoromethoxy)phenyl)ethan-1-one (3.0 g, 13.51 mmol, 75% yield).

¹H NMR (400 MHz, DMSO-d₆) δ ppm: 8.06-8.12 (m, 2H), 7.70 (t, J=8.8 Hz, 1H), 2.62 (s, 3H).

Step (iii)

### 2-Bromo-1-(4-fluoro-3-(trifluoromethoxy)phenyl)ethan-1-one

To a stirred solution of 1-(4-fluoro-3-(trifluoromethoxy)phenyl)ethan-1-one (3.0 g, 13.51 mmol) in THF (30 mL) was added pyridinium tribromide (4.75 g, 14.86 mmol) in portions at rt and stirred at rt for 16 h. The mixture was poured into water (150 mL) and extracted with EtOAc (2×200 mL). The combined organic phases were dried over Na₂SO₄ and concentrated under reduced pressure. The residue was purified by flash column chromatography (silica gel, 100% n-hexanes) to yield 2-bromo-1-(4-fluoro-3-(trifluoromethoxy)phenyl)ethan-1-one (2.0 g, 6.66 mmol, 49% yield). The material was forwarded to the next step.

### Step (iv)

### 2-Amino-1-(4-fluoro-3-(trifluoromethoxy)phenyl)ethan-1-one HCl salt

To a stirred solution of 2-bromo-1-(4-fluoro-3-(trifluoromethoxy)phenyl)ethan-1-one (2.0 g, 6.66 mmol) in MeCN (20 mL) was added sodium diformylamide (0.76 g, 7.99 mmol) and heated at 80° C. for 16 h. The mixture was cooled to rt and concentrated under reduced pressure. The residue was diluted with MeOH (20 mL) and conc. HCl (2 mL). The mixture was further heated at 80° C. for 16 h, then allowed to cool to rt. The mixture was concentrated under reduced pressure, and the residue was stirred with diethyl ether (20 mL) to form a precipitate. The solid was collected by filtration under reduced pressure to yield 2-amino-1-(4-fluoro-3-(trifluoromethoxy)phenyl)ethan-1-one HCl salt (2.9 g, quantitative yield).

LCMS: Method J1, 2.47 min, MS: ES+ 238.0.

### Step (v)

### Ethyl 2-((2-(4-fluoro-3-(trifluoromethoxy)phenyl)-2-oxoethyl)amino)-2-oxoacetate

To a stirred solution of 2-amino-1-(4-fluoro-3-(trifluoromethoxy)phenyl)ethan-1-one HCl salt (2.90 g, 10.60 mmol) in DCM (30 mL) at 0° C. was added K₂CO₃ (4.39 g, 31.8 mmol). Ethyl oxalyl chloride (2.17 g, 1.77 mL, 15.9 mmol) was added dropwise at 0° C. The mixture was allowed to warm to rt, stirred for 4 h, then poured into water (50 mL) and extracted with DCM (2×100 mL). The combined organic phases were dried over Na₂SO₄ and concentrated under reduced pressure to yield ethyl 2-((2-(4-fluoro-3-(trifluoromethoxy)phenyl)-2-oxoethyl)amino)-2-oxoacetate (1.70 g, 5.04 mmol, 75% yield over two steps).

LCMS: Method J1, 3.80 min, MS: ES+ 337.8.

### Step (vi)

### Ethyl 5-(4-fluoro-3-(trifluoromethoxy)phenyl)oxazole-2-carboxylate

A stirred solution of ethyl 2-((2-(4-fluoro-3-(trifluoromethoxy)phenyl)-2-oxoethyl)amino)-2-oxoacetate (1.70 g, 5.04 mmol) in POCl₃ (9 mL, 5.3 vol) was heated at 120° C. for 16 h. The mixture was cooled to rt, slowly poured into crushed ice and extracted with EtOAc (2×100 mL). The combined organic phases were dried over Na₂SO₄ and concentrated under reduced pressure. The residue was purified by flash column chromatography (silica gel, 20% EtOAc in n-hexanes) to yield ethyl 5-(4-fluoro-3-(trifluoromethoxy)phenyl)oxazole-2-carboxylate (0.96 g, 3.01 mmol, 60% yield).

LCMS: Method J1, 3.86 min, MS: ES+ 320.1.

### Intermediate Z2

### Ethyl 5-(2-(azetidin-1-yl)-5-cyanopyridin-3-yl)-1,3,4-oxadiazole-2-carboxylate

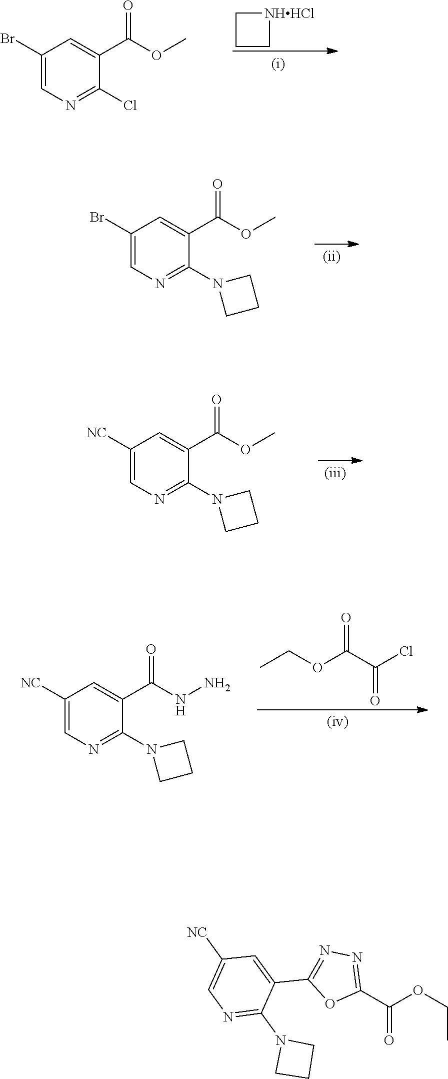

### Step (i)

### Methyl 2-(azetidin-1-yl)-5-bromonicotinate

To a stirred solution of azetidine hydrochloride (CAS 36520-39-5, from Combi-Blocks, 1.79 g, 19.27 mmol) in DMSO (40 mL) was added K₂CO₃ (6.64 g, 48.18 mmol) at rt and stirred for 5 min. Methyl 5-bromo-2-chloronicotinate (CAS 78686-79-0, from Combi-Blocks, 4.0 g, 16.06 mmol) at rt and the mixture was heated at 80° C. for 2 h. The mixture was poured into ice-cold water (100 mL) to form a precipitate. The solid was collected by filtration under reduced pressure to yield methyl 2-(azetidin-1-yl)-5-bromonicotinate (3.8 g, 14.07 mmol, 87% yield).

LCMS: Method C1, 1.35 min, MS: ES+ 271.1, 273.1.

### Step (ii)

### Methyl 2-(azetidin-1-yl)-5-cyanonicotinate

To a stirred solution of methyl 2-(azetidin-1-yl)-5-bromonicotinate (1.90 g, 7.03 mmol) in DMF (19 mL) was added zinc cyanide (2.46 g, 21.09 mmol) and zinc dust (0.23 g, 3.51 mmol) at rt. The mixture was degassed with N₂ gas for 10 min, followed by addition of [1,1′-bis(diphenylphosphino)ferrocene]dichloropalladium(II) (0.26 g, 0.35 mmol) and 1,1′-bis(diphenylphosphino)ferrocene (0.39 g, 0.70 mmol). The mixture was heated at 130° C. for 2 h. One more identical batch was carried out in similar manner and both reaction mixtures were mixed. The resulting mixture was poured into ice-cold water (50 mL), extracted with EtOAc (3×50 mL). The combined organic phases were dried over Na₂SO₄ and concentrated under reduced pressure. The residue was purified using flash column chromatography (silica gel, 14% EtOAc in n-hexanes) to yield methyl 2-(azetidin-1-yl)-5-cyanonicotinate (1.3 g, 5.99 mmol, 42% yield).

LCMS: Method C1, 1.14 min, MS: ES+: 218.1.

Step (iii)

### 2-(Azetidin-1-yl)-5-cyanonicotinohydrazide

Methyl 2-(azetidin-1-yl)-5-cyanonicotinate (1.2 g, 5.52 mmol) was added to hydrazine hydrate (99%) (31.2 mL, 26 vol) at 0° C. The mixture was stirred at rt for 8 h, then poured into water (20 mL) to form a precipitate. The solid was collected by filtration under reduced pressure to yield 2-(azetidin-1-yl)-5-cyanonicotinohydrazide (0.90 g, 4.14 mmol, 75% yield).

LCMS: Method C1, 0.79 min, MS: ES+ 218.2.

### Step (iv)

### Ethyl 5-(2-(azetidin-1-yl)-5-cyanopyridin-3-yl)-1,3,4-oxadiazole-2-carboxylate

To a stirred solution of 2-(azetidin-1-yl)-5-cyanonicotinohydrazide (0.9 g, 4.14 mmol) in DCM (10 mL) were added TEA (1.25 g, 1.72 mL, 12.42 mmol) and ethyl oxalyl chloride (0.85 g, 0.69 mL, 6.21 mmol) dropwise at 0° C. The mixture was allowed to warm to rt and stirred for 1 h. TEA (1.25 g, 1.72 mL, 12.42 mmol) and TsCl (0.94 g, 4.96 mmol) was added in portions at 0° C. The mixture was allowed to warm to rt and stirred for 1 h. The resulting mixture was poured into water (30 mL) and extracted with EtOAc (3×30 mL). The combined organic phases were dried over Na₂SO₄, filtered and concentrated under reduced pressure. The residue was purified using flash column chromatography (silica gel, 27% EtOAc in n-hexanes) to yield ethyl 5-(2-(azetidin-1-yl)-5-cyanopyridin-3-yl)-1,3,4-oxadiazole-2-carboxylate (0.55 g, 1.83 mmol, 44% yield).

LCMS: Method C1, 1.20 min, MS: ES+ 300.2.

### Intermediate Z3

### Ethyl 5-(5-cyano-2-(pyrrolidin-1-yl)pyridin-3-yl)-1,3,4-oxadiazole-2-carboxylate

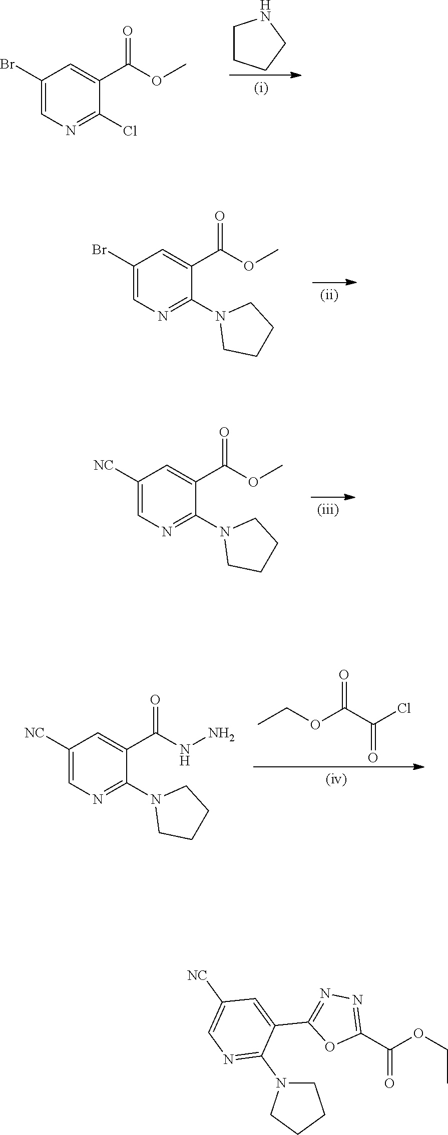

### Step (i)

### Methyl 5-bromo-2-(pyrrolidin-1-yl)nicotinate

To a stirred solution of pyrrolidine (3.56 g, 50.2 mmol) in DMSO (50 mL) was added K₂CO₃ (8.31 g, 60.24 mmol) at rt and stirred for 5 min. Methyl 5-bromo-2-chloronicotinate (CAS 78686-79-0, from Combi-Blocks, 5.0 g, 20.08 mmol) was added at rt into the reaction mixture and heated at 80° C. for 2 h.

The mixture was poured into ice-cold water (100 mL) to form a precipitate. The solid was collected by filtration under reduced pressure to yield methyl 5-bromo-2-(pyrrolidin-1-yl)nicotinate (4.8 g, 16.90 mmol, 84% yield). LCMS: Method C1, 1.37 min, MS: ES+ 285.1, 287.0.

### Step (ii)

### Methyl 5-cyano-2-(pyrrolidin-1-yl)nicotinate

To a stirred solution of methyl 5-bromo-2-(pyrrolidin-1-yl)nicotinate (0.50 g, 1.76 mmol) in DMF (5 mL) was added zinc cyanide (0.62 g, 5.28 mmol) and zinc dust (0.06 g, 0.88 mmol) at rt. The reaction mixture was degassed with N₂ gas for 10 min, followed by addition of [1,1′-bis(diphenylphosphino)ferrocene]dichloropalladium(II) (0.06 g, 0.09 mmol) and 1,1′-bis(diphenylphosphino)ferrocene (0.14 g, 0.26 mmol). The reation mixture was heated at 130° C. for 2 h. Eight more identical batches were carried out in similar manner and all nine reaction mixtures were combined for workup. The resulting mixture was poured into ice-cold water (100 mL), extracted with EtOAc (2×100 mL). The combined organic phases were washed with ice-cold water (4×100 mL), dried over Na₂SO₄ and concentrated under reduced pressure. The residue was purified using flash column chromatography (silica gel, 34% EtOAc in n-hexanes) to yield methyl 5-cyano-2-(pyrrolidin-1-yl)nicotinate (2.0 g, 8.65 mmol, 54% yield).

LCMS: Method C1, 1.20 min, MS: ES+: 232.1.

Step (iii)

### 5-Cyano-2-(pyrrolidin-1-yl)nicotinohydrazide

To a stirred solution of methyl 5-cyano-2-(pyrrolidin-1-yl)nicotinate (2.0 g, 8.65 mmol) in ethanol (20 mL) was added hydrazine hydrate (99%) (20 mL, 10 vol) at rt. The reaction mixture was heated at 80° C. for 16 h. The mixture was cooled to rt and concentrated under reduced pressure. The residue was suspended in water (20 mL) to form a precipitate. The solid was collected by filtration under reduced pressure to yield 5-cyano-2-(pyrrolidin-1-yl)nicotinohydrazide (0.72 g, 3.11 mmol, 36% yield).

LCMS: Method C1, 0.90 min, MS: ES+ 232.1.

### Step (iv)

### Ethyl 5-(5-cyano-2-(pyrrolidin-1-yl)pyridin-3-yl)-1,3,4-oxadiazole-2-carboxylate

To a stirred solution of 5-cyano-2-(pyrrolidin-1-yl)nicotinohydrazide (0.72 g, 3.11 mmol) in DCM (7 mL) were added TEA (0.94 g, 1.29 mL, 9.33 mmol) and ethyl oxalyl chloride (0.46 g, 0.38 mL, 3.42 mmol) dropwise at 0° C. The mixture was allowed to warm to rt and stirred for 1 h. TEA (0.94 g, 1.29 mL, 9.33 mmol) and TsCl (0.71 g, 3.73 mmol) was added in portions at 0° C. The mixture was allowed to warm to rt and stirred at rt for 1 h, then poured into water (15 mL) and extracted with EtOAc (3×15 mL). The combined organic phases were dried over anhydrous Na₂SO₄, filtered and concentrated under reduced pressure. The residue was purified using flash column chromatography (silica gel, 30% EtOAc in n-hexanes) to yield ethyl 5-(5-cyano-2-(pyrrolidin-1-yl)pyridin-3-yl)-1,3,4-oxadiazole-2-carboxylate (0.23 g, 0.73 mmol, 23% yield).

LCMS: Method C1, 1.25 min, MS: ES+ 314.1.

### Intermediate Z4

### Ethyl 5-(2-chloro-5-(trifluoromethoxy)phenyl)-1,3,4-oxadiazole-2-carboxylate

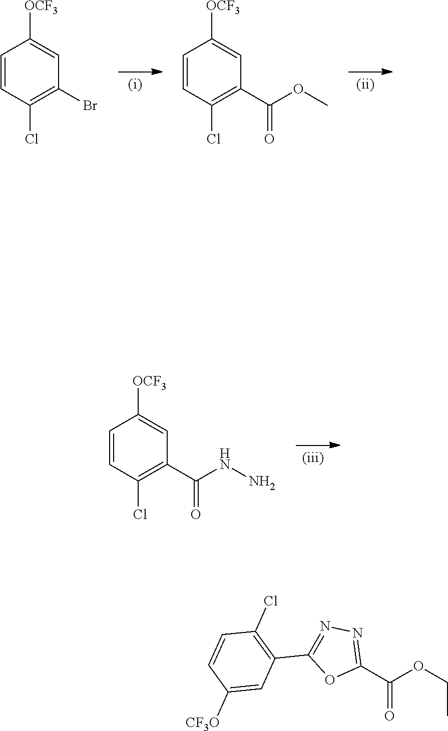

### Step (i)

### Methyl 2-chloro-5-(trifluoromethoxy)benzoate

To an autoclave, charged with a solution of 2-bromo-1-chloro-4-(trifluoromethoxy)benzene (CAS 468075-00-5, from Combi-Blocks, 5.0 g, 18.25 mmol) in MeOH (100 mL) were added TEA (5.54 g, 7.63 mL, 54.76 mmol) and [1,1′-bis(diphenylphosphino)ferrocene]dichloropalladium(II) (1.33 g, 1.82 mmol). The mixture was heated at 80° C. for 16 h under pressure of CO gas (50 psi). The mixture was filtered through Celite Hyflow® and the filtrate was concentrated under reduced pressure. The residue was poured into water (50 mL) and extracted with EtOAc (3×50 mL). The combined organic phases were dried over anhydrous Na₂SO₄, filtered and concentrated under reduced pressure. The residue was purified by flash column chromatography (silica gel, 5% EtOAc in n-hexanes) to yield methyl 2-chloro-5-(trifluoromethoxy)benzoate (3.0 g, 11.81 mmol, 65% yield).

¹H NMR (400 MHz, DMSO-d6) δ ppm: 7.79 (s, 1H), 7.73 (d, J=8.4 Hz, 1H), 7.62 (d, J=7.6 Hz, 1H), 3.88 (s, 3H).

### Step (ii)

### 2-Chloro-5-(trifluoromethoxy)benzohydrazide

To a stirred solution of methyl 2-chloro-5-(trifluoromethoxy)benzoate (3.0 g, 3.81 mmol) in MeOH (30 mL) was added hydrazine hydrate (99%) (15 mL, 5 vol) dropwise at 0° C. The mixture was allowed to warm to rt and stirred for 1 h, then concentrated under reduced pressure to yield 2-chloro-5-(trifluoromethoxy)benzohydrazide (2.50 g, 9.84 mmol, 83% yield).

LCMS: Method H1, 2.37 min, MS: ES+ 254.9.

Step (iii)

### Ethyl 5-(2-chloro-5-(trifluoromethoxy)phenyl)-1,3,4-oxadiazole-2-carboxylate

To a stirred solution of 2-chloro-5-(trifluoromethoxy)benzohydrazide (2.50 g, 9.84 mmol) in DCM (25 mL) were added TEA (2.98 g, 4.11 mL, 29.52 mmol) and ethyl oxalyl chloride (1.47 g, 1.56 mL, 10.82 mmol) dropwise at 0° C. The mixture was allowed to warm to rt and stirred for 1 h. TsCl (2.24 g, 11.81 mmol) was added in portions at 0° C. The mixture was allowed to warm to rt and stirred at rt for 2 h, then poured into water (50 mL) and extracted with DCM (3×50 mL). The combined organic phases were dried over anhydrous Na₂SO₄, filtered and concentrated under reduced pressure. The residue was purified using flash column chromatography (silica gel, 5% EtOAc in n-hexanes) to yield ethyl 5-(2-chloro-5-(trifluoromethoxy)phenyl)-1,3,4-oxadiazole-2-carboxylate (1.5 g, 4.46 mmol, 45% yield).

LCMS: Method C1, 1.34 min, MS: ES+ 337.0.

### Intermediate Z5

### Ethyl 5-(2-bromo-5-(trifluoromethoxy)phenyl)-1,3,4-oxadiazole-2-carboxylate

### Step (i)

### 2-Bromo-5-(trifluoromethoxy)benzohydrazide

To a stirred solution of methyl 2-bromo-5-(trifluoromethoxy)benzoate (CAS 1150114-81-0, from Enamine, 7.0 g, 23.49 mmol) in MeOH (70 mL) was added hydrazine hydrate (99%) (21 mL, 3 vol) dropwise at 0° C. The mixture was allowed to warm to rt and stirred for 2 h, then concentrated under reduced pressure to yield 2-bromo-5-(trifluoromethoxy)benzohydrazide (5.10 g, 17.11 mmol, 73% yield). LCMS: Method C, 1.39 min, MS: ES+ 299.0, 301.0.

### Step (ii)

### Ethyl 2-(2-(2-bromo-5-(trifluoromethoxy)benzoyl)hydrazineyl)-2-oxoacetate

To a stirred solution of 2-bromo-5-(trifluoromethoxy)benzohydrazide (5.10 g, 17.11 mmol) in DCM (51 mL) were added TEA (5.19 g, 7.2 mL, 51.34 mmol) and ethyl oxalyl chloride (2.80 g, 2.29 mL, 20.53 mmol) dropwise at 0° C. The mixture was allowed to warm to rt, stirred for 2 h, then poured into water (50 mL) and extracted with DCM (3×30 mL). The combined organic phases were dried over anhydrous Na₂SO₄, filtered and concentrated under reduced pressure to yield ethyl 2-(2-(2-bromo-5-(trifluoromethoxy)benzoyl)hydrazineyl)-2-oxoacetate (8.0 g, quantitative yield).

LCMS: Method C1, 1.12 min, MS: ES+ 399.0, 401.0.

Step (iii)

### Ethyl 5-(2-bromo-5-(trifluoromethoxy)phenyl)-1,3,4-oxadiazole-2-carboxylate

To a stirred solution of ethyl 2-(2-(2-bromo-5-(trifluoromethoxy)benzoyl)hydrazineyl)-2-oxoacetate (8.0 g, 20.10 mmol) in DCM (80 mL) were added TEA (6.10 g, 8.40 mL, 60.30 mmol) and TsCl (3.81 g, 20.10 mmol) in portions at 0° C. The mixture was allowed to warm to rt and stirred for 2 h, then poured into water (50 mL) and extracted with DCM (3×30 mL). The combined organic phases were dried over anhydrous Na₂SO₄, filtered and concentrated under reduced pressure. The residue was purified using flash column chromatography (silica gel, 12% EtOAc in n-hexanes) to yield ethyl 5-(2-bromo-5-(trifluoromethoxy)phenyl)-1,3,4-oxadiazole-2-carboxylate (3.63 g, 9.55 mmol, 56% yield over two steps). LCMS: Method C1, 1.35 min, MS: ES+ 380.9, 382.9.

### Intermediate Z6

### Ethyl 3-(2-cyclopropyl-5-(trifluoromethoxy)phenyl)-1,2,4-oxadiazole-5-carboxylate

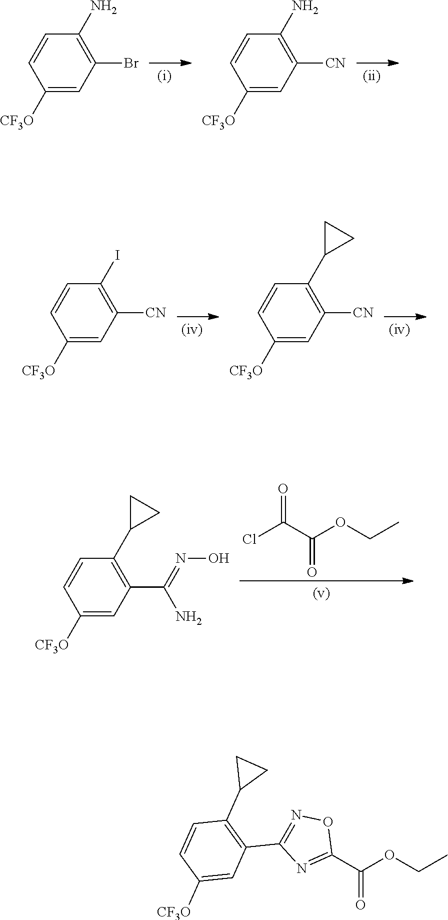

### Step (i)

### 2-Amino-5-(trifluoromethoxy)benzonitrile

To a stirred solution of 2-bromo-4-(trifluoromethoxy)aniline (CAS 175278-17-8, from BLDpharm, 20.0 g, 78.44 mmol) in NMP (150 mL) was added CuCN (14.0 g, 156.80 mmol) and heated at 165° C. for 18 h. The mixture was poured into aqueous ammonia solution (300 mL) and extracted with EtOAc (3×300 mL). The combined organic phases were dried over anhydrous Na₂SO₄ and concentrated under reduced pressure. The residue was purified by column chromatography (silica gel, 8% EtOAc in n-hexanes) to yield 2-amino-5-(trifluoromethoxy)benzonitrile (11.2 g, 55.43 mmol, 70% yield).

LCMS: Method H1, 3.04 min, MS: ES− 201.0.

### Step (ii)

### 2-Iodo-5-(trifluoromethoxy)benzonitrile

To a stirred solution of 2-amino-5-(trifluoromethoxy)benzonitrile (6.0 g, 29.69 mmol) in concentrated HCl (60 mL) was added NaNO₂ (4.0 g, 57.97 mmol) in portions at 0° C. and stirred for 10 min at 0° C. A solution of KI (24.63 g, 74.09 mmol) in water (60 mL) was added dropwise at 0° C. and stirred for 0.5 h. The mixture was poured into water (300 mL) and extracted with EtOAc (3×100 mL). The combined organic phases were washed with 10% sodium thiosulphate solution (2×150 mL), dried over anhydrous Na₂SO₄ and concentrated under reduced pressure. The residue was purified by column chromatography (silica gel, 6% EtOAc in n-hexanes) to yield 2-iodo-5-(trifluoromethoxy)benzonitrile (7.79 g, 24.89 mmol, 84% yield).

¹H NMR (400 MHz, DMSO-d₆) δ ppm: 8.13-8.16 (m, 1H), 8.01 (s, 1H), 7.48 (d, J=8.4 Hz, 1H).

Step (iii)

### 2-Cyclopropyl-5-(trifluoromethoxy)benzonitrile

To a stirred solution of 2-iodo-5-(trifluoromethoxy)benzonitrile (2.6 g, 8.30 mmol) and cyclopropylboronic acid (CAS 411235-57-9, from Combi-Blocks, 1.42 g, 16.53 mmol) in toluene:water (10 mL, 4:1) was added K₃PO₄ (3.52 g, 16.60 mmol). The mixture was purged with N₂ gas for 10 min, followed by addition of [1,1′-bis(diphenylphosphino)ferrocene]dichloropalladium(II) (0.67 g, 0.91 mmol) and heated at 100° C. for 1 h. Two more identical batches were carried out in similar manner and all three reaction mixtures were mixed and poured into water (500 mL) and extracted with EtOAc (3×200 mL). The combined organic phases were dried over anhydrous Na₂SO₄ and concentrated under reduced pressure. The residue was purified by column chromatography (silica gel, 0.5% EtOAc in n-hexanes) to yield 2-cyclopropyl-5-(trifluoromethoxy)benzonitrile (8.2 g, quantitative yield).

¹H NMR (400 MHz, DMSO-d₆) δ ppm: 7.90 (s, 1H), 7.60 (d, J=8.4 Hz, 1H), 7.21 (d, J=8.8 Hz, 1H), 2.16-2.19 (m, 1H), 1.12-1.14 (m, 2H), 0.84-0.85 (m, 2H).

### Step (iv)

### (Z)-2-Cyclopropyl-N′-hydroxy-5-(trifluoromethoxy)benzimidamide

To a stirred solution of 2-cyclopropyl-5-(trifluoromethoxy)benzonitrile (4.0 g, 17.62 mmol) and sodium hydroxide (2.11 g, 52.75 mmol) in EtOH:water (23 mL, 2:1) was added hydroxylamine HCl (3.67 g, 52.81 mmol) and heated at 40° C. for 48 h. The mixture was concentrated under reduced pressure and poured into water (100 mL), and extracted with EtOAc (3×100 mL). The combined organic phases were dried over anhydrous Na₂SO₄ and concentrated under reduced pressure to yield (Z)-2-cyclopropyl-N-hydroxy-5-(trifluoromethoxy)benzimidamide (4.40 g, 16.92 mmol, 96% yield).

LCMS: Method H1, 2.79 min, MS: ES+ 261.0.

### Step (v)

### Ethyl 3-(2-cyclopropyl-5-(trifluoromethoxy)phenyl)-1,2,4-oxadiazole-5-carboxylate

To a stirred solution of (Z)-2-cyclopropyl-N-hydroxy-5-(trifluoromethoxy)benzimidamide (1.50 g, 5.76 mmol) in pyridine (5 mL) was added ethyl oxalyl chloride (1.18 g, 0.98 mL, 8.64 mmol) dropwise at 0° C. The mixture was heated at 80° C. for 3 h, then cooled to rt and concentrated under reduced pressure. The obtained crude was diluted with water (100 mL) and extracted with EtOAc (3×100 mL). The combined organic phases were dried over anhydrous Na₂SO₄ and concentrated under reduced pressure to yield ethyl 3-(2-cyclopropyl-5-(trifluoromethoxy)phenyl)-1,2,4-oxadiazole-5-carboxylate (1.44 g, 4.20 mmol, 73% yield). LCMS: Method C, 2.05 min, MS: ES+ 343.1.

### Intermediate Z7

### Ethyl 5-(2-cyclopropyl-5-(trifluoromethoxy)phenyl)-1,2,4-oxadiazole-3-carboxylate

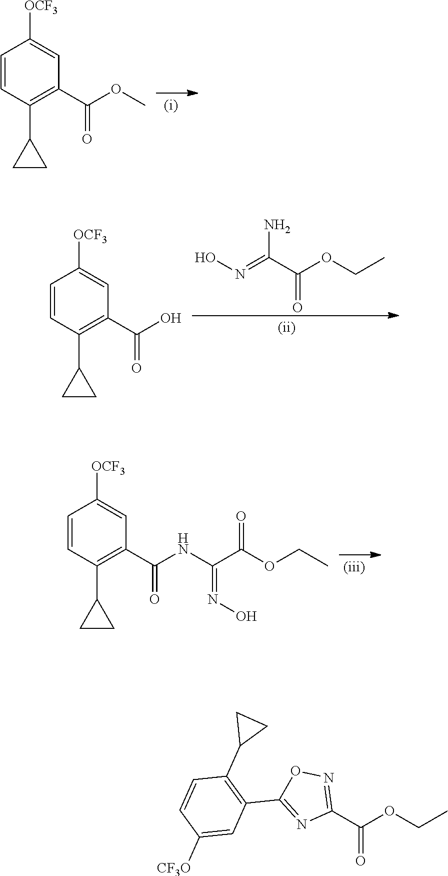

### Step (i)

### 2-Cyclopropyl-5-(trifluoromethoxy)benzoic acid

To a stirred solution of methyl 2-cyclopropyl-5-(trifluoromethoxy)benzoate (1.10 g, 4.23 mmol) in THF:water (10 mL, 1:1) was added LiOH·H₂O (0.35 g, 8.46 mmol) in portions at rt and stirred at rt for 4 h. The mixture was then poured into water (30 mL), acidified with citric acid to pH ˜4 and extracted with EtOAc (2×70 mL). The combined organic phases were dried over anhydrous Na₂SO₄, filtered and concentrated under reduced pressure to yield 2-cyclopropyl-5-(trifluoromethoxy)benzoic acid (1.08 g, quantitative yield). LCMS: Method J1, 3.91 min, MS: ES+ 244.8.

### Step (ii)

### Ethyl (Z)-2-(2-cyclopropyl-5-(trifluoromethoxy)benzamido)-2-(hydroxyimino)acetate

To a stirred solution of 2-cyclopropyl-5-(trifluoromethoxy)benzoic acid (1.20 g, 4.87 mmol) in THF (12 mL) were added DIPEA (1.88 g, 2.5 mL, 14.63 mmol) and HATU (2.78 g, 7.31 mmol) in portions at 0° C. After 30 min, ethyl (Z)-2-amino-2-(hydroxyimino)acetate (CAS 10489-74-4, from Combi-Blocks, 0.77 g, 5.85 mmol) was added at 0° C. The mixture was slowly warmed to rt and stirred at rt for 4 h, then poured into ice-cold water (30 mL) and extracted with EtOAc (2×70 mL). The combined organic phases were dried over Na₂SO₄, filtered and concentrated under reduced pressure to yield ethyl (Z)-2-(2-cyclopropyl-5-(trifluoromethoxy)benzamido)-2-(hydroxyimino)acetate (0.90 g, 2.50 mmol, 59% yield over two steps). LCMS: Method J1, 3.71 min, MS: ES+ 361.2.

Step (iii)

### Ethyl 5-(2-cyclopropyl-5-(trifluoromethoxy)phenyl)-1,2,4-oxadiazole-3-carboxylate

A solution of ethyl (Z)-2-(2-cyclopropyl-5-(trifluoromethoxy)benzamido)-2-(hydroxyimino)acetate (0.90 g, 2.50 mmol) in pyridine (5 mL) was heated at 110° C. for 16 h. The mixture was warmed to rt and then poured into water (30 mL) and extracted with EtOAc (2×50 mL). The combined organic phases were dried over Na₂SO₄, filtered and concentrated under reduced pressure. The residue was purified by flash column chromatography (silica gel, 12% EtOAc in n-hexanes) to yield ethyl 5-(2-cyclopropyl-5-(trifluoromethoxy)phenyl)-1,2,4-oxadiazole-3-carboxylate (0.17 g, 0.49 mmol, 20% yield over two steps). LCMS: Method J1, 4.50 min, MS: ES+ 342.9.

### SYNTHESIS OF EXAMPLES

### Example 1

### N-((3R,5S)-5-((1H-1,2,3-Triazol-1-yl)methyl)-1-cyanopyrrolidin-3-yl)-5-(5-cyano-2-cyclopropylphenyl)-N-cyclopropyloxazole-2-carboxamide

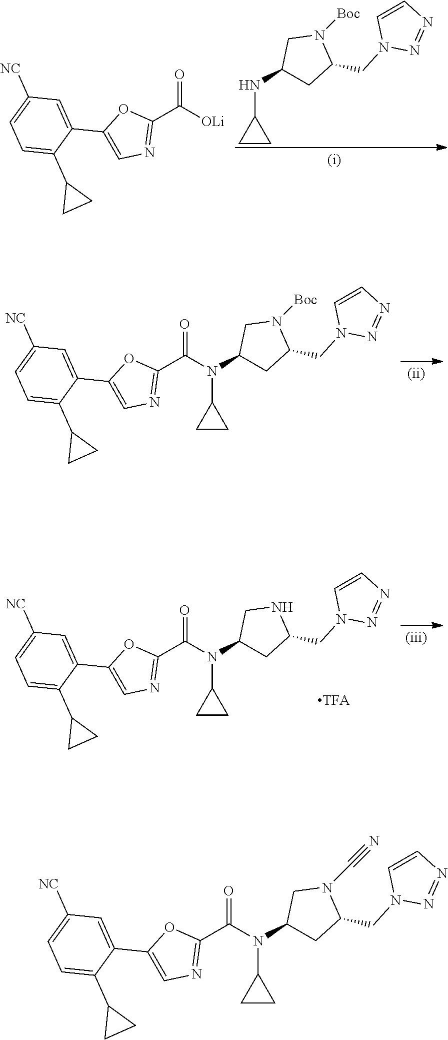

### Step (i)

### tert-Butyl (2S,4R)-2-((1H-1,2,3-triazol-1-yl)methyl)-4-(5-(5-cyano-2-cyclopropylphenyl)-N-cyclopropyloxazole-2-carboxamido)pyrrolidine-1-carboxylate

To a stirred solution of lithium 5-(5-cyano-2-cyclopropylphenyl)oxazole-2-carboxylate (0.3 g, 1.15 mmol) and tert-butyl (2S,4R)-2-((1H-1,2,3-triazol-1-yl)methyl)-4-(cyclopropylamino)pyrrolidine-1-carboxylate (0.35 g, 1.13 mmol) in pyridine (5 mL) at 0° C. was added POCl₃ (0.53 g, 0.3 mL, 3.45 mmol) dropwise. The mixture was stirred at rt for 2 h, then poured into water (50 mL) and extracted with EtOAc (2×50 mL). The combined organic phases were dried over anhydrous Na₂SO₄ and concentrated under reduced pressure. The residue was purified by flash column chromatography (silica gel, 70% EtOAc in n-hexanes) to yield tert-butyl (2S,4R)-2-((1H-1,2,3-triazol-1-yl)methyl)-4-(5-(5-cyano-2-cyclopropylphenyl)-N-cyclopropyloxazole-2-carboxamido)pyrrolidine-1-carboxylate (0.22 g, 0.41 mmol, 36% yield).

LCMS: Method H1, 3.24 min, MS: ES+ 544.2.

### Step (ii)

### N-((3R,5S)-5-((1H-1,2,3-Triazol-1-yl)methyl)pyrrolidin-3-yl)-5-(5-cyano-2-cyclopropylphenyl)-N-cyclopropyloxazole-2-carboxamide TFA salt

To a stirred solution of tert-butyl (2S,4R)-2-((1H-1,2,3-triazol-1-yl)methyl)-4-(5-(5-cyano-2-cyclopropylphenyl)-N-cyclopropyloxazole-2-carboxamido)pyrrolidine-1-carboxylate (0.22 g, 0.40 mmol) in DCM (5 mL) was added TFA (1.1 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(5-cyano-2-cyclopropylphenyl)-N-cyclopropyloxazole-2-carboxamide TFA salt (0.25 g, quantitative yield).

LCMS: Method H1, 2.62 min, MS: ES+ 444.2.

Step (iii)

### N-((3R,5S)-5-((1H-1,2,3-Triazol-1-yl)methyl)-1-cyanopyrrolidin-3-yl)-5-(5-cyano-2-cyclopropylphenyl)-N-cyclopropyloxazole-2-carboxamide

To a stirred solution of N-((3R,5S)-5-((1H-1,2,3-triazol-1-yl)methyl)pyrrolidin-3-yl)-5-(5-cyano-2-cyclopropylphenyl)-N-cyclopropyloxazole-2-carboxamide TFA salt (0.25 g, 0.45 mmol) in THF (5 mL) was added K₂CO₃ (0.18 g, 1.34 mmol) at rt and stirred for 5 min. The mixture was cooled to 0° C., then cyanogen bromide (0.05 g, 0.45 mmol) was added. The mixture was allowed to warm to rt, stirred for 1 h, then poured into water (50 mL) and extracted with EtOAc (2×50 mL). The combined organic phases were dried over anhydrous Na₂SO₄ and concentrated under reduced pressure. The residue was purified by flash column chromatography (silica gel, 80-90% EtOAc in n-hexanes) to yield N-((3R,5S)-5-((1H-1,2,3-triazol-1-yl)methyl)-1-cyanopyrrolidin-3-yl)-5-(5-cyano-2-cyclopropylphenyl)-N-cyclopropyloxazole-2-carboxamide (0.02 g, 0.05 mmol, 13% yield over two steps).

LCMS: Method H1, 2.88 min, MS: ES+ 469.2; ¹H NMR (400 MHz, DMSO-d₆) δ ppm: 8.23 (s, 1H), 8.13 (s, 1H), 7.92 (s, 1H), 7.83 (d, J=8.4 Hz, 1H), 7.79 (s, 1H), 7.41 (d, J=8.4 Hz, 1H), 4.61-4.62 (m, 2H), 4.30-4.45 (m, 2H), 3.76-3.79 (m, 1H), 3.62-3.66 (m, 1H), 2.90-2.31 (m, 1H), 2.19-2.20 (m, 3H), 1.14-1.16 (m, 2H), 0.83-0.85 (m, 2H), 0.57-0.70 (m, 4H); Chiral SFC: Method Y21, 9.34 min.

### Example 2

### N-((3R,5S)-5-((1H-1,2,3-Triazol-1-yl)methyl)-1-cyanopyrrolidin-3-yl)-5-(5-cyano-2-cyclopropylphenyl)-N-cyclopropyloxazole-2-carboxamide

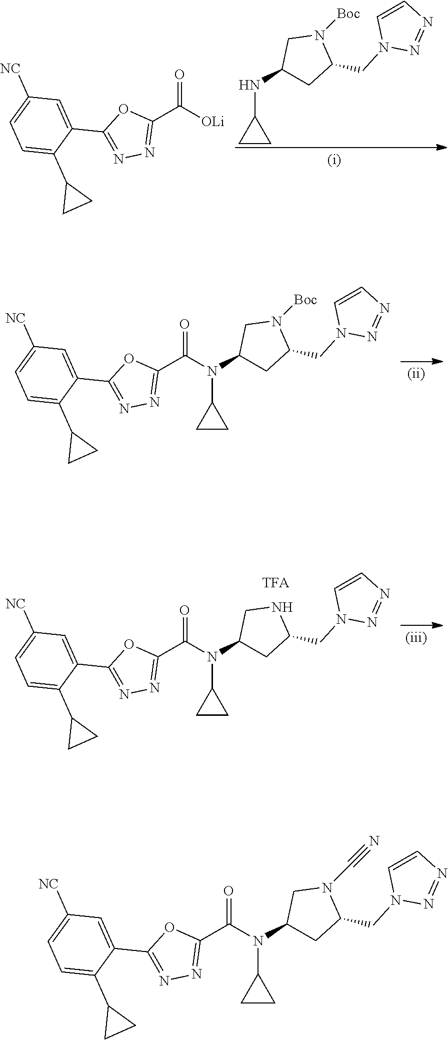

### Step (i)

### tert-Butyl (2S,4R)-2-((1H-1,2,3-triazol-1-yl)methyl)-4-(5-(5-cyano-2-cyclopropylphenyl)-N-cyclopropyl-1,3,4-oxadiazole-2-carboxamido)pyrrolidine-1-carboxylate

To a stirred solution of lithium 5-(5-cyano-2-cyclopropylphenyl)-1,3,4-oxadiazole-2-carboxylate (0.3 g, 1.14 mmol) and tert-butyl (2S,4R)-2-((1H-1,2,3-triazol-1-yl)methyl)-4-(cyclopropylamino) pyrrolidine-1-carboxylate (0.35 g, 1.14 mmol) in pyridine (3 mL) at 0° C. was added POCl₃ (0.52 g, 0.32 mL, 3.42 mmol) dropwise. The mixture was stirred at rt for 20 min, then poured into water (50 mL) and extracted with EtOAc (2×50 mL). The combined organic phases were dried over anhydrous Na₂SO₄ and concentrated under reduced pressure. The residue was purified by flash column chromatography (silica gel, 65% EtOAc in n-hexanes) to yield tert-butyl (2S,4R)-2-((1H-1,2,3-triazol-1-yl)methyl)-4-(5-(5-cyano-2-cyclopropylphenyl)-N-cyclopropyl-1,3,4-oxadiazole-2-carboxamido)pyrrolidine-1-carboxylate (0.30 g, 0.55 mmol, 48% yield).

LCMS: Method H1, 3.96 min, MS: ES+ 545.2.

### Step (ii)

### N-((3R,5S)-5-((1H-1,2,3-Triazol-1-yl)methyl)pyrrolidin-3-yl)-5-(5-cyano-2-cyclopropylphenyl)-N-cyclopropyl-1,3,4-oxadiazole-2-carboxamide TFA salt

To a stirred solution of tert-butyl (2S,4R)-2-((1H-1,2,3-triazol-1-yl)methyl)-4-(5-(5-cyano-2-cyclopropylphenyl)-N-cyclopropyl-1,3,4-oxadiazole-2-carboxamido)pyrrolidine-1-carboxylate (0.30 g, 0.55 mmol) in DCM (3 mL) was added TFA (1.5 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(5-cyano-2-cyclopropylphenyl)-N-cyclopropyl-1,3,4-oxadiazole-2-carboxamide TFA salt (0.40 g, quantitative yield).

LCMS: Method J1, 2.75 min, MS: ES+ 445.3.

Step (iii)

### N-((3R,5S)-5-((1H-1,2,3-Triazol-1-yl)methyl)-1-cyanopyrrolidin-3-yl)-5-(5-cyano-2-cyclopropylphenyl)-N-cyclopropyl-1,3,4-oxadiazole-2-carboxamide

To a stirred solution of N-((3R,5S)-5-((1H-1,2,3-triazol-1-yl)methyl)pyrrolidin-3-yl)-5-(5-cyano-2-cyclopropylphenyl)-N-cyclopropyl-1,3,4-oxadiazole-2-carboxamide TFA salt (0.40 g, 0.71 mmol) in THF (4 mL) was added K₂CO₃ (0.29 g, 2.15 mmol) at rt and stirred for 5 min. Cyanogen bromide (0.07 g, 0.71 mmol) was added at 0° C. The mixture was stirred at rt for 1 h, then poured into water (30 mL) and extracted with EtOAc (2×30 mL). The combined organic phases were dried over anhydrous Na₂SO₄ and concentrated under reduced pressure. The residue was purified by flash column chromatography (silica gel, 70% EtOAc in n-hexanes) to yield N-((3R,5S)-5-((1H-1,2,3-triazol-1-yl)methyl)-1-cyanopyrrolidin-3-yl)-5-(5-cyano-2-cyclopropylphenyl)-N-cyclopropyl-1,3,4-oxadiazole-2-carboxamide (0.09 g, 0.18 mmol, 35% yield over two steps).

LCMS: Method H1, 2.77 min, MS: ES+ 470.2; ¹H NMR (400 MHz, DMSO-d₆) δ ppm: 8.34 (s, 1H), 8.24 (s, 1H), 8.0 (d, J=8.0 Hz, 1H), 7.80 (s, 1H), 7.36 (d, J=8.0 Hz, 1H), 4.62-4.63 (m, 2H), 4.35-4.42 (m, 2H), 3.78-3.82 (m, 1H), 3.65-3.69 (m, 1H), 3.05-3.17 (m, 1H), 2.80-2.91 (m, 1H), 2.59-2.64 (m, 1H), 2.17-2.25 (m, 1H), 1.14-1.23 (m, 2H), 0.9-1.0 (m, 2H), 0.60-0.83 (m, 4H); Chiral SFC: Method Y28, 14.39 min.

### Example 3

### N-((3R,5S)-5-((1H-1,2,3-Triazol-1-yl)methyl)-1-cyanopyrrolidin-3-yl)-5-(3-cyanophenyl)oxazole-2-carboxamide

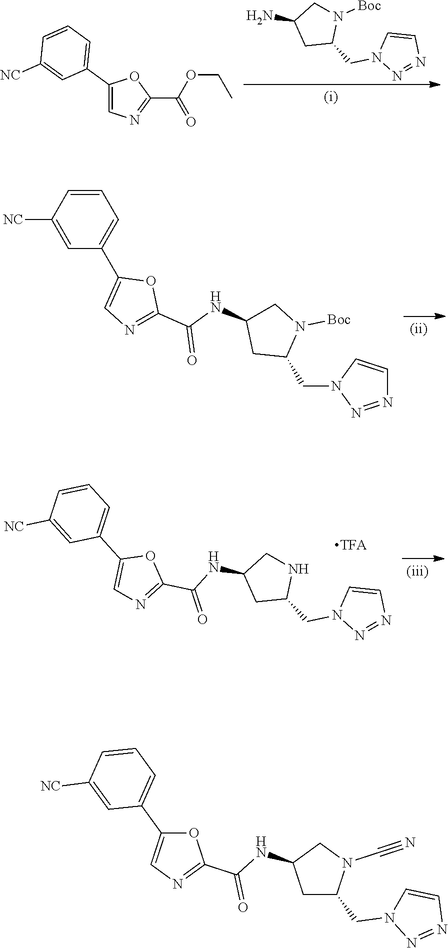

### Step (i)

### tert-Butyl (2S,4R)-2-((1H-1,2,3-triazol-1-yl)methyl)-4-(5-(3-cyanophenyl)oxazole-2-carboxamido)pyrrolidine-1-carboxylate

To a stirred solution of ethyl 5-(3-cyanophenyl)oxazole-2-carboxylate (0.54 g, 2.24 mmol) and tert-butyl (2S,4R)-2-((1H-1,2,3-triazol-1-yl)methyl)-4-aminopyrrolidine-1-carboxylate (0.30 g, 1.12 mmol) in THF (6 mL) was added TBD (0.31 g, 2.24 mmol) in portions at 0° C. The mixture was allowed to warm to rt and stirred for 24 h and heated at 70° C. for 24 h. The mixture was poured into water (100 mL) and extracted with EtOAc (4×100 mL). The combined organic phases were dried over anhydrous Na₂SO₄, filtered and concentrated under reduced pressure. The residue was purified by flash column chromatography (silica gel, 3.5% MeOH in DCM) to yield tert-butyl (2S,4R)-2-((1H-1,2,3-triazol-1-yl)methyl)-4-(5-(3-cyanophenyl)oxazole-2-carboxamido)pyrrolidine-1-carboxylate (0.14 g, 0.30 mmol, 27% yield).

LCMS: Method C, 1.52 min, MS: ES+ 464.5.

### Step (ii)

### N-((3R,5S)-5-((1H-1,2,3-Triazol-1-yl)methyl)pyrrolidin-3-yl)-5-(3-cyanophenyl)oxazole-2-carboxamide TFA salt

To a stirred solution of tert-butyl (2S,4R)-2-((1H-1,2,3-triazol-1-yl)methyl)-4-(5-(3-cyanophenyl)oxazole-2-carboxamido)pyrrolidine-1-carboxylate (0.14 g, 0.30 mmol) in DCM (2.8 mL) was added TFA (0.7 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(3-cyanophenyl)oxazole-2-carboxamide TFA salt (0.15 g, quantitative yield).

LCMS: Method C, 1.33 min, MS: ES+ 364.5.

Step (iii)

### N-((3R,5S)-5-((1H-1,2,3-Triazol-1-yl)methyl)-1-cyanopyrrolidin-3-yl)-5-(3-cyanophenyl)oxazole-2-carboxamide

To a stirred solution of N-((3R,5S)-5-((1H-1,2,3-triazol-1-yl)methyl)pyrrolidin-3-yl)-5-(3-cyanophenyl)oxazole-2-carboxamide TFA salt (0.15 g, 0.31 mmol) in THF (3 mL) was added K₂CO₃ (0.13 g, 0.94 mmol) at rt and stirred for 5 min. Cyanogen bromide (0.03 g, 0.31 mmol) was added into the reaction mixture at 0° C. The mixture was stirred at rt for 1 h, then poured into ice-cold water (100 mL) and the solid filtered through a Buchner funnel, washed with n-hexanes (2×100 mL). The solid residue was purified by trituration using diethylether (2×5 mL) to yield N-((3R,5S)-5-((1H-1,2,3-triazol-1-yl)methyl)-1-cyanopyrrolidin-3-yl)-5-(3-cyanophenyl)oxazole-2-carboxamide (0.07 g, 0.18 mmol, 59% yield over two steps).

LCMS: Method H, 2.27 min, MS: ES+ 389.1; ¹H NMR (400 MHz, DMSO-d₆) δ ppm: 9.39 (d, J=6.8 Hz, 1H), 8.34 (s, 1H), 8.20 (s, 1H), 8.14 (d, J=8.0 Hz, 1H), 8.10 (s, 1H), 7.92 (d, J=7.6 Hz, 1H), 7.78 (s, 1H), 7.75 (t, J=8.0 Hz, 1H), 4.61-4.63 (m, 2H), 4.33-4.38 (m, 2H), 3.65-3.69 (m, 1H), 3.39-3.43 (m, 1H), 2.16-2.25 (m, 1H), 1.95-2.03 (m, 1H); Chiral SFC: Method Y12A, 4.55 min.

### Example 4

### N-((3R,5S)-5-((1H-1,2,3-Triazol-1-yl)methyl)-1-cyanopyrrolidin-3-yl)-5-(3-(trifluoromethoxy)phenyl)oxazole-2-carboxamide

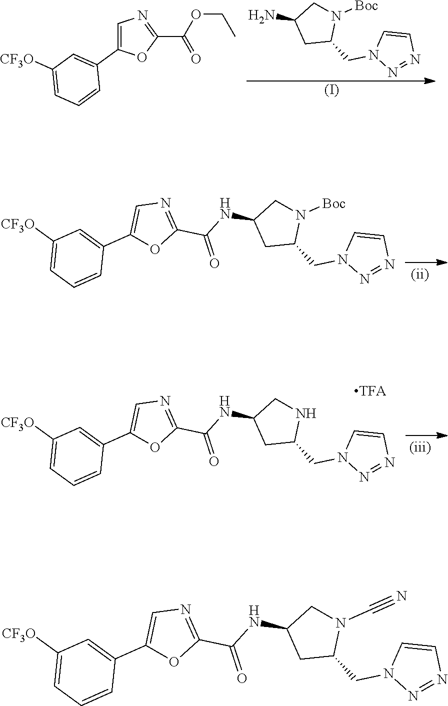

### Step (i)

### tert-Butyl (2S,4R)-2-((1H-1,2,3-triazol-1-yl)methyl)-4-(5-(3-(trifluoromethoxy)phenyl)oxazole-2-carboxamido)pyrrolidine-1-carboxylate

To a stirred solution of ethyl 5-(3-(trifluoromethoxy)phenyl)oxazole-2-carboxylate (0.20 g, 0.66 mmol) and tert-butyl (2S,4R)-2-((1H-1,2,3-triazol-1-yl)methyl)-4-aminopyrrolidine-1-carboxylate (0.18 g, 0.66 mmol) in THF (10 mL) was added DBU (1 mL, 5 vol) at rt and stirred for 16 h. The mixture was poured into water (30 mL) and extracted with EtOAc (3×30 mL). The combined organic phases were dried over anhydrous Na₂SO₄, filtered and concentrated under reduced pressure. The residue was purified by flash column chromatography (silica gel, 50% EtOAc in n-hexanes) to yield tert-butyl (2S,4R)-2-((1H-1,2,3-triazol-1-yl)methyl)-4-(5-(3-(trifluoromethoxy)phenyl)oxazole-2-carboxamido)pyrrolidine-1-carboxylate (0.08 g, 0.15 mmol, 23% yield).

LCMS: Method C1, 1.28 min, MS: ES+ 523.4.

### Step (ii)

### N-((3R,5S)-5-((1H-1,2,3-Triazol-1-yl)methyl)pyrrolidin-3-yl)-5-(3-(trifluoromethoxy)phenyl)oxazole-2-carboxamide TFA salt

To a stirred solution of tert-butyl (2S,4R)-2-((1H-1,2,3-triazol-1-yl)methyl)-4-(5-(3-(trifluoromethoxy)phenyl)oxazole-2-carboxamido)pyrrolidine-1-carboxylate (0.08 g, 0.15 mmol) in DCM (3 mL) was added TFA (0.4 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(3-(trifluoromethoxy)phenyl)oxazole-2-carboxamide TFA salt (0.14 g, quantitative yield).

LCMS: Method C1, 1.07 min, MS: ES+ 423.3.

Step (iii)

### N-((3R,5S)-5-((1H-1,2,3-Triazol-1-yl)methyl)-1-cyanopyrrolidin-3-yl)-5-(3-(trifluoromethoxy)phenyl)oxazole-2-carboxamide

To a stirred solution of N-((3R,5S)-5-((1H-1,2,3-triazol-1-yl)methyl)pyrrolidin-3-yl)-5-(3-(trifluoromethoxy)phenyl)oxazole-2-carboxamide TFA salt (0.14 g, 0.25 mmol) in THF (3 mL) was added K₂CO₃ (0.07 g, 0.51 mmol) at rt and stirred for 5 min. Cyanogen bromide (0.04 g, 0.38 mmol) was added into the reaction mixture at 0° C. The mixture was stirred at rt for 1 h, then poured into water (20 mL) and extracted with EtOAc (3×20 mL). The combined organic phases were dried over anhydrous Na₂SO₄, filtered and concentrated under reduced pressure. The residue was purified by flash column chromatography (silica gel, 68% EtOAc in n-hexanes) to yield N-((3R,5S)-5-((1H-1,2,3-triazol-1-yl)methyl)-1-cyanopyrrolidin-3-yl)-5-(3-(trifluoromethoxy)phenyl)oxazole-2-carboxamide (0.03 g, 0.08 mmol, 51% yield over two steps).

LCMS: Method H1, 2.80 min, MS: ES+ 448.2; ¹H NMR (400 MHz, DMSO-d₆) δ ppm: 9.38 (d, J=6.4 Hz, 1H), 8.20 (s, 1H), 8.10 (s, 1H), 7.88 (d, J=7.6 Hz, 1H), 7.83 (s, 1H), 7.79 (s, 1H), 7.69 (t, J=8.0 Hz, 1H), 7.47 (d, J=8.0 Hz, 1H), 4.62-4.65 (m, 2H), 4.33-4.41 (m, 2H), 3.66-3.70 (m, 1H), 3.40-3.44 (m, 1H), 2.18-2.25 (m, 1H), 1.97-2.03 (m, 1H); Chiral SFC: Method Y12A, 3.77 min.

### Example 5

### N-((3R,5S)-5-((1H-1,2,3-Triazol-1-yl)methyl)-1-cyanopyrrolidin-3-yl)-5-(3-chlorophenyl)oxazole-2-carboxamide

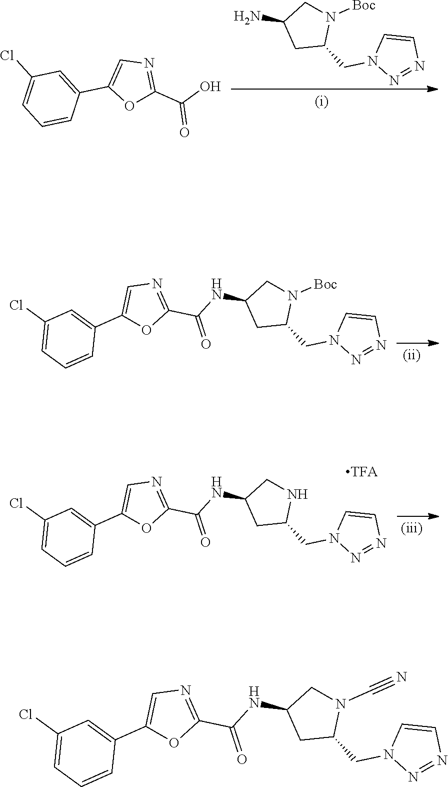 [SMILES: O=C(O)c1ncc(-c2cccc(Cl)c2)o1 ; CC(C)(C)OC(=O)N1C[C@H](N)C[C@H]1Cn1ccnn1 ; CC(C)(C)OC(=O)N1C[C@H](NC(=O)c2ncc(-c3cccc(Cl)c3)o2)C[C@H]1Cn1ccnn1 ; O=C(N[C@H]1CN[C@H](Cn2ccnn2)C1)c1ncc(-c2cccc(Cl)c2)o1 ; N#CN1C[C@H](NC(=O)c2ncc(-c3cccc(Cl)c3)o2)C[C@H]1Cn1ccnn1]

### Step (i)

### tert-Butyl (2S,4R)-2-((1H-1,2,3-triazol-1-yl)methyl)-4-(5-(3-chlorophenyl)oxazole-2-carboxamido) pyrrolidine-1-carboxylate

To a stirred solution of 5-(3-chlorophenyl)oxazole-2-carboxylic acid (0.1 g, 0.44 mmol) and tert-butyl (2S,4R)-2-((1H-1,2,3-triazol-1-yl)methyl)-4-aminopyrrolidine-1-carboxylate (0.10 g, 0.37 mmol) in THF (5 mL) were added DIPEA (0.14 g, 0.19 mL, 1.11 mmol) and HATU (0.35 g, 0.92 mmol) at 0° C.

The mixture was stirred at rt for 2 h, then poured into ice-cold water (30 mL) and extracted with EtOAc (2×30 mL). The combined organic phases were dried over anhydrous Na₂SO₄ and concentrated under reduced pressure. The residue was purified by flash column chromatography (silica gel, 50% EtOAc in n-hexanes) to yield tert-butyl (2S,4R)-2-((1H-1,2,3-triazol-1-yl)methyl)-4-(5-(3-chlorophenyl)-oxazole-2-carboxamido)pyrrolidine-1-carboxylate (0.15 g, 0.32 mmol, 84% yield).

LCMS: Method C1, 1.27 min, MS: ES+ 473.4.

### Step (ii)

### N-((3R,5S)-5-((1H-1,2,3-Triazol-1-yl)methyl)pyrrolidin-3-yl)-5-(3-chlorophenyl)oxazole-2-carboxamide TFA salt

To a stirred solution of tert-butyl (2S,4R)-2-((1H-1,2,3-triazol-1-yl)methyl)-4-(5-(3-chlorophenyl)oxazole-2-carboxamido)pyrrolidine-1-carboxylate (0.15 g, 0.32 mmol) in DCM (3 mL) was added TFA (2.25 mL, 15 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(3-chlorophenyl)oxazole-2-carboxamide TFA salt (0.10 g, 0.20 mmol, 64% yield).

LCMS: Method C1, 1.04 min, MS: ES+ 373.3.

Step (iii)

### N-((3R,5S)-5-((1H-1,2,3-Triazol-1-yl)methyl)-1-cyanopyrrolidin-3-yl)-5-(3-chlorophenyl)oxazole-2-carboxamide

To a stirred solution of N-((3R,5S)-5-((1H-1,2,3-triazol-1-yl)methyl)pyrrolidin-3-yl)-5-(3-chlorophenyl)oxazole-2-carboxamide TFA salt (0.10 g, 0.20 mmol) in MeCN (3 mL) was added K₂CO₃ (0.08 g, 0.62 mmol) at rt and stirred for 5 min. Cyanogen bromide (0.03 g, 0.24 mmol) was added into the reaction mixture at 0° C. The mixture was stirred at rt for 1 h, then poured into water (20 mL) and extracted with EtOAc (2×20 mL). The combined organic phases were dried over anhydrous Na₂SO₄, filtered and concentrated under reduced pressure. The residue was purified by flash column chromatography (silica gel, 70% EtOAc in n-hexanes) to yield N-((3R,5S)-5-((1H-1,2,3-triazol-1-yl)methyl)-1-cyanopyrrolidin-3-yl)-5-(3-chlorophenyl)oxazole-2-carboxamide (0.04 g, 0.10 mmol, 49% yield) which was further purified by reverse phase preparative HPLC (Method X1) to yield N-((3R,5S)-5-((1H-1,2,3-triazol-1-yl)methyl)-1-cyanopyrrolidin-3-yl)-5-(3-chlorophenyl)oxazole-2-carboxamide (0.02 g, 0.05 mmol, 23% yield).

LCMS: Method H1, 2.64 min, MS: ES+ 398.0; ¹H NMR (400 MHz, DMSO-d₆) δ ppm: 9.37 (d, J=6.8 Hz, 1H), 8.20 (s, 1H), 8.06 (s, 1H), 7.93 (s, 1H), 7.79-7.81 (m, 2H), 7.51-7.59 (m, 2H), 4.62-4.64 (m, 2H), 4.34-4.38 (m, 2H), 3.66-3.70 (m, 1H), 3.40-3.44 (m, 1H), 2.18-2.24 (m, 1H), 1.96-2.03 (m, 1H); Chiral SFC: Method Y20, 5.44 min.

### Example 6

### N-((3R,5S)-5-((1H-1,2,3-Triazol-1-yl)methyl)-1-cyanopyrrolidin-3-yl)-5-(2-methoxyphenyl)oxazole-2-carboxamide

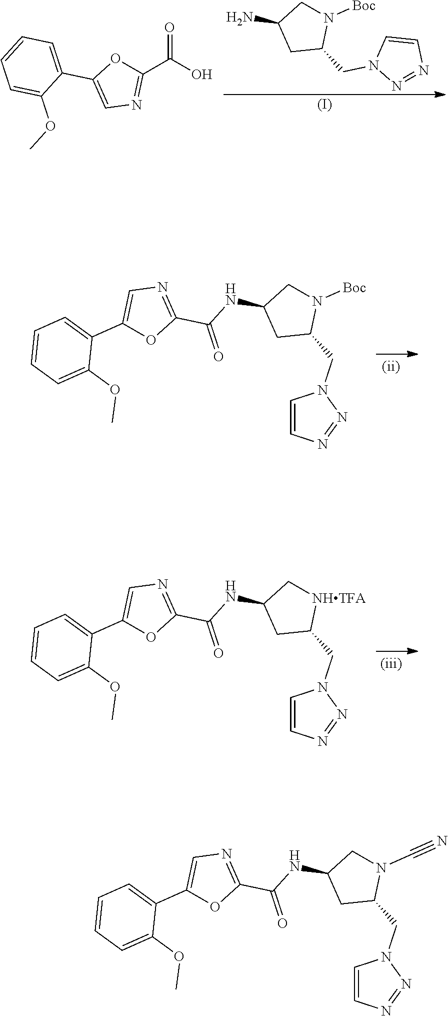

### Step (i)

### tert-Butyl (2S,4R)-2-((1H-1,2,3-triazol-1-yl)methyl)-4-(5-(2-methoxyphenyl)oxazole-2-carboxamido)pyrrolidine-1-carboxylate

To a stirred solution of 5-(2-methoxyphenyl)oxazole-2-carboxylic acid (0.2 g, 0.91 mmol) and tert-butyl (2S,4R)-2-((1H-1,2,3-triazol-1-yl)methyl)-4-aminopyrrolidine-1-carboxylate (0.21 g, 1.01 mmol) in THF (5 mL) were added DIPEA (0.35.g, 0.49 mL, 1.11 mmol) and HATU (0.52 g, 1.36 mmol) at 0° C. The mixture was stirred at rt for 12 h, then poured into ice-cold water (30 mL) and extracted with EtOAc (2×30 mL). The combined organic phases were dried over anhydrous Na₂SO₄, filtered and concentrated under reduced pressure. The residue was purified by flash column chromatography (silica gel, 50% EtOAc in n-hexanes) to yield tert-butyl (2S,4R)-2-((1H-1,2,3-triazol-1-yl)methyl)-4-(5-(2-methoxyphenyl)oxazole-2-carboxamido)pyrrolidine-1-carboxylate (0.18 g, 0.38 mmol, 42% yield).

LCMS: Method C1, 1.22 min, MS: ES+ 469.8.

### Step (ii)

### N-((3R,5S)-5-((1H-1,2,3-Triazol-1-yl)methyl)pyrrolidin-3-yl)-5-(2-methoxyphenyl)oxazole-2-carboxamide TFA salt

To a stirred solution of tert-butyl (2S,4R)-2-((1H-1,2,3-triazol-1-yl)methyl)-4-(5-(2-methoxyphenyl) oxazole-2-carboxamido)pyrrolidine-1-carboxylate (0.18 g, 0.38 mmol) in DCM (3 mL) was added TFA (1.08 mL, 6 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(2-methoxyphenyl)oxazole-2-carboxamide TFA salt (0.15 g, quantitative yield).

LCMS: Method C1, 1.00 min, MS: ES+ 369.4.

Step (iii)

### N-((3R,5S)-5-((1H-1,2,3-Triazol-1-yl)methyl)-1-cyanopyrrolidin-3-yl)-5-(2-methoxyphenyl)oxazole-2-carboxamide

To a stirred solution of N-((3R,5S)-5-((1H-1,2,3-triazol-1-yl)methyl)pyrrolidin-3-yl)-5-(2-methoxyphenyl)oxazole-2-carboxamide TFA salt (0.15 g, 0.31 mmol) in THF (5 mL) was added K₂CO₃ (0.12 g, 0.93 mmol) at rt and stirred for 5 min. Cyanogen bromide (0.03 g, 0.34 mmol) was added into the reaction mixture at 0° C. The mixture was stirred at rt for 1 h, then poured into water (20 mL) and extracted with EtOAc (2×20 mL). The combined organic phases were dried over anhydrous Na₂SO₄, filtered and concentrated under reduced pressure. The residue was purified by flash column chromatography (silica gel, 100% EtOAc in n-hexanes) to yield N-((3R,5S)-5-((1H-1,2,3-triazol-1-yl)methyl)-1-cyanopyrrolidin-3-yl)-5-(2-methoxyphenyl)oxazole-2-carboxamide (0.09 g, 0.22 mmol, 59% yield over two steps).

LCMS: Method H1, 2.47 min, MS: ES+ 394.2; ¹H NMR (400 MHz, DMSO-d₆) δ ppm: 9.33 (d, J=6.4 Hz, 1H), 8.19 (s, 1H), 7.82 (dd, J=1.2, 7.6 Hz, 1H), 7.78 (s, 1H), 7.71 (s, 1H), 7.42-7.47 (m, 1H), 7.21 (d, J=8.4, Hz 1H), 7.13 (t, J=7.6 Hz, 1H), 4.61-4.62 (m, 2H), 4.34-4.39 (m, 2H), 3.97 (s, 3H), 3.64-3.68 (m, 1H), 3.38-3.43 (m, 1H), 2.17-2.24 (m, 1H), 1.95-2.01 (m, 1H); Chiral SFC: Method Y4, 5.36 min.

### Example 7

### N-((3R,5S)-5-((1H-1,2,3-Triazol-1-yl)methyl)-1-cyanopyrrolidin-3-yl)-5-(2-cyclopropylphenyl)-1,3,4-oxadiazole-2-carboxamide

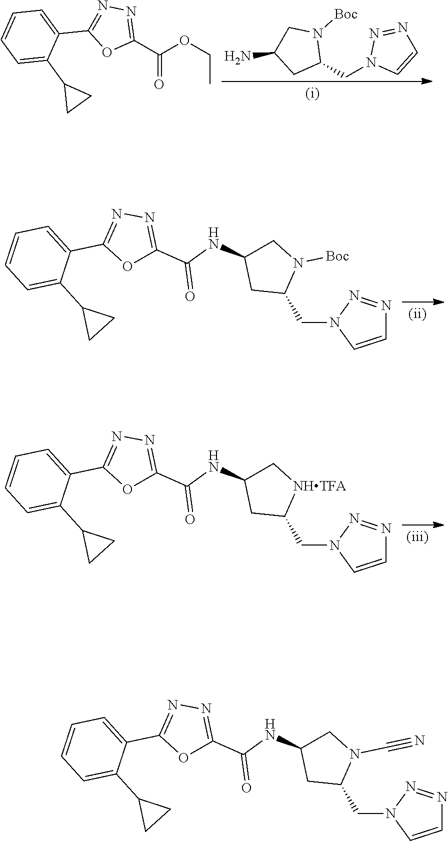

### Step (i)

### tert-Butyl (2S,4R)-2-((1H-1,2,3-triazol-1-yl)methyl)-4-(5-(2-cyclopropylphenyl)-1,3,4-oxadiazole-2-carboxamido)pyrrolidine-1-carboxylate

A stirred solution of ethyl 5-(2-cyclopropylphenyl)-1,3,4-oxadiazole-2-carboxylate (0.7 g, 2.71 mmol) and tert-butyl (2S,4R)-2-((1H-1,2,3-triazol-1-yl)methyl)-4-aminopyrrolidine-1-carboxylate (0.76 g, 2.84 mmol) in THF (7 mL) was purged with N₂ for 10 min. TBD (0.41 g, 2.98 mmol) was added in portions at 0° C. The mixture was allowed to warm to rt and stirred for 16 h at rt. The mixture was poured into water (50 mL), precipitated solid was collected by filtration under reduced pressure, washed with water (2×10 mL), n-hexanes (2×20 mL) and dried under reduced pressure to yield tert-butyl (2S,4R)-2-((1H-1,2,3-triazol-1-yl)methyl)-4-(5-(2-cyclopropylphenyl)-1,3,4-oxadiazole-2-carboxamido)pyrrolidine-1-carboxylate (0.46 g, 0.97 mmol, 36% yield).

LCMS: Method C1, 1.29 min, MS: ES+ 480.41.

### Step (ii)

### N-((3R,5S)-5-((1H-1,2,3-Triazol-1-yl)methyl)pyrrolidin-3-yl)-5-(2-cyclopropylphenyl)-1,3,4-oxadiazole-2-carboxamide TFA salt

To a stirred solution of tert-butyl (2S,4R)-2-((1H-1,2,3-triazol-1-yl)methyl)-4-(5-(2-cyclopropylphenyl)-1,3,4-oxadiazole-2-carboxamido)pyrrolidine-1-carboxylate (0.46 g, 0.95 mmol) in DCM (5 mL) was added TFA (2.3 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(2-cyclopropylphenyl)-1,3,4-oxadiazole-2-carboxamide TFA salt (0.48 g, quantitative yield).

LCMS: Method C1, 1.03 min, MS: ES+ 380.2.

Step (iii)

### N-((3R,5S)-5-((1H-1,2,3-Triazol-1-yl)methyl)-1-cyanopyrrolidin-3-yl)-5-(2-cyclopropylphenyl)-1,3,4-oxadiazole-2-carboxamide

To a stirred solution of N-((3R,5S)-5-((1H-1,2,3-triazol-1-yl)methyl)pyrrolidin-3-yl)-5-(2-cyclopropylphenyl)-1,3,4-oxadiazole-2-carboxamide TFA salt (0.48 g, 0.97 mmol) in THF (5 mL) was added K₂CO₃ (0.40 g, 2.92 mmol) at rt and stirred for 5 min. Cyanogen bromide (0.10 g, 0.97 mmol) was added into the reaction mixture at 0° C. The mixture was stirred at rt for 1 h, then poured into water (50 mL) and extracted with EtOAc (3×50 mL). The combined organic phases were dried over anhydrous Na₂SO₄ and concentrated under reduced pressure. The residue was purified by flash column chromatography (silica gel, 100% EtOAc) to yield N-((3R,5S)-5-((1H-1,2,3-triazol-1-yl)methyl)-1-cyanopyrrolidin-3-yl)-5-(2-cyclopropylphenyl)-1,3,4-oxadiazole-2-carboxamide (0.1 g, 0.24 mmol, 26% yield over two steps).

LCMS: Method H1, 2.72 min, MS: ES+ 405.2; ¹H NMR (400 MHz, DMSO-d₆) δ 9.77 (d, J=6.4 Hz, 1H), 8.21 (s, 1H), 7.89 (d, J=7.6 Hz, 1H), 7.80 (s, 1H), 7.56 (t, J=7.6 Hz, 1H), 7.41 (t, J=7.6 Hz, 1H), 7.19 (d, J=7.6 Hz, 1H), 4.64-4.65 (m, 2H), 4.36-4.39 (m, 2H), 3.67-3.71 (m, 1H), 3.43-3.46 (m, 1H), 2.69-2.71 (m, 1H), 2.19-2.25 (m, 1H), 1.99-2.04 (m, 1H), 1.03-1.04 (m, 2H), 0.77-0.78 (m, 2H); Chiral HPLC: Method Y21, 10.58 min.

### Example 8

### N-((3R,5S)-5-((1H-1,2,3-Triazol-1-yl)methyl)-1-cyanopyrrolidin-3-yl)-5-(2-cyclopropylphenyl)oxazole-2-carboxamide

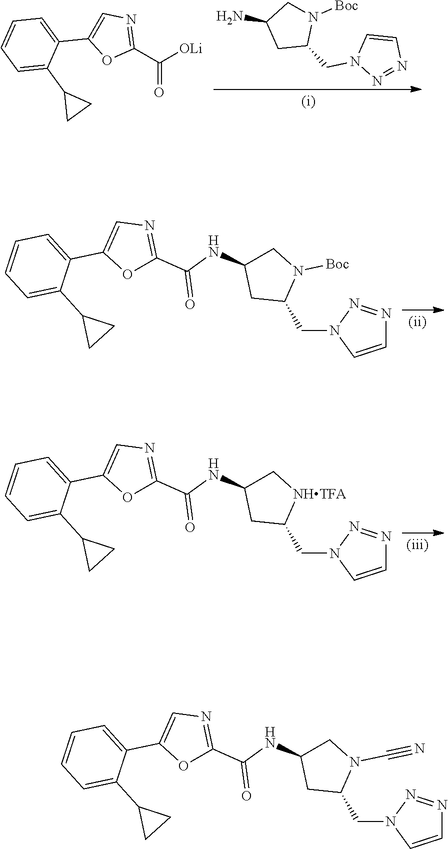

### Step (i)

### tert-Butyl (2S,4R)-2-((1H-1,2,3-triazol-1-yl)methyl)-4-(5-(2-cyclopropylphenyl)oxazole-2-carboxamido)pyrrolidine-1-carboxylate

To a stirred solution of lithium 5-(2-cyclopropylphenyl)oxazole-2-carboxylate (0.5 g, 2.12 mmol) and tert-butyl (2S,4R)-2-((1H-1,2,3-triazol-1-yl)methyl)-4-aminopyrrolidine-1-carboxylate (0.45 g, 1.70 mmol) in THF (10 mL) was added DIPEA (0.82 g, 1.09 mL, 6.38 mmol) at 0° C. After 10 min, T₃P (50% in EtOAc) (1.01 g, 2.02 mL, 3.19 mmol) was added dropwise at 0° C. The mixture was stirred at rt for 5 h, then poured into water (50 mL) and extracted with EtOAc (2×50 mL). The combined organic phases were dried over anhydrous Na₂SO₄ and concentrated under reduced pressure. The residue was purified by flash column chromatography (silica gel, 90% EtOAc in n-hexanes) to yield tert-butyl (2S,4R)-2-((1H-1,2,3-triazol-1-yl)methyl)-4-(5-(2-cyclopropylphenyl)oxazole-2-carboxamido)pyrrolidine-1-carboxylate (0.15 g, 0.31 mmol, 16% yield over two steps).

LCMS: Method H1, 3.32 min, MS: ES− 477.2.

### Step (ii)

### N-((3R,5S)-5-((1H-1,2,3-Triazol-1-yl)methyl)pyrrolidin-3-yl)-5-(2-cyclopropylphenyl)oxazole-2-carboxamide TFA salt

To a stirred solution of tert-butyl (2S,4R)-2-((1H-1,2,3-triazol-1-yl)methyl)-4-(5-(2-cyclopropylphenyl)oxazole-2-carboxamido)pyrrolidine-1-carboxylate (0.15 g, 0.31 mmol) in DCM (5 mL) was added TFA (1.5 mL, 10 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(2-cyclopropylphenyl)oxazole-2-carboxamide TFA salt (0.17 g, quantitative yield). LCMS: Method H1, 3.00 min, MS: ES+ 379.0.

Step (iii)

### N-((3R,5S)-5-((1H-1,2,3-Triazol-1-yl)methyl)-1-cyanopyrrolidin-3-yl)-5-(2-cyclopropylphenyl)oxazole-2-carboxamide

To a stirred solution of N-((3R,5S)-5-((1H-1,2,3-triazol-1-yl)methyl)pyrrolidin-3-yl)-5-(2-cyclopropylphenyl)oxazole-2-carboxamide TFA salt (0.17 g, 0.34 mmol) in THF (5 mL) was added K₂CO₃ (0.14 g, 1.03 mmol) at rt and stirred for 5 min. Cyanogen bromide (0.04 g, 0.34 mmol) was added into the reaction mixture at 0° C. The mixture was stirred at rt for 1 h, then poured into water (50 mL) and extracted with EtOAc (2×50 mL). The combined organic phases were dried over anhydrous Na₂SO₄, filtered and concentrated under reduced pressure. The residue was purified by flash column chromatography (silica gel, 100% EtOAc) to yield N-((3R,5S)-5-((1H-1,2,3-triazol-1-yl)methyl)-1-cyanopyrrolidin-3-yl)-5-(2-cyclopropylphenyl)oxazole-2-carboxamide (0.08 g, 0.19 mmol, 63% yield over two steps) which was further purified by reverse phase preparative HPLC (Method X4) to yield N-((3R,5S)-5-((1H-1,2,3-triazol-1-yl)methyl)-1-cyanopyrrolidin-3-yl)-5-(2-cyclopropylphenyl)oxazole-2-carboxamide (0.04 g, 0.11 mmol, 35% yield over two steps).

LCMS: Method H1, 2.68 min, MS: ES+ 404.0; ¹H NMR (400 MHz, DMSO-d₆) δ ppm: 9.38 (d, J=6.8 Hz, 1H), 8.21 (s, 1H), 7.75-7.82 (m, 3H), 7.38-7.39 (m, 2H), 7.24-7.26 (m, 1H), 4.62-4.64 (m, 2H), 4.34-4.39 (m, 2H), 3.65-3.69 (m, 1H), 3.41-3.45 (m, 1H), 2.19-2.23 (m, 1H), 2.11-2.15 (m, 1H), 1.98-2.02 (m, 1H), 1.02-1.07 (m, 2H), 0.69-0.73 (m, 2H); Chiral SFC: Method Y12A, 4.52 min.

### Example 9

### N-((3R,5S)-5-((1H-1,2,3-Triazol-1-yl)methyl)-1-cyanopyrrolidin-3-yl)-5-(5-cyano-2-cyclopropylphenyl)oxazole-2-carboxamide

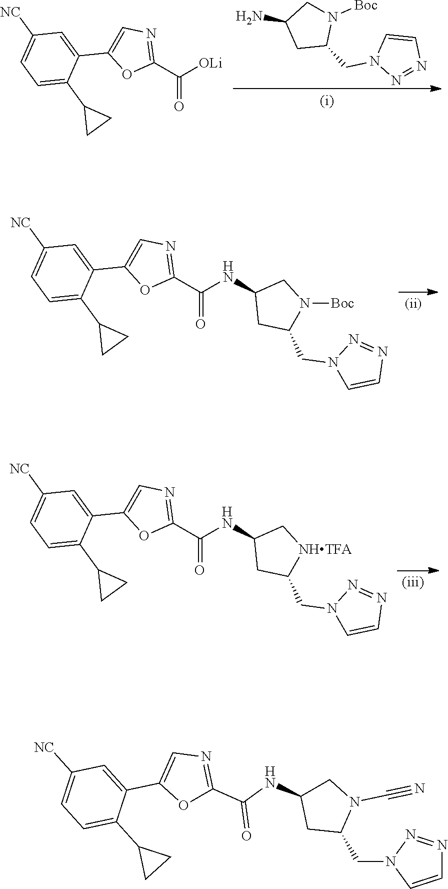

### Step (i)

### tert-Butyl (2S,4R)-2-((1H-1,2,3-Triazol-1-yl)methyl)-4-(5-(5-cyano-2-cyclopropylphenyl)oxazole-2-carboxamido)pyrrolidine-1-carboxylate

To a stirred solution of lithium 5-(5-cyano-2-cyclopropylphenyl)oxazole-2-carboxylate (0.5 g, 1.92 mmol) and tert-butyl (2S,4R)-2-((1H-1,2,3-triazol-1-yl)methyl)-4-aminopyrrolidine-1-carboxylate (0.36 g, 1.34 mmol) in pyridine (5 mL) was added POCl₃ (0.88 g, 0.54 mL, 5.76 mmol) dropwise at 0° C. and the mixture was stirred for 2 h at rt. The mixture was then poured into ice-cold water (50 mL) and extracted with EtOAc (2×50 mL). The combined organic phases were dried over anhydrous Na₂SO₄ and concentrated under reduced pressure. The residue was purified by flash column chromatography (silica gel, 90% EtOAc in n-hexanes) to yield tert-butyl (2S,4R)-2-((1H-1,2,3-triazol-1-yl)methyl)-4-(5-(5-cyano-2-cyclopropylphenyl)oxazole-2-carboxamido)pyrrolidine-1-carboxylate (0.07 g, 0.14 mmol, 8% yield over two steps).

LCMS: Method H1, 3.09 min, MS: ES− 502.2.

### Step (ii)

### N-((3R,5S)-5-((1H-1,2,3-Triazol-1-yl)methyl)pyrrolidin-3-yl)-5-(5-cyano-2-cyclopropylphenyl)oxazole-2-carboxamide TFA salt

To a stirred solution of tert-butyl (2S,4R)-2-((1H-1,2,3-triazol-1-yl)methyl)-4-(5-(5-cyano-2-cyclopropylphenyl)oxazole-2-carboxamido)pyrrolidine-1-carboxylate (0.07 g, 0.14 mmol) in DCM (5 mL) was added TFA (0.7 mL, 10 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(5-cyano-2-cyclopropylphenyl)oxazole-2-carboxamide TFA salt (0.09 g, quantitative yield).

LCMS: Method H1, 2.50 min, MS: ES+ 404.2.

Step (iii)

### N-((3R,5S)-5-((1H-1,2,3-Triazol-1-yl)methyl)-1-cyanopyrrolidin-3-yl)-5-(5-cyano-2-cyclopropylphenyl)oxazole-2-carboxamide

To a stirred solution of N-((3R,5S)-5-((1H-1,2,3-triazol-1-yl)methyl)pyrrolidin-3-yl)-5-(5-cyano-2-cyclopropylphenyl)oxazole-2-carboxamide TFA salt (0.09 g, 0.17 mmol) in THF (5 mL) was added K₂CO₃ (0.07 g, 0.52 mmol) at rt and stirred for 5 min. Cyanogen bromide (0.02 g, 0.17 mmol) was added into the reaction mixture at 0° C. The mixture was stirred at rt for 1 h, then poured into water (50 mL) and extracted with EtOAc (2×30 mL). The combined organic phases were dried over anhydrous Na₂SO₄, filtered and concentrated under reduced pressure. The residue was purified by flash column chromatography (silica gel, 100% EtOAc) to yield N-((3R,5S)-5-((1H-1,2,3-triazol-1-yl)methyl)-1-cyanopyrrolidin-3-yl)-5-(5-cyano-2-cyclopropylphenyl)oxazole-2-carboxamide (0.05 g, 0.10 mmol, 75% yield over two steps) which was further purified by reverse phase preparative HPLC (Method X3) to yield N-((3R,5S)-5-((1H-1,2,3-triazol-1-yl)methyl)-1-cyanopyrrolidin-3-yl)-5-(5-cyano-2-cyclopropylphenyl)oxazole-2-carboxamide (0.02 g, 0.06 mmol, 42% yield over two steps). LCMS: Method H1, 2.92 min, MS: ES+ 429.2; ¹H NMR (400 MHz, DMSO-d₆) δ ppm: 9.42 (d, J=5.6 Hz, 1H), 8.21 (s, 1H), 8.18 (d, J=1.2 Hz, 1H), 7.93 (s, 1H), 7.85 (dd, J=8.0, 1.2 Hz, 1H), 7.79 (s, 1H), 7.40 (d, J=8.0 Hz, 1H), 4.63-4.64 (m, 2H), 4.36-4.39 (m, 2H), 3.67-3.71 (m, 1H), 3.41-3.45 (m, 1H), 2.21-2.26 (m, 2H), 1.98-2.04 (m, 1H), 1.12-1.17 (m, 2H), 0.82-0.86 (m, 2H); Chiral SFC: Method Y9, 5.54 min.

### Example 10

### N-((3R,5S)-5-((1H-1,2,3-Triazol-1-yl)methyl)-1-cyanopyrrolidin-3-yl)-5-(2-(trifluoromethoxy)phenyl)oxazole-2-carboxamide

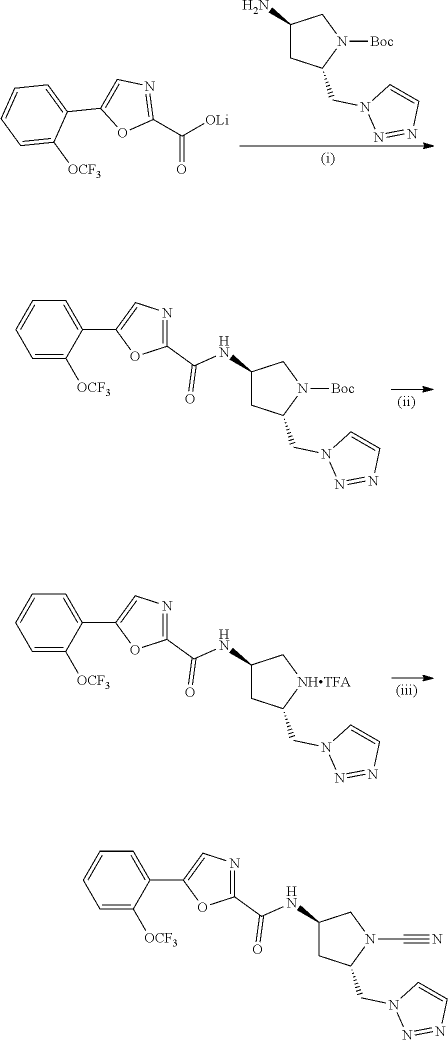

### Step (i)

### tert-Butyl (2S,4R)-2-((1H-1,2,3-triazol-1-yl)methyl)-4-(5-(2-(trifluoromethoxy)phenyl)oxazole-2-carboxamido)pyrrolidine-1-carboxylate

To a stirred solution of lithium 5-(2-(trifluoromethoxy)phenyl)oxazole-2-carboxylate (0.3 g, 1.09 mmol) and tert-butyl (2S,4R)-2-((1H-1,2,3-triazol-1-yl)methyl)-4-aminopyrrolidine-1-carboxylate (0.33 g, 1.20 mmol) in THF (5 mL) were added DIPEA (0.42 g, 0.56 mL, 3.29 mmol) and T₃P (50% in EtOAc) (0.69 g, 2.19 mmol) at 0° C. The mixture was stirred at rt for 3 h, then poured into ice-cold water (30 mL) and extracted with EtOAc (3×50 mL). The combined organic phases were dried over anhydrous Na₂SO₄, filtered and concentrated under reduced pressure. The residue was purified by flash column chromatography (silica gel, 80% EtOAc in n-hexanes) to yield tert-butyl (2S,4R)-2-((1H-1,2,3-triazol-1-yl)methyl)-4-(5-(2-(trifluoromethoxy)phenyl)oxazole-2-carboxamido)pyrrolidine-1-carboxylate (0.18 g, 0.34 mmol, 34% yield).

LCMS: Method C1, 1.38 min, MS: ES+ 523.3.

### Step (ii)

### N-((3R,5S)-5-((1H-1,2,3-Triazol-1-yl)methyl)pyrrolidin-3-yl)-5-(2-(trifluoromethoxy)phenyl)oxazole-2-carboxamide TFA salt

To a stirred solution of tert-butyl (2S,4R)-2-((1H-1,2,3-triazol-1-yl)methyl)-4-(5-(2-(trifluoromethoxy)phenyl)oxazole-2-carboxamido)pyrrolidine-1-carboxylate (0.18 g, 0.34 mmol) in DCM (5 mL) was added TFA (0.9 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(2-(trifluoromethoxy)phenyl)oxazole-2-carboxamide TFA salt (0.2 g, quantitative yield).

LCMS: Method C1, 1.13 min, MS: ES+ 423.3.

Step (iii)

### N-((3R,5S)-5-((1H-1,2,3-Triazol-1-yl)methyl)-1-cyanopyrrolidin-3-yl)-5-(2-(trifluoromethoxy)phenyl)oxazole-2-carboxamide

To a stirred solution of N-((3R,5S)-5-((1H-1,2,3-triazol-1-yl)methyl)pyrrolidin-3-yl)-5-(2-(trifluoromethoxy)phenyl)oxazole-2-carboxamide TFA salt (0.2 g, 0.37 mmol) in MeCN:THF (6 mL, 1:1) was added K₂CO₃ (0.15 g, 1.11 mmol) at rt and stirred for 5 min. Cyanogen bromide (0.04 g, 0.41 mmol) was added into the reaction mixture at 0° C. The mixture was stirred at rt for 1 h, then poured into water (20 mL) and extracted with EtOAc (3×50 mL). The combined organic phases were dried over anhydrous Na₂SO₄, filtered and concentrated under reduced pressure. The residue was purified by flash column chromatography (silica gel, 80% EtOAc in n-hexanes) to yield N-((3R,5S)-5-((1H-1,2,3-triazol-1-yl)methyl)-1-cyanopyrrolidin-3-yl)-5-(2-(trifluoromethoxy)phenyl)oxazole-2-carboxamide (0.12 g, 0.26 mmol, 77% yield over two steps).

LCMS: Method H1, 2.79 min, MS: ES+ 448.0; ¹H NMR (400 MHz, DMSO-d₆) δ ppm: 9.42 (d, J=6.4 Hz, 1H), 8.20 (s, 1H), 8.00 (d, J=7.2 Hz, 1H), 7.79 (s, 1H), 7.71 (s, 1H), 7.61 (d, J=6.0 Hz, 3H), 4.62-4.64 (m, 2H), 4.36-4.39 (m, 2H), 3.66-3.70 (m, 1H), 3.41-3.44 (m, 1H), 2.18-2.25 (m, 1H), 1.97-2.03 (m, 1H); Chiral SFC: Method Y9, 4.77 min.

### Example 11

### N-((3R,5S)-5-((1H-1,2,3-Triazol-1-yl)methyl)-1-cyanopyrrolidin-3-yl)-5-(3-ethylphenyl)oxazole-2-carboxamide

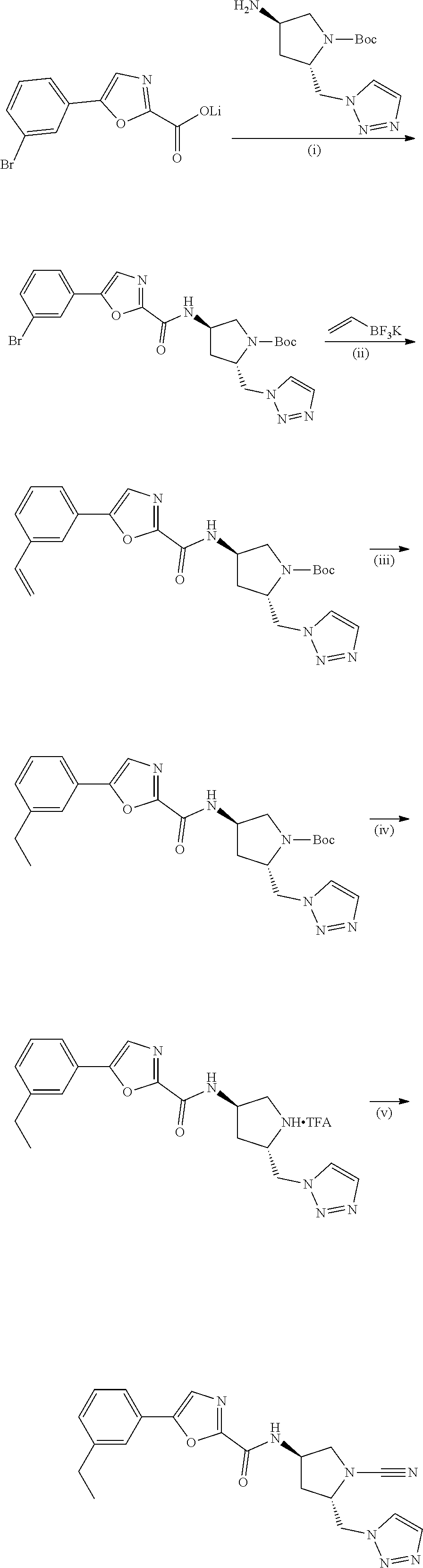

### Step (i)

### tert-Butyl (2S,4R)-2-((1H-1,2,3-triazol-1-yl)methyl)-4-(5-(3-bromophenyl)oxazole-2-carboxamido)pyrrolidine-1-carboxylate

To a stirred solution of lithium 5-(3-bromophenyl)oxazole-2-carboxylate (1.05 g, 3.92 mmol) and tert-butyl (2S,4R)-2-((1H-1,2,3-triazol-1-yl)methyl)-4-aminopyrrolidine-1-carboxylate (0.7 g, 2.62 mmol) in THF (10 mL) were added DIPEA (1.01 g, 1.38 mL, 7.86 mmol) and T₃P (50% in EtOAc) (1.66 g, 5.24 mmol) dropwise at 0° C. The mixture was allowed to warm to rt and stirred for 2 h, then poured into water (50 mL) and extracted with EtOAc (2×100 mL). The combined organic phases were dried over Na₂SO₄, filtered and concentrated under reduced pressure. The residue was purified by flash column chromatography (silica gel, 80% EtOAc in n-hexanes) to yield tert-butyl (2S,4R)-2-((1H-1,2,3-triazol-1-yl)methyl)-4-(5-(3-bromophenyl)oxazole-2-carboxamido)pyrrolidine-1-carboxylate (0.6 g, 1.16 mmol, 29% yield over two steps).

LCMS: Method C1, 1.26 min, MS: ES+ 517.1, 519.1.

### Step (ii)

### tert-Butyl (2S,4R)-2-((1H-1,2,3-triazol-1-yl)methyl)-4-(5-(3-vinylphenyl)oxazole-2-carboxamido)pyrrolidine-1-carboxylate

To a stirred solution of tert-butyl (2S,4R)-2-((1H-1,2,3-triazol-1-yl)methyl)-4-(5-(3-bromophenyl)oxazole-2-carboxamido)pyrrolidine-1-carboxylate (0.30 g, 0.58 mmol) and potassium vinyltrifluoroborate (CAS 13682-77-4, from Combi-Blocks, 0.10 g, 0.75 mmol) in EtOH (9 mL) was added TEA (0.076 g, 0.1 mL, 0.75 mmol). The mixture was purged with N₂ gas for 20 min, followed by addition of [1,1′-bis(diphenylphosphino)ferrocene]dichloropalladium(II) (0.04 g, 0.06 mmol) and heated at 80° C. for 3 h. The mixture was poured into water (50 mL) and extracted with EtOAc (2×40 mL). The combined organic phases were dried over anhydrous Na₂SO₄, filtered and concentrated under reduced pressure. The residue was purified by flash column chromatography (silica gel, 80% EtOAc in n-hexanes) to yield tert-butyl (2S,4R)-2-((1H-1,2,3-triazol-1-yl)methyl)-4-(5-(3-vinylphenyl)oxazole-2-carboxamido)pyrrolidine-1-carboxylate (0.25 g, 0.53 mmol, 93% yield).

LCMS: Method C1, 1.25 min, MS: ES+ 465.3.

Step (iii)

### tert-Butyl (2S,4R)-2-((1H-1,2,3-triazol-1-yl)methyl)-4-(5-(3-ethylphenyl)oxazole-2-carboxamido)pyrrolidine-1-carboxylate

To a stirred solution of tert-butyl (2S,4R)-2-((1H-1,2,3-triazol-1-yl)methyl)-4-(5-(3-vinylphenyl)-oxazole-2-carboxamido)pyrrolidine-1-carboxylate (0.25 g, 0.53 mmol) in MeOH (10 mL) was added 10% Pd/C (50% moisture) (0.12 g, 0.5 w/w). The mixture was purged with H₂ gas for 1 h, then filtered through Celite Hyflow® and the filtrate was concentrated under reduced pressure to yield tert-butyl (2S,4R)-2-((1H-1,2,3-triazol-1-yl)methyl)-4-(5-(3-ethylphenyl)oxazole-2-carboxamido)pyrrolidine-1-carboxylate (0.22 g, 0.47 mmol, 88%).

LCMS: Method C1, 1.44 min, MS: ES+ 467.4.

### Step (iv)

### N-((3R,5S)-5-((1H-1,2,3-Triazol-1-yl)methyl)pyrrolidin-3-yl)-5-(3-ethylphenyl)oxazole-2-carboxamide TFA salt

To a stirred solution of tert-butyl (2S,4R)-2-((1H-1,2,3-triazol-1-yl)methyl)-4-(5-(3-ethylphenyl)oxazole-2-carboxamido)pyrrolidine-1-carboxylate (0.22 g, 0.47 mmol) in DCM (10 mL) was added TFA (2.2 mL, 10 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(3-ethylphenyl)oxazole-2-carboxamide TFA salt (0.20 g, 0.41 mmol, 88% yield).

LCMS: Method C1, 1.07 min, MS: ES+ 367.2.

### Step (v)

### N-((3R,5S)-5-((1H-1,2,3-Triazol-1-yl)methyl)-1-cyanopyrrolidin-3-yl)-5-(3-ethylphenyl)oxazole-2-carboxamide

To a stirred solution of N-((3R,5S)-5-((1H-1,2,3-triazol-1-yl)methyl)pyrrolidin-3-yl)-5-(3-ethylphenyl)oxazole-2-carboxamide TFA salt (0.20 g, 0.41 mmol) in THF (5 mL) was added K₂CO₃ (0.17 g, 1.24 mmol) at rt and the mixture was stirred for 10 min. Cyanogen bromide (0.05 g, 0.45 mmol) was added at 0° C. and the mixture was stirred at rt for 1 h, then poured into water (20 mL) and extracted with EtOAc (3×50 mL). The combined organic phases were dried over Na₂SO₄ and concentrated under reduced pressure. The residue was purified by flash column chromatography (silica gel, 100% EtOAc in n-hexanes) to yield N-((3R,5S)-5-((1H-1,2,3-triazol-1-yl)methyl)-1-cyanopyrrolidin-3-yl)-5-(3-ethylphenyl)oxazole-2-carboxamide (0.08 g, 0.20 mmol, 43% yield over two steps).

LCMS: Method H, 2.81 min, MS: ES+ 392.0; ¹H NMR (400 MHz, DMSO-d₆) δ ppm: 9.35 (d, J=6.4 Hz, 1H), 8.20 (s, 1H), 7.94 (s, 1H), 7.79 (s, 1H), 7.68 (s, 1H), 7.65 (d, J=7.6 Hz, 1H), 7.45 (t, J=7.6 Hz, 1H), 7.31 (d, J=7.6 Hz, 1H), 4.62-4.64 (m, 2H), 4.35-4.37 (m, 2H), 3.63-3.72 (m, 1H), 3.42-3.44 (m, 1H), 2.67-2.70 (m, 2H), 2.18-2.25 (m, 1H), 1.97-2.02 (m, 1H), 1.24 (t, J=7.6 Hz, 3H); Chiral SFC: Method Y9, 5.71 min.

### Example 12

### N-((3R,5S)-5-((1H-1,2,3-Triazol-1-yl)methyl)-1-cyanopyrrolidin-3-yl)-5-(3-cyclopropylphenyl)oxazole2-carboxamide

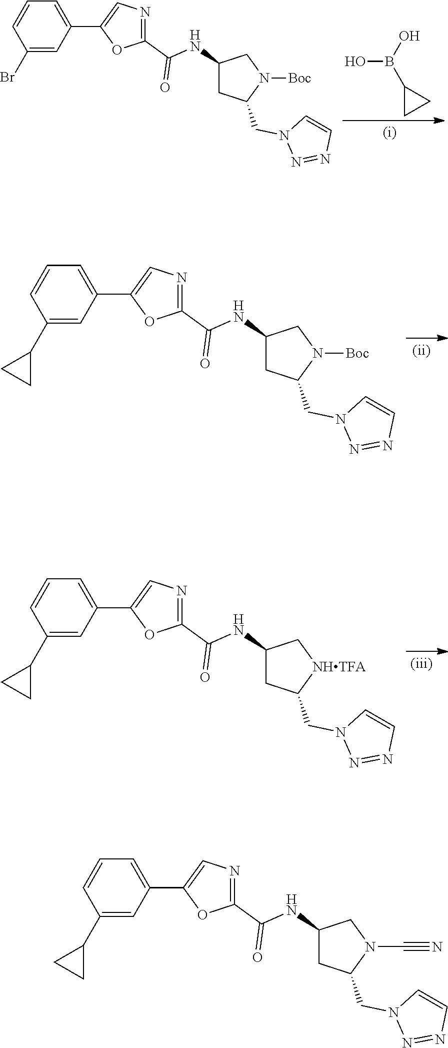

### Step (i)

### tert-Butyl (2S,4R)-2-((1H-1,2,3-triazol-1-yl)methyl)-4-(5-(3-cyclopropylphenyl)oxazole-2-carboxamido)pyrrolidine-1-carboxylate

To a stirred solution of tert-butyl (2S,4R)-2-((1H-1,2,3-triazol-1-yl)methyl)-4-(5-(3-bromophenyl)-oxazole-2-carboxamido)pyrrolidine-1-carboxylate (0.18 g, 0.34 mmol) and cyclopropylboronic acid (CAS 411235-57-9, from Combi-Blocks, 0.044 g, 0.52 mmol) in toluene:water (8 mL, 9:1) was added K₃PO₄ (0.22 g, 1.04 mmol). The mixture was purged with N₂ gas for 20 min, followed by addition of [1,1′-bis(diphenylphosphino)ferrocene]dichloropalladium(II) (0.025 g, 0.034 mmol) and heated at 100° C. for 3 h. The mixture was poured into water (20 mL) and extracted with EtOAc (3×50 mL). The combined organic phases were dried over anhydrous Na₂SO₄, filtered and concentrated under reduced pressure. The residue was purified by flash column chromatography (silica gel, 70% EtOAc in n-hexanes) to yield tert-butyl (2S,4R)-2-((1H-1,2,3-triazol-1-yl)methyl)-4-(5-(3-cyclopropylphenyl)-oxazole-2-carboxamido)pyrrolidine-1-carboxylate (0.1 g, 0.20 mmol, 60% yield).

LCMS: Method C1, 1.30 min, MS: ES+ 479.3.

### Step (ii)

### N-((3R,5S)-5-((1H-1,2,3-Triazol-1-yl)methyl)pyrrolidin-3-yl)-5-(3-cyclopropylphenyl)oxazole-2-carboxamide TFA salt

To a stirred solution of tert-butyl (2S,4R)-2-((1H-1,2,3-triazol-1-yl)methyl)-4-(5-(3-cyclopropylphenyl)oxazole-2-carboxamido)pyrrolidine-1-carboxylate (0.1 g, 0.20 mmol) in DCM (2.5 mL) was added TFA (0.5 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(3-cyclopropylphenyl)oxazole-2-carboxamide TFA salt (0.08 g, 0.16 mmol, 78% yield).

LCMS: Method C1, 1.06 min, MS: ES+ 379.2.

Step (iii)

### N-((3R,5S)-5-((1H-1,2,3-Triazol-1-yl)methyl)-1-cyanopyrrolidin-3-yl)-5-(2-cyclopropylphenyl)-1,3,4-oxadiazole-2-carboxamide

To a stirred solution of N-((3R,5S)-5-((1H-1,2,3-triazol-1-yl)methyl)pyrrolidin-3-yl)-5-(3-cyclopropylphenyl)oxazole-2-carboxamide TFA salt (0.08 g, 0.16 mmol) in THF (5 mL) was added K₂CO₃ (0.07 g, 0.48 mmol) at rt and stirred for 5 min. Cyanogen bromide (0.02 g, 0.17 mmol) was added into the reaction mixture at 0° C. The mixture was stirred at rt for 1 h, then poured into water (15 mL) and extracted with EtOAc (3×15 mL). The combined organic phases were dried over anhydrous Na₂SO₄ and concentrated under reduced pressure. The residue was purified by flash column chromatography (silica gel, 97% EtOAc in n-hexanes) to yield N-((3R,5S)-5-((1H-1,2,3-triazol-1-yl)methyl)-1-cyanopyrrolidin-3-yl)-5-(2-cyclopropylphenyl)-1,3,4-oxadiazole-2-carboxamide (0.02 g, 0.06 mmol, 38% yield).

LCMS: Method H1, 2.81 min, MS: ES+ 404.2; ¹H NMR (400 MHz, DMSO-d₆) δ 9.34 (d, J=6.4 Hz, 1H), 8.19 (s, 1H), 7.94 (s, 1H), 7.78 (s, 1H), 7.58 (d, J=7.6 Hz, 1H), 7.51 (s, 1H), 7.39 (t, J=7.6 Hz, 1H), 7.15 (d, J=8.0 Hz, 1H), 4.61-4.62 (m, 2H), 4.32-4.41 (m, 2H), 3.62-3.7 (m, 1H), 3.40-3.41 (m, 1H), 2.67-2.69 (m, 1H), 2.18-2.20 (m, 1H), 1.98-1.99 (m, 1H), 0.99-1.01 (m, 2H), 0.75-0.76 (m, 2H); Chiral HPLC: Method Y10, 10.27 min.

### Example 13

### N-((3R,5S)-5-((1H-1,2,3-Triazol-1-yl)methyl)-1-cyanopyrrolidin-3-yl)-5-(2-cyclopropyl-5-(trifluoromethoxy)phenyl)-1,3,4-oxadiazole-2-carboxamide

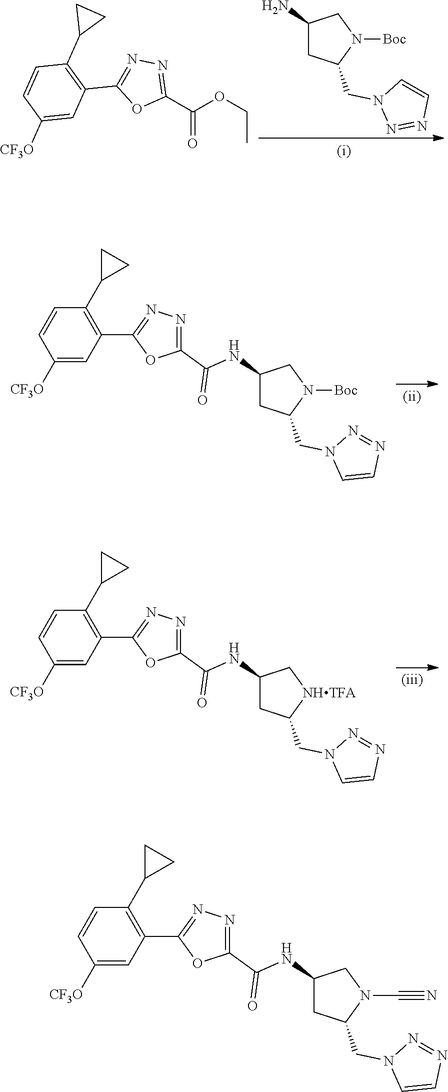

### Step (i)

### tert-Butyl (2S,4R)-2-((1H-1,2,3-triazol-1-yl)methyl)-4-(5-(2-cyclopropyl-5-(trifluoromethoxy)phenyl)-1,3,4-oxadiazole-2-carboxamido)pyrrolidine-1-carboxylate

A stirred solution of ethyl 5-(2-cyclopropyl-5-(trifluoromethoxy)phenyl)-1,3,4-oxadiazole-2-carboxylate (0.40 g, 1.16 mmol) and tert-butyl (2S,4R)-2-((1H-1,2,3-triazol-1-yl)methyl)-4-aminopyrrolidine-1-carboxylate (0.31 g, 1.16 mmol) in THF (4 mL) was purged with N₂ for 10 min. DBU (1.2 mL, 3 vol) was added dropwise at 0° C. The mixture was allowed to warm to rt and stirred for 4 h at rt. The mixture was poured into water (20 mL) and extracted with EtOAc (3×15 mL). The combined organic phases were dried over anhydrous Na₂SO₄, filtered and concentrated under reduced pressure. The residue was purified using flash column chromatography (silica gel, 78% EtOAc in n-hexanes) to yield tert-butyl (2S,4R)-2-((1H-1,2,3-triazol-1-yl)methyl)-4-(5-(2-cyclopropyl-5-(trifluoromethoxy)phenyl)-1,3,4-oxadiazole-2-carboxamido)pyrrolidine-1-carboxylate (0.08 g, 0.14 mmol, 12% yield). LCMS: Method C1, 1.32 min, MS: ES+ 564.3.

### Step (ii)

### N-((3R,5S)-5-((1H-1,2,3-Triazol-1-yl)methyl)pyrrolidin-3-yl)-5-(2-cyclopropyl-5-(trifluoromethoxy)phenyl)-1,3,4-oxadiazole-2-carboxamide TFA salt

To a stirred solution of tert-butyl (2S,4R)-2-((1H-1,2,3-triazol-1-yl)methyl)-4-(5-(2-cyclopropyl-5-(trifluoromethoxy)phenyl)-1,3,4-oxadiazole-2-carboxamido)pyrrolidine-1-carboxylate (0.08 g, 0.14 mmol) in DCM (0.8 mL) was added TFA (0.4 mL, 5 vol) dropwise at 0° C. The mixture was allowed to warm to rt and stirred for 2 h, then concentrated under reduced pressure to yield N-((3R,5S)-5-((1H-1,2,3-triazol-1-yl)methyl)pyrrolidin-3-yl)-5-(2-cyclopropyl-5-(trifluoromethoxy)phenyl)-1,3,4-oxadiazole-2-carboxamide TFA salt (0.09 g, quantitative yield).

LCMS: Method C1, 1.13 min, MS: ES+ 464.3.

Step (iii)

### N-((3R,5S)-5-((1H-1,2,3-Triazol-1-yl)methyl)-1-cyanopyrrolidin-3-yl)-5-(2-cyclopropyl-5-(trifluoromethoxy)phenyl)-1,3,4-oxadiazole-2-carboxamide

To a stirred solution of N-((3R,5S)-5-((1H-1,2,3-triazol-1-yl)methyl)pyrrolidin-3-yl)-5-(2-cyclopropyl-5-(trifluoromethoxy)phenyl)-1,3,4-oxadiazole-2-carboxamide TFA salt (0.095 g, 0.16 mmol) in THF (1 mL) was added K₂CO₃ (0.068 g, 0.49 mmol) at rt and stirred for 5 min. Cyanogen bromide (0.017 g, 0.16 mmol) was added into the reaction mixture at 0° C. The mixture was stirred at rt for 2 h, then poured into water (15 mL) and extracted with EtOAc (3×15 mL). The combined organic phases were dried over anhydrous Na₂SO₄ and concentrated under reduced pressure. The residue was purified by reverse phase preparative HPLC (Method X8) to yield N-((3R,5S)-5-((1H-1,2,3-triazol-1-yl)methyl)-1-cyanopyrrolidin-3-yl)-5-(2-cyclopropyl-5-(trifluoromethoxy)phenyl)-1,3,4-oxadiazole-2-carboxamide (0.03 g, 0.06 mmol, 41% yield over two steps).

LCMS: Method H1, 3.01 min, MS: ES+ 489.2; ¹H NMR (400 MHz, DMSO-d₆) δ 9.77 (d, J=6.0 Hz, 1H), 8.19 (s, 1H), 7.78-7.83 (m, 2H), 7.57 (d, J=8.0 Hz, 1H), 7.31 (d, J=8.8 Hz, 1H), 4.62-4.64 (m, 2H), 4.35-4.38 (m, 2H), 3.66-3.70 (m, 1H), 3.41-3.45 (m, 1H), 2.67-2.69 (m, 1H), 2.17-2.24 (m, 1H), 1.97-2.04 (m, 1H), 1.05-1.07 (m, 2H), 0.80-0.81 (m, 2H); Chiral HPLC: Method Y9, 4.98 min.

### Example 14

### N-((3R,5S)-5-((1H-1,2,3-Triazol-1-yl)methyl)-1-cyanopyrrolidin-3-yl)-5-(5-cyano-2-cyclopropylphenyl)-1,3,4-oxadiazole-2-carboxamide

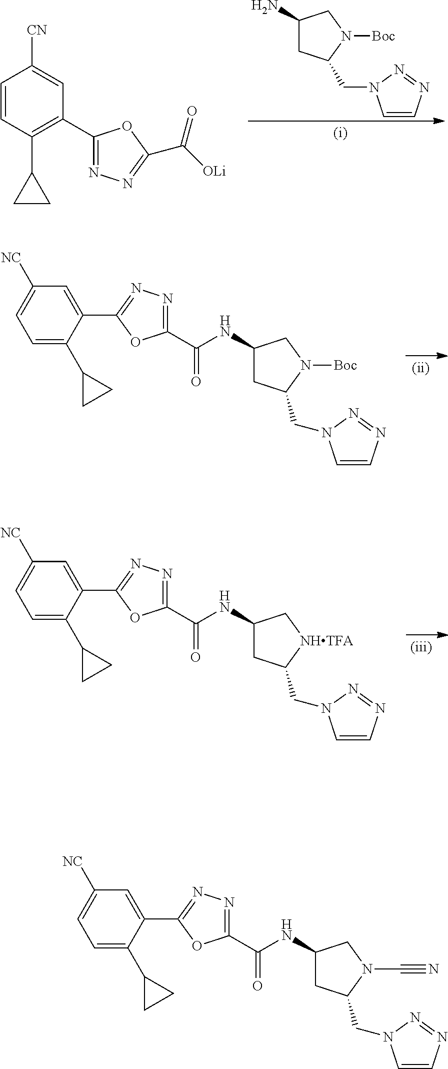

### Step (i)

### tert-Butyl (2S,4R)-2-((1H-1,2,3-triazol-1-yl)methyl)-4-(5-(5-cyano-2-cyclopropylphenyl)-1,3,4-oxadiazole-2-carboxamido) pyrrolidine-1-carboxylate

To a stirred solution of lithium 5-(5-cyano-2-cyclopropylphenyl)-1,3,4-oxadiazole-2-carboxylate (0.25 g, 0.98 mmol) and tert-butyl (2S,4R)-2-((1H-1,2,3-triazol-1-yl)methyl)-4-aminopyrrolidine-1-carboxylate (0.34 g, 1.27 mmol) in THF (5 mL) were added DIPEA (2.5 mL, 2.94 mmol) and HATU (0.74 g, 1.96 mmol) at 0° C. The mixture was stirred at rt for 3 h, then poured into ice-cold water (100 mL) and extracted with EtOAc (3×100 mL). The combined organic phases were dried over anhydrous Na₂SO₄, filtered and concentrated under reduced pressure. The residue was purified by flash column chromatography (silica gel, 83.6% EtOAc in n-hexanes) to yield tert-butyl (2S,4R)-2-((1H-1,2,3-triazol-1-yl)methyl)-4-(5-(5-cyano-2-cyclopropylphenyl)-1,3,4-oxadiazole-2-carboxamido)-pyrrolidine-1-carboxylate (0.47 g, quantitative yield).

LCMS: Method C1, 1.21 min, MS: ES+ 505.3.

### Step (ii)

### N-((3R,5S)-5-((1H-1,2,3-Triazol-1-yl)methyl)pyrrolidin-3-yl)-5-(5-cyano-2-cyclopropylphenyl)-1,3,4-oxadiazole-2-carboxamide TFA salt

To a stirred solution of tert-butyl (2S,4R)-2-((1H-1,2,3-triazol-1-yl)methyl)-4-(5-(5-cyano-2-cyclopropylphenyl)-1,3,4-oxadiazole-2-carboxamido)pyrrolidine-1-carboxylate (0.47 g, 0.93 mmol) in DCM (3 mL) was added TFA (2.3 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(5-cyano-2-cyclopropylphenyl)-1,3,4-oxadiazole-2-carboxamide TFA salt (0.32 g, 0.62 mmol, 87% yield over three steps).

LCMS: Method H1, 2.38 min, MS: ES+ 405.2.

Step (iii)

### N-((3R,5S)-5-((1H-1,2,3-Triazol-1-yl)methyl)-1-cyanopyrrolidin-3-yl)-5-(5-cyano-2-cyclopropylphenyl)-1,3,4-oxadiazole-2-carboxamide

To a stirred solution of N-((3R,5S)-5-((1H-1,2,3-triazol-1-yl)methyl)pyrrolidin-3-yl)-5-(5-cyano-2-cyclopropylphenyl)-1,3,4-oxadiazole-2-carboxamide TFA salt (0.31 g, 0.60 mmol) in THF (5 mL) was added K₂CO₃ (0.41 g, 3.04 mmol) at rt and stirred for 5 min. Cyanogen bromide (0.07 g, 0.68 mmol) was added into the reaction mixture at 0° C. The mixture was stirred at rt for 30 min, then poured into water (100 mL) and extracted with EtOAc (3×100 mL). The combined organic phases were dried over anhydrous Na₂SO₄, filtered and concentrated under reduced pressure. The residue was purified by flash column chromatography (silica gel, 82% EtOAc in n-hexanes) to yield N-((3R,5S)-5-((1H-1,2,3-triazol-1-yl)methyl)-1-cyanopyrrolidin-3-yl)-5-(5-cyano-2-cyclopropylphenyl)-1,3,4-oxadiazole-2-carboxamide (0.05 g, 0.12 mmol, 17% yield over three steps), which was further purified by reverse phase preparative HPLC (Method X10) to yield N-((3R,5S)-5-((1H-1,2,3-triazol-1-yl)methyl)-1-cyanopyrrolidin-3-yl)-5-(5-cyano-2-cyclopropylphenyl)-1,3,4-oxadiazole-2-carboxamide (0.01 g, 0.03 mmol, 5% yield over three steps).

LCMS: Method H1, 2.55 min, MS: ES+ 430.2; ¹H NMR (400 MHz, DMSO-d₆) δ ppm: 9.77 (s, 1H), 8.30 (s, 1H), 8.20 (s, 1H), 7.9 (d, J=8.0 Hz, 1H), 7.78 (s, 1H), 7.32 (d, J=8.0 Hz, 1H), 4.62-4.64 (m, 2H), 4.33-4.44 (m, 2H), 3.65-3.72 (s, 1H), 3.42-3.45 (m, 1H), 2.77-2.9 (m, 1H), 2.16-2.25 (m, 1H), 1.98-2.05 (s, 1H), 1.16-1.17 (m, 2H), 0.93 (s, 2H); Chiral SFC: Method Y4, 5.15 min.

### Example 15

### N-((3R,5S)-5-((1H-1,2,3-Triazol-1-yl)methyl)-1-cyanopyrrolidin-3-yl)-5-(2-cyclopropoxy-5-(trifluoromethoxy)phenyl)-1,3,4-oxadiazole-2-carboxamide

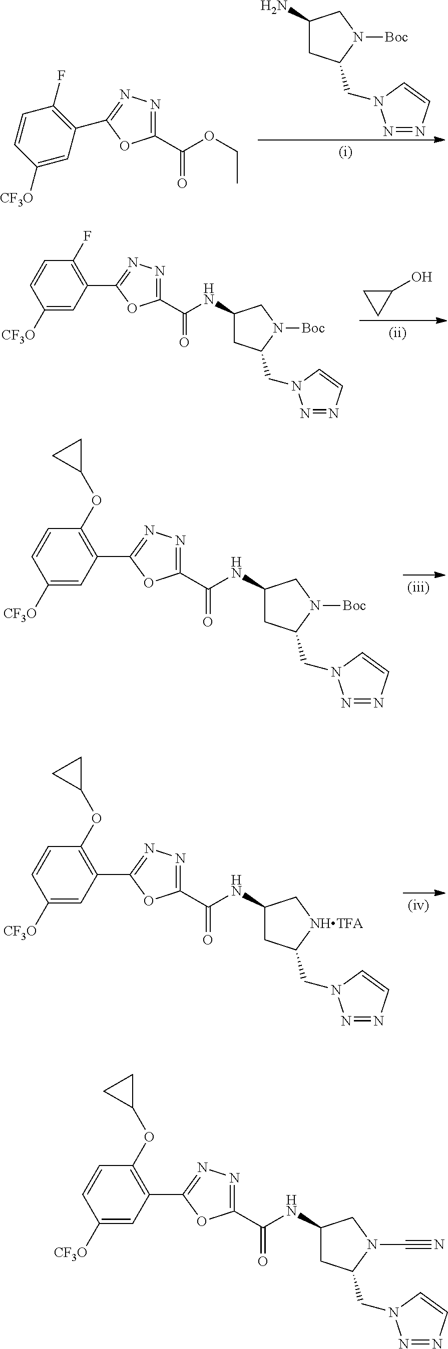

### Step (i)

### tert-Butyl (2S,4R)-2-((1H-1, 2, 3-triazol-1-yl)methyl)-4-(5-(2-fluoro-5-(trifluoromethoxy)phenyl)-1,3,4-oxadiazole-2-carboxamido) pyrrolidine-1-carboxylate

To a stirred solution of ethyl 5-(2-fluoro-5-(trifluoromethoxy)phenyl)-1,3,4-oxadiazole-2-carboxylate (1.2 g, 4.49 mmol) and tert-butyl (2S,4R)-2-((1H-1,2,3-triazol-1-yl)methyl)-4-aminopyrrolidine-1-carboxylate (1.43 g, 4.49 mmol) in THF (12 mL) was added TBD (0.75 g, 5.38 mmol) in portions at 0° C. The mixture was allowed to warm to rt and stirred for 6 h, then poured into water (50 mL) and extracted with EtOAc (2×60 mL). The combined organic phases were dried over anhydrous Na₂SO₄, filtered and concentrated under reduced pressure. The residue was purified by flash column chromatography (silica gel, 70% EtOAc in n-hexanes) to yield tert-butyl (2S,4R)-2-((1H-1,2,3-triazol-1-yl)methyl)-4-(5-(2-fluoro-5-(trifluoromethoxy)phenyl)-1,3,4-oxadiazole-2-carboxamido)pyrrolidine-1-carboxylate (0.13 g, 0.24 mmol, 6% yield).

LCMS: Method J1, 3.90 min, MS: ES− 540.1.

### Step (ii)

### tert-Butyl (2S,4R)-2-((1H-1,2,3-triazol-1-yl)methyl)-4-(5-(2-cyclopropoxy-5-(trifluoromethoxy)phenyl)-1,3,4-oxadiazole-2-carboxamido)pyrrolidine-1-carboxylate

To a stirred solution of cyclopropanol (CAS 16545-68-9, from Synthonix, 0.03 g, 0.48 mmol) in THF (1.3 mL) was added NaH (60% in oil) (0.02 g, 0.48 mmol) in portions at 0° C. and stirred at 0° C. for 45 min. A solution of tert-butyl (2S,4R)-2-((1H-1,2,3-triazol-1-yl)methyl)-4-(5-(2-fluoro-5-(trifluoromethoxy)phenyl)-1,3,4-oxadiazole-2-carboxamido)pyrrolidine-1-carboxylate (0.13 g, 0.24 mmol) in THF (1.3 mL) was added dropwise at 0° C. The mixture was allowed to warm to rt and stirred for 6 h, then poured into ice-cold water (20 mL) and extracted with EtOAc (3×40 mL). The combined organic phases were dried over Na₂SO₄, filtered and concentrated under reduced pressure. The residue was purified by flash column chromatography (silica gel, 40% EtOAc in n-hexanes) to yield tert-butyl (2S,4R)-2-((1H-1,2,3-triazol-1-yl)methyl)-4-(5-(2-cyclopropoxy-5-(trifluoromethoxy)-phenyl)-1,3,4-oxadiazole-2-carboxamido)pyrrolidine-1-carboxylate (0.08 g, 0.14 mmol, 57% yield).

LCMS: Method J1, 3.66 min, MS: ES+ 580.4.

Step (iii)

### N-((3R,5S)-5-((1H-1,2,3-Triazol-1-yl)methyl)pyrrolidin-3-yl)-5-(2-cyclopropoxy(trifluoromethoxy)phenyl)-1,3,4-oxadiazole-2-carboxamide TFA salt

To a stirred solution of tert-butyl (2S,4R)-2-((1H-1,2,3-triazol-1-yl)methyl)-4-(5-(2-cyclopropoxy-5-(trifluoromethoxy)phenyl)-1,3,4-oxadiazole-2-carboxamido)pyrrolidine-1-carboxylate (0.08 g, 0.13 mmol) in DCM (0.8 mL) was added TFA (0.4 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(2-cyclopropoxy-5-(trifluoromethoxy)phenyl)-1,3,4-oxadiazole-2-carboxamide TFA salt (0.1 g, quantitative yield).

LCMS: Method J1, 2.91 min, MS: ES+ 480.2.

### Step (iv)

### N-((3R,5S)-5-((1H-1,2,3-Triazol-1-yl)methyl)-1-cyanopyrrolidin-3-yl)-5-(2-cyclopropoxy-5-(trifluoromethoxy)phenyl)-1,3,4-oxadiazole-2-carboxamide

To a stirred solution of N-((3R,5S)-5-((1H-1,2,3-triazol-1-yl)methyl)pyrrolidin-3-yl)-5-(2-cyclopropoxy-5-(trifluoromethoxy)phenyl)-1,3,4-oxadiazole-2-carboxamide TFA salt (0.1 g, 0.16 mmol) in THF (1 mL) was added K₂CO₃ (0.11 g, 0.84 mmol) at rt and stirred for 5 min. Cyanogen bromide (0.02 g, 0.24 mmol) was added into the reaction mixture at 0° C. The mixture was stirred at rt for 1 h, then poured into water (20 mL) and extracted with EtOAc (3×50 mL). The combined organic phases were dried over Na₂SO₄ and concentrated under reduced pressure. The residue was purified by flash column chromatography (silica gel, 96% EtOAc in n-hexanes) to yield N-((3R,5S)-5-((1H-1,2,3-triazol-1-yl)methyl)-1-cyanopyrrolidin-3-yl)-5-(2-cyclopropoxy-5-(trifluoromethoxy)phenyl)-1,3,4-oxadiazole-2-carboxamide (0.02 g, 0.03 mmol, 24% yield over two steps).

LCMS: Method H1, 2.88 min, MS: ES+ 505.2; ¹H NMR (400 MHz, DMSO-d₆) δ ppm: 9.78 (d, J=4.0 Hz, 1H), 8.20 (s, 1H), 7.87 (s, 1H), 7.78 (s, 1H), 7.73-7.76 (m, 1H), 7.67-7.69 (m, 1H), 4.62-4.63 (m, 2H), 4.33-4.45 (m, 2H), 4.07-4.13 (m, 1H), 3.65-3.69 (m, 1H), 3.41-3.44 (m, 1H), 2.18-2.21 (m, 1H), 1.97-2.04 (m, 1H), 0.86-0.88 (m, 2H), 0.72-0.77 (m, 2H); Chiral SFC: Method Y21, 9.32 min.

### Example 16

### N-((3R,5S)-5-((1H-1,2,3-Triazol-1-yl)methyl)-1-cyanopyrrolidin-3-yl)-5-(5-cyano-2-(oxetan-3-yloxy)phenyl)-N-cyclopropyloxazole-2-carboxamide

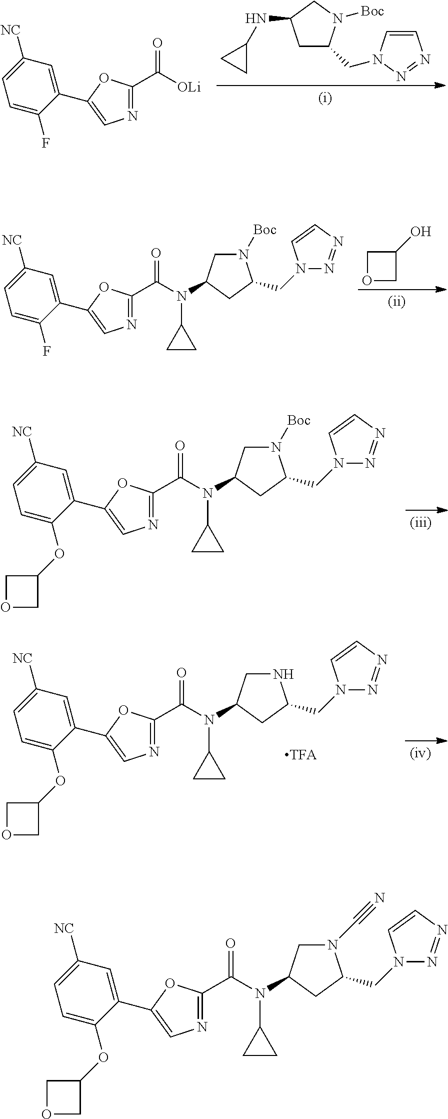

### Step (i)

### tert-Butyl (2S,4R)-2-((1H-1,2,3-triazol-1-yl)methyl)-4-(5-(5-cyano-2-fluorophenyl)-N-cyclopropyloxazole-2-carboxamido)pyrrolidine-1-carboxylate

To a stirred solution of lithium 5-(5-cyano-2-fluorophenyl)oxazole-2-carboxylate (0.25 g, 0.81 mmol) and tert-butyl (2S,4R)-2-((1H-1,2,3-triazol-1-yl)methyl)-4-aminopyrrolidine-1-carboxylate (0.28 g, 1.22 mmol) in pyridine (2.5 mL) was added POCl₃ (0.37 g, 0.22 mL, 2.44 mmol) dropwise at 0° C. The mixture was stirred at rt for 30 min, then poured into water (30 mL) and extracted with EtOAc (2×30 mL). The combined organic phases were dried over anhydrous Na₂SO₄, filtered and concentrated under reduced pressure. The residue was purified by flash column chromatography (silica gel, 90% EtOAc in n-hexanes) to yield tert-butyl (2S,4R)-2-((1H-1,2,3-triazol-1-yl)methyl)-4-(5-(5-cyano-2-fluorophenyl)-N-cyclopropyloxazole-2-carboxamido)pyrrolidine-1-carboxylate (0.15 g, 0.28 mmol, 26% yield over two steps). LCMS: Method C1, 1.25 min, MS: ES+ 522.3.

### Step (ii)

### tert-Butyl (2S,4R)-2-((1H-1,2,3-triazol-1-yl)methyl)-4-(5-(5-cyano-2-(oxetan-3-yloxy)phenyl)-N-cyclopropyloxazole-2-carboxamido)pyrrolidine-1-carboxylate

To a stirred solution of oxetan-3-ol (0.02 g, 0.38 mmol) in THF (2 mL) was added NaH (60% in oil) (0.01 g, 0.38 mmol) in portions under N₂ atmosphere at 0° C. After 15 min, a solution of tert-butyl (2S,4R)-2-((1H-1,2,3-triazol-1-yl)methyl)-4-(5-(5-cyano-2-fluorophenyl)-N-cyclopropyloxazole-2-carboxamido)pyrrolidine-1-carboxylate (0.18 g, 0.34 mmol) in THF (1 mL) was added dropwise at 0° C. The mixture was stirred at rt for 2 h, then poured into water (20 mL) and extracted with EtOAc (2×20 mL). The combined organic phases were dried over anhydrous Na₂SO₄, filtered and concentrated under reduced pressure to yield tert-butyl (2S,4R)-2-((1H-1,2,3-triazol-1-yl)methyl)-4-(5-(5-cyano-2-(oxetan-3-yloxy)phenyl)-N-cyclopropyloxazole-2-carboxamido)pyrrolidine-1-carboxylate (0.13 g, 0.22 mmol, 75% yield).

LCMS: Method C1, 1.17 min, MS: ES+ 476.3 (M-100).

Step (iii)

### N-((3R,5S)-5-((1H-1,2,3-Triazol-1-yl)methyl)pyrrolidin-3-yl)-5-(5-cyano-2-(oxetan-3-yloxy)phenyl)-N-cyclopropyloxazole-2-carboxamide TFA salt

To a stirred solution of tert-butyl (2S,4R)-2-((1H-1,2,3-triazol-1-yl)methyl)-4-(5-(5-cyano-2-(oxetan-3-yloxy)phenyl)-N-cyclopropyloxazole-2-carboxamido)pyrrolidine-1-carboxylate (0.13 g, 0.22 mmol) in DCM (2 mL) was added TFA (0.65 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(5-cyano-2-(oxetan-3-yloxy)phenyl)-N-cyclopropyloxazole-2-carboxamide TFA salt (0.14 g, quantitative yield).

LCMS: Method C1, 0.99 min, MS: ES+ 476.2.

### Step (iv)

### N-((3R,5S)-5-((1H-1,2,3-Triazol-1-yl)methyl)-1-cyanopyrrolidin-3-yl)-5-(5-cyano-2-(oxetan-3-yloxy)phenyl)-N-cyclopropyloxazole-2-carboxamide

To a stirred solution of N-((3R,5S)-5-((1H-1,2,3-triazol-1-yl)methyl)pyrrolidin-3-yl)-5-(5-cyano-2-(oxetan-3-yloxy)phenyl)-N-cyclopropyloxazole-2-carboxamide TFA salt (0.14 g, 0.23 mmol) in THF (2 mL) was added K₂CO₃ (0.09 g, 0.71 mmol) at rt and stirred for 5 min. Cyanogen bromide (0.02 g, 0.23 mmol) was added into the reaction mixture at 0° C. The mixture was stirred at rt for 1 h, then poured into water (30 mL) and extracted with EtOAc (2×40 mL). The combined organic phases were dried over anhydrous Na₂SO₄, filtered and concentrated under reduced pressure. The residue was purified by reverse phase preparative HPLC (Method X9) to yield N-((3R,5S)-5-((1H-1,2,3-triazol-1-yl)methyl)-1-cyanopyrrolidin-3-yl)-5-(5-cyano-2-(oxetan-3-yloxy)phenyl)-N-cyclopropyloxazole-2-carboxamide (0.01 g, 0.02 mmol, 13% yield over two steps).

LCMS: Method H1, 2.43 min, MS: ES+ 501.2; ¹H NMR (400 MHz, DMSO-d₆) δ ppm: 8.20-8.23 (m, 2H), 7.93 (s, 1H), 7.87 (d, J=8.8 Hz, 1H), 7.79 (s, 1H), 7.01 (d, J=8.8 Hz, 1H), 5.57-5.60 (m, 1H), 4.99-5.03 (m, 2H), 4.75-4.78 (m, 2H), 4.60-4.61 (m, 2H), 4.40 (br s, 1H), 4.32 (br s, 1H), 3.76-3.80 (m, 1H), 3.61-3.65 (m, 1H), 3.13 (br s, 1H), 2.50-2.54 (m, 1H), 2.18-2.20 (m, 1H), 0.65-0.72 (m, 2H), 0.54-0.57 (m, 2H); Chiral HPLC: Method Y21, 11.44 min.

### Example 17

### N-((3R,5S)-5-((2H-1,2,3-Triazol-2-yl)methyl)-1-cyanopyrrolidin-3-yl)-5-(3-cyanophenyl)oxazole-2-carboxamide

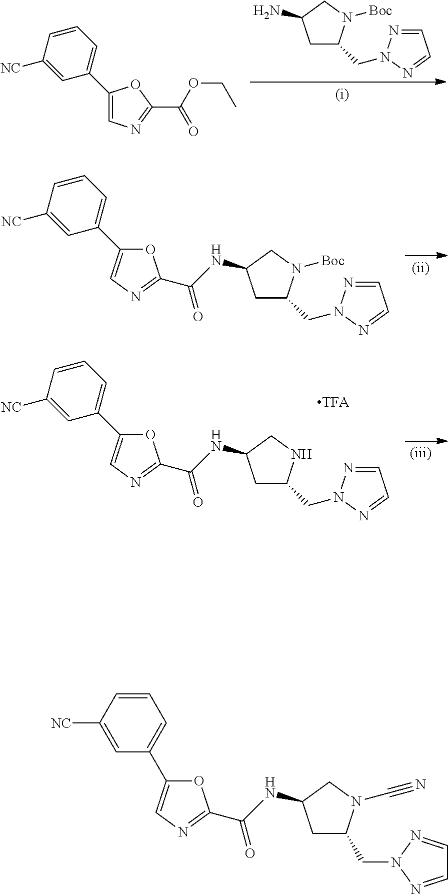 [SMILES: CCOC(=O)c1ncc(-c2cccc(C#N)c2)o1 ; CC(C)(C)OC(=O)N1C[C@H](N)C[C@H]1Cn1nccn1 ; CC(C)(C)OC(=O)N1C[C@H](NC(=O)c2ncc(-c3cccc(C#N)c3)o2)C[C@H]1Cn1nccn1 ; N#Cc1cccc(-c2cnc(C(=O)N[C@H]3CN[C@H](Cn4nccn4)C3)o2)c1 ; N#Cc1cccc(-c2cnc(C(=O)N[C@@H]3C[C@@H](Cn4nccn4)N(C#N)C3)o2)c1]

### Step (i)

### tert-Butyl (2S,4R)-2-((2H-1,2,3-triazol-2-yl)methyl)-4-(5-(3-cyanophenyl)oxazole-2-carboxamido)pyrrolidine-1-carboxylate

To a stirred solution of ethyl 5-(3-cyanophenyl)oxazole-2-carboxylate (0.54 g, 2.24 mmol) and tert-butyl (2S,4R)-2-((2H-1,2,3-triazol-2-yl)methyl)-4-aminopyrrolidine-1-carboxylate (0.30 g, 1.12 mmol) in THF (6 mL) was added TBD (0.31 g, 2.24 mmol) in portions at 0° C. The mixture was allowed to warm to rt and stirred for 24 h and heated at 70° C. for 24 h. The mixture was poured into water (100 mL) and extracted with EtOAc (4×100 mL). The combined organic phases were dried over anhydrous Na₂SO₄, filtered and concentrated under reduced pressure. The residue was purified by flash column chromatography (silica gel, 3% MeOH in DCM) to yield tert-butyl (2S,4R)-2-((2H-1,2,3-triazol-2-yl)methyl)-4-(5-(3-cyanophenyl)oxazole-2-carboxamido)pyrrolidine-1-carboxylate (0.15 g, 0.32 mmol, 29% yield).

LCMS: Method C, 1.62 min, MS: ES+ 464.5.

### Step (ii)

### N-((3R,5S)-5-((2H-1,2,3-Triazol-2-yl)methyl)pyrrolidin-3-yl)-5-(3-cyanophenyl)oxazole-2-carboxamide TFA salt

To a stirred solution of tert-butyl (2S,4R)-2-((2H-1,2,3-triazol-2-yl)methyl)-4-(5-(3-cyanophenyl)-oxazole-2-carboxamido)pyrrolidine-1-carboxylate (0.15 g, 0.32 mmol) in DCM (3 mL) was added TFA (0.75 mL, 5 vol) dropwise at 0° C. The mixture was allowed to warm to rt and stirred for 1 h, then concentrated under reduced pressure to yield N-((3R,5S)-5-((2H-1,2,3-triazol-2-yl)methyl)pyrrolidin-3-yl)-5-(3-cyanophenyl)oxazole-2-carboxamide TFA salt (0.15 g, 0.31 mmol, 97% yield).

LCMS: Method C, 1.33 min, MS: ES+ 364.5.

Step (iii)

### N-((3R,5S)-5-((2H-1,2,3-Triazol-2-yl)methyl)-1-cyanopyrrolidin-3-yl)-5-(3-cyanophenyl)oxazole-2-carboxamide

To a stirred solution of N-((3R,5S)-5-((2H-1,2,3-triazol-2-yl)methyl)pyrrolidin-3-yl)-5-(3-cyanophenyl)oxazole-2-carboxamide TFA salt (0.15 g, 0.31 mmol) in THF (3 mL) was added K₂CO₃ (0.13 g, 0.94 mmol) at rt and stirred for 5 min. Cyanogen bromide (0.03 g, 0.31 mmol) was added into the reaction mixture at 0° C. The mixture was stirred at rt for 1 h, then poured into ice-cold water (100 mL) and solid filtered through a Buchner funnel, washed with n-hexanes (2×100 mL) to yield N-((3R,5S)-5-((2H-1,2,3-triazol-2-yl)methyl)-1-cyanopyrrolidin-3-yl)-5-(3-cyanophenyl)oxazole-2-carboxamide (0.07 g, 0.17 mmol, 56% yield).

LCMS: Method H, 2.47 min, MS: ES+ 389.1; ¹H NMR (400 MHz, DMSO-d₆) δ ppm: 9.40 (d, J=6.4 Hz, 1H), 8.34 (s, 1H), 8.14 (d, J=8.0 Hz, 1H), 8.10 (s, 1H), 7.92 (d, J=8.0 Hz, 1H), 7.86 (s, 2H), 7.75 (t, J=8.0 Hz, 1H), 4.63-4.65 (m, 2H), 4.37-4.46 (m, 1H), 4.26-4.35 (m, 1H), 3.61-3.65 (m, 1H), 3.38-3.41 (m, 1H), 2.17-2.25 (m, 1H), 2.01-2.08 (m, 1H); Chiral SFC: Method Y12A, 3.99 min.

### Example 18

### N-((3R,5S)-5-((1H-1,2,4-Triazol-1-yl)methyl)-1-cyanopyrrolidin-3-yl)-5-(3-cyanophenyl)oxazole-2-carboxamide

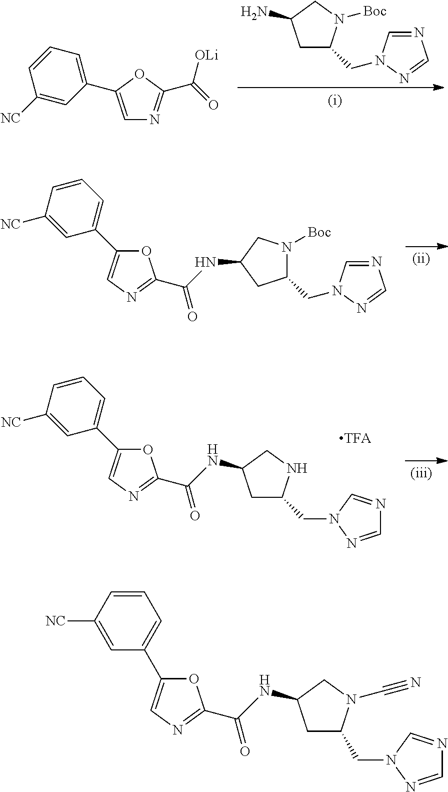

### Step (i)

### tert-Butyl (2S,4R)-2-((1H-1,2,4-triazol-1-yl)methyl)-4-(5-(3-cyanophenyl)oxazole-2-carboxamido)pyrrolidine-1-carboxylate

To a stirred solution of lithium 5-(3-cyanophenyl)oxazole-2-carboxylate (0.31 g, 1.40 mmol) and tert-butyl (2S,4R)-2-((1H-1,2,4-triazol-1-yl)methyl)-4-aminopyrrolidine-1-carboxylate (0.25 g, 0.93 mmol) in pyridine (2.5 mL) was added POCl₃ (0.43 g, 0.27 mL, 2.81 mmol) dropwise at 0° C. and the mixture was stirred for 2 h at 0° C. The mixture was then poured into ice-cold water (100 mL) and extracted with DCM (4×100 mL). The combined organic phases were dried over anhydrous Na₂SO₄ and concentrated under reduced pressure. The residue was purified by flash column chromatography (silica gel, 2% MeOH in DCM) to yield tert-butyl (2S,4R)-2-((1H-1,2,4-triazol-1-yl)methyl)-4-(5-(3-cyanophenyl)oxazole-2-carboxamido)pyrrolidine-1-carboxylate (0.06 g, 0.13 mmol, 14% yield).

LCMS: Method C, 1.531 min, MS: ES+ 464.4.

### Step (ii)

### N-((3R,5S)-5-((1H-1,2,4-Triazol-1-yl)methyl)pyrrolidin-3-yl)-5-(3-cyanophenyl)oxazole-2-carboxamide TFA salt

To a stirred solution of tert-butyl (2S,4R)-2-((1H-1,2,4-triazol-1-yl)methyl)-4-(5-(3-cyanophenyl)oxazole-2-carboxamido)pyrrolidine-1-carboxylate (0.06 g, 0.13 mmol) in DCM (1.2 mL) was added TFA (0.3 mL, 5 vol) dropwise at 0° C. The mixture was allowed to warm to rt and stirred for 1 h, then concentrated under reduced pressure to yield N-((3R,5S)-5-((1H-1,2,4-triazol-1-yl)methyl)pyrrolidin-3-yl)-5-(3-cyanophenyl)oxazole-2-carboxamide TFA salt (0.07 g, quantitative yield). LCMS: Method C, 1.316 min, MS: ES+ 364.3.

Step (iii)

### N-((3R,5S)-5-((1H-1,2,4-Triazol-1-yl)methyl)-1-cyanopyrrolidin-3-yl)-5-(3-cyanophenyl)oxazole-2-carboxamide

To a stirred solution of N-((3R,5S)-5-((1H-1,2,4-triazol-1-yl)methyl)pyrrolidin-3-yl)-5-(3-cyanophenyl)oxazole-2-carboxamide TFA salt (0.07 g, 0.15 mmol) in THF (1.4 mL) was added K₂CO₃ (0.06 g, 0.44 mmol) at rt and stirred for 5 min. Cyanogen bromide (0.01 g, 0.15 mmol) was added into the reaction mixture at 0° C. The mixture was stirred at rt for 1 h, then poured into ice-cold water (100 mL) and extracted with DCM (3×100 mL). The combined organic phases were dried over anhydrous Na₂SO₄, filtered and concentrated under reduced pressure. The residue was purified by flash column chromatography (silica gel, 5.6% MeOH in DCM) to yield N-((3R,5S)-5-((1H-1,2,4-triazol-1-yl)methyl)-1-cyanopyrrolidin-3-yl)-5-(3-cyanophenyl)oxazole-2-carboxamide (0.01 g, 0.02 mmol, 30% yield over two steps).

LCMS: Method N, 13.05 min, MS: ES+ 389.2; ¹H NMR (400 MHz, DMSO-d₆) δ ppm: 9.40 (d, J=6.4 Hz, 1H), 8.58 (s, 1H), 8.34 (s, 1H), 8.10-8.15 (m, 2H), 8.04 (s, 1H), 7.91-7.93 (m, 1H), 7.74-7.77 (m, 1H), 4.30-4.42 (m, 4H), 3.67-3.70 (m, 1H), 3.40-3.43 (m, 1H), 2.16-2.23 (m, 1H), 1.99-2.07 (m, 1H); Chiral SFC: Method Y12A, 4.12 min.

### Example 19

### N-((3R,5S)-5-((4H-1,2,4-Triazol-4-yl)methyl)-1-cyanopyrrolidin-3-yl)-5-(3-cyanophenyl)oxazole-2-carboxamide

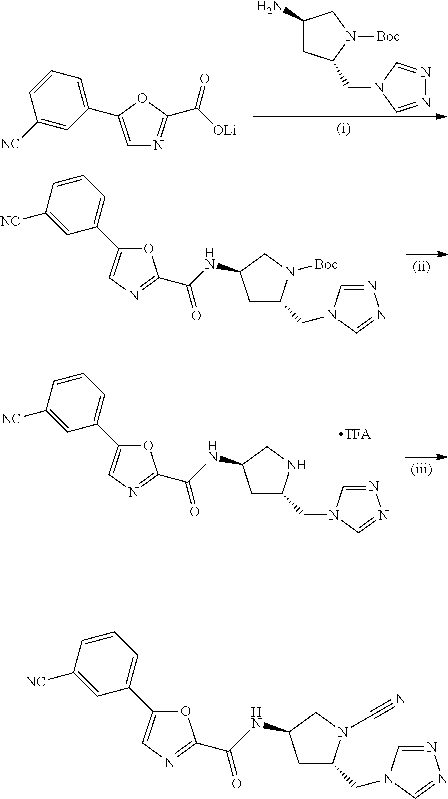

### Step (i)

### tert-Butyl (2S,4R)-2-((4H-1,2,4-triazol-4-yl)methyl)-4-(5-(3-cyanophenyl)oxazole-2-carboxamido)pyrrolidine-1-carboxylate

To a stirred solution of tert-butyl (2S,4R)-2-((4H-1,2,4-triazol-4-yl)methyl)-4-aminopyrrolidine-1-carboxylate (0.26 g, 0.97 mmol) and lithium 5-(3-cyanophenyl)oxazole-2-carboxylate (0.25 g, 1.16 mmol) in DMF (2.6 mL) were added DIPEA (0.25 g, 0.33 mL, 1.94 mmol) and T₃P (50% in EtOAc) (0.46 g, 0.92 mL, 1.45 mmol) at 0° C. The mixture was stirred at rt for 15 h. The mixture was poured into water (20 mL) and extracted with EtOAc (3×50 mL). The combined organic phases were dried over anhydrous Na₂SO₄, filtered and concentrated under reduced pressure. The residue was purified by trituration using diethyl ether (2×10 mL) to yield tert-butyl (2S,4R)-2-((4H-1,2,4-triazol-4-yl)methyl)-4-(5-(3-cyanophenyl)oxazole-2-carboxamido)pyrrolidine-1-carboxylate (0.13 g, 0.28 mmol, 29% yield).

LCMS: Method J, 3.79 min, MS: ES+ 464.2.

### Step (ii)

### N-((3R,5S)-5-((4H-1,2,4-Triazol-4-yl)methyl)pyrrolidin-3-yl)-5-(3-cyanophenyl)oxazole-2-carboxamide TFA salt

To a stirred solution of tert-butyl (2S,4R)-2-((4H-1,2,4-triazol-4-yl)methyl)-4-(5-(3-cyanophenyl)oxazole-2-carboxamido)pyrrolidine-1-carboxylate (0.13 g, 0.28 mmol) in DCM (2.6 mL) was added TFA (0.65 mL, 5 vol) dropwise at 0° C. The mixture was allowed to warm to rt and stirred for 1 h, then concentrated under reduced pressure to yield N-((3R,5S)-5-((4H-1,2,4-triazol-4-yl)methyl)pyrrolidin-3-yl)-5-(3-cyanophenyl)oxazole-2-carboxamide TFA salt (0.13 g, 0.27 mmol, 97% yield).

LCMS: Method J, 2.81 min, MS: ES+ 364.0.

Step (iii)

### N-((3R,5S)-5-((4H-1,2,4-Triazol-4-yl)methyl)-1-cyanopyrrolidin-3-yl)-5-(3-cyanophenyl)oxazole-2-carboxamide

To a stirred solution of N-((3R,5S)-5-((4H-1,2,4-triazol-4-yl)methyl)pyrrolidin-3-yl)-5-(3-cyanophenyl)oxazole-2-carboxamide TFA salt (0.13 g, 0.27 mmol) in THF (5 mL) was added K₂CO₃ (0.11 g, 0.81 mmol) at rt and stirred for 5 min. Cyanogen bromide (0.028 g, 0.27 mmol) was added into the reaction mixture at 0° C. The mixture was allowed to warm to rt, stirred for 1 h, then poured into water (50 mL) and extracted with DCM (4×100 mL). The combined organic phases were dried over anhydrous Na₂SO₄, filtered and concentrated under reduced pressure. The residue was purified by flash column chromatography (silica gel, 8% MeOH in DCM) to yield N-((3R,5S)-5-((4H-1,2,4-triazol-4-yl)methyl)-1-cyanopyrrolidin-3-yl)-5-(3-cyanophenyl)oxazole-2-carboxamide (0.04 g, 0.10 mmol, 38% yield).

LCMS: Method H, 2.14 min, MS: ES− 387.1; ¹H NMR (400 MHz, DMSO-d₆) δ ppm: 9.38 (d, J=6.8 Hz, 1H), 8.57 (s, 2H), 8.34 (s, 1H), 8.14 (d, J=8.0 Hz, 1H), 8.11 (s, 1H), 7.92 (d, J=7.6 Hz, 1H), 7.75 (t, J=8.0 Hz, 1H), 4.38-4.48 (m, 1H), 4.23-4.31 (m, 3H), 3.69-3.73 (m, 1H), 3.40-3.43 (m, 1H), 2.15-2.20 (m, 1H), 1.90-1.93 (m, 1H); Chiral SFC: Method Y12A, 4.3 min.

### Example 24

### N-((3R,5S)-5-((1H-1,2,3-Triazol-1-yl)methyl)-1-cyanopyrrolidin-3-yl)-4-(3-(trifluoromethyl)phenyl)picolinamide

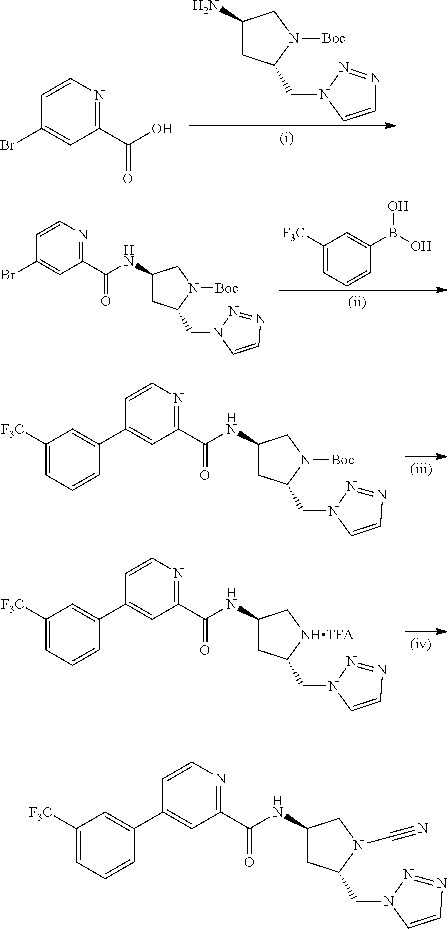

### Step i)

### tert-Butyl (2S,4R)-2-((1H-1, 2, 3-triazol-1-yl)methyl)-4-(4-bromopicolinamido)pyrrolidine-1-carboxylate

To a stirred solution of 4-bromopicolinic acid (0.22 g, 1.12 mmol) and tert-butyl (2S,4R)-2-((1H-1,2,3-triazol-1-yl)methyl)-4-aminopyrrolidine-1-carboxylate (0.20 g, 0.74 mmol) in DCM (6.8 mL) were added DIPEA (0.28 g, 0.37 mL, 2.24 mmol) and HATU (0.42 g, 1.12 mmol) at 0° C. The mixture was stirred at rt for 2 h, then poured into water (30 mL) and extracted with DCM (2×30 mL). The combined organic phases were dried over anhydrous Na₂SO₄ and concentrated under reduced pressure. The residue was purified by flash column chromatography (silica gel, 11% EtOAc in n-hexanes) to yield tert-butyl (2S,4R)-2-((1H-1,2,3-triazol-1-yl)methyl)-4-(4-bromopicolinamido)pyrrolidine-1-carboxylate (0.2 g, 0.44 mmol, 59% yield).

LCMS: Method C1, 1.18 min, MS: ES+ 451.1, 453.1.

### Step (ii)

### tert-Butyl (2S,4R)-2-((1H-1,2,3-triazol-1-yl)methyl)-4-(4-(3-(trifluoromethyl)phenyl)picolinamido) pyrrolidine-1-carboxylate

To a stirred solution of tert-butyl (2S,4R)-2-((1H-1,2,3-triazol-1-yl)methyl)-4-(4-bromopicolinamido)-pyrrolidine-1-carboxylate (0.27 g, 0.59 mmol) and (3-(trifluoromethyl)phenyl)boronic acid (CAS 1423-26-3, from Combi-Blocks, 0.14 g, 0.72 mmol) intoluene:water (10 mL, 1:1) was added K₃PO₄ (0.38 g, 1.79 mmol). The mixture was purged with N₂ gas for 15 min, followed by addition of [1,1′-bis(diphenylphosphino)ferrocene]dichloropalladium(II) (0.04 g, 0.06 mmol) and heated at 90° C. for 1 h. The mixture was poured into water (30 mL) and extracted with EtOAc (3×30 mL). The combined organic phases were dried over anhydrous Na₂SO₄ and concentrated under reduced pressure. The residue was purified by column chromatography (silica gel, 56% EtOAc in n-hexanes) to yield tert-butyl (2S,4R)-2-((1H-1,2,3-triazol-1-yl)methyl)-4-(4-(3-(trifluoromethyl)phenyl)picolinamido)-pyrrolidine-1-carboxylate (0.27 g, 0.52 mmol, 87% yield).

LCMS: Method H1, 1.33 min, MS: ES+ 517.4.

Step (iii)

### N-((3R,5S)-5-((1H-1,2,3-Triazol-1-yl)methyl)pyrrolidin-3-yl)-4-(3-(trifluoromethyl)phenyl) picolinamide TFA salt

To a stirred solution of tert-butyl (2S,4R)-2-((1H-1,2,3-triazol-1-yl)methyl)-4-(4-(3-(trifluoromethyl)-phenyl)picolinamido)pyrrolidine-1-carboxylate (0.27 g, 0.52 mmol) in DCM (2.7 mL) was added TFA (1.35 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-4-(3-(trifluoromethyl)phenyl)picolinamide TFA salt (0.35 g, quantitative yield).

### Step (iv)

### N-((3R,5S)-5-((1H-1,2,3-Triazol-1-yl)methyl)-1-cyanopyrrolidin-3-yl)-4-(3-(trifluoromethyl)phenyl) picolinamide

To a stirred solution of N-((3R,5S)-5-((1H-1,2,3-triazol-1-yl)methyl)pyrrolidin-3-yl)-4-(3-(trifluoromethyl)phenyl)picolinamide TFA salt (0.35 g, 0.66 mmol) in THF (4 mL) was added K₂CO₃ (0.36 g, 2.64 mmol) at rt and stirred for 5 min. Cyanogen bromide (0.08 g, 0.79 mmol) was added into the reaction mixture at 0° C. The mixture was stirred at rt for 1.5 h, then poured into water (20 mL) and extracted with EtOAc (3×30 mL). The combined organic phases were dried over anhydrous Na₂SO₄, filtered and concentrated under reduced pressure. The residue was purified by flash column chromatography (silica gel, 80% EtOAc in n-hexanes) to yield N-((3R,5S)-5-((1H-1,2,3-triazol-1-yl)methyl)-1-cyanopyrrolidin-3-yl)-5-(3-chlorophenyl)oxazole-2-carboxamide (0.03 g, 0.06 mmol, 13% yield over 2 steps).

LCMS: Method H1, 2.89 min, MS: ES+ 442.0; ¹H NMR (400 MHz, DMSO-d₆) δ ppm: 9.21 (d, J=6.8 Hz, 1H), 8.76 (d, J=4.8 Hz, 1H), 8.33 (s, 1H), 8.18-8.21 (m, 3H), 8.06 (d, J=3.2 Hz, 1H), 7.88-7.90 (m, 1H), 7.78-7.82 (m, 2H), 4.61-4.63 (m, 2H), 4.35-4.40 (m, 2H), 3.65-3.69 (m, 1H), 3.43-3.46 (m, 1H), 2.23-2.33 (m, 1H), 1.96-2.02 (m, 1H); Chiral SFC: Method Y9, 5.07 min.

### Example 25

### N-((3R,5S)-5-((1H-1,2,3-Triazol-1-yl)methyl)-1-cyanopyrrolidin-3-yl)-4-(3-(trifluoromethoxy)phenyl)picolinamide

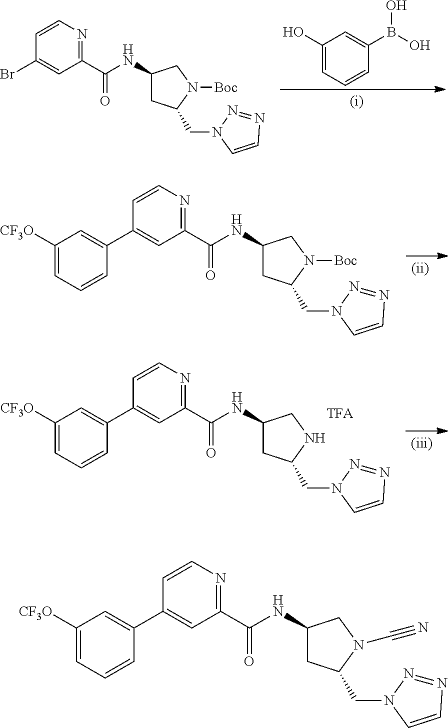

### Step (i)

### tert-Butyl (2S,4R)-2-((1H-1,2,3-triazol-1-yl)methyl)-4-(4-(3-(trifluoromethoxy)phenyl)picolinamido)-pyrrolidine-1-carboxylate

To a stirred solution of tert-butyl (2S,4R)-2-((1H-1,2,3-triazol-1-yl)methyl)-4-(4-bromopicolinamido)-pyrrolidine-1-carboxylate (0.20 g, 0.44 mmol) and (3-(trifluoromethoxy)phenyl)boronic acid (CAS #179113-90-7, from Combi-Blocks, 0.11 g, 0.53 mmol) in toluene:water (3 mL, 1:1) was added K₃PO₄ (0.28 g, 1.32 mmol). The mixture was purged with N₂ gas for 20 min, followed by addition of [1,1′-bis(diphenylphosphino)ferrocene]dichloropalladium(II) (0.02 g, 0.02 mmol) and heated at 80° C. for 2 h. The mixture was poured into water (30 mL) and extracted with EtOAc (3×30 mL). The combined organic phases were dried over anhydrous Na₂SO₄ and concentrated under reduced pressure. The residue was purified by column chromatography (silica gel, 60% EtOAc in n-hexanes) to yield tert-butyl (2S,4R)-2-((1H-1,2,3-triazol-1-yl)methyl)-4-(4-(3-(trifluoromethoxy)phenyl)picolinamido)-pyrrolidine-1-carboxylate (0.15 g, 0.28 mmol, 63% yield).

LCMS: Method C1, 1.33 min, MS: ES+ 533.3.

### Step (ii)

### N-((3R,5S)-5-((1H-1,2,3-Triazol-1-yl)methyl)pyrrolidin-3-yl)-4-(3-(trifluoromethoxy)phenyl)-picolinamide TFA Salt

To a stirred solution of tert-butyl (2S,4R)-2-((1H-1,2,3-triazol-1-yl)methyl)-4-(4-(3-(trifluoromethoxy)phenyl)picolinamido)pyrrolidine-1-carboxylate (0.14 g, 0.27 mmol) in DCM (1.5 mL) was added TFA (0.72 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-4-(3-(trifluoromethoxy)phenyl)picolinamide TFA salt (0.17 g, quantitative yield).

LCMS: Method C1, 1.11 min, MS: ES+ 433.3.

Step (iii)

### N-((3R,5S)-5-((1H-1,2,3-Triazol-1-yl)methyl)-1-cyanopyrrolidin-3-yl)-4-(3-(trifluoromethoxy)-phenyl)picolinamide

To a stirred solution of N-((3R,5S)-5-((1H-1,2,3-triazol-1-yl)methyl)pyrrolidin-3-yl)-4-(3-(trifluoromethoxy)phenyl)picolinamide TFA salt (0.17 g, 0.31 mmol) in THF (6 mL) was added K₂CO₃ (0.17 g, 1.24 mmol) at rt and stirred for 5 min. Cyanogen bromide (0.04 g, 0.37 mmol) was added into the reaction mixture at 0° C. The mixture was stirred at rt for 1 h, then poured into water (20 mL) and extracted with EtOAc (3×30 mL). The combined organic phases were dried over anhydrous Na₂SO₄, filtered and concentrated under reduced pressure. The residue was purified by flash column chromatography (silica gel, 82% EtOAc in n-hexanes) to yield N-((3R,5S)-5-((1H-1,2,3-triazol-1-yl)methyl)-1-cyanopyrrolidin-3-yl)-4-(3-(trifluoromethoxy)phenyl)picolinamide (0.025 g, 0.05 mmol, 20% yield over 2 steps).

LCMS: Method H1, 2.95 min, MS: ES+ 458.2; ¹H NMR (400 MHz, DMSO-d₆) δ ppm: 9.19 (d, J=6.8 Hz, 1H), 8.75 (d, J=4.8 Hz, 1H), 8.29 (s, 1H), 8.20 (s, 1H), 8.01 (d, J=4.8 Hz, 1H), 7.88-7.93 (m, 2H), 7.78 (s, 1H), 7.70 (t, J=8.0 Hz, 1H), 7.54 (d, J=7.6 Hz, 1H), 4.61-4.63 (m, 2H), 4.36-4.38 (m, 2H), 3.65-3.69 (m, 1H), 3.42-3.46 (m, 1H), 2.24-2.28 (m, 1H), 1.98-2.01 (m, 1H); Chiral SFC: Method Y10, 8.67 min.

### Example 26

### N-((3R,5S)-5-((1H-Pyrazol-1-yl)methyl)-1-cyanopyrrolidin-3-yl)-5-(3-cyanophenyl)oxazole-2-carboxamide

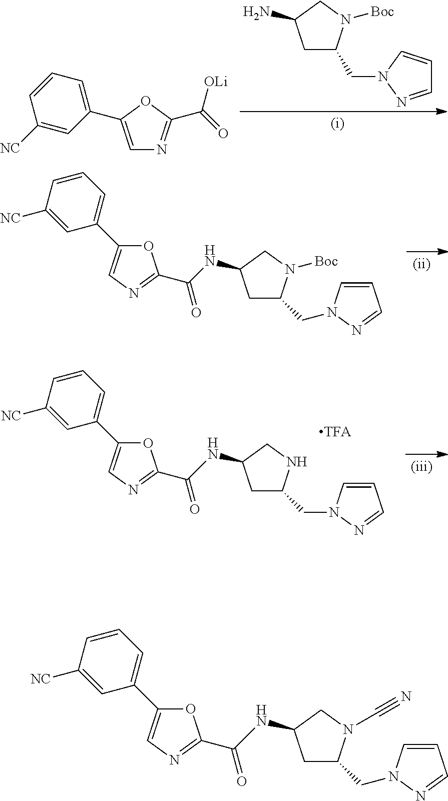

### Step (i)

### tert-Butyl (2S,4R)-2-((1H-pyrazol-1-yl)methyl)-4-(5-(3-cyanophenyl)oxazole-2-carboxamido)-pyrrolidine-1-carboxylate

To a stirred solution of lithium 5-(3-cyanophenyl)oxazole-2-carboxylate (0.25 g, 1.13 mmol) and tert-butyl (2S,4R)-2-((1H-pyrazol-1-yl)methyl)-4-aminopyrrolidine-1-carboxylate (0.30 g, 1.13 mmol) in DMF (5 mL) were added DIPEA (0.44 g, 0.58 mL, 3.41 mmol) and HATU (0.65 g, 1.70 mmol) at 0° C. The mixture was stirred at rt for 3 h, then poured into water (30 mL) and extracted with EtOAc (3×30 mL). The combined organic phases were dried over anhydrous Na₂SO₄ and concentrated under reduced pressure. The residue was purified by flash column chromatography (silica gel, 85% EtOAc in n-hexanes) to yield tert-butyl (2S,4R)-2-((1H-pyrazol-1-yl)methyl)-4-(5-(3-cyanophenyl)oxazole-2-carboxamido) pyrrolidine-1-carboxylate (0.08 g, 0.18 mmol, 16% yield).

LCMS: Method C1, 1.59 min, MS: ES+ 463.4.

### Step (ii)

### N-((3R,5S)-5-((1H-Pyrazol-1-yl)methyl)pyrrolidin-3-yl)-5-(3-cyanophenyl)oxazole-2-carboxamide TFA salt

To a stirred solution of tert-butyl (2S,4R)-2-((1H-pyrazol-1-yl)methyl)-4-(5-(3-cyanophenyl)oxazole-2-carboxamido)pyrrolidine-1-carboxylate (0.08 g, 0.17 mmol) in DCM (3 mL) was added TFA (0.24 mL, 3 vol) dropwise at 0° C. The mixture was allowed to warm to rt and stirred for 1 h, then concentrated under reduced pressure to yield N-((3R,5S)-5-((1H-pyrazol-1-yl)methyl)pyrrolidin-3-yl)-5-(3-cyanophenyl)oxazole-2-carboxamide TFA salt (0.09 g, quantitative yield).

LCMS: Method C1, 1.33 min, MS: ES+ 363.2.

Step (iii)

### N-((3R,5S)-5-((1H-Pyrazol-1-yl)methyl)-1-cyanopyrrolidin-3-yl)-5-(3-cyanophenyl)oxazole-2-carboxamide

To a stirred solution of N-((3R,5S)-5-((1H-pyrazol-1-yl)methyl)pyrrolidin-3-yl)-5-(3-cyanophenyl)oxazole-2-carboxamide TFA salt (0.08 g, 0.17 mmol) in THF (3 mL) was added K₂CO₃ (0.07 g, 0.50 mmol) at rt and stirred for 5 min. Cyanogen bromide (0.03 g, 0.25 mmol) was added into the reaction mixture at 0° C. The mixture was stirred at rt for 1 h, then poured into water (30 mL) and extracted with EtOAc (3×30 mL). The combined organic phases were dried over anhydrous Na₂SO₄, filtered and concentrated under reduced pressure. The residue was purified by flash column chromatography (silica gel, 89% EtOAc in n-hexanes) to yield N-((3R,5S)-5-((1H-pyrazol-1-yl)methyl)-1-cyanopyrrolidin-3-yl)-5-(3-cyanophenyl)oxazole-2-carboxamide (0.01 g, 0.02 mmol, 15% yield over two steps).

LCMS: Method H, 2.46 min, MS: ES+ 388.1; ¹H NMR (400 MHz, DMSO-d₆) δ ppm: 9.36 (d, J=6.4 Hz, 1H), 8.35 (s, 1H), 8.16 (d, J=7.6 Hz, 1H), 8.10 (s, 1H), 7.93 (d, J=7.2 Hz, 1H), 7.74-7.79 (m, 2H), 7.51 (s, 1H), 6.29 (s, 1H), 4.25-4.37 (m, 4H), 3.62-3.66 (m, 1H), 3.40-3.42 (m, 1H), 2.10-2.18 (m, 1H), 2.00-2.04 (m, 1H); Chiral SFC: Method Y12A, 3.99 min.

### Example 27

### N-((3R,5S)-5-((1H-Pyrazol-1-yl)methyl)-1-cyanopyrrolidin-3-yl)-5-(3-(trifluoromethoxy)phenyl)oxazole-2-carboxamide

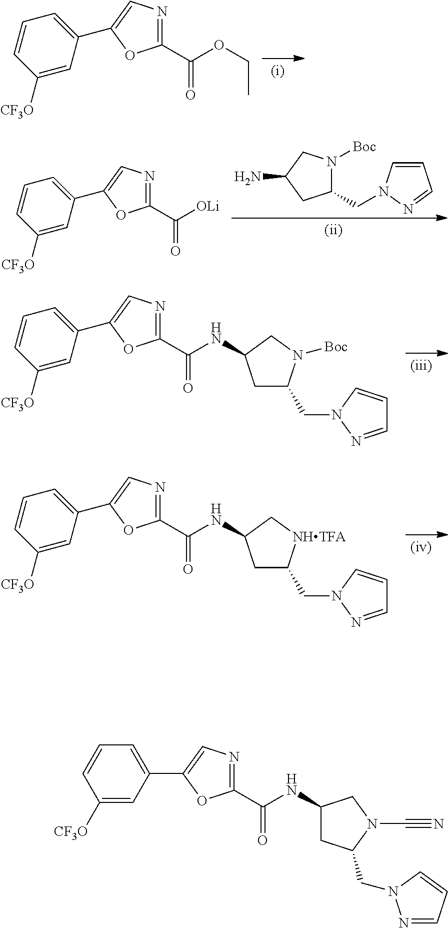

### Step (i)

### Lithium 5-(3-(trifluoromethoxy)phenyl)oxazole-2-carboxylate

To a stirred solution of ethyl 5-(3-(trifluoromethoxy)phenyl)oxazole-2-carboxylate (0.3 g, 0.99 mmol) in THF:water (3:2, 5 mL) was added lithium hydroxide monohydrate (0.08 g, 1.99 mmol) in portions at 0° C. The mixture was stirred at rt for 2 h, then concentrated under reduced pressure to yield lithium 5-(3-(trifluoromethoxy)phenyl)oxazole-2-carboxylate (0.3 g, quantitative yield).

LCMS: Method C1, 1.15 min, MS: m/z not supportive.

### Step (ii)

### tert-Butyl (2S,4R)-2-((1H-pyrazol-1-yl)methyl)-4-(5-(3-(trifluoromethoxy)phenyl)oxazole-2-carboxamido)pyrrolidine-1-carboxylate

To a stirred solution of lithium 5-(3-(trifluoromethoxy)phenyl)oxazole-2-carboxylate (0.30 g, 1.07 mmol) and tert-butyl (2S,4R)-2-((1H-pyrazol-1-yl)methyl)-4-aminopyrrolidine-1-carboxylate (0.29 g, 1.07 mmol) in pyridine (3 mL) was added POCl₃ (0.49 g, 0.29 mL, 3.22 mmol) dropwise at 0° C. The mixture was stirred at rt for 30 min, then poured into water (50 mL) and extracted with EtOAc (2×30 mL). The combined organic phases were dried over anhydrous Na₂SO₄ and concentrated under reduced pressure. The residue was purified by flash column chromatography (silica gel, 55% EtOAc in n-hexanes) to yield tert-butyl (2S,4R)-2-((1H-pyrazol-1-yl)methyl)-4-(5-(3-(trifluoromethoxy)phenyl)oxazole-2-carboxamido)pyrrolidine-1-carboxylate (0.14 g, 5.83 mmol, 26% yield). LCMS: Method C1, 1.36 min, MS: ES+: 522.3.

Step (iii)

### N-((3R,5S)-5-((1H-Pyrazol-1-yl)methyl)pyrrolidin-3-yl)-5-(3-(trifluoromethoxy)phenyl)oxazole-2-carboxamide TFA salt

To a stirred solution of tert-butyl (2S,4R)-2-((1H-pyrazol-1-yl)methyl)-4-(5-(3-(trifluoromethoxy)phenyl)oxazole-2-carboxamido)pyrrolidine-1-carboxylate (0.14 g, 0.26 mmol) in DCM (5 mL) was added TFA (1.2 mL, 8.5 vol) dropwise at 0° C. The mixture was allowed to warm to rt and stirred for 1 h, then concentrated under reduced pressure to yield N-((3R,5S)-5-((1H-pyrazol-1-yl)methyl)pyrrolidin-3-yl)-5-(3-(trifluoromethoxy)phenyl)oxazole-2-carboxamide TFA salt (0.15 g, quantitative yield). LCMS: Method C1, 1.11 min, MS: ES+ 422.3.

### Step (iv)

### N-((3R,5S)-5-((1H-Pyrazol-1-yl)methyl)-1-cyanopyrrolidin-3-yl)-3-(3-(trifluoromethoxy)phenyl)pyrrolidine-1-carboxamide

To a stirred solution of N-((3R,5S)-5-((1H-pyrazol-1-yl)methyl)pyrrolidin-3-yl)-5-(3-(trifluoromethoxy)phenyl)oxazole-2-carboxamide TFA salt (0.15 g, 0.35 mmol) in THF (3.0 mL) was added K₂CO₃ (0.14 g, 1.06 mmol) at rt and stirred for 5 min. Cyanogen bromide (0.03 g, 0.35 mmol) was added into the reaction mixture at 0° C. The mixture was stirred at rt for 1 h, then poured into water (30 mL) and extracted with EtOAc (3×30 mL). The combined organic phases were dried over anhydrous Na₂SO₄, filtered and concentrated under reduced pressure. The residue was purified by flash column chromatography (silica gel, 20% EtOAc in n-hexanes) to yield N-((3R,5S)-5-((1H-pyrazol-1-yl)methyl)-1-cyanopyrrolidin-3-yl)-5-(3-(trifluoromethoxy)phenyl)oxazole-2-carboxamide (0.03 g, 0.33 mmol, 21% yield over two steps).

LCMS: Method H1, 2.95 min, MS: ES+ 447.0; ¹H NMR (400 MHz, DMSO-d₆) δ ppm: 9.30 (s, 1H), 8.07 (s, 1H), 7.77-7.87 (m, 3H), 7.65-7.69 (m, 1H), 7.46-7.49 (m, 2H), 6.28 (s, 1H), 4.22-4.29 (m, 4H), 3.61-3.65 (m, 1H), 3.38-3.40 (m, 1H), 2.1-2.21 (m, 1H), 1.97-2.08 (m, 1H); Chiral SFC: Method Y10, 7.24 min.

### Example 28 and 29

### N-((3R,5S)-1-Cyano-5-((3-methyl-1H-pyrazol-1-yl)methyl)pyrrolidin-3-yl)-5-(3-(trifluoromethoxy)phenyl)oxazole-2-carboxamide &

### N-((3R,5S)-1-Cyano-5-((5-methyl-1H-pyrazol-1-yl)methyl)pyrrolidin-3-yl)-5-(3-(trifluoromethoxy)phenyl)oxazole-2-carboxamide

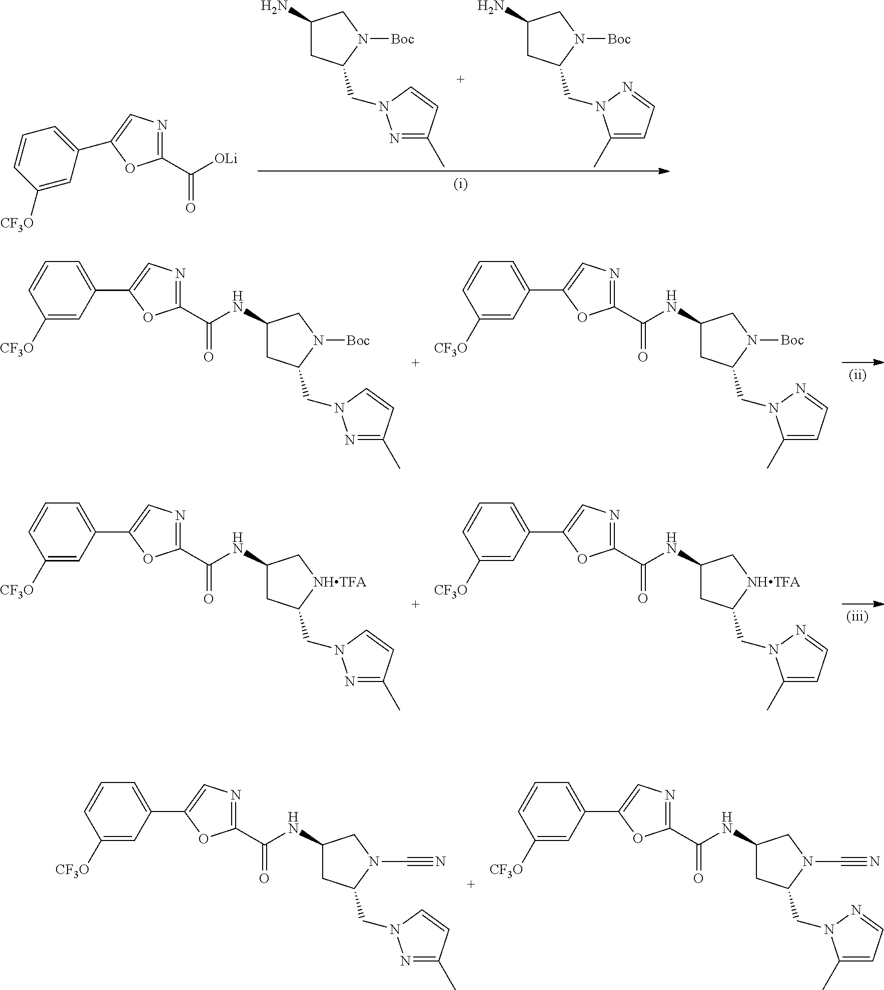

### Step (i)

### tert-Butyl (2S,4R)-2-((3-methyl-1H-pyrazol-1-yl)methyl)-4-(5-(3-(trifluoromethoxy)phenyl)oxazole-2-carboxamido)pyrrolidine-1-carboxylate &

### tert-Butyl (2S,4R)-2-((5-methyl-1H-pyrazol-1-yl)methyl)-4-(5-(3-(trifluoromethoxy)phenyl)oxazole-2-carboxamido)pyrrolidine-1-carboxylate

To a stirred solution of lithium 5-(3-(trifluoromethoxy)phenyl)oxazole-2-carboxylate (0.29 g, 1.03 mmol) and tert-butyl (2S,4R)-4-amino-2-((3-methyl-1H-pyrazol-1-yl)methyl)pyrrolidine-1-carboxylate & tert-butyl (2S,4R)-4-amino-2-((5-methyl-1H-pyrazol-1-yl)methyl)pyrrolidine-1-carboxylate (0.29 g, 1.03 mmol) in pyridine (0.28 mL) was added POCl₃ (0.29 mL, 3.11 mmol) dropwise at 0° C. and stirred for 15 min at 0° C. The mixture was poured into saturated NaHCO₃ solution (100 mL) and extracted with EtOAc (3×100 mL). The combined organic phases were dried over anhydrous Na₂SO₄, filtered and concentrated under reduced pressure. The residue was purified by flash column chromatography (silica gel, 30-35% EtOAc in n-hexanes) to yield tert-butyl (2S,4R)-2-((3-methyl-1H-pyrazol-1-yl)methyl)-4-(5-(3-(trifluoromethoxy)phenyl)oxazole-2-carboxamido)-pyrrolidine-1-carboxylate & tert-butyl (2S,4R)-2-((5-methyl-1H-pyrazol-1-yl)methyl)-4-(5-(3-(trifluoromethoxy)phenyl)oxazole-2-carboxamido)pyrrolidine-1-carboxylate as a mixture (0.09 g, 0.17 mmol, 17% yield). LCMS: Method H1, 3.48 min, MS: ES+ 536.1 & 3.59 min, MS: ES+ 536.0.

### Step (ii)

### N-((3R,5S)-5-((3-Methyl-1H-pyrazol-1-yl)methyl)pyrrolidin-3-yl)-5-(3-(trifluoromethoxy)phenyl)-oxazole-2-carboxamide TFA salt &

### N-((3R,5S)-5-((5-Methyl-1H-pyrazol-1-yl)methyl)pyrrolidin-3-yl)-5-(3-(trifluoromethoxy)phenyl)-oxazole-2-carboxamide TFA salt

To a stirred solution of tert-butyl (2S,4R)-2-((3-methyl-1H-pyrazol-1-yl)methyl)-4-(5-(3-(trifluoromethoxy)phenyl)oxazole-2-carboxamido)pyrrolidine-1-carboxylate & tert-butyl (2S,4R)-2-((5-methyl-1H-pyrazol-1-yl)methyl)-4-(5-(3-(trifluoromethoxy)phenyl)oxazole-2-carboxamido)-pyrrolidine-1-carboxylate (0.09 g, 0.17 mmol) in DCM (2 mL) was added TFA (0.48 mL, 5 vol) dropwise at 0° C. The mixture was allowed to warm to rt and stirred for 30 min, then concentrated under reduced pressure to yield N-((3R,5S)-5-((3-methyl-1H-pyrazol-1-yl)methyl)pyrrolidin-3-yl)-5-(3-(trifluoromethoxy)phenyl)oxazole-2-carboxamide & N-((3R,5S)-5-((5-methyl-1H-pyrazol-1-yl)methyl)pyrrolidin-3-yl)-5-(3-(trifluoromethoxy)phenyl)oxazole-2-carboxamide TFA salt as a mixture (0.11 g, quantitative yield).

LCMS: Method C1, 1.18 min, MS: ES+ 436.2.

Step (iii)

### N-((3R,5S)-1-Cyano-5-((3-methyl-1H-pyrazol-1-yl)methyl)pyrrolidin-3-yl)-5-(3-(trifluoromethoxy)phenyl)oxazole-2-carboxamide &

### N-((3R,5S)-1-Cyano-5-((5-methyl-1H-pyrazol-1-yl)methyl)pyrrolidin-3-yl)-5-(3-(trifluoromethoxy)phenyl)oxazole-2-carboxamide

To a stirred solution of N-((3R,5S)-5-((3-methyl-1H-pyrazol-1-yl)methyl)pyrrolidin-3-yl)-5-(3-(trifluoromethoxy)phenyl)oxazole-2-carboxamide & N-((3R,5S)-5-((5-methyl-1H-pyrazol-1-yl)methyl)pyrrolidin-3-yl)-5-(3-(trifluoromethoxy)phenyl)oxazole-2-carboxamide TFA salt (0.11 g, 0.20 mmol) in THF (3 mL) was added K₂CO₃ (0.13 g, 1.00 mmol) at rt and stirred for 5 min. Cyanogen bromide (0.03 g, 0.20 mmol) was added into the reaction mixture at 0° C. The mixture was stirred at 0° C. for 30 min, then poured into ice-cold water (100 mL) extracted with EtOAc (3×50 mL). The combined organic phases were dried over anhydrous Na₂SO₄, filtered and concentrated under reduced pressure to yield N-((3R,5S)-1-cyano-5-((5-methyl-1H-pyrazol-1-yl)methyl)pyrrolidin-3-yl)-5-(3-(trifluoromethoxy)phenyl)oxazole-2-carboxamide & N-((3R,5S)-1-cyano-5-((3-methyl-1H-pyrazol-1-yl)methyl)pyrrolidin-3-yl)-5-(3-(trifluoromethoxy)phenyl)oxazole-2-carboxamide as a mixture (0.04 g, 0.10 mmol, 55% yield).

The two compounds were separated by chiral preparative HPLC (Method Z1, UV spectra recorded at 287 nm lambda max) to yield N-((3R,5S)-1-cyano-5-((3-methyl-1H-pyrazol-1-yl)methyl)pyrrolidin-3-yl)-5-(3-(trifluoromethoxy)phenyl)oxazole-2-carboxamide (0.02 g, 0.04 mmol, 22% yield) & N-((3R,5S)-1-cyano-5-((5-methyl-1H-pyrazol-1-yl)methyl)pyrrolidin-3-yl)-5-(3-(trifluoromethoxy)phenyl)oxazole-2-carboxamide (0.01 g, 0.03 mmol, 16% yield).

Regioisomer assignments were made on the basis of nOe NMR experiments in which the part of the ¹H NMR spectrum corresponding to the methylene attached to the pyrazole was irradiated and the effect on the part of the ¹H NMR spectrum corresponding to the methyl group was measured.a N-((3R,5S)-1-Cyano-5-((3-methyl-1H-pyrazol-1-yl)methyl)pyrrolidin-3-yl)-5-(3-(trifluoromethoxy)phenyl)oxazole-2-carboxamide (Example 28) LCMS: Method H1, 2.97 min, MS: ES+ 461.0; ¹H NMR (400 MHz, DMSO-d₆) δ ppm: 9.34 (d, J=6.8 Hz, 1H), 8.09 (s, 1H), 7.87 (d, J=8.0 Hz, 1H), 7.82 (s, 1H), 7.68 (t, J=8.0 Hz, 1H), 7.62 (s, 1H), 7.46 (d, J=8.0 Hz, 1H), 6.04 (s, 1H), 4.28-4.31 (m, 1H), 4.21-4.23 (m, 3H), 3.62-3.66 (m, 1H), 3.2-3.3 (m, 1H), 2.16 (s, 3H), 2.08-2.13 (m, 1H), 1.91-2.03 (m, 1H); Chiral HPLC: Method Y30, 7.05 min.

### N-((3R,5S)-1-Cyano-5-((5-methyl-1H-pyrazol-1-yl)methyl)pyrrolidin-3-yl)-5-(3-(trifluoromethoxy)phenyl)oxazole-2-carboxamide (Example 29)

LCMS: Method H1, 2.95 min MS: ES− 459.0; ¹H NMR (400 MHz, DMSO-d₆) δ ppm: 9.36 (d, J=6.8 Hz, 1H), 8.09 (s, 1H), 7.87 (d, J=7.6 Hz, 1H), 7.82 (s, 1H), 7.68 (t, J=8.0 Hz, 1H), 7.46 (d, J=7.6 Hz, 1H), 7.36 (s, 1H), 6.04 (s, 1H), 4.36-4.37 (m, 1H), 4.26-4.31 (m, 1H), 4.19-4.20 (m, 2H), 3.66-3.70 (m, 1H), 3.2-3.3 (m, 1H), 2.28 (s, 3H), 2.12-2.16 (m, 1H), 2.02-2.08 (m, 1H); Chiral HPLC: Method Y30, 7.65 min.

### Example 30

### N-((3R,5S)-5-((1H-Pyrazol-1-yl)methyl)-1-cyanopyrrolidin-3-yl)(3(trifluoromethyl)phenyl)oxazole-2-carboxamide

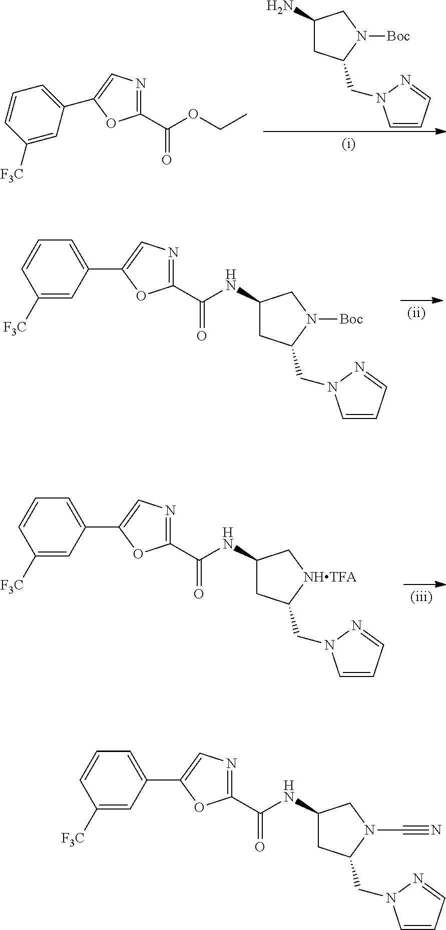

### Step (i)

### tert-Butyl (2S,4R)-2-((1H-pyrazol-1-yl)methyl)-4-(5-(3-(trifluoromethyl)phenyl)oxazole-2-carboxamido)pyrrolidine-1-carboxylate

To a stirred solution of ethyl 5-(3-(trifluoromethyl)phenyl)oxazole-2-carboxylate (0.5 g, 1.75 mmol) and tert-butyl (2S,4R)-2-((1H-pyrazol-1-yl)methyl)-4-aminopyrrolidine-1-carboxylate (0.46 g, 1.75 mmol) in THF (5 mL) was added DBU (1.5 mL, 3 vol) dropwise at 0° C. The mixture was allowed to warm to rt and heated at 65° C. for 2 h. The mixture was poured into water (30 mL) and extracted with EtOAc (2×50 mL). The combined organic phases were dried over anhydrous Na₂SO₄, filtered and concentrated under reduced pressure. The residue was purified by flash column chromatography (silica gel, 30% EtOAc in n-hexanes) to yield tert-butyl (2S,4R)-2-((1H-pyrazol-1-yl)methyl)-4-(5-(3-(trifluoromethyl)phenyl)oxazole-2-carboxamido)pyrrolidine-1-carboxylate (0.21 g, 0.41 mmol, 13% yield).

LCMS: Method J1, 3.72 min, MS: ES+ 506.3.

### Step (ii)

### N-((3R,5S)-5-((1H-Pyrazol-1-yl)methyl)pyrrolidin-3-yl)-5-(3-(trifluoromethyl)phenyl)oxazole-2-carboxamide TFA salt

To a stirred solution of tert-butyl (2S,4R)-2-((1H-pyrazol-1-yl)methyl)-4-(5-(3-(trifluoromethyl)phenyl)oxazole-2-carboxamido)pyrrolidine-1-carboxylate (0.21 g, 0.41 mmol) in DCM (2.1 mL) was added TFA (2.1 mL, 10 vol) dropwise at 0° C. The mixture was allowed to warm to rt and stirred for 1 h, then concentrated under reduced pressure to yield N-((3R,5S)-5-((1H-pyrazol-1-yl)methyl)pyrrolidin-3-yl)-5-(3-(trifluoromethyl)phenyl)oxazole-2-carboxamide TFA salt (0.34 g, quantitative yield).

LCMS: Method C1, 1.08 min, MS: ES+ 406.3.

Step (iii)

### N-((3R,5S)-5-((1H-Pyrazol-1-yl)methyl)-1-cyanopyrrolidin-3-yl)-5-(-3-(trifluoromethyl)phenyl)oxazole-2-carboxamide

To a stirred solution of N-((3R,5S)-5-((1H-pyrazol-1-yl)methyl)pyrrolidin-3-yl)-5-(3-(trifluoromethyl)phenyl)oxazole-2-carboxamide TFA salt (0.34 g, 0.65 mmol) in THF (3.4 mL) was added K₂CO₃ (0.27 g, 1.96 mmol) at rt and stirred for 5 min. Cyanogen bromide (0.08 g, 0.72 mmol) was added into the reaction mixture at 0° C. The mixture was stirred at rt for 1.5 h, then poured into ice-cold water (100 mL) to form a precipitate. Solid was collected by filtration through a Buchner funnel, washed with n-hexanes (3×20 mL) to yield N-((3R,5S)-5-((1H-pyrazol-1-yl)methyl)-1-cyanopyrrolidin-3-yl)-5-(3-(trifluoromethyl)phenyl)oxazole-2-carboxamide (0.15 g, 0.35 mmol, 82% yield over two steps).

LCMS: Method H1, 2.87 min, MS: ES+ 431.0; ¹H NMR (400 MHz, DMSO-d₆) δ ppm: 9.39 (d, J=6.4 Hz, 1H), 8.13-8.17 (m, 3H), 7.79-7.82 (m, 3H), 7.52 (s, 1H), 6.30 (s, 1H), 4.29-4.32 (m, 4H), 3.63-3.66 (m, 1H), 3.41-3.42 (m, 1H), 2.14-2.19 (m, 1H), 2.01-2.05 (m, 1H); Chiral SFC: Method Y8, 6.27 min.

### Example 34

### N-((3R,5S)-5-((1H-Pyrazol-1-yl)methyl)-1-cyanopyrrolidin-3-yl)-2-fluoro-4-(1-methyl-1H-indazol-5-yl)benzamide

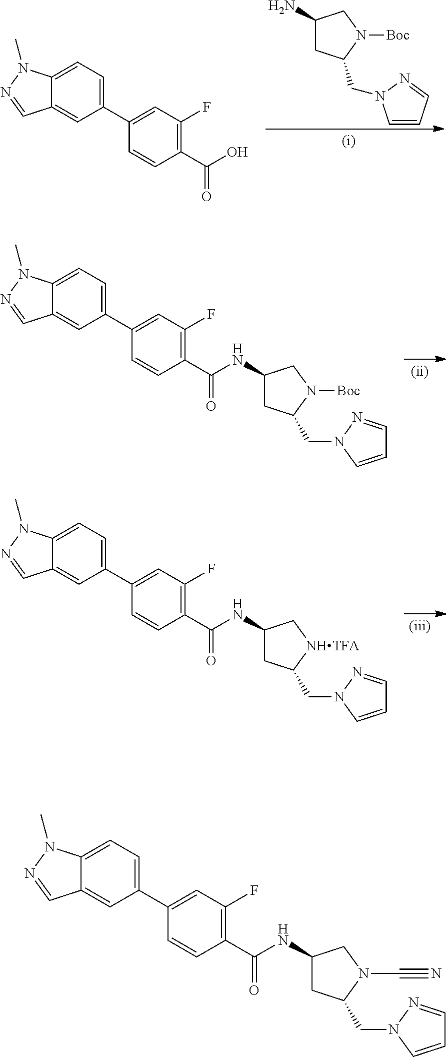

### Step (i)

### tert-Butyl (2S,4R)-2-((1H-pyrazol-1-yl)methyl)-4-(2-fluoro-4-(1-methyl-1h-indazol-5-yl)benzamido)pyrrolidine-1-carboxylate

To a stirred solution of 2-fluoro-4-(1-methyl-1H-indazol-5-yl)benzoic acid (0.3 g, 1.11 mmol) in THF (3 mL) were added DIPEA (0.43 g, 0.58 mL, 3.33 mmol) and HATU (0.84 g, 2.22 mmol) in portions at 0° C. After 30 min, tert-butyl (2S,4R)-2-((1H-pyrazol-1-yl)methyl)-4-aminopyrrolidine-1-carboxylate (0.32 g, 1.22 mmol) was added at 0° C. The mixture was slowly warmed to rt and stirred for 3 h, then poured into ice-cold water (15 mL) and extracted with EtOAc (3×30 mL). The combined organic phases were dried over Na₂SO₄ and concentrated under reduced pressure. The residue was purified by flash column chromatography (silica gel, 70% EtOAc in n-hexanes) to yield tert-butyl (2S,4R)-2-((1H-pyrazol-1-yl)methyl)-4-(2-fluoro-4-(1-methyl-1H-indazol-5-yl)benzamido)pyrrolidine-1-carboxylate (0.50 g, 0.96 mmol, 86% yield).

LCMS: Method J1, 3.90 min, MS: ES+: 519.1.

### Step (ii)

### N-((3R,5S)-5-((1H-Pyrazol-1-yl)methyl)pyrrolidin-3-yl)-2-fluoro-4-(1-methyl-1H-indazol-5-yl)benzamide TFA salt

To a stirred solution of tert-butyl (2S,4R)-2-((1H-pyrazol-1-yl)methyl)-4-(2-fluoro-4-(1-methyl-1H-indazol-5-yl)benzamido)pyrrolidine-1-carboxylate (0.50 g, 0.96 mmol) in DCM (5 mL) was added TFA (2.5 mL, 5 vol) dropwise at 0° C. The mixture was allowed to warm to rt and stirred for 1 h, then concentrated under reduced pressure to yield N-((3R,5S)-5-((1H-pyrazol-1-yl)methyl)pyrrolidin-3-yl)-2-fluoro-4-(1-methyl-1H-indazol-5-yl)benzamide TFA salt (0.35 g, 0.83 mmol, 86% yield).

LCMS: Method J1, 2.97 min, MS: ES+ 419.0.

Step (iii)

### N-((3R,5S)-5-((1H-Pyrazol-1-yl)methyl)-1-cyanopyrrolidin-3-yl)-2-fluoro-4-(1-methyl-1H-indazol-5-yl)benzamide

To a stirred solution of N-((3R,5S)-5-((1H-pyrazol-1-yl)methyl)pyrrolidin-3-yl)-2-fluoro-4-(1-methyl-1H-indazol-5-yl)benzamide TFA salt (0.35 g, 0.83 mmol) in THF (3.5 mL) was added K₂CO₃ (0.46 g, 3.34 mmol) at rt and stirred for 5 min. Cyanogen bromide (0.09 g, 0.92 mmol) was added into the reaction mixture at 0° C. The mixture was stirred at rt for 1 h, then poured into water (30 mL) and extracted with EtOAc (3×30 mL). The combined organic phases were dried over anhydrous Na₂SO₄, filtered and concentrated under reduced pressure. The residue was purified by flash column chromatography (silica gel, 5% MeOH in DCM) to yield N-((3R,5S)-5-((1H-pyrazol-1-yl)methyl)-1-cyanopyrrolidin-3-yl)-2-fluoro-4-(1-methyl-1H-indazol-5-yl)benzamide (0.07 g, 0.17 mmol, 29% yield).

LCMS: Method H1, 2.58 min, MS: ES+ 444.2; ¹H NMR (400 MHz, DMSO-d₆) δ ppm: 8.71 (d, J=6.0 Hz, 1H), 8.14 (d, J=10.4 Hz, 2H), 7.75-7.82 (m, 3H), 7.66-7.70 (m, 3H), 7.51 (s, 1H), 6.29 (s, 1H), 4.23-4.33 (m, 4H), 4.09 (s, 3H), 3.61-3.65 (m, 1H), 3.31-3.34 (m, 1H), 1.97-2.09 (m, 2H); Chiral SFC: Method Y9, 5.60 min.

### Example 35

### N-((3R,5S)-5-((1H-1,2,3-Triazol-1-yl)methyl)-1-cyanopyrrolidin-3-yl)-2-fluoro-4-(1-methyl-1H-indazol-5-yl)benzamide

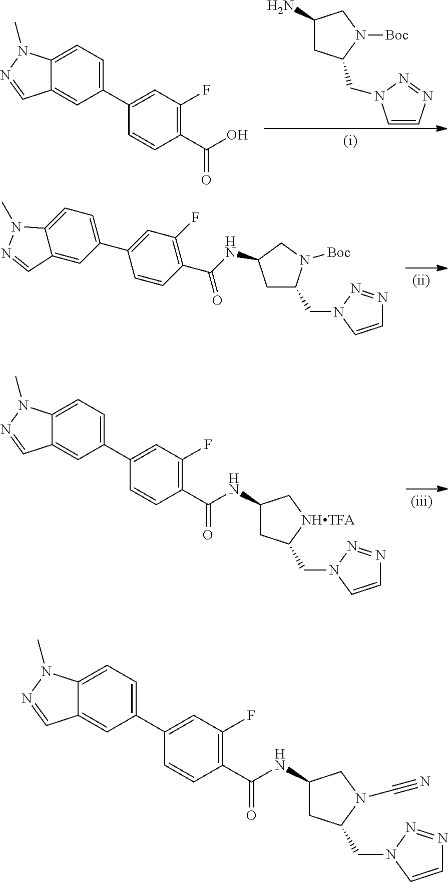

### Step (i)

### tert-Butyl (2S,4R)-2-((1H-1,2,3-triazol-1-yl)methyl)-4-(2-fluoro-4-(1-methyl-1H-indazol-5-yl)benzamido)pyrrolidine-1-carboxylate

To a stirred solution of 2-fluoro-4-(1-methyl-1H-indazol-5-yl)benzoic acid (0.3 g, 1.11 mmol) in THF (3 mL) were added DIPEA (0.43 g, 0.58 mL, 3.33 mmol) and HATU (0.84 g, 2.22 mmol) in portions at 0° C. After 30 min, tert-butyl (2S,4R)-2-((1H-1,2,3-triazol-1-yl)methyl)-4-aminopyrrolidine-1-carboxylate (0.32 g, 1.22 mmol) was added at 0° C. The mixture was slowly warmed to rt and stirred for 3 h, then poured into ice-cold water (15 mL) and extracted with EtOAc (3×30 mL). The combined organic phases were dried over Na₂SO₄ and concentrated under reduced pressure. The residue was purified by flash column chromatography (silica gel, 90% EtOAc in n-hexanes) to yield tert-butyl (2S,4R)-2-((1H-1,2,3-triazol-1-yl)methyl)-4-(2-fluoro-4-(1-methyl-1H-indazol-5-yl)benzamido)pyrrolidine-1-carboxylate (0.50 g, 0.96 mmol, 86% yield).

LCMS: Method C1, 1.20 min, MS: ES+: 520.3.

### Step (ii)

### N-((3R,5S)-5-((1H-1,2,3-Triazol-1-yl)methyl)pyrrolidin-3-yl)-2-fluoro-4-(1-methyl-1H-indazol-5-yl)benzamide TFA salt

To a stirred solution of tert-butyl (2S,4R)-2-((1H-1,2,3-triazol-1-yl)methyl)-4-(2-fluoro-4-(1-methyl-1H-indazol-5-yl)benzamido)pyrrolidine-1-carboxylate (0.50 g, 0.96 mmol) in DCM (5 mL) was added TFA (2.5 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-2-fluoro-4-(1-methyl-1H-indazol-5-yl)benzamide TFA salt (0.35 g, 0.65 mmol, 86% yield).

LCMS: Method C1, 0.99 min, MS: ES+ 420.5.

Step (iii)

### N-((3R,5S)-5-((1H-1,2,3-Triazol-1-yl)methyl)-1-cyanopyrrolidin-3-yl)-2-fluoro-4-(1-methyl-1H-indazol-5-yl)benzamide

To a stirred solution of N-((3R,5S)-5-((1H-1,2,3-triazol-1-yl)methyl)pyrrolidin-3-yl)-2-fluoro-4-(1-methyl-1H-indazol-5-yl)benzamide TFA salt (0.35 g, 0.65 mmol) in THF (3.5 mL) was added K₂CO₃ (0.45 g, 3.28 mmol) at rt and stirred for 5 min. Cyanogen bromide (0.07 g, 0.72 mmol) was added into the reaction mixture at 0° C. The mixture was stirred at rt for 1 h, then poured into water (30 mL) and extracted with EtOAc (3×30 mL). The combined organic phases were dried over anhydrous Na₂SO₄, filtered and concentrated under reduced pressure. The residue was purified by flash column chromatography (silica gel, 10% MeOH in DCM) to yield N-((3R,5S)-5-((1H-1,2,3-triazol-1-yl)methyl)-1-cyanopyrrolidin-3-yl)-2-fluoro-4-(1-methyl-1H-indazol-5-yl)benzamide (0.08 g, 0.18 mmol, 21% yield).

LCMS: Method H1, 2.45 min, MS: ES+ 445.2; ¹H NMR (400 MHz, DMSO-d₆) δ ppm: 8.79 (d, J=5.2 Hz, 1H), 8.15-8.21 (m, 3H), 7.77-7.84 (m, 3H), 7.68-7.72 (m, 3H), 4.64-4.66 (m, 2H), 4.31-4.42 (m, 2H), 4.10 (s, 3H), 3.66-3.70 (m, 1H), 3.35 (s, 1H), 2.09-2.19 (m, 1H), 1.97-2.01 (m, 1H); Chiral SFC: Method Y9, 6.07 min.

### Example 36

### N-((3R,5S)-5-((1H-Pyrazol-1-yl)methyl)-1-cyanopyrrolidin-3-yl)-3-(3-(trifluoromethoxy)phenyl) pyrrolidine-1-carboxamide

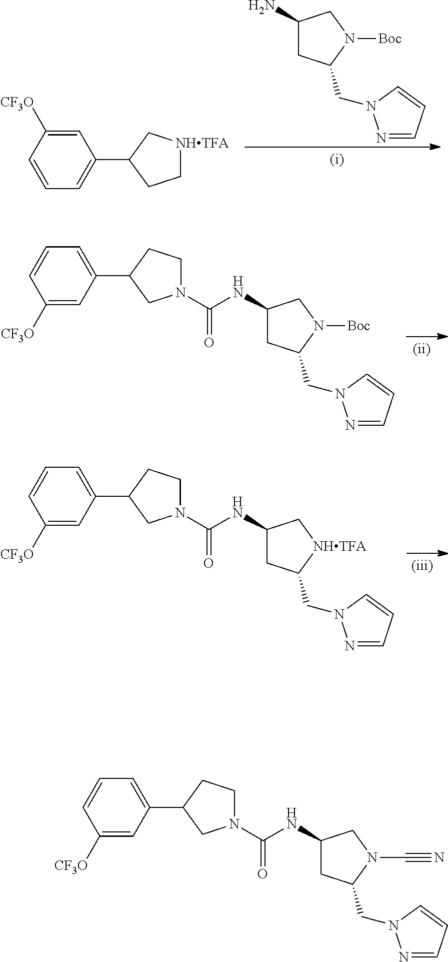

### Step (i)

### tert-Butyl (2S,4R)-2-((1H-pyrazol-1-yl)methyl)-4-(3-(3-(trifluoromethoxy)phenyl)pyrrolidine-1-carboxamido)pyrrolidine-1-carboxylate

To a stirred solution of 3-(3-(trifluoromethoxy)phenyl) pyrrolidine TFA salt (0.5 g, 2.16 mmol) in THF (4 mL) and tert-butyl (2S,4R)-2-((1H-pyrazol-1-yl)methyl)-4-aminopyrrolidine-1-carboxylate (0.57 g, 3.16 mmol) was added TEA (0.89 mL, 6.49 mmol) at 0° C. and stirred for 10 min. Triphosgene (0.32 g, 1.08 mmol) was added in portions into the reaction mixture at 0° C. The mixture was stirred at rt for 5 h, then poured into water (20 mL) and extracted with DCM (3×20 mL). The combined organic phases were dried over anhydrous Na₂SO₄, filtered and concentrated under reduced pressure. The residue was purified by flash column chromatography (silica gel, 3% MeOH in DCM) to yield tert-butyl (2S,4R)-2-((1H-pyrazol-1-yl)methyl)-4-(3-(3-(trifluoromethoxy)phenyl)pyrrolidine-1-carboxamido)pyrrolidine-1-carboxylate (0.14 g, 0.34 mmol, 13% yield).

LCMS: Method C1, 1.41 min, MS: ES+ 524.4.

### Step (ii)

### N-((3R,5S)-5-((1H-Pyrazol-1-yl)methyl)pyrrolidin-3-yl)-3-(3-(trifluoromethoxy)phenyl)pyrrolidine-1-carboxamide TFA salt

To a stirred solution of tert-butyl (2S,4R)-2-((1H-pyrazol-1-yl)methyl)-4-(3-(3-(trifluoromethoxy)-phenyl)pyrrolidine-1-carboxamido)pyrrolidine-1-carboxylate (0.14 g, 0.34 mmol) in DCM (3 mL) was added TFA (0.73 mL, 5 vol) dropwise at 0° C. The mixture was allowed to warm to rt and stirred for 1 h, then concentrated under reduced pressure to yield N-((3R,5S)-5-((1H-pyrazol-1-yl)methyl)pyrrolidin-3-yl)-3-(3-(trifluoromethoxy)phenyl)pyrrolidine-1-carboxamide TFA salt (0.1 g, 0.22 mmol, 70% yield). LCMS: Method C1, 1.17 min, MS: ES+ 424.2.

Step (iii)

### N-((3R,5S)-5-((1H-Pyrazol-1-yl)methyl)-1-cyanopyrrolidin-3-yl)-3-(3-(trifluoromethoxy)phenyl) pyrrolidine-1-carboxamide

To a stirred solution of N-((3R,5S)-5-((1H-pyrazol-1-yl)methyl)pyrrolidin-3-yl)-3-(3-(trifluoromethoxy)phenyl)pyrrolidine-1-carboxamide TFA salt (0.1 g, 0.22 mmol) in THF (2 mL) was added K₂CO₃ (0.09 g, 068 mmol) at rt and stirred for 5 min. Cyanogen bromide (0.04 g, 0.34 mmol) was added into the reaction mixture at 0° C. The mixture was stirred at rt for 1 h, then poured into water (30 mL) and extracted with EtOAc (3×30 mL). The combined organic phases were dried over anhydrous Na₂SO₄, filtered and concentrated under reduced pressure. The residue was purified by flash column chromatography (silica gel, 95% EtOAc in n-hexanes) to yield N-((3R,5S)-5-((1H-pyrazol-1-yl)methyl)-1-cyanopyrrolidin-3-yl)-3-(3-(trifluoromethoxy)phenyl)pyrrolidine-1-carboxamide (0.04 g, 0.08 mmol, 14% yield).

LCMS: Method H1, 2.90 min, MS: ES+ 449.0; ¹H NMR (400 MHz, DMSO-d₆) δ ppm: 7.77 (s, 1H), 7.46-7.50 (m, 2H), 7.36 (d, J=7.2 Hz, 1H), 7.31 (s, 1H), 7.25 (d, J=7.6 Hz, 1H), 6.28 (s, 2H), 4.19-4.29 (m, 3H), 3.97-4.05 (m, 1H), 3.69-3.77 (m, 1H), 3.40-3.56 (m, 4H), 3.15-3.23 (m, 2H), 2.20-2.30 (s, 1H), 1.86-2.00 (m, 3H); Chiral SFC: Method Y31, 15.52 min and 17.04 min.

### Example 37 & Example 38

### (S)—N-((3R,5S)-5-((1H-pyrazol-1-yl)methyl)-1-cyanopyrrolidin-3-yl)-3-(3-(trifluoromethoxy)phenyl)pyrrolidine-1-carboxamide

### (R)—N-((3R,5S)-5-((1H-pyrazol-1-yl)methyl)-1-cyanopyrrolidin-3-yl)-3-(3-(trifluoromethoxy)phenyl)pyrrolidine-1-carboxamide

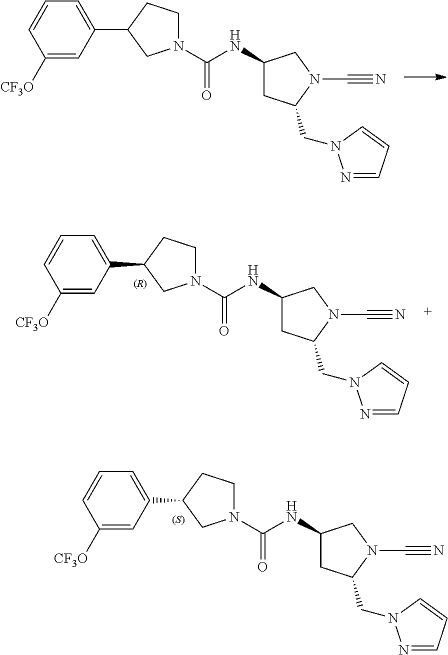

The diastereomers were separated by chiral preparative HPLC (Method Z2, UV spectra recorded at 220 nm lambda max) to yield Diastereomer 1 of the title compounds as the first eluting isomer (0.008 g, 0.02 mmol, 18% yield) & Diastereomer 2 of the title compounds as the second eluting isomer (0.009 g, 0.02 mmol, 20% yield). The absolute configuration of Diastereomer 1 and Diasteromer 2 is not determined. Example 37 is designated as Diastereomer 1. Example 38 is designated as Diastereomer 2.

### Example 37

### Diastereomer 1:

LCMS: Method H1, 2.88 min, MS: ES+ 449.2; ¹H NMR (400 MHz, DMSO-d₆) δ ppm: 7.77 (s, 1H), 7.46-7.50 (m, 2H), 7.36 (d, J=8.0 Hz, 1H), 7.30 (s, 1H), 7.25 (d, J=8.0 Hz, 1H), 6.28 (s, 2H), 4.19-4.29 (m, 3H), 3.97-4.05 (m, 1H), 3.69-3.77 (m, 1H), 3.42-3.56 (m, 4H), 3.15-3.23 (m, 2H), 2.20-2.30 (s, 1H), 1.86-2.00 (m, 3H); Chiral SFC: Method Y31, 15.99 min.

### Example 38

### Diastereomer 2:

LCMS: Method H1, 2.88 min, MS: ES+ 449.2; ¹H NMR (400 MHz, DMSO-d₆) δ ppm: 7.76 (d, J=4.0 Hz, 1H), 7.46-7.50 (m, 2H), 7.36 (d, J=8.0 Hz, 1H), 7.30 (s, 1H), 7.25 (d, J=8.0 Hz, 1H), 6.28 (s, 2H), 4.19-4.29 (m, 3H), 3.97-4.05 (m, 1H), 3.71-3.77 (m, 1H), 3.42-3.56 (m, 4H), 3.14-3.23 (m, 2H), 2.20-2.28 (s, 1H), 1.86-2.00 (m, 3H); Chiral SFC: Method Y31, 16.99 min.

### Example 39

### N-((3R,5S)-5-((1H-1,2,3-Triazol-1-yl)methyl)-1-cyanopyrrolidin-3-yl)-5-(4-fluoro-3-(trifluoromethoxy)phenyl)oxazole-2-carboxamide

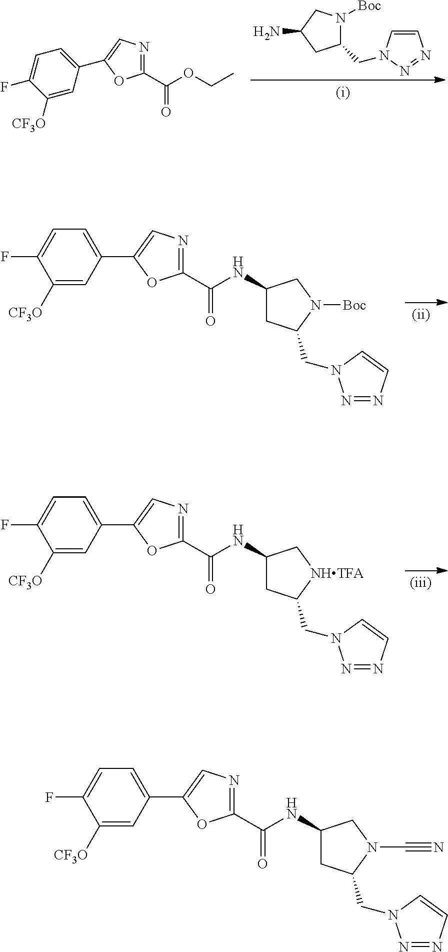

### Step (i)

### tert-Butyl (2S,4R)-2-((1H-1,2,3-triazol-1-yl)methyl)-4-(5-(4-fluoro-3-(trifluoromethoxy)phenyl)oxazole-2-carboxamido)pyrrolidine-1-carboxylate

To a stirred solution of ethyl 5-(4-fluoro-3-(trifluoromethoxy)phenyl)oxazole-2-carboxylate (0.50 g, 1.56 mmol) and tert-butyl (2S,4R)-2-((1H-1,2,3-triazol-1-yl)methyl)-4-aminopyrrolidine-1-carboxylate (0.49 g, 1.87 mmol) in THF (5 mL) was added TBD (0.26 g, 1.87 mmol) in portions at 0° C. The mixture was allowed to warm to rt and stirred for 6 h. The mixture was poured into water (100 mL) and extracted with EtOAc (2×150 mL). The combined organic phases were dried over anhydrous Na₂SO₄, filtered and concentrated under reduced pressure. The residue was purified by flash column chromatography (silica gel, 80% EtOAc in n-hexanes) to yield tert-butyl (2S,4R)-2-((1H-1,2,3-triazol-1-yl)methyl)-4-(5-(4-fluoro-3-(trifluoromethoxy)phenyl)oxazole-2-carboxamido)pyrrolidine-1-carboxylate (0.23 g, 0.42 mmol, 27% yield).

LCMS: Method J1, 3.99 min, MS: ES+ 541.0.

### Step (ii)

### N-((3R,5S)-5-((1H-1,2,3-Triazol-1-yl)methyl)pyrrolidin-3-yl)-5-(4-fluoro-3-(trifluoromethoxy)phenyl)oxazole-2-carboxamide TFA salt

To a stirred solution of tert-butyl (2S,4R)-2-((1H-1,2,3-triazol-1-yl)methyl)-4-(5-(4-fluoro-3-(trifluoromethoxy)phenyl)oxazole-2-carboxamido)pyrrolidine-1-carboxylate (0.23 g, 0.42 mmol) in DCM (5 mL) was added TFA (1.15 mL, 5 vol) dropwise at 0° C. The mixture was allowed to warm to rt and stirred for 0.5 h, then concentrated under reduced pressure to yield N-((3R,5S)-5-((1H-1,2,3-triazol-1-yl)methyl)pyrrolidin-3-yl)-5-(4-fluoro-3-(trifluoromethoxy)phenyl)oxazole-2-carboxamide TFA salt (0.40 g, quantitative yield).

LCMS: Method J1, 2.83 min, MS: ES+ 441.2.

Step (iii)

### N-((3R,5S)-5-((1H-1,2,3-Triazol-1-yl)methyl)-1-cyanopyrrolidin-3-yl)-5-(4-fluoro-3-(trifluoromethoxy)phenyl)oxazole-2-carboxamide

To a stirred solution of N-((3R,5S)-5-((1H-1,2,3-triazol-1-yl)methyl)pyrrolidin-3-yl)-5-(4-fluoro-3-(trifluoromethoxy)phenyl)oxazole-2-carboxamide TFA salt (0.40 g, 0.72 mmol) in THF (4 mL) was added K₂CO₃ (0.49 g, 3.6 mmol) at rt and stirred for 5 min. The mixture was cooled to 0° C., then cyanogen bromide (0.08 g, 0.79 mmol) was added. The mixture was allowed to warm to rt, stirred for 0.5 h, then poured into water (50 mL) and extracted with EtOAc (2×75 mL). The combined organic phases were dried over anhydrous Na₂SO₄ and concentrated under reduced pressure. The residue was purified by flash column chromatography (silica gel, 96% EtOAc in n-hexanes) to yield N-((3R,5S)-5-((1H-1,2,3-triazol-1-yl)methyl)-1-cyanopyrrolidin-3-yl)-5-(4-fluoro-3-(trifluoromethoxy)phenyl) oxazole-2-carboxamide (0.04 g, 0.09 mmol, 21% yield over two steps).

LCMS: Method H1, 2.85 min, MS: ES+ 466.2; ¹H NMR (400 MHz, DMSO-d₆) δ ppm: 9.37 (d, J=4.0 Hz, 1H), 8.20 (s, 1H), 8.04-8.07 (m, 2H), 7.93 (br s, 1H), 7.71-7.79 (m, 2H), 4.63-4.64 (m, 2H), 4.30-4.40 (m, 2H), 3.66-3.70 (m, 1H), 3.40-3.43 (m, 1H), 2.20-2.23 (m, 1H), 1.99-2.02 (m, 1H); Chiral SFC: Method Y32, 4.35 min.

### Example 40

### N-((3R,5S)-5-((1H-1,2,3-Triazol-1-yl)methyl)-1-cyanopyrrolidin-3-yl)-5-(2-(azetidin-1-yl)-5-cyanopyridin-3-yl)-1,3,4-oxadiazole-2-carboxamide

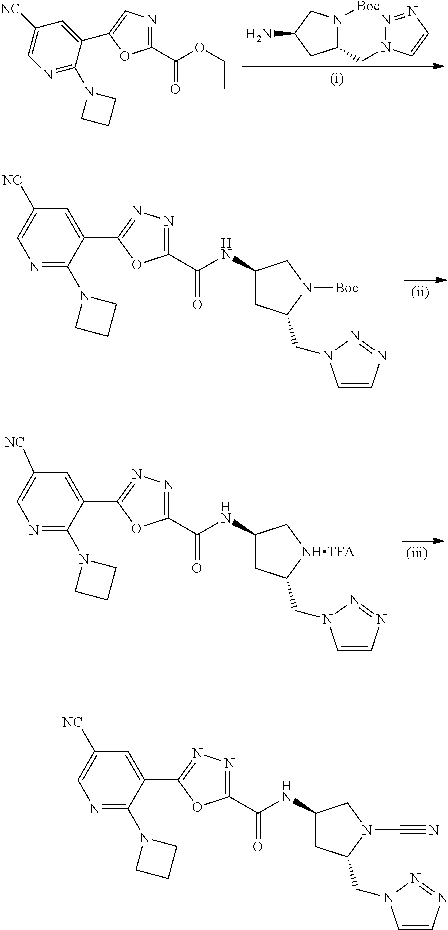

### Step (i)

### tert-Butyl (2S,4R)-2-((1H-1,2,3-triazol-1-yl)methyl)-4-(5-(2-(azetidin-1-yl)-5-cyanopyridin-3-yl)-1,3,4-oxadiazole-2-carboxamido)pyrrolidine-1-carboxylate

A stirred solution of ethyl 5-(2-(azetidin-1-yl)-5-cyanopyridin-3-yl)-1,3,4-oxadiazole-2-carboxylate (0.25 g, 0.83 mmol) and tert-butyl (2S,4R)-2-((1H-1,2,3-triazol-1-yl)methyl)-4-aminopyrrolidine-1-carboxylate (0.26 g, 0.99 mmol) in THF (4 mL) was added TBD (0.17 g, 1.24 mmol) in portions at 0° C. The mixture was allowed to warm to rt and stirred for 1 h at rt. The mixture was poured into water (30 mL) and extracted with EtOAc (3×30 mL). The combined organic phases were dried over anhydrous Na₂SO₄, filtered and concentrated under reduced pressure. The residue was purified by flash column chromatography (silica gel, 24% EtOAc in n-hexanes) to yield tert-butyl (2S,4R)-2-((1H-1,2,3-triazol-1-yl)methyl)-4-(5-(2-(azetidin-1-yl)-5-cyanopyridin-3-yl)-1,3,4-oxadiazole-2-carboxamido)pyrrolidine-1-carboxylate (0.07 g, 0.13 mmol, 15% yield).

LCMS: Method C1, 1.18 min, MS: ES+ 521.4.

### Step (ii)

### N-((3R,5S)-5-((1H-1,2,3-Triazol-1-yl)methyl)pyrrolidin-3-yl)-5-(2-(azetidin-1-yl)-5-cyanopyridin-3-yl)-1,3,4-oxadiazole-2-carboxamide TFA salt

To a stirred solution of tert-butyl (2S,4R)-2-((1H-1,2,3-triazol-1-yl)methyl)-4-(5-(2-(azetidin-1-yl)-5-cyanopyridin-3-yl)-1,3,4-oxadiazole-2-carboxamido)pyrrolidine-1-carboxylate (0.07 g, 0.13 mmol) in DCM (1 mL) was added TFA (0.35 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(2-(azetidin-1-yl)-5-cyanopyridin-3-yl)-1,3,4-oxadiazole-2-carboxamide TFA salt (0.10 g, quantitative yield).

LCMS: Method C1, 0.95 min, MS: ES+ 421.1.

Step (iii)

### N-((3R,5S)-5-((1H-1,2,3-Triazol-1-yl)methyl)-1-cyanopyrrolidin-3-yl)-5-(2-(azetidin-1-yl)-5-cyanopyridin-3-yl)-1,3,4-oxadiazole-2-carboxamide

To a stirred solution of N-((3R,5S)-5-((1H-1,2,3-triazol-1-yl)methyl)pyrrolidin-3-yl)-5-(2-(azetidin-1-yl)-5-cyanopyridin-3-yl)-1,3,4-oxadiazole-2-carboxamide TFA salt (0.10 g, 0.19 mmol) in THF (1 mL) was added K₂CO₃ (0.08 g, 0.57 mmol) at rt and stirred for 5 min. Cyanogen bromide (0.03 g, 0.28 mmol) was added into the reaction mixture at 0° C. The mixture was stirred at rt for 1 h, then poured into water (30 mL) and extracted with EtOAc (3×50 mL). The combined organic phases were dried over anhydrous Na₂SO₄ and concentrated under reduced pressure. The residue was purified by reverse phase preparative HPLC (Method X11) to yield N-((3R,5S)-5-((1H-1,2,3-triazol-1-yl)methyl)-1-cyanopyrrolidin-3-yl)-5-(2-(azetidin-1-yl)-5-cyanopyridin-3-yl)-1,3,4-oxadiazole-2-carboxamide (0.04 g, 0.09 mmol, 66% yield over two steps).

LCMS: Method H1, 2.23 min, MS: ES+ 446.2; ¹H NMR (400 MHz, DMSO-d₆) δ 9.77 (d, J=6.4 Hz, 1H), 8.72 (d, J=2.0 Hz, 1H), 8.32 (d, J=2.0 Hz, 1H), 8.19 (s, 1H), 7.78 (s, 1H), 4.62-4.64 (m, 2H), 4.35-4.38 (m, 2H), 4.02-4.11 (m, 4H), 3.66-3.70 (m, 1H), 3.41-3.44 (m, 1H), 2.26-2.33 (m, 2H), 2.18-2.23 (m, 1H), 1.98-2.03 (m, 1H); Chiral HPLC: Method Y4, 5.32 min.

### Example 41

### N-((3R,5S)-5-((1H-1,2,3-Triazol-1-yl)methyl)-1-cyanopyrrolidin-3-yl)-5-(5-cyano-2-(pyrrolidin-1-yl)pyridin-3-yl)-1,3,4-oxadiazole-2-carboxamide

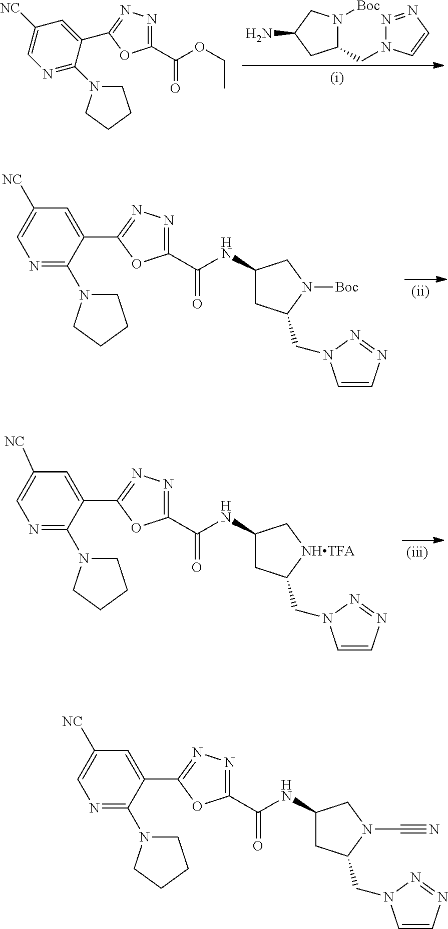

### Step (i)

### tert-Butyl (2S,4R)-2-((1H-1,2,3-triazol-1-yl)methyl)-4-(5-(5-cyano-2-(pyrrolidin-1-yl)pyridin-3-yl)-1,3,4-oxadiazole-2-carboxamido)pyrrolidine-1-carboxylate

A stirred solution of ethyl 5-(5-cyano-2-(pyrrolidin-1-yl)pyridin-3-yl)-1,3,4-oxadiazole-2-carboxylate (0.23 g, 0.73 mmol) and tert-butyl (2S,4R)-2-((1H-1,2,3-triazol-1-yl)methyl)-4-aminopyrrolidine-1-carboxylate (0.19 g, 0.73 mmol) in THF (2.3 mL) was added TBD (0.10 g, 0.73 mmol) in portions at 0° C. The mixture was allowed to warm to rt and stirred for 0.5 h at rt. The mixture was poured into water (15 mL) and extracted with EtOAc (3×15 mL). The combined organic phases were dried over anhydrous Na₂SO₄, filtered and concentrated under reduced pressure. The residue was purified by flash column chromatography (silica gel, 95% EtOAc in n-hexanes) to yield tert-butyl (2S,4R)-2-((1H-1,2,3-triazol-1-yl)methyl)-4-(5-(5-cyano-2-(pyrrolidin-1-yl)pyridin-3-yl)-1,3,4-oxadiazole-2-carboxamido)pyrrolidine-1-carboxylate (0.10 g, 0.19 mmol, 25% yield).

LCMS: Method C1, 1.18 min, MS: ES+ 534.4.

### Step (ii)

### N-((3R,5S)-5-((1H-1,2,3-Triazol-1-yl)methyl)pyrrolidin-3-yl)-5-(5-cyano-2-(pyrrolidin-1-yl)pyridin-3-yl)-1,3,4-oxadiazole-2-carboxamide TFA salt

To a stirred solution of tert-butyl (2S,4R)-2-((1H-1,2,3-triazol-1-yl)methyl)-4-(5-(5-cyano-2-(pyrrolidin-1-yl)pyridin-3-yl)-1,3,4-oxadiazole-2-carboxamido)pyrrolidine-1-carboxylate (0.10 g, 0.19 mmol) in DCM (1 mL) was added TFA (0.3 mL, 3 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(5-cyano-2-(pyrrolidin-1-yl)pyridin-3-yl)-1,3,4-oxadiazole-2-carboxamide TFA salt (0.12 g, quantitative yield).

LCMS: Method C1, 0.98 min, MS: ES− 433.3.

Step (iii)

### N-((3R,5S)-5-((1H-1,2,3-Triazol-1-yl)methyl)-1-cyanopyrrolidin-3-yl)-5-(5-cyano-2-(pyrrolidin-1-yl)pyridin-3-yl)-1,3,4-oxadiazole-2-carboxamide

To a stirred solution of N-((3R,5S)-5-((1H-1,2,3-triazol-1-yl)methyl)pyrrolidin-3-yl)-5-(5-cyano-2-(pyrrolidin-1-yl)pyridin-3-yl)-1,3,4-oxadiazole-2-carboxamide TFA salt (0.12 g, 0.22 mmol) in THF (1.2 mL) was added K₂CO₃ (0.09 g, 0.66 mmol) at rt and stirred for 5 min. Cyanogen bromide (0.02 g, 0.22 mmol) was added into the reaction mixture at 0° C. The mixture was stirred at rt for 1 h, then poured into water (15 mL) and extracted with EtOAc (3×15 mL). The combined organic phases were dried over anhydrous Na₂SO₄ and concentrated under reduced pressure. The residue was purified by flash column chromatography (silica gel, 4% MeOH in DCM) to yield N-((3R,5S)-5-((1H-1,2,3-triazol-1-yl)methyl)-1-cyanopyrrolidin-3-yl)-5-(5-cyano-2-(pyrrolidin-1-yl)pyridin-3-yl)-1,3,4-oxadiazole-2-carboxamide (0.02 g, 0.05 mmol, 26% yield over two steps).

LCMS: Method H1, 2.40 min, MS: ES+ 460.2; ¹H NMR (400 MHz, DMSO-d₆) δ 9.77 (d, J=6.4 Hz, 1H), 8.72 (d, J=2.0 Hz, 1H), 8.30 (d, J=2.0 Hz, 1H), 8.19 (s, 1H), 7.78 (s, 1H), 4.58-4.63 (m, 2H), 4.34-4.37 (m, 2H), 3.65-3.69 (m, 1H), 3.40-3.44 (m, 1H), 3.29-3.34 (m, 4H), 2.16-2.22 (m, 1H), 1.96-2.03 (m, 1H), 1.85 (br s, 4H); Chiral HPLC: Method Y4, 5.45 min.

### Example 42

### N-((3R,5S)-5-((1H-1,2,3-Triazol-1-yl)methyl)-1-cyanopyrrolidin-3-yl)-5-(2-chloro-5-(trifluoromethoxy)phenyl)-1,3,4-oxadiazole-2-carboxamide

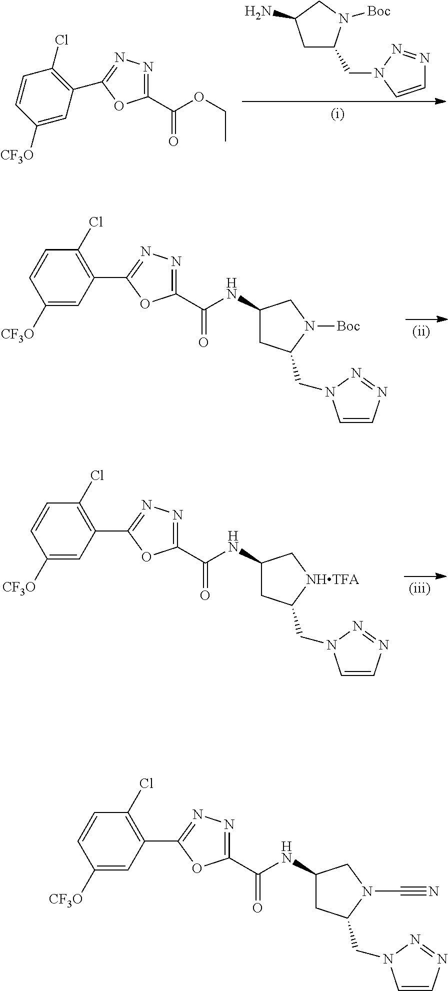

### Step (i)

### tert-Butyl (2S,4R)-2-((1H-1,2,3-triazol-1-yl)methyl)-4-(5-(2-chloro-5-(trifluoromethoxy)phenyl)-1,3,4-oxadiazole-2-carboxamido)pyrrolidine-1-carboxylate

To a stirred solution of ethyl 5-(2-chloro-5-(trifluoromethoxy)phenyl)-1,3,4-oxadiazole-2-carboxylate (0.50 g, 1.48 mmol) and tert-butyl (2S,4R)-2-((1H-1,2,3-triazol-1-yl)methyl)-4-aminopyrrolidine-1-carboxylate (0.39 g, 1.48 mmol) in THF (5 mL) was added TBD (0.20 g, 1.48 mmol) in portions at 0° C. The mixture was allowed to warm to rt and stirred at rt for 0.5 h, then poured into water (50 mL) and extracted with EtOAc (2×50 mL). The combined organic phases were dried over anhydrous Na₂SO₄, filtered and concentrated under reduced pressure. The residue was purified by flash column chromatography (silica gel, 70% EtOAc in n-hexanes) to yield tert-butyl (2S,4R)-2-((1H-1,2,3-triazol-1-yl)methyl)-4-(5-(2-chloro-5-(trifluoromethoxy)phenyl)-1,3,4-oxadiazole-2-carboxamido)pyrrolidine-1-carboxylate (0.30 g, 0.53 mmol, 36% yield).

LCMS: Method C1, 1.28 min, MS: ES+ 558.1.

### Step (ii)

### N-((3R,5S)-5-((1H-1,2,3-Triazol-1-yl)methyl)pyrrolidin-3-yl)-5-(2-chloro-5-(trifluoromethoxy)phenyl)-1,3,4-oxadiazole-2-carboxamide TFA salt

To a stirred solution of tert-butyl (2S,4R)-2-((1H-1,2,3-triazol-1-yl)methyl)-4-(5-(2-chloro-5-(trifluoromethoxy)phenyl)-1,3,4-oxadiazole-2-carboxamido)pyrrolidine-1-carboxylate (0.30 g, 0.53 mmol) in DCM (3 mL) was added TFA (1.5 mL, 5 vol) dropwise at 0° C. The mixture was allowed to warm to rt and stirred for 2 h, then concentrated under reduced pressure to yield N-((3R,5S)-5-((1H-1,2,3-triazol-1-yl)methyl)pyrrolidin-3-yl)-5-(2-chloro-5-(trifluoromethoxy)phenyl)-1,3,4-oxadiazole-2-carboxamide TFA salt (0.32 g, quantitative yield).

LCMS: Method C, 1.36 min, MS: ES+ 458.1.

Step (iii)

### N-((3R,5S)-5-((1H-1,2,3-Triazol-1-yl)methyl)-1-cyanopyrrolidin-3-yl)-5-(2-chloro-5-(trifluoromethoxy)phenyl)-1,3,4-oxadiazole-2-carboxamide

To a stirred solution of N-((3R,5S)-5-((1H-1,2,3-triazol-1-yl)methyl)pyrrolidin-3-yl)-5-(2-chloro-5-(trifluoromethoxy)phenyl)-1,3,4-oxadiazole-2-carboxamide TFA salt (0.32 g, 0.56 mmol) in THF (3.2 mL) was added K₂CO₃ (0.23 g, 1.68 mmol) at rt and stirred for 5 min. Cyanogen bromide (0.06 g, 0.56 mmol) was added at 0° C. The mixture was stirred at rt for 1 h, then poured into water (30 mL) and extracted with EtOAc (2×30 mL). The combined organic phases were dried over anhydrous Na₂SO₄, filtered and concentrated under reduced pressure. The residue was purified by flash column chromatography (silica gel, 85% EtOAc in n-hexanes) to yield N-((3R,5S)-5-((1H-1,2,3-triazol-1-yl)methyl)-1-cyanopyrrolidin-3-yl)-5-(2-chloro-5-(trifluoromethoxy)phenyl)-1,3,4-oxadiazole-2-carboxamide (0.06 g, 0.12 mmol, 25% yield over two steps).

LCMS: Method H1, 2.81 min, MS: ES+ 483.0; ¹H NMR (400 MHz, DMSO-d₆) δ ppm: 9.88 (d, J=6.0 Hz, 1H), 8.22 (s, 1H), 8.05 (s, 1H), 7.95 (d, J=8.8 Hz, 1H), 7.81 (s, 1H), 7.78 (s, 1H), 4.65-4.66 (m, 2H), 4.39-4.41 (m, 2H), 3.69-3.73 (m, 1H), 3.45-3.47 (m, 1H), 2.21-2.24 (m, 1H), 2.01-2.05 (m, 1H); Chiral SFC: Method Y21, 10.11 min.

### Example 43

### N-((3R,5S)-5-((1H-1,2,3-Triazol-1-yl)methyl)-1-cyanopyrrolidin-3-yl)-5-(3-(trifluoromethoxy)phenyl)-1,3,4-oxadiazole-2-carboxamide

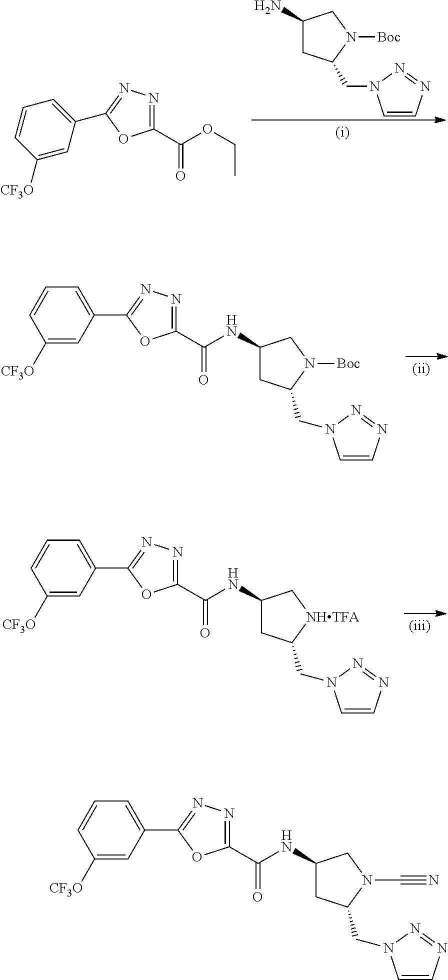

### Step (i)

### tert-Butyl (2S,4R)-2-((1H-1,2,3-triazol-1-yl)methyl)-4-(5-(3-(trifluoromethoxy)phenyl)-1,3,4-oxadiazole-2-carboxamido)pyrrolidine-1-carboxylate

To a stirred solution of ethyl 5-(3-(trifluoromethoxy)phenyl)-1,3,4-oxadiazole-2-carboxylate (0.47 g, 1.56 mmol) and tert-butyl (2S,4R)-2-((1H-1,2,3-triazol-1-yl)methyl)-4-aminopyrrolidine-1-carboxylate (0.42 g, 1.56 mmol) in THF (6 mL) was added TBD (0.32 g, 2.33 mmol) in portions at 0° C. and stirred at 0° C. for 15 min. The mixture was poured into water (100 mL) and extracted with EtOAc (2×120 mL). The combined organic phases were dried over anhydrous Na₂SO₄, filtered and concentrated under reduced pressure. The residue was purified by flash column chromatography (silica gel, 60% EtOAc in n-hexanes) to yield tert-butyl (2S,4R)-2-((1H-1,2,3-triazol-1-yl)methyl)-4-(5-(3-(trifluoromethoxy)phenyl)-1,3,4-oxadiazole-2-carboxamido)pyrrolidine-1-carboxylate (0.19 g, 0.36 mmol, 23% yield). LCMS: Method C1, 1.25 min, MS: ES+ 524.2.

### Step (ii)

### N-((3R,5S)-5-((1H-1,2,3-Triazol-1-yl)methyl)pyrrolidin-3-yl)-5-(3-(trifluoromethoxy)phenyl)-1,3,4-oxadiazole-2-carboxamide TFA salt

To a stirred solution of tert-butyl (2S,4R)-2-((1H-1,2,3-triazol-1-yl)methyl)-4-(5-(3-(trifluoromethoxy)phenyl)-1,3,4-oxadiazole-2-carboxamido)pyrrolidine-1-carboxylate (0.18 g, 0.34 mmol) in DCM (1.8 mL) was added TFA (1.8 mL, 10 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(3-(trifluoromethoxy)phenyl)-1,3,4-oxadiazole-2-carboxamide TFA salt (0.18 g, quantitative yield).

LCMS: Method C1, 1.05 min, MS: ES+ 424.2.

Step (iii)

### N-((3R,5S)-5-((1H-1,2,3-Triazol-1-yl)methyl)-1-cyanopyrrolidin-3-yl)-5-(3-(trifluoromethoxy)phenyl)-1,3,4-oxadiazole-2-carboxamide

To a stirred solution of N-((3R,5S)-5-((1H-1,2,3-triazol-1-yl)methyl)pyrrolidin-3-yl)-5-(3-(trifluoromethoxy)phenyl)-1,3,4-oxadiazole-2-carboxamide TFA salt (0.17 g, 0.32 mmol) in THF (1.7 mL) was added K₂CO₃ (0.13 g, 0.95 mmol) at rt and stirred for 5 min. Cyanogen bromide (0.04 g, 0.35 mmol) was added to the mixture at 0° C. The mixture was stirred at 0° C. for 1 h, then poured into water (30 mL) and extracted with EtOAc (2×50 mL). The combined organic phases were dried over anhydrous Na₂SO₄, filtered and concentrated under reduced pressure. The residue was purified by flash column chromatography (silica gel, 70% EtOAc in n-hexanes) to yield N-((3R,5S)-5-((1H-1,2,3-triazol-1-yl)methyl)-1-cyanopyrrolidin-3-yl)-5-(3-(trifluoromethoxy)phenyl)-1,3,4-oxadiazole-2-carboxamide (0.05 g, 0.11 mmol, 32% yield over two steps).

LCMS: Method H1, 3.50 min, MS: ES+ 449.0; ¹H NMR (400 MHz, DMSO-d₆) δ ppm: 9.80 (d, J=4.0 Hz, 1H), 8.22 (s, 1H), 8.15 (d, J=8.0 Hz, 1H), 8.01 (s, 1H), 7.81-7.85 (m, 2H), 7.75-7.76 (m, 1H), 4.65-4.66 (m, 2H), 4.40-4.41 (m, 2H), 3.70-3.74 (m, 1H), 3.47-3.48 (m, 1H), 2.20-2.27 (m, 1H), 2.00-2.07 (m, 1H); Chiral SFC: Method Y4, 4.14 min.

### Example 44

### N-((3R,5S)-5-((1H-Pyrazol-1-yl)methyl)-1-cyanopyrrolidin-3-yl)-5-(2-cyclopropyl-5-(trifluoromethoxy)phenyl)-1,3,4-oxadiazole-2-carboxamide

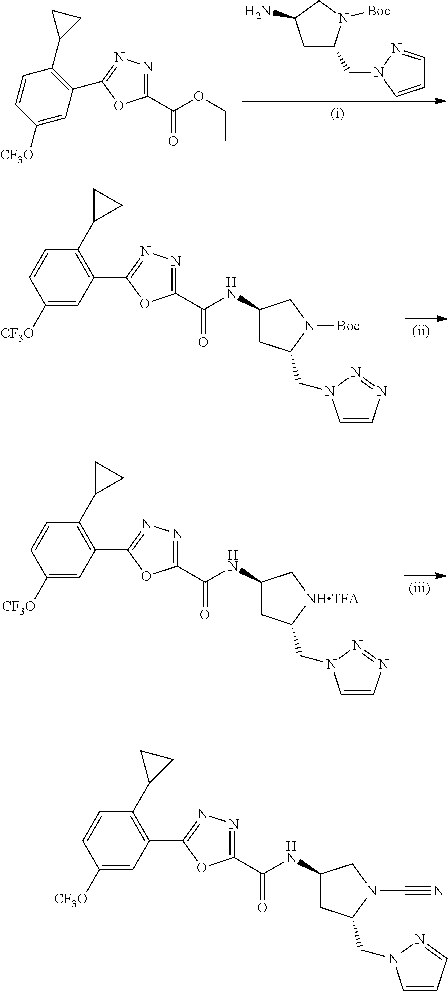

### Step (i)

### tert-Butyl (2S,4R)-2-((1H-pyrazol-1-yl)methyl)-4-(5-(2-cyclopropyl-5-(trifluoromethoxy)phenyl)-1,3,4-oxadiazole-2-carboxamido)pyrrolidine-1-carboxylate

To a stirred solution of ethyl 5-(2-cyclopropyl-5-(trifluoromethoxy)phenyl)-1,3,4-oxadiazole-2-carboxylate (0.36 g, 1.05 mmol) and tert-butyl (2S,4R)-2-((1H-pyrazol-1-yl)methyl)-4-aminopyrrolidine-1-carboxylate (0.28 g, 1.05 mmol) in THF (3.6 mL) was added TBD (0.17 g, 1.26 mmol) in portions at 0° C. The mixture was allowed to warm to rt and stirred for 0.5 h at rt, then poured into water (20 mL) and extracted with EtOAc (3×15 mL). The combined organic phases were dried over anhydrous Na₂SO₄, filtered and concentrated under reduced pressure. The residue was purified by flash column chromatography (silica gel, 60% EtOAc in n-hexanes) to yield tert-butyl (2S,4R)-2-((1H-pyrazol-1-yl)methyl)-4-(5-(2-cyclopropyl-5-(trifluoromethoxy)phenyl)-1,3,4-oxadiazole-2-carboxamido)pyrrolidine-1-carboxylate (0.17 g, 0.31 mmol, 30% yield).

LCMS: Method C1, 1.40 min, MS: ES+ 563.1.

### Step (ii)

### N-((3R,5S)-5-((1H-Pyrazol-1-yl)methyl)pyrrolidin-3-yl)-5-(2-cyclopropyl-5(trifluoromethoxy)phenyl)-1,3,4-oxadiazole-2-carboxamide TFA salt

To a stirred solution of tert-butyl (2S,4R)-2-((1H-pyrazol-1-yl)methyl)-4-(5-(2-cyclopropyl-5-(trifluoromethoxy)phenyl)-1,3,4-oxadiazole-2-carboxamido)pyrrolidine-1-carboxylate (0.17 g, 3.10 mmol) in DCM (2 mL) was added TFA (0.57 mL, 5 vol) dropwise at 0° C. The mixture was allowed to warm to rt and stirred for 1 h, then concentrated under reduced pressure to yield N-((3R,5S)-5-((1H-pyrazol-1-yl)methyl)pyrrolidin-3-yl)-5-(2-cyclopropyl-5-(trifluoromethoxy)phenyl)-1,3,4-oxadiazole-2-carboxamide TFA salt (0.23 g, quantitative yield).

LCMS: Method C1, 1.13 min, MS: ES+ 463.2.

Step (iii)

### N-((3R,5S)-5-((1H-Pyrazol-1-yl)methyl)-1-cyanopyrrolidin-3-yl)-5-(2-cyclopropyl-5-(trifluoromethoxy)phenyl)-1,3,4-oxadiazole-2-carboxamide

To a stirred solution of N-((3R,5S)-5-((1H-pyrazol-1-yl)methyl)pyrrolidin-3-yl)-5-(2-cyclopropyl-5-(trifluoromethoxy)phenyl)-1,3,4-oxadiazole-2-carboxamide TFA salt (0.23 g, 0.41 mmol) in THF (3 mL) was added K₂CO₃ (0.28 g, 2.06 mmol) at rt and stirred for 5 min. Cyanogen bromide (0.13 g, 1.20 mmol) was added into the reaction mixture at 0° C. The mixture was stirred at rt for 1 h, then poured into water (10 mL) and extracted with EtOAc (3×15 mL). The combined organic phases were dried over anhydrous Na₂SO₄ and concentrated under reduced pressure. The residue was purified by flash column chromatography (silica gel, 50% EtOAc in n-hexanes) to yield N-((3R,5S)-5-((1H-pyrazol-1-yl)methyl)-1-cyanopyrrolidin-3-yl)-5-(2-cyclopropyl-5-(trifluoromethoxy)phenyl)-1,3,4-oxadiazole-2-carboxamide (0.065 g, 0.13 mmol, 43% yield over two steps).

LCMS: Method H1, 3.14 min, MS: ES+ 488.2; ¹H NMR (400 MHz, DMSO-d₆) δ 9.76 (s, 1H), 7.83 (s, 1H), 7.78 (s, 1H), 7.58 (d, J=8.0 Hz 1H), 7.51 (s, 1H), 7.31 (d, J=8.0 Hz, 1H), 6.29 (s, 1H), 4.29-4.32 (m, 4H), 3.63-3.67 (m, 1H), 3.39-3.42 (m, 1H), 2.68-2.70 (m, 1H), 2.13-2.15 (m, 1H), 2.02-2.05 (m, 1H), 1.05-1.07 (m, 2H), 0.78-0.82 (m, 2H); Chiral HPLC: Method Y14, 3.75 min.

### Example 45

### N-((3R,5S)-5-((2H-1,2,3-Triazol-2-yl)methyl)-1-cyanopyrrolidin-3-yl)-5-(2-cyclopropyl-5-(trifluoromethoxy)phenyl)-1,3,4-oxadiazole-2-carboxamide

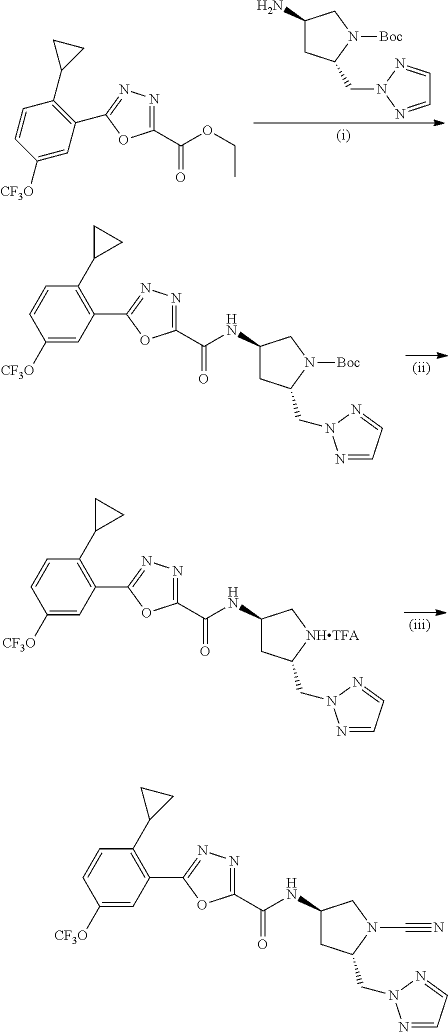

### Step (i)

### tert-Butyl (2S,4R)-2-((2H-1,2,3-triazol-2-yl)methyl)-4-(5-(2-cyclopropyl-5-(trifluoromethoxy)phenyl)-1,3,4-oxadiazole-2-carboxamido)pyrrolidine-1-carboxylate

To a stirred solution of ethyl 5-(2-cyclopropyl-5-(trifluoromethoxy)phenyl)-1,3,4-oxadiazole-2-carboxylate (0.33 g, 0.96 mmol) and tert-butyl (2S,4R)-2-((2H-1,2,3-triazol-2-yl)methyl)-4-aminopyrrolidine-1-carboxylate (0.30 g, 1.15 mmol) in THF (5 mL) was added TBD (0.20 g, 1.44 mmol) in portions at 0° C. and stirred at 0° C. for 0.5 h. The mixture was poured into water (100 mL) and extracted with EtOAc (3×100 mL). The combined organic phases were dried over anhydrous Na₂SO₄, filtered and concentrated under reduced pressure. The residue was purified by flash column chromatography (silica gel, 37% EtOAc in n-hexanes) to yield tert-butyl (2S,4R)-2-((2H-1,2,3-triazol-2-yl)methyl)-4-(5-(2-cyclopropyl-5-(trifluoromethoxy)phenyl)-1,3,4-oxadiazole-2-carboxamido)pyrrolidine-1-carboxylate (0.23 g, 0.40 mmol, 42% yield).

LCMS: Method H1, 3.61 min, MS: ES− 562.2.

### Step (ii)

### N-((3R,5S)-5-((2H-1,2,3-Triazol-2-yl)methyl)pyrrolidin-3-yl)-5-(2-cyclopropyl-5-(trifluoromethoxy)phenyl)-1,3,4-oxadiazole-2-carboxamide TFA salt

To a stirred solution of tert-butyl (2S,4R)-2-((2H-1,2,3-triazol-2-yl)methyl)-4-(5-(2-cyclopropyl-5-(trifluoromethoxy)phenyl)-1,3,4-oxadiazole-2-carboxamido)pyrrolidine-1-carboxylate (0.23 g, 0.39 mmol) in DCM (3 mL) was added TFA (1.1 mL, 5 vol) dropwise at 0° C. The mixture was allowed to warm to rt and stirred for 1 h, then concentrated under reduced pressure to yield N-((3R,5S)-5-((2H-1,2,3-triazol-2-yl)methyl)pyrrolidin-3-yl)-5-(2-cyclopropyl-5-(trifluoromethoxy)phenyl)-1,3,4-oxadiazole-2-carboxamide TFA salt (0.30 g, quantitative yield).

LCMS: Method H1, 2.97 min, MS: ES+ 464.2.

Step (iii)

### N-((3R,5S)-5-((2H-1,2,3-Triazol-2-yl)methyl)-1-cyanopyrrolidin-3-yl)-5-(2-cyclopropyl-5-(trifluoromethoxy)phenyl)-1,3,4-oxadiazole-2-carboxamide

To a stirred solution of N-((3R,5S)-5-((2H-1,2,3-triazol-2-yl)methyl)pyrrolidin-3-yl)-5-(2-cyclopropyl-5-(trifluoromethoxy)phenyl)-1,3,4-oxadiazole-2-carboxamide TFA salt (0.28 g, 0.49 mmol) in THF (5 mL) was added K₂CO₃ (0.34 g, 2.46 mmol) at rt and stirred for 5 min. Cyanogen bromide (0.05 g, 0.49 mmol) was added into the reaction mixture at 0° C. The mixture was allowed to warm to rt, and stirred at rt for 15 min, then poured into water (100 mL) and extracted with EtOAc (3×100 mL). The combined organic phases were dried over anhydrous Na₂SO₄, filtered and concentrated under reduced pressure. The residue was purified by flash column chromatography (silica gel, 37% EtOAc in n-hexanes) to yield N-((3R,5S)-5-((2H-1,2,3-triazol-2-yl)methyl)-1-cyanopyrrolidin-3-yl)-5-(2-cyclopropyl-5-(trifluoromethoxy)phenyl)-1,3,4-oxadiazole-2-carboxamide (0.07 g, 0.15 mmol, 38% yield over two steps).

LCMS: Method H1, 3.15 min, MS: ES+ 489.2; ¹H NMR (400 MHz, DMSO-d₆) δ ppm: 9.78 (d, J 6.8 Hz, 1H), 7.81-7.84 (m, 3H), 7.56 (d, J=7.6 Hz, 1H), 7.29 (d, J=8.8 Hz, 1H), 4.62-4.64 (m, 2H), 4.40-4.43 (m, 1H), 4.30-4.32 (m, 1H), 3.61-3.65 (m, 1H), 3.38-3.42 (m, 1H), 2.66-2.70 (m, 1H), 2.18-2.24 (m, 1H), 2.03-2.08 (m, 1H), 1.03-1.05 (m, 2H), 0.79-0.80 (m, 2H), Chiral SFC: Method Y10, 7.61 min.

### Example 46

### N-((3R,5S)-5-((1H-Pyrazol-1-yl)methyl)-1-cyanopyrrolidin-3-yl)-5-(5-cyano-2-cyclopropylphenyl)-1,3,4-oxadiazole-2-carboxamide

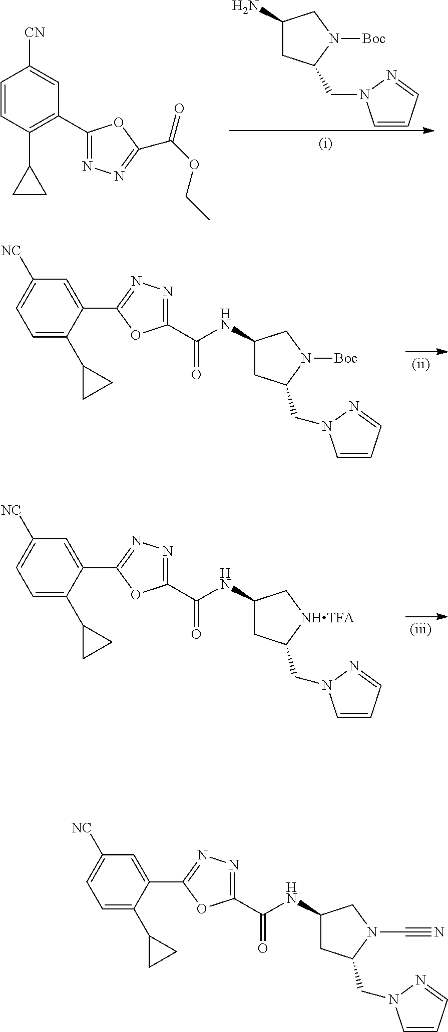

### Step (i)

### tert-Butyl (2S,4R)-2-((1H-pyrazol-1-yl)methyl)-4-(5-(5-cyano-2-cyclopropylphenyl)-1,3,4-oxadiazole-2-carboxamido)pyrrolidine-1-carboxylate

To a stirred solution of ethyl 5-(5-cyano-2-cyclopropylphenyl)-1,3,4-oxadiazole-2-carboxylate (0.44 g, 1.55 mmol) and tert-butyl (2S,4R)-2-((1H-pyrazol-1-yl)methyl)-4-aminopyrrolidine-1-carboxylate (0.49 g, 1.86 mmol) in THF (6 mL) was added TBD (0.32 g, 2.32 mmol) in portions at 0° C. and the mixture was stirred at 0° C. for 15 min, then poured into water (80 mL) and extracted with EtOAc (2×100 mL). The combined organic phases were dried over anhydrous Na₂SO₄, filtered and concentrated under reduced pressure. The residue was purified by flash column chromatography (silica gel, 40% EtOAc in n-hexanes) to yield tert-butyl (2S,4R)-2-((1H-pyrazol-1-yl)methyl)-4-(5-(5-cyano-2-cyclopropylphenyl)-1,3,4-oxadiazole-2-carboxamido)pyrrolidine-1-carboxylate (0.17 g, 0.33 mmol, 21% yield).

LCMS: Method C, 1.72 min, MS: ES+ 504.4.

### Step (ii)

### N-((3R,5S)-5-((1H-Pyrazol-1-yl)methyl)pyrrolidin-3-yl)-5-(5-cyano-2-cyclopropylphenyl)-1,3,4-oxadiazole-2-carboxamide TFA salt

To a stirred solution of tert-butyl (2S,4R)-2-((1H-pyrazol-1-yl)methyl)-4-(5-(5-cyano-2-cyclopropylphenyl)-1,3,4-oxadiazole-2-carboxamido)pyrrolidine-1-carboxylate (0.16 g, 0.32 mmol) in DCM (1.6 mL) was added TFA (1.6 mL, 10 vol) dropwise at 0° C. The mixture was allowed to warm to rt and stirred for 1 h, then concentrated under reduced pressure to yield N-((3R,5S)-5-((1H-pyrazol-1-yl)methyl)pyrrolidin-3-yl)-5-(5-cyano-2-cyclopropylphenyl)-1,3,4-oxadiazole-2-carboxamide TFA salt (0.23 g, quantitative yield).

LCMS: Method C1, 1.04 min, MS: ES+ 404.3.

Step (iii)

### N-((3R,5S)-5-((1H-Pyrazol-1-yl)methyl)-1-cyanopyrrolidin-3-yl)-5-(5-cyano-2-cyclopropylphenyl)-1,3,4-oxadiazole-2-carboxamide

To a stirred solution of N-((3R,5S)-5-((1H-pyrazol-1-yl)methyl)pyrrolidin-3-yl)-5-(5-cyano-2-cyclopropylphenyl)-1,3,4-oxadiazole-2-carboxamide TFA salt (0.23 g, 0.45 mmol) in THF (2.3 mL) was added K₂CO₃ (0.18 g, 1.33 mmol) at rt and stirred for 5 min. Cyanogen bromide (0.05 g, 0.49 mmol) was added into the reaction mixture at 0° C. The mixture was stirred at 0° C. for 1 h, then poured into water (50 mL) and extracted with EtOAc (2×80 mL). The combined organic phases were dried over anhydrous Na₂SO₄, filtered and concentrated under reduced pressure. The residue was purified by flash column chromatography (silica gel, 50% EtOAc in n-hexanes) to yield N-((3R,5S)-5-((1H-pyrazol-1-yl)methyl)-1-cyanopyrrolidin-3-yl)-5-(5-cyano-2-cyclopropylphenyl)-1,3,4-oxadiazole-2-carboxamide (0.06 g, 0.14 mmol, 44% yield over two steps).

LCMS: Method H1, 2.68 min, MS: ES+ 429.2; ¹H NMR (400 MHz, DMSO-d₆) δ ppm: 9.71 (d, J=6.4 Hz, 1H), 8.29 (d, J=1.6 Hz, 1H), 7.98 (dd, J=1.2, 8.4 Hz, 1H), 7.77 (d, J=2.0 Hz, 1H), 7.49 (d, J=1.2 Hz, 1H), 7.31 (d, J=8.0 Hz, 1H), 6.27 (s, 1H), 4.25-4.32 (m, 4H), 3.62-3.66 (m, 1H), 3.38-3.41 (m, 1H), 2.77-2.85 (m, 1H), 2.10-2.16 (m, 1H), 1.99-2.05 (m, 1H), 1.13-1.17 (m, 2H), 0.90-0.91 (m, 2H); Chiral SFC: Method Y10, 9.37 min.

### Example 47

### N-((3R,5S)-5-((1H-1,2,4-Triazol-1-yl)methyl)-1-cyanopyrrolidin-3-yl)-5-(2-cyclopropyl-5-(trifluoromethoxy)phenyl)-1,3,4-oxadiazole-2-carboxamide

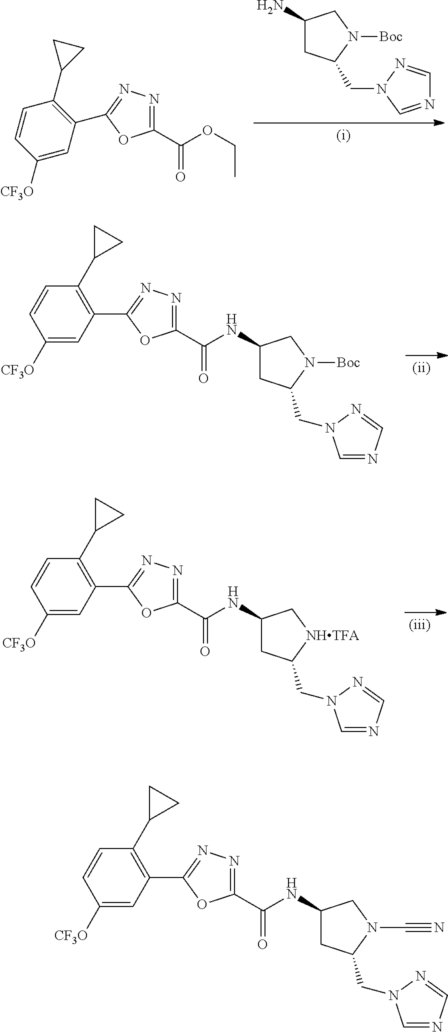

### Step (i)

### tert-Butyl (2S,4R)-2-((1H-1,2,4-triazol-1-yl)methyl)-4-(5-(2-cyclopropyl-5-(trifluoromethoxy)phenyl)-1,3,4-oxadiazole-2-carboxamido)pyrrolidine-1-carboxylate

To a stirred solution of ethyl 5-(2-cyclopropyl-5-(trifluoromethoxy)phenyl)-1,3,4-oxadiazole-2-carboxylate (0.40 g, 1.17 mmol) and tert-butyl (2S,4R)-2-((1H-1,2,4-triazol-1-yl)methyl)-4-aminopyrrolidine-1-carboxylate (0.46 g, 1.75 mmol) in THF (6 mL) was added TBD (0.24 g, 1.75 mmol) in portions at 0° C. and stirred at 0° C. for 15 min. The mixture was poured into water (80 mL) and extracted with EtOAc (2×100 mL). The combined organic phases were dried over anhydrous Na₂SO₄, filtered and concentrated under reduced pressure. The residue was purified by flash column chromatography (silica gel, 85% EtOAc in n-hexanes) to yield tert-butyl (2S,4R)-2-((1H-1,2,4-triazol-1-yl)methyl)-4-(5-(2-cyclopropyl-5-(trifluoromethoxy)phenyl)-1,3,4-oxadiazole-2-carboxamido)pyrrolidine-1-carboxylate (0.22 g, 0.39 mmol, 33% yield).

LCMS: Method C, 1.78 min, MS: ES+ 564.4.

### Step (ii)

### N-((3R,5S)-5-((1H-1,2,4-Triazol-1-yl)methyl)pyrrolidin-3-yl)-5-(2-cyclopropyl-5-(trifluoromethoxy)phenyl)-1,3,4-oxadiazole-2-carboxamide TFA salt

To a stirred solution of tert-butyl (2S,4R)-2-((1H-1,2,4-triazol-1-yl)methyl)-4-(5-(2-cyclopropyl-5-(trifluoromethoxy)phenyl)-1,3,4-oxadiazole-2-carboxamido)pyrrolidine-1-carboxylate (0.22 g, 0.39 mmol) in DCM (2.2 mL) was added TFA (2.2 mL, 10 vol) dropwise at 0° C. The mixture was allowed to warm to rt and stirred for 1 h, then concentrated under reduced pressure to yield N-((3R,5S)-5-((1H-1,2,4-triazol-1-yl)methyl)pyrrolidin-3-yl)-5-(2-cyclopropyl-5-(trifluoromethoxy)phenyl)-1,3,4-oxadiazole-2-carboxamide TFA salt (0.32 g, quantitative yield).

LCMS: Method C1, 1.08 min, MS: ES+ 464.2.

Step (iii)

### N-((3R,5S)-5-((1H-1,2,4-Triazol-1-yl)methyl)-1-cyanopyrrolidin-3-yl)-5-(2-cyclopropyl-5-(trifluoromethoxy)phenyl)-1,3,4-oxadiazole-2-carboxamide

To a stirred solution of N-((3R,5S)-5-((1H-1,2,4-triazol-1-yl)methyl)pyrrolidin-3-yl)-5-(2-cyclopropyl-5-(trifluoromethoxy)phenyl)-1,3,4-oxadiazole-2-carboxamide TFA salt (0.32 g, 0.55 mmol) in THF (3.2 mL) was added K₂CO₃ (0.23 g, 1.66 mmol) at rt and stirred for 5 min. Cyanogen bromide (0.06 g, 0.61 mmol) was added into the mixture at 0° C. The mixture was stirred at 0° C. for 1 h, then poured into water (50 mL) and extracted with EtOAc (2×80 mL). The combined organic phases were dried over anhydrous Na₂SO₄, filtered and concentrated under reduced pressure. The residue was purified by flash column chromatography (silica gel, 85% EtOAc in n-hexanes) to yield N-((3R,5S)-5-((1H-1,2,4-triazol-1-yl)methyl)-1-cyanopyrrolidin-3-yl)-5-(2-cyclopropyl-5-(trifluoromethoxy)phenyl)-1,3,4-oxadiazole-2-carboxamide (0.05 g, 0.10 mmol, 26% yield over two steps), which was further purified by reverse phase preparative HPLC purification (Method X15) to yield N-((3R,5S)-5-((1H-1,2,4-triazol-1-yl)methyl)-1-cyanopyrrolidin-3-yl)-5-(2-cyclopropyl-5-(trifluoromethoxy)phenyl)-1,3,4-oxadiazole-2-carboxamide (0.01 g, 0.03 mmol, 8% yield over two steps).

LCMS: Method H1, 2.95 min, MS: ES+ 489.2; ¹H NMR (400 MHz, DMSO-d₆) δ ppm: 9.74 (br s, 1H), 8.53-8.56 (m, 1H), 7.98-8.01 (m, 1H), 7.78-7.81 (m, 1H), 7.54-7.56 (m, 1H), 7.27-7.29 (m, 1H), 4.30-4.40 (m, 4H), 3.51-3.63 (m, 1H), 3.35-3.42 (m, 1H), 2.61-2.72 (m, 1H), 2.13-2.23 (m, 1H), 1.98-2.07 (s, 1H), 0.98-1.09 (m, 2H), 0.72-0.83 (m, 2H); Chiral SFC: Method Y14, 3.7 min.

### Example 48

### N-((3R,5S)-5-((1H-1,2,3-Triazol-1-yl)methyl)-1-cyanopyrrolidin-3-yl)-5-(2-propyl-5-(trifluoromethoxy)phenyl)-1,3,4-oxadiazole-2-carboxamide

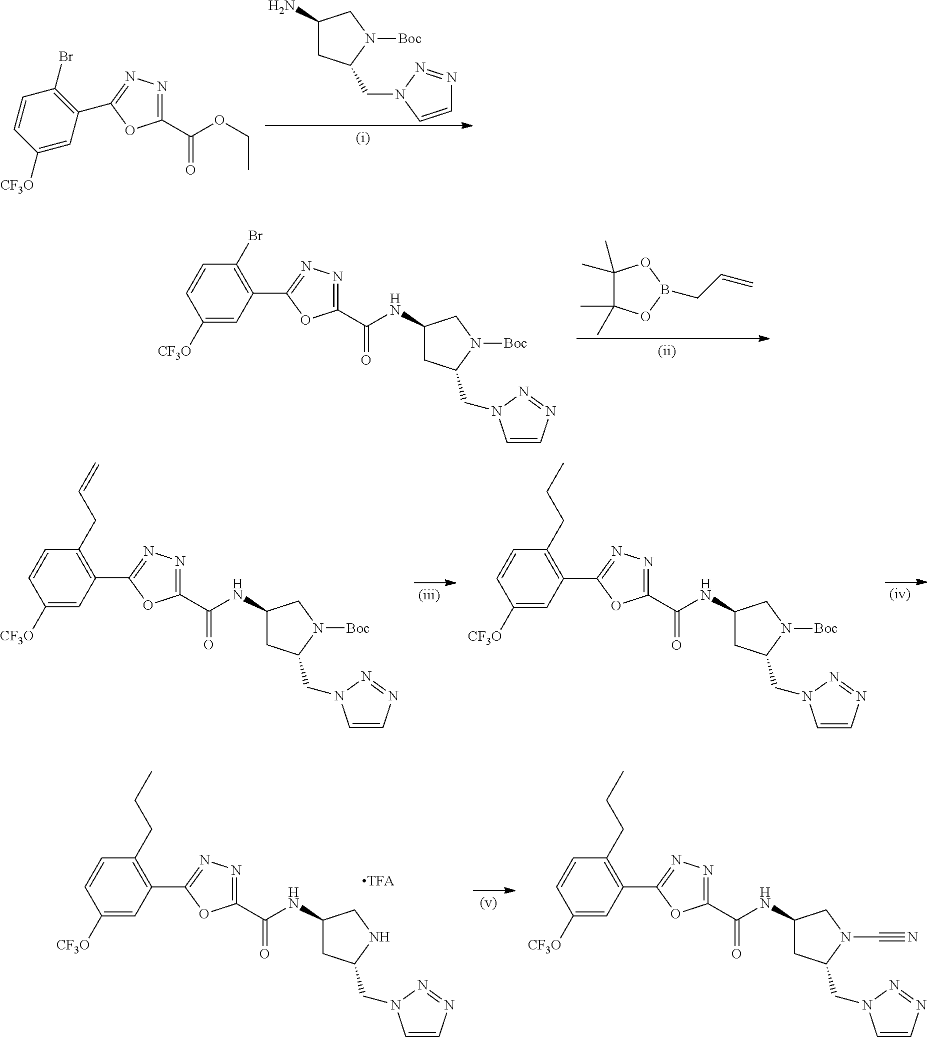

### Step (i)

### tert-Butyl (2S,4R)-2-((1H-1,2,3-triazol-1-yl)methyl)-4-(5-(2-bromo-5-(trifluoromethoxy)phenyl)-1,3,4-oxadiazole-2-carboxamido)pyrrolidine-1-carboxylate

To a stirred solution of ethyl 5-(2-bromo-5-(trifluoromethoxy)phenyl)-1,3,4-oxadiazole-2-carboxylate (1.5 g, 3.94 mmol) and tert-butyl (2S,4R)-2-((1H-1,2,3-triazol-1-yl)methyl)-4-aminopyrrolidine-1-carboxylate (1.05 g, 3.94 mmol) in THF (15 mL) was added DBU (4.5 mL, 3 vol) dropwise at 0° C. The mixture was allowed to warm to rt and stirred for 1 h, then poured into water (20 mL) and extracted with EtOAc (3×20 mL). The combined organic phases were dried over anhydrous Na₂SO₄, filtered and concentrated under reduced pressure. The residue was purified by flash column chromatography (silica gel, 100% EtOAc) to yield tert-butyl (2S,4R)-2-((1H-1,2,3-triazol-1-yl)methyl)-4-(5-(2-bromo-5-(trifluoromethoxy)phenyl)-1,3,4-oxadiazole-2-carboxamido)pyrrolidine-1-carboxylate (0.45 g, 0.70 mmol, 18% yield). LCMS: Method C1, 1.28 min, MS: ES+ 602.2, 604.1.

### Step (ii)

### tert-Butyl (2S,4R)-2-((1H-1,2,3-triazol-1-yl)methyl)-4-(5-(2-allyl-5-(trifluoromethoxy)phenyl)-1,3,4-oxadiazole-2-carboxamido)pyrrolidine-1-carboxylate

To a stirred solution of tert-butyl (2S,4R)-2-((1H-1,2,3-triazol-1-yl)methyl)-4-(5-(2-bromo-5-(trifluoromethoxy)phenyl)-1,3,4-oxadiazole-2-carboxamido)pyrrolidine-1-carboxylate (0.40 g, 0.66 mmol) and 2-allyl-4,4,5,5-tetramethyl-1,3,2-dioxaborolane (CAS 72824-04-5, from Combi-Blocks, 0.13 g, 0.79 mmol) in 1,4-Dioxane:water (4 mL, 4:1) was added K₃PO₄ (0.42 g, 1.99 mmol). The mixture was purged with N₂ gas for 10 min, followed by addition of [1,1′-bis(diphenylphosphino)ferrocene]dichloropalladium(II) (0.04 g, 0.05 mmol) and heated at 110° C. for 2 h. The mixture was poured into water (25 mL) and extracted with EtOAc (3×25 mL). The combined organic phases were dried over anhydrous Na₂SO₄, filtered and concentrated under reduced pressure. The residue was purified by flash column chromatography (silica gel, 80% EtOAc in n-hexanes) to yield tert-butyl (2S,4R)-2-((1H-1,2,3-triazol-1-yl)methyl)-4-(5-(2-allyl-5-(trifluoromethoxy)phenyl)-1,3,4-oxadiazole-2-carboxamido)pyrrolidine-1-carboxylate (0.15 g, 0.26 mmol, 40% yield).

LCMS: Method C, 1.84 min, MS: ES+ 564.4.

Step (iii)

### tert-Butyl (2S,4R)-2-((1H-1,2,3-triazol-1-yl)methyl)-4-(5-(2-propyl-5-(trifluoromethoxy)phenyl)-1,3,4-oxadiazole-2-carboxamido)pyrrolidine-1-carboxylate

To a stirred solution of tert-butyl (2S,4R)-2-((1H-1,2,3-triazol-1-yl)methyl)-4-(5-(2-allyl-5-(trifluoromethoxy)phenyl)-1,3,4-oxadiazole-2-carboxamido)pyrrolidine-1-carboxylate (0.14 g, 0.24 mmol) in MeOH (1.4 mL) was added 10% Pd/C (50% moisture) (0.03 g, 0.2 w/w). The mixture was purged with H₂ gas for 3 h, then filtered through Celite Hyflow® and the filtrate was concentrated under reduced pressure to yield tert-butyl (2S,4R)-2-((1H-1,2,3-triazol-1-yl)methyl)-4-(5-(2-propyl-5-(trifluoromethoxy)phenyl)-1,3,4-oxadiazole-2-carboxamido)pyrrolidine-1-carboxylate (0.06 g, 0.11 mmol, 46%).

LCMS: Method C1, 1.38 min, MS: ES+ 566.4.

### Step (iv)

### N-((3R,5S)-5-((1H-1,2,3-Triazol-1-yl)methyl)pyrrolidin-3-yl)-5-(2-propyl-5-(trifluoromethoxy)phenyl)-1,3,4-oxadiazole-2-carboxamide TFA salt

To a stirred solution of tert-butyl (2S,4R)-2-((1H-1,2,3-triazol-1-yl)methyl)-4-(5-(2-propyl-5-(trifluoromethoxy)phenyl)-1,3,4-oxadiazole-2-carboxamido)pyrrolidine-1-carboxylate (0.06 g, 0.11 mmol) in DCM (1 mL) was added TFA (0.2 mL, 3 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(2-propyl-5-(trifluoromethoxy)phenyl)-1,3,4-oxadiazole-2-carboxamide TFA salt (0.08 g, quantitative yield).

LCMS: Method C, 1.43 min, MS: ES+ 466.3.

### Step (v)

### N-((3R,5S)-5-((1H-1,2,3-Triazol-1-yl)methyl)-1-cyanopyrrolidin-3-yl)-5-(2-propyl-5-(trifluoromethoxy)phenyl)-1,3,4-oxadiazole-2-carboxamide

To a stirred solution of N-((3R,5S)-5-((1H-1,2,3-triazol-1-yl)methyl)pyrrolidin-3-yl)-5-(2-propyl-5-(trifluoromethoxy)phenyl)-1,3,4-oxadiazole-2-carboxamide TFA salt (0.08 g, 0.14 mmol) in THF (1 mL) was added K₂CO₃ (0.06 g, 0.41 mmol) at rt and stirred for 5 min. Cyanogen bromide (0.01 g, 0.14 mmol) was added into the reaction mixture at 0° C. The mixture was stirred at rt for 1 h, then poured into water (20 mL) and extracted with EtOAc (3×10 mL). The combined organic phases were dried over anhydrous Na₂SO₄, filtered and concentrated under reduced pressure. The residue was purified by reverse phase preparative HPLC (Method X16) to yield N-((3R,5S)-5-((1H-1,2,3-triazol-1-yl)methyl)-1-cyanopyrrolidin-3-yl)-5-(2-propyl-5-(trifluoromethoxy)phenyl)-1,3,4-oxadiazole-2-carboxamide (0.01 g, 0.02 mmol, 6% yield over two steps).

LCMS: Method H1, 3.15 min, MS: ES+ 491.2; ¹H NMR (400 MHz, DMSO-d₆) δ ppm: 9.75 (d, J=5.6 Hz, 1H), 8.18 (s, 1H), 7.86 (s, 1H), 7.77 (s, 1H), 7.62 (s, 2H), 4.62-4.63 (m, 2H), 4.36-4.37 (m, 2H), 3.66-3.70 (m, 1H), 3.41-3.44 (m, 1H), 3.00 (t, J=8.0 Hz, 2H), 2.18-2.21 (m, 1H), 1.98-2.03 (m, 1H), 1.57 (q, J=6.8 Hz, 2H), 0.91 (t, J=6.8 Hz, 3H); Chiral SFC: Method Y14, 3.61 min.

### Example 49

### N-((3R,5S)-5-((4H-1,2,4-Triazol-4-yl)methyl)-1-cyanopyrrolidin-3-yl)-5-(2-cyclopropyl-5-(trifluoromethoxy)phenyl)-1,3,4-oxadiazole-2-carboxamide

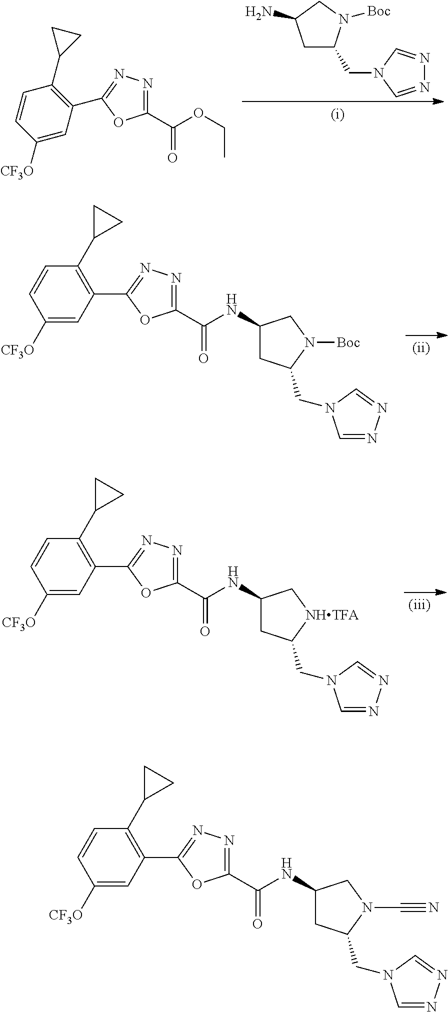

### Step (i)

### tert-Butyl (2S,4R)-2-((4H-1,2,4-triazol-4-yl)methyl)-4-(5-(2-cyclopropyl-5-(trifluoromethoxy)phenyl)-1,3,4-oxadiazole-2-carboxamido)pyrrolidine-1-carboxylate

To a stirred solution of ethyl 5-(2-cyclopropyl-5-(trifluoromethoxy)phenyl)-1,3,4-oxadiazole-2-carboxylate (0.40 g, 1.16 mmol) and tert-butyl (2S,4R)-2-((4H-1,2,4-triazol-4-yl)methyl)-4-aminopyrrolidine-1-carboxylate (0.31 g, 1.68 mmol) in MeOH (7 mL) was added DMAP (0.17 g, 1.40 mmol) in portions at 0° C. The mixture was heated at 75° C. for 16 h. The mixture was concentrated under reduced pressure to yield tert-butyl (2S,4R)-2-((4H-1,2,4-triazol-4-yl)methyl)-4-(5-(2-cyclopropyl-5-(trifluoromethoxy)phenyl)-1,3,4-oxadiazole-2-carboxamido)pyrrolidine-1-carboxylate (0.29 g, 0.52 mmol, 44% yield). LCMS: Method C, 1.68 min, MS: ES+ 564.2.

### Step (ii)

### N-((3R,5S)-5-((4H-1,2,4-Triazol-4-yl)methyl)pyrrolidin-3-yl)-5-(2-cyclopropyl-5-(trifluoromethoxy)phenyl)-1,3,4-oxadiazole-2-carboxamide TFA salt

To a stirred solution of tert-butyl (2S,4R)-2-((4H-1,2,4-triazol-4-yl)methyl)-4-(5-(2-cyclopropyl-5-(trifluoromethoxy)phenyl)-1,3,4-oxadiazole-2-carboxamido)pyrrolidine-1-carboxylate (0.29 g, 0.52 mmol) in DCM (3 mL) was added TFA (1.5 mL, 5 vol) dropwise at 0° C. The mixture was allowed to warm to rt and stirred for 1 h, then concentrated under reduced pressure to yield N-((3R,5S)-5-((4H-1,2,4-triazol-4-yl)methyl)pyrrolidin-3-yl)-5-(2-cyclopropyl-5-(trifluoromethoxy)phenyl)-1,3,4-oxadiazole-2-carboxamide TFA salt (0.40 g, quantitative yield).

LCMS: Method C, 1.42 min, MS: ES+ 464.2.

Step (iii)

### N-((3R,5S)-5-((4H-1,2,4-Triazol-4-yl)methyl)-1-cyanopyrrolidin-3-yl)-5-(2-cyclopropyl-5-(trifluoromethoxy)phenyl)-1,3,4-oxadiazole-2-carboxamide

To a stirred solution of N-((3R,5S)-5-((4H-1,2,4-triazol-4-yl)methyl)pyrrolidin-3-yl)-5-(2-cyclopropyl-5-(trifluoromethoxy)phenyl)-1,3,4-oxadiazole-2-carboxamide TFA salt (0.40 g, 0.69 mmol) in THF (4 mL) was added K₂CO₃ (0.28 g, 2.07 mmol) at rt and stirred for 5 min. Cyanogen bromide (0.11 g, 1.03 mmol) was added into the reaction mixture at 0° C. The mixture was stirred at rt for 1 h, then poured into water (20 mL) and extracted with EtOAc (3×30 mL). The combined organic phases were dried over anhydrous Na₂SO₄, filtered and concentrated under reduced pressure. The residue was purified by reverse phase preparative HPLC (Method X14) to yield N-((3R,5S)-5-((4H-1,2,4-triazol-4-yl)methyl)-1-cyanopyrrolidin-3-yl)-5-(2-cyclopropyl-5-(trifluoromethoxy)phenyl)-1,3,4-oxadiazole-2-carboxamide (0.07 g, 0.12 mmol, 27% yield over two steps).

LCMS: Method H1, 2.79 min, MS: ES+ 489.2; ¹H NMR (400 MHz, DMSO-d₆) δ ppm: 9.77 (br s, 1H), 8.57 (s, 2H), 7.83 (s, 1H), 7.57 (d, J=8.0 Hz, 1H), 7.31 (d, J=8.8 Hz, 1H), 4.41-4.49 (m, 1H), 4.24-4.32 (m, 3H), 3.71-3.75 (m, 1H), 3.42-3.46 (m, 1H), 2.67-2.69 (m, 1H), 2.15-2.20 (m, 1H), 1.91-1.96 (m, 1H), 1.05-1.07 (m, 2H), 0.80-0.81 (m, 2H); Chiral SFC: Method Y4, 5.48 min.

### Example 50

### N-((3R,5S)-5-((1H-1,2,3-Triazol-1-yl)methyl)-1-cyanopyrrolidin-3-yl)-3-(2-cyclopropyl-5-(trifluoromethoxy)phenyl)-1,2,4-oxadiazole-5-carboxamide

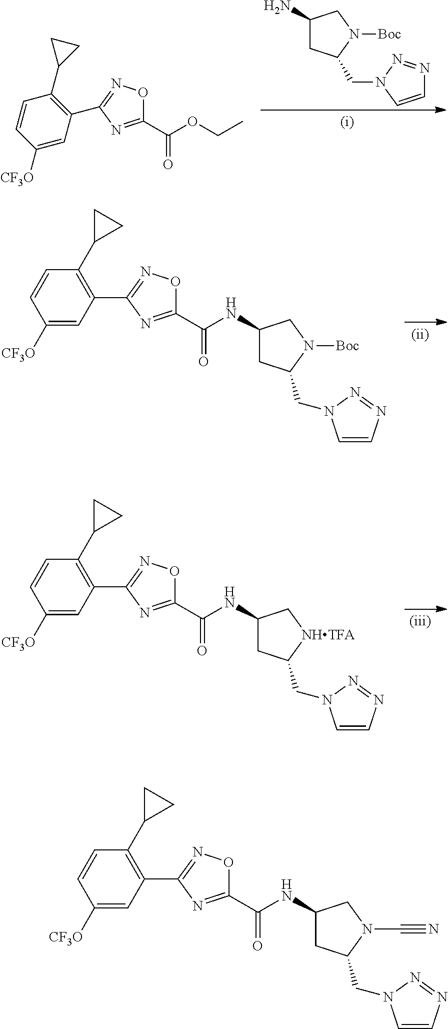

### Step (i)

### tert-Butyl (2S,4R)-2-((1H-1,2,3-triazol-1-yl)methyl)-4-(3-(2-cyclopropyl-5-(trifluoromethoxy)phenyl)-1,2,4-oxadiazole-5-carboxamido)pyrrolidine-1-carboxylate

To a stirred solution of ethyl 3-(2-cyclopropyl-5-(trifluoromethoxy)phenyl)-1,2,4-oxadiazole-5-carboxylate (0.70 g, 2.04 mmol) and tert-butyl (2S,4R)-2-((1H-1,2,3-triazol-1-yl)methyl)-4-aminopyrrolidine-1-carboxylate (0.54 g, 2.04 mmol) in THF (5 mL) was added TBD (0.42 g, 3.06 mmol) in portions at 0° C. and stirred at 0° C. for 30 min. The mixture was poured into water (100 mL) and extracted with EtOAc (3×100 mL). The combined organic phases were dried over anhydrous Na₂SO₄, filtered and concentrated under reduced pressure. The residue was purified by flash column chromatography (silica gel, 40% EtOAc in n-hexanes) to yield tert-butyl (2S,4R)-2-((1H-1,2,3-triazol-1-yl)methyl)-4-(3-(2-cyclopropyl-5-(trifluoromethoxy)phenyl)-1,2,4-oxadiazole-5-carboxamido)pyrrolidine-1-carboxylate (0.14 g, 0.30 mmol, 27% yield).

LCMS: Method C, 1.85 min, MS: ES+ 564.4.

### Step (ii)

### N-((3R,5S)-5-((1H-1,2,3-Triazol-1-yl)methyl)pyrrolidin-3-yl)-3-(2-cyclopropyl-5-(trifluoromethoxy)phenyl)-1,2,4-oxadiazole-5-carboxamide TFA salt

To a stirred solution of tert-butyl (2S,4R)-2-((1H-1,2,3-triazol-1-yl)methyl)-4-(3-(2-cyclopropyl-5-(trifluoromethoxy)phenyl)-1,2,4-oxadiazole-5-carboxamido)pyrrolidine-1-carboxylate (0.40 g, 0.73 mmol) in DCM (5 mL) was added TFA (2 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-3-(2-cyclopropyl-5-(trifluoromethoxy)phenyl)-1,2,4-oxadiazole-5-carboxamide TFA salt (0.50 g, quantitative yield).

LCMS: Method F, 5.28 min, MS: ES+ 464.0.

Step (iii)

### N-((3R,5S)-5-((1H-1,2,3-Triazol-1-yl)methyl)-1-cyanopyrrolidin-3-yl)-3-(2-cyclopropyl-5-(trifluoromethoxy)phenyl)-1,2,4-oxadiazole-5-carboxamide

To a stirred solution of N-((3R,5S)-5-((1H-1,2,3-triazol-1-yl)methyl)pyrrolidin-3-yl)-3-(2-cyclopropyl-5-(trifluoromethoxy)phenyl)-1,2,4-oxadiazole-5-carboxamide TFA salt (0.50 g, 0.86 mmol) in THF (5 mL) was added K₂CO₃ (0.59 g, 4.33 mmol) at rt and stirred for 5 min. Cyanogen bromide (0.09 g, 0.86 mmol) was added into the reaction mixture at 0° C. The mixture was stirred at rt for 15 min, then poured into water (100 mL) and extracted with EtOAc (3×100 mL). The combined organic phases were dried over anhydrous Na₂SO₄, filtered and concentrated under reduced pressure. The residue was purified by flash column chromatography (silica gel, 87% EtOAc in n-hexanes) to yield N-((3R,5S)-5-((1H-1,2,3-triazol-1-yl)methyl)-1-cyanopyrrolidin-3-yl)-3-(2-cyclopropyl-5-(trifluoromethoxy)phenyl)-1,2,4-oxadiazole-5-carboxamide (0.12 g, 0.25 mmol, 35% yield over two steps).

LCMS: Method H1, 3.19 min, MS: ES+ 489.2; ¹H NMR (400 MHz, DMSO-d₆) δ ppm: 9.85 (d, J 6.8 Hz, 1H), 8.21 (s, 1H), 7.80-7.81 (m, 2H), 7.55 (d, J=7.6 Hz, 1H), 7.30 (d, J=8.8 Hz, 1H), 4.64-4.66 (m, 2H), 4.36-4.41 (m, 2H), 3.68-3.72 (m, 1H), 3.45-3.48 (m, 1H), 3.35 (m, 1H), 2.19-2.24 (m, 1H), 2.01-2.06 (m, 1H), 1.02-1.06 (m, 2H), 0.77-0.80 (m, 2H); Chiral SFC: Method Y21, 7.47 min.

### Example 51

### N-((3R,5S)-5-((1H-1,2,3-Triazol-1-yl)methyl)-1-cyanopyrrolidin-3-yl)-5-(2-cyclopropyl-5-(trifluoromethoxy)phenyl)-1,2,4-oxadiazole-3-carboxamide

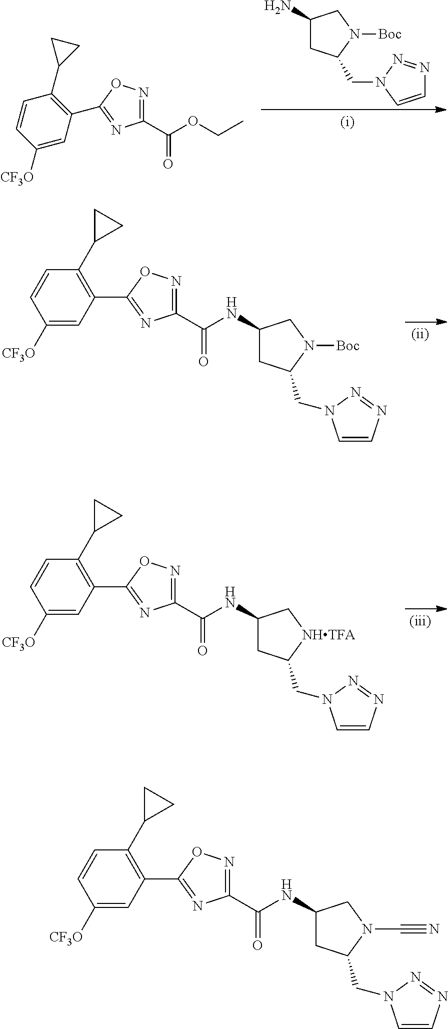

### Step (iv)

### tert-Butyl (2S,4R)-2-((1H-1,2,3-triazol-1-yl)methyl)-4-(5-(2-cyclopropyl-5-(trifluoromethoxy)phenyl)-1,2,4-oxadiazole-3-carboxamido)pyrrolidine-1-carboxylate

To a stirred solution of ethyl 5-(2-cyclopropyl-5-(trifluoromethoxy)phenyl)-1,2,4-oxadiazole-3-carboxylate (0.15 g, 0.43 mmol) and tert-butyl (2S,4R)-2-((1H-1,2,3-triazol-1-yl)methyl)-4-aminopyrrolidine-1-carboxylate (0.13 g, 0.48 mmol) in THF (1.5 mL) was added TBD (0.09 g, 0.65 mmol) in portions at 0° C. The mixture was allowed to warm to rt and stirred for 2 h, then poured into water (10 mL) and extracted with EtOAc (2×30 mL). The combined organic phases were dried over anhydrous Na₂SO₄, filtered and concentrated under reduced pressure. The residue was purified by flash column chromatography (silica gel, 70% EtOAc in n-hexanes) to yield tert-butyl (2S,4R)-2-((1H-1,2,3-triazol-1-yl)methyl)-4-(5-(2-cyclopropyl-5-(trifluoromethoxy)phenyl)-1,2,4-oxadiazole-3-carboxamido)pyrrolidine-1-carboxylate (0.08 g, 0.14 mmol, 33% yield).

LCMS: Method C1, 1.35 min, MS: ES+ 564.2.

### Step (ii)

### N-((3R,5S)-5-((1H-1,2,3-Triazol-1-yl)methyl)pyrrolidin-3-yl)-5-(2-cyclopropyl-5-(trifluoromethoxy)phenyl)-1,2,4-oxadiazole-3-carboxamide TFA salt

To a stirred solution of tert-butyl (2S,4R)-2-((1H-1,2,3-triazol-1-yl)methyl)-4-(5-(2-cyclopropyl-5-(trifluoromethoxy)phenyl)-1,2,4-oxadiazole-3-carboxamido)pyrrolidine-1-carboxylate (0.08 g, 0.14 mmol) in DCM (0.8 mL) was added TFA (0.4 mL, 5 vol) dropwise at 0° C. The mixture was allowed to warm to rt and stirred for 1 h, then concentrated under reduced pressure to yield N-((3R,5S)-5-((1H-1,2,3-triazol-1-yl)methyl)pyrrolidin-3-yl)-5-(2-cyclopropyl-5-(trifluoromethoxy)phenyl)-1,2,4-oxadiazole-3-carboxamide TFA salt (0.10 g, quantitative yield).

LCMS: Method C1, 1.16 min, MS: ES+ 464.2.

Step (iii)

### N-((3R,5S)-5-((1H-1,2,3-Triazol-1-yl)methyl)-1-cyanopyrrolidin-3-yl)-5-(2-cyclopropyl-5-(trifluoromethoxy)phenyl)-1,2,4-oxadiazole-3-carboxamide

To a stirred solution of N-((3R,5S)-5-((1H-1,2,3-triazol-1-yl)methyl)pyrrolidin-3-yl)-5-(2-cyclopropyl-5-(trifluoromethoxy)phenyl)-1,2,4-oxadiazole-3-carboxamide TFA salt (0.10 g, 0.21 mmol) in THF (1.0 mL) was added K₂CO₃ (0.12 g, 0.83 mmol) at rt and stirred for 5 min. Cyanogen bromide (0.02 g, 0.21 mmol) was added at 0° C. The mixture was stirred at rt for 1 h, then poured into water (10 mL) and extracted with EtOAc (2×15 mL). The combined organic phases were dried over anhydrous Na₂SO₄ and concentrated under reduced pressure. The residue was purified by reverse phase preparative HPLC (Method X17) to yield N-((3R,5S)-5-((1H-1,2,3-triazol-1-yl)methyl)-1-cyanopyrrolidin-3-yl)-5-(2-cyclopropyl-5-(trifluoromethoxy)phenyl)-1,2,4-oxadiazole-3-carboxamide (0.02 g, 0.045 mmol, 32% yield over two steps).

LCMS: Method F, 5.93 min, MS: ES+ 489.0; ¹H NMR (400 MHz, DMSO-d₆) δ ppm: 9.50 (d, J=4.0 Hz, 1H), 8.20 (s, 1H), 7.94 (s, 1H), 7.78 (s, 1H), 7.63 (d, J=8.0 Hz, 1H), 7.36 (d, J=8.0 Hz, 1H), 4.58-4.69 (m, 2H), 4.35-4.38 (m, 2H), 3.67-3.70 (m, 1H), 3.40-3.43 (m, 1H), 2.57-2.65 (m, 1H), 2.19-2.25 (m, 1H), 1.99-2.02 (m, 1H), 1.06-1.08 (m, 2H), 0.77-0.85 (m, 2H); Chiral SFC: Method Y14, 4.11 min.

### Example 52

### N-((3R,5S)-5-((1H-Pyrazol-1-yl)methyl)-1-cyanopyrrolidin-3-yl)-6-(1H-indazol-4-yl)nicotinamide

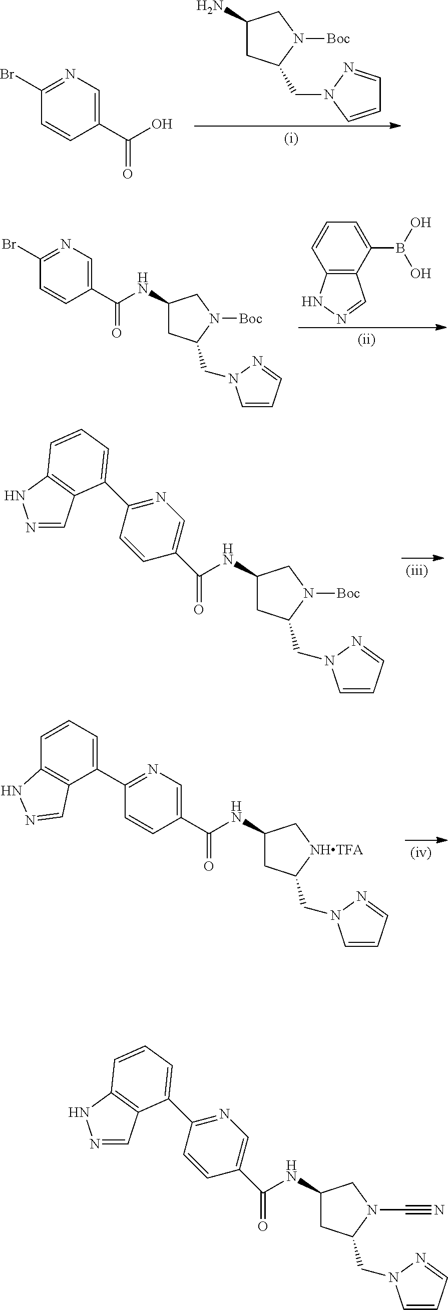

### Step (i)

### tert-Butyl (2S,4R)-2-((1H-pyrazol-1-yl)methyl)-4-(6-bromonicotinamido)pyrrolidine-1-carboxylate

To a stirred solution of 6-bromonicotinic acid (CAS 6311-35-9, from Combi-Blocks, 0.60 g, 2.97 mmol) and tert-butyl (2S,4R)-2-((1H-pyrazol-1-yl)methyl)-4-aminopyrrolidine-1-carboxylate (0.79 g, 2.97 mmol) in THF (6 mL) were added DIPEA (1.14 g, 1.58 mL, 0.07 mmol) and HATU (1.69 g, 2.97 mmol) at 0° C. The mixture was allowed to warm to rt and stirred at rt for 16 h, then poured into water (20 mL) and extracted with EtOAc (3×20 mL). The combined organic phases were dried over anhydrous Na₂SO₄ and concentrated under reduced pressure. The residue was purified by flash column chromatography (silica gel, 60% EtOAc in n-hexanes) to yield tert-butyl (2S,4R)-2-((1H-pyrazol-1-yl)methyl)-4-(6-bromonicotinamido)pyrrolidine-1-carboxylate (0.63 g, 1.40 mmol, 49% yield). LCMS: Method C, 1.58 min, MS: ES+ 450.2, 452.2.

### Step (ii)

### tert-Butyl (2S,4R)-4-(6-(1H-indazol-4-yl) nicotinamido)-2-((1H-pyrazol-1-yl)methyl)pyrrolidine-1-carboxylate

To a stirred solution of tert-butyl (2S,4R)-2-((1H-pyrazol-1-yl)methyl)-4-(6-bromonicotinamido) pyrrolidine-1-carboxylate (0.30 g, 0.66 mmol) and (1H-indazol-4-yl)boronic acid (CAS 1023595-17-6, from Combi-Blocks, 0.10 g, 0.66 mmol) in 1,4-dioxane:water (3 mL, 2:1) was added K₃PO₄ (0.28 g, 1.33 mmol). The mixture was purged with N₂ gas for 15 min, followed by addition of [1,1′-bis(diphenylphosphino)ferrocene]dichloropalladium(II) (0.05 g, 0.06 mmol) and heated at 100° C. for 2 h. The mixture was poured into water (20 mL) and extracted with EtOAc (3×15 mL). The combined organic phases were dried over anhydrous Na₂SO₄ and concentrated under reduced pressure. The residue was purified by flash column chromatography (silica gel, 80% EtOAc in n-hexanes) to yield tert-butyl (2S,4R)-4-(6-(1H-indazol-4-yl)nicotinamido)-2-((1H-pyrazol-1-yl)methyl)pyrrolidine-1-carboxylate (0.20 g, 0.41 mmol, 77% yield). LCMS: Method C, 1.43 min, MS: ES+ 488.3.

Step (iii)

### N-((3R,5S)-5-((1H-Pyrazol-1-yl)methyl)pyrrolidin-3-yl)-6-(1H-indazol-4-yl)nicotinamide TFA salt

To a stirred solution of tert-butyl (2S,4R)-4-(6-(1H-indazol-4-yl)nicotinamido)-2-((1H-pyrazol-1-yl)methyl)pyrrolidine-1-carboxylate (0.20 g, 0.41 mmol) in DCM (2 mL) was added TFA (1.0 mL, 5 vol) dropwise at 0° C. The mixture was allowed to warm to rt and stirred for 1 h, then concentrated under reduced pressure to yield N-((3R,5S)-5-((1H-pyrazol-1-yl)methyl)pyrrolidin-3-yl)-6-(1H-indazol-4-yl)nicotinamide TFA salt (0.20 g, quantitative yield).

LCMS: Method C, 1.23 min, MS: ES+ 388.3.

### Step (iv)

### N-((3R,5S)-5-((1H-Pyrazol-1-yl)methyl)-1-cyanopyrrolidin-3-yl)-6-(1H-indazol-4-yl)nicotinamide

To a stirred solution of N-((3R,5S)-5-((1H-pyrazol-1-yl)methyl)pyrrolidin-3-yl)-6-(1H-indazol-4-yl)nicotinamide TFA salt (0.20 g, 0.36 mmol) in THF (2 mL) was added K₂CO₃ (0.16 g, 1.19 mmol) at rt and stirred for 5 min. Cyanogen bromide (0.05 g, 0.47 mmol) was added into the reaction mixture at 0° C. The reaction mixture was stirred at rt for 1 h, then poured into water (20 mL) and extracted with EtOAc (2×15 mL). The combined organic phases were dried over anhydrous Na₂SO₄, filtered and concentrated under reduced pressure. The residue was purified by flash column chromatography (silica gel, 8% MeOH in DCM) to yield N-((3R,5S)-5-((1H-pyrazol-1-yl)methyl)-1-cyanopyrrolidin-3-yl)-6-(1H-indazol-4-yl)nicotinamide (0.10 g, 0.24 mmol, 39% yield over two steps).

LCMS: Method H1, 2.14 min, MS: ES− 411.2; ¹H NMR (400 MHz, DMSO-d₆) δ ppm: 13.29 (s, 1H), 9.21 (d, J=1.6 Hz, 1H), 8.90 (d, J=6.4 Hz, 1H), 8.66 (s, 1H), 8.33 (dd, J=8.4, 2.0 Hz, 1H), 8.21 (d, J=8.4 Hz, 1H), 7.81-7.83 (m, 2H), 7.70 (d, J=8.4 Hz, 1H), 7.49-7.53 (m, 2H), 6.31 (s, 1H), 4.33-4.35 (m, 3H), 3.67-3.71 (m, 1H), 3.39-3.44 (m, 2H), 2.07-2.14 (m, 1H), 2.01-2.06 (m, 1H).

Chiral SFC: Method Y32, 6.08 min.

### Biological Activity of Compounds of the Invention

### Abbreviations

TAMRA carboxytetramethylrhodamine
Tris 2-amino-2-(hydroxymethyl)-1,3-propanediol
BSA bovine serum albumin

### USP30 Biochemical IC₅₀ Assay

Dilution plates were prepared at 21 times the final concentration (2100 μM for a final concentration of 100 μM) in 50% DMSO in a 96-well polypropylene V-bottom plate (Greiner #651201). A typical 8-point dilution series would be 100, 30, 10, 3, 1, 0.3, 0.1, 0.03 μM final. Reactions were performed in duplicate in black 384 well plates (small volume, Greiner 784076) in a final reaction volume of 21 μl.

Either lpl of 50% DMSO or diluted compound was added to the plate. USP30 (Boston Biochem #E582) was diluted in reaction buffer (40 mM Tris, pH 7.5, 0.005% Tween 20, 0.5 mg/ml BSA, 5 mM beta-mercaptoethanol) to achieve a final assay concentration of 4 nM, and 10 μl of diluted USP30 was added to the compound. Enzyme and compound were incubated for 30 min at room temp. Reactions were initiated by the addition of 50 nM of TAMRA labelled peptide linked to ubiquitin via an isopeptide bond as fluorescence polarisation substrate. Reactions were read immediately after addition of substrate and following a 2-hour incubation at room temperature. Readings were performed on a Pherastar Plus (BMG Labtech). λ Excitation 540 nm; λ Emission 590 nm.

Activity of exemplary compounds in USP30 biochemical assay (IC₅₀ geomean):

| | IC₅₀ |
|---|---|
| Example | (nM) |
| 1 | 5 |
| 2 | 11 |
| 3 | 2 |
| 4 | 3 |
| 5 | 4 |
| 6 | 23 |
| 7 | 35 |
| 8 | 14 |
| 9 | 2 |
| 10 | 45 |
| 11 | 4 |
| 12 | 8 |
| 13 | 5 |
| 14 | 4 |
| 15 | 8 |
| 16 | 28 |
| 17 | 4 |
| 18 | 2 |
| 19 | 2 |
| 24 | 9 |
| 25 | 9 |
| 26 | 5 |
| 27 | 9 |
| 28 | 10 |
| 29 | 55 |
| 30 | 11 |
| 34 | 44 |
| 35 | 27 |
| 36 | 100 |
| 37 | 120 |
| 38 | 210 |
| 39 | 8 |
| 40 | 12 |
| 41 | 9 |
| 42 | 6 |
| 43 | 12 |
| 44 | 5 |
| 45 | 7 |
| 46 | 9 |
| 47 | 5 |
| 48 | 5 |
| 49 | 6 |
| 50 | 5 |
| 51 | 14 |
| 52 | 15 |

TOM20-Ubiquitylation Assay Human cell lines can be challenged with mitochondrial depolarizing agents (ionophores (eg. CCCP, valinomycin), mitochondrial complex inhibitors (oligomycin, antimycin A)) to induce ubiquitylation of TOM20, which is then further promoted in the presence of USP30 inhibitors. TOM20 ubiquitylation is subsequently assessed through western blotting of the cell lysates, with TOM20 ubiquitylation adduct detection possible due to an 8 kDa molecule weight increase for each molecule of ubiquitin added, resulting in laddering of a TOM20 immunoreactive band. TOM20-ubiquitylation levels can be quantified using chemiluminescence densitometry of laddered immunoreactive bands.

Activity of exemplary compounds in TOM20-ubiquitylation assay (TOM20-Ub 1.5-fold gain, antimycin A/oligomycin mitophagy trigger EC1.5×):

| | EC1.5x | | EC1.5x | | EC1.5x |
|---|---|---|---|---|---|
| | (Geomean, | | (Geomean, | | (Geomean, |
| Example | nM) | Example | nM) | Example | nM) |
| 1 | 17 | 8 | 41 | 18 | 16 |
| 2 | 15 | 12 | 25 | 24 | 620 |
| 3 | 8 | 13 | 6 | 26 | 14 |
| 5 | 9 | 16 | 950 | 30 | 14 |
| 6 | 180 | 17 | 16 | 46 | 19 |

### Preclinical In Vivo Models

Compounds of the invention may be tested for efficacy in representative in vivo disease models, using standard study procedures from the published literature, including, for example:
(a) Bleomycin-induced lung fibrosis model, which is a leading preclinical in vivo model of Idiopathic Pulmonary Fibrosis. [Kobayashi et al, 2016, J Immunol, 197(2):504-516]
(b) Diet-induced model of NAFLD and glucose homeostasis. [Nishida et al, 2013, Lab Invest; February; 93(2):230-41]
(c) MPTP Model of Parkinson's Disease, which is a commonly used paradigm for looking at neurodegeneration in the dopaminergic system of the brain which is triggered by chemically-induced mitochondrial dysfunction. [Karuppagouner et al, 2014, Sci Rep. 2014 May 2; 4:4874]
(d) Ndufs4KO Leigh syndrome model. [Kruse et al, 2008, Cell Metab. April; 7(4):312-20]
(e) Aged mice model: effects on cognitive and motor function. [Kobilo et al, 2014, Learn Mem. January 17; 21(2):119-26; Creed et al, 2019, Neuroscience. June 15; 409:169-179]
(f) The unilateral ureteral obstructive kidney disease model (UUO). [Chevalier et al, 2009, Kidney Int 75(11): 1145-1152]
(g) The ischemia-induced acute kidney injury model (AKI).

1. A compound of formula (I): 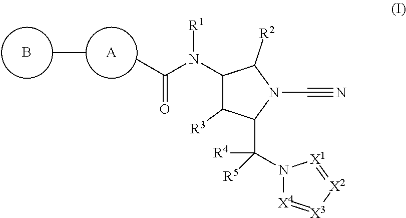
a tautomer thereof, or a pharmaceutically acceptable salt of said compound or tautomer, wherein either:
(a) X¹ is N; and
X², X³ and X⁴ are CR⁶; or
(b) X¹ is N;
one of X², X³ and X⁴ are N; and
two of X², X³ and X⁴ are CR⁶; or
(c) X¹ and X⁴ are CR⁶; and
X² and X³ are N;
ring A is selected from:
(i) a 5-membered monocyclic heteroaryl ring comprising 1 to 3 heteroatoms, each independently selected from N and O;
(ii) a 6-membered monocyclic heteroaryl ring comprising 1 to 3 heteroatoms, each independently selected from N, O and S;
(iii) a 4 to 6-membered saturated or partially saturated monocyclic heterocyclyl ring comprising 1 to 3 heteroatoms, each independently selected from N, O and S; and
(iv) phenyl or naphthyl;
ring A is either unsubstituted or substituted by 1 or 2 R⁷ substituents;
ring B is selected from:
(i) phenyl or naphthyl;
(ii) a 5 to 6-membered monocyclic heteroaryl ring comprising 1 to 3 heteroatoms, each independently selected from N, O and S; and
(iii) a 9 to 10-membered bicyclic heteroaryl ring comprising 1 to 4 heteroatoms, each independently selected from N, O and S;
ring B is either unsubstituted or substituted by 1 to 5 substituents, each independently selected from halo, CN, hydroxy, oxo, (C₁-C₆)alkyl, (C₁-C₆)alkoxy, halo(C₁-C₆)alkyl, halo(C₁-C₆)alkoxy, (C₃-C₆)cycloalkyl, O(C₃-C₆)cycloalkyl, (C₁-C₆)alkoxy(C₁-C₆)alkyl, oxetanyloxy, azetidinyl, pyrrolidinyl, piperidinyl, NH(C₁-C₆)alkyl, N((C₁-C₆)alkyl)₂, C(O)NH(C₁-C₆)alkyl, C(O)N((C₁-C₆)alkyl)₂, NHC(O)(C₁-C₆)alkyl, N(C₁-C₆)alkyl)C(O)(C₁-C₆)alkyl), C(O)(C₁-C₆)alkyl, C(O)O(C₁-C₆)alkyl, CO₂H, CONH₂, SO₂NH(C₁-C₆)alkyl and SO₂N((C₁-C₆)alkyl)₂;
R¹ is selected from hydrogen, (C₁-C₆)alkyl and (C₃-C₆)cycloalkyl;
R² and R³ are each independently selected from hydrogen, halo, (C₁-C₄)alkyl and (C₁-C₄)alkoxy;
R⁴ and R⁵ are each independently selected from hydrogen and (C₁-C₄)alkyl;
R⁶ is hydrogen or (C₁-C₄)alkyl; and
each R⁷ is independently selected from halo, (C₁-C₆)alkyl and (C₁-C₆)alkoxy;
with the proviso that when ring A is unsubstituted oxazolyl or oxadiazolyl, and ring B is phenyl, and R¹, R², R³, R⁴ and R⁵ are each hydrogen, and two of X¹, X², X³ and X⁴ are N, then ring B is not substituted by CF₃ in the position meta to ring A.

2. The compound according to claim 1 having the formula (IA): 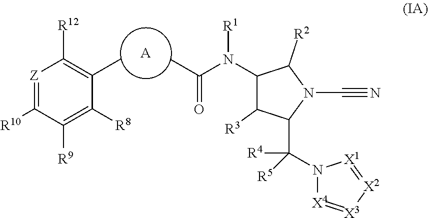
a tautomer thereof, or a pharmaceutically acceptable salt of said compound or tautomer, wherein:
Z is N or CR¹¹;
R¹, R¹¹ and R¹² are each independently selected from hydrogen, halo, CN, hydroxy, oxo, (C₁-C₆)alkyl, (C₁-C₆)alkoxy, halo(C₁-C₆)alkyl, halo(C₁-C₆)alkoxy, (C₃-C₆)cycloalkyl, O(C₃-C₆)cycloalkyl, (C₁-C₆)alkoxy(C₁-C₆)alkyl, oxetanyloxy, azetidinyl, pyrrolidinyl and piperidinyl;
R⁹ and R¹⁰ are each independently selected from hydrogen, halo, CN, hydroxy, oxo, (C₁-C₆)alkyl, (C₁-C₆)alkoxy, halo(C₁-C₆)alkyl, halo(C₁-C₆)alkoxy, (C₃-C₆)cycloalkyl, O(C₃-C₆)cycloalkyl, (C₁-C₆)alkoxy(C₁-C₆)alkyl, oxetanyloxy, azetidinyl, pyrrolidinyl and piperidinyl;
or R⁹ and R¹⁰ together form a 5 to 6-membered saturated, partially saturated, or aromatic ring comprising 1 to 2 heteroatoms, each independently selected from N, O and S, wherein the ring is either unsubstituted or substituted with 1 to 2 substituents, each independently selected from halo, CN, hydroxy, oxo, (C₁-C₆)alkyl, (C₁-C₆)alkoxy, halo(C₁-C₆)alkyl and halo(C₁-C₆)alkoxy;
with the proviso that when ring A is unsubstituted oxazolyl or oxadiazolyl, and R¹, R², R³, R⁴ and R⁵ are each hydrogen, and two of X¹, X², X³ and X⁴ are N, and Z is CR¹¹, and R¹¹ is hydrogen or halo, then R⁹ is not CF₃.

3. The compound according to claim 2, wherein Z is CR¹¹.

4. The compound according to claim 2, wherein Z is N.

5. The compound according to claim 1, wherein ring A is a 5-membered monocyclic heteroaryl ring comprising 1 to 3 heteroatoms, each independently selected from N and O.

6. The compound according to claim 5, wherein ring A is selected from oxazolyl and oxadiazolyl.

7. The compound according to claim 1, wherein ring A is a 6-membered monocyclic heteroaryl ring comprising 1 to 3 heteroatoms, each independently selected from N, O and S.

8. The compound according to claim 7, wherein ring A is pyridinyl.

9. The compound according to claim 1, wherein ring A is pyrrolidinyl.

10. The compound according to claim 1, wherein ring A is phenyl.

11. The compound according to claim 1, wherein R¹ is selected from hydrogen, methyl and cyclopropyl.

12. The compound according to claim 1, wherein R², R³, R⁴ and R⁵ are each hydrogen.

13. The compound according to claim 1, wherein R⁶ is hydrogen or methyl.

14. The compound according to claim 1, wherein ring A is unsubstituted.

15. The compound according to claim 1, wherein each R⁷ is independently selected from fluoro, chloro, methyl and methoxy.

16. The compound according to claim 1, wherein R⁸ and R¹¹ are each independently selected from hydrogen, CN and halo.

17. The compound according to claim 16, wherein R⁸ and R¹¹ are each hydrogen.

18. The compound according to claim 1, wherein R⁹ is selected from hydrogen, halo, CN, (C₁-C₃)alkyl, (C₁-C₃)alkoxy, cyclopropyl, cyclopropoxy, CF₃, OCF₃ and oxetan-3-yloxy.

19. The compound according to claim 18, wherein R⁹ is selected from hydrogen, chloro, CN, ethyl, cyclopropyl, CF₃ and OCF₃.

20. The compound according to claim 1, wherein R¹⁰ is selected from hydrogen, fluoro and CN.

21. The compound according to claim 20, wherein R¹⁰ is hydrogen.

22. The compound according claim 1, wherein R⁹ and R¹⁰ together form a 5-membered partially saturated or aromatic ring comprising 1 to 2 heteroatoms, each independently selected from N, O and S, wherein the ring is either unsubstituted or substituted with 1 to 2 substituents, each independently selected from fluoro, chloro, methyl and methoxy.

23. The compound according to claim 1, wherein R¹² is selected from hydrogen, halo, CN, (C₁-C₃)alkyl, (C₁-C₃)alkoxy, cyclopropyl, cyclopropoxy, CF₃, OCF₃ and oxetan-3-yloxy.

24. The compound according to claim 23, wherein R¹² is selected from hydrogen, methoxy, cyclopropyl, cyclopropoxy, OCF₃ and oxetan-3-yloxy.

25. The compound according to claim 1, which is selected from:
N-((3R,5S)-5-((1H-1,2,3-triazol-1-yl)methyl)-1-cyanopyrrolidin-3-yl)-5-(5-cyano-2-cyclopropylphenyl)-N-cyclopropyloxazole-2-carboxamide;
N-((3R,5S)-5-((1H-1,2,3-triazol-1-yl)methyl)-1-cyanopyrrolidin-3-yl)-5-(5-cyano-2-cyclopropylphenyl)-N-cyclopropyloxazole-2-carboxamide;
N-((3R,5S)-5-((1H-1,2,3-triazol-1-yl)methyl)-1-cyanopyrrolidin-3-yl)-5-(3-cyanophenyl)oxazole-2-carboxamide;
N-((3R,5S)-5-((1H-1,2,3-triazol-1-yl)methyl)-1-cyanopyrrolidin-3-yl)-5-(3-(trifluoromethoxy)phenyl)oxazole-2-carboxamide;
N-((3R,5S)-5-((1H-1,2,3-triazol-1-yl)methyl)-1-cyanopyrrolidin-3-yl)-5-(3-chlorophenyl)oxazole-2-carboxamide;
N-((3R,5S)-5-((1H-1,2,3-triazol-1-yl)methyl)-1-cyanopyrrolidin-3-yl)-5-(2-methoxyphenyl)oxazole-2-carboxamide;
N-((3R,5S)-5-((1H-1,2,3-triazol-1-yl)methyl)-1-cyanopyrrolidin-3-yl)-5-(2-cyclopropylphenyl)-1,3,4-oxadiazole-2-carboxamide;
N-((3R,5S)-5-((1H-1,2,3-triazol-1-yl)methyl)-1-cyanopyrrolidin-3-yl)-5-(2-cyclopropylphenyl)oxazole-2-carboxamide;
N-((3R,5S)-5-((1H-1,2,3-triazol-1-yl)methyl)-1-cyanopyrrolidin-3-yl)-5-(5-cyano-2-cyclopropylphenyl)oxazole-2-carboxamide;
N-((3R,5S)-5-((1H-1,2,3-triazol-1-yl)methyl)-1-cyanopyrrolidin-3-yl)-5-(2-(trifluoromethoxy)phenyl)oxazole-2-carboxamide;
N-((3R,5S)-5-((1H-1,2,3-triazol-1-yl)methyl)-1-cyanopyrrolidin-3-yl)-5-(3-ethylphenyl)oxazole-2-carboxamide;
N-((3R,5S)-5-((1H-1,2,3-triazol-1-yl)methyl)-1-cyanopyrrolidin-3-yl)-5-(3-cyclopropylphenyl)oxazole2-carboxamide;
N-((3R,5S)-5-((1H-1,2,3-triazol-1-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-5-(5-cyano-2-cyclopropylphenyl)-1,3,4-oxadiazole-2-carboxamide;
N-((3R,5S)-5-((1H-1,2,3-triazol-1-yl)methyl)-1-cyanopyrrolidin-3-yl)-5-(2-cyclopropoxy-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-5-(5-cyano-2-(oxetan-3-yloxy)phenyl)-N-cyclopropyloxazole-2-carboxamide;
N-((3R,5S)-5-((2H-1,2,3-triazol-2-yl)methyl)-1-cyanopyrrolidin-3-yl)-5-(3-cyanophenyl)oxazole-2-carboxamide;
N-((3R,5S)-5-((1H-1,2,4-triazol-1-yl)methyl)-1-cyanopyrrolidin-3-yl)-5-(3-cyanophenyl)oxazole-2-carboxamide;
N-((3R,5S)-5-((4H-1,2,4-triazol-4-yl)methyl)-1-cyanopyrrolidin-3-yl)-5-(3-cyanophenyl)oxazole-2-carboxamide;
N-((3R,5S)-5-((1H-1,2,3-triazol-1-yl)methyl)-1-cyanopyrrolidin-3-yl)-4-(3-(trifluoromethyl)phenyl)picolinamide;
N-((3R,5S)-5-((1H-1,2,3-triazol-1-yl)methyl)-1-cyanopyrrolidin-3-yl)-4-(3-(trifluoromethoxy)phenyl)picolinamide;
N-((3R,5S)-5-((1H-pyrazol-1-yl)methyl)-1-cyanopyrrolidin-3-yl)-5-(3-cyanophenyl)oxazole-2-carboxamide;
N-((3R,5S)-5-((1H-pyrazol-1-yl)methyl)-1-cyanopyrrolidin-3-yl)-5-(3-(trifluoromethoxy)phenyl)oxazole-2-carboxamide;
N-((3R,5S)-1-cyano-5-((3-methyl-1H-pyrazol-1-yl)methyl)pyrrolidin-3-yl)-5-(3-(trifluoromethoxy)phenyl)oxazole-2-carboxamide;
N-((3R,5S)-1-cyano-5-((5-methyl-1H-pyrazol-1-yl)methyl)pyrrolidin-3-yl)-5-(3-(trifluoromethoxy)phenyl)oxazole-2-carboxamide;
N-((3R,5S)-5-((1H-pyrazol-1-yl)methyl)-1-cyanopyrrolidin-3-yl)(3(trifluoromethyl)phenyl)oxazole-2-carboxamide;
N-((3R,5S)-5-((1H-pyrazol-1-yl)methyl)-1-cyanopyrrolidin-3-yl)-2-fluoro-4-(1-methyl-1H-indazol-5-yl)benzamide;
N-((3R,5S)-5-((1H-1,2,3-triazol-1-yl)methyl)-1-cyanopyrrolidin-3-yl)-2-fluoro-4-(1-methyl-1H-indazol-5-yl)benzamide;
N-((3R,5S)-5-((1H-pyrazol-1-yl)methyl)-1-cyanopyrrolidin-3-yl)-3-(3-(trifluoromethoxy)phenyl)pyrrolidine-1-carboxamide;
(S)—N-((3R,5S)-5-((1H-pyrazol-1-yl)methyl)-1-cyanopyrrolidin-3-yl)-3-(3-(trifluoromethoxy)phenyl)pyrrolidine-1-carboxamide;
(R)—N-((3R,5S)-5-((1H-pyrazol-1-yl)methyl)-1-cyanopyrrolidin-3-yl)-3-(3-(trifluoromethoxy)phenyl)pyrrolidine-1-carboxamide;
N-((3R,5S)-5-((1H-1,2,3-triazol-1-yl)methyl)-1-cyanopyrrolidin-3-yl)-5-(4-fluoro-3-(trifluoromethoxy)phenyl)oxazole-2-carboxamide;
N-((3R,5S)-5-((1H-1,2,3-triazol-1-yl)methyl)-1-cyanopyrrolidin-3-yl)-5-(2-(azetidin-1-yl)-5-cyanopyridin-3-yl)-1,3,4-oxadiazole-2-carboxamide;
N-((3R,5S)-5-((1H-1,2,3-triazol-1-yl)methyl)-1-cyanopyrrolidin-3-yl)-5-(5-cyano-2-(pyrrolidin-1-yl)pyridin-3-yl)-1,3,4-oxadiazole-2-carboxamide;
N-((3R,5S)-5-((1H-1,2,3-triazol-1-yl)methyl)-1-cyanopyrrolidin-3-yl)-5-(2-chloro-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-5-(3-(trifluoromethoxy)phenyl)-1,3,4-oxadiazole-2-carboxamide;
N-((3R,5S)-5-((1H-pyrazol-1-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((2H-1,2,3-triazol-2-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((1H-pyrazol-1-yl)methyl)-1-cyanopyrrolidin-3-yl)-5-(5-cyano-2-cyclopropylphenyl)-1,3,4-oxadiazole-2-carboxamide;
N-((3R,5S)-5-((1H-1,2,4-triazol-1-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-5-(2-propyl-5-(trifluoromethoxy)phenyl)-1,3,4-oxadiazole-2-carboxamide;
N-((3R,5S)-5-((4H-1,2,4-triazol-4-yl)methyl)-1-cyanopyrrolidin-3-yl)-5-(2-cyclopropyl-5-(trifluoromethoxy)phenyl)-1,3,4-oxadiazole-2-carboxamide;
N-((3R,5S)-5-((1H-1,2,3-triazol-1-yl)methyl)-1-cyanopyrrolidin-3-yl)-3-(2-cyclopropyl-5-(trifluoromethoxy)phenyl)-1,2,4-oxadiazole-5-carboxamide;
N-((3R,5S)-5-((1H-1,2,3-triazol-1-yl)methyl)-1-cyanopyrrolidin-3-yl)-5-(2-cyclopropyl-5-(trifluoromethoxy)phenyl)-1,2,4-oxadiazole-3-carboxamide; and
N-((3R,5S)-5-((1H-pyrazol-1-yl)methyl)-1-cyanopyrrolidin-3-yl)-6-(1H-indazol-4-yl)nicotinamide;
a tautomer thereof, or a pharmaceutically acceptable salt of said compound or tautomer.

26. A compound according claim 1, a tautomer thereof, or a pharmaceutically acceptable salt of said compound or tautomer, for use as a medicament.

27. (canceled)

28. (canceled)

29. A method for the treatment or prevention of a condition involving mitochondrial dysfunction, cancer, or fibrosis, comprising the step of administering an effective amount of a compound according to claim 1, a tautomer thereof, or a pharmaceutically acceptable salt of said compound or tautomer, to a patient in need thereof.

30. The method according to claim 29, wherein the condition involving mitochondrial dysfunction is selected from: a CNS disorder; neurodegenerative disease; Parkinson's disease; Alzheimer's disease; amyotrophic lateral sclerosis; Huntington's disease; ischemia; stroke; dementia with Lewy bodies; frontotemporal dementia; multiple sclerosis; mitochondrial encephalopathy, lactic acidosis and stroke-like episodes syndrome; materially-inherited diabetes and deafness; Leber's hereditary optic neuropathy; cancer; neuropathy, ataxia, retinitis pigmentosa-maternally inherited Leigh syndrome; Danon disease; diabetes; diabetic nephropathy; metabolic disorders; heart failure; ischemic heart disease leading to myocardial infarction; psychiatric diseases, schizophrenia; multiple sulfatase deficiency; mucolipidosis II; mucolipidosis III; mucolipidosis IV; GM1-gangliosidosis; neuronal ceroid-lipofuscinoses; Alpers disease; Barth syndrome; beta-oxidation defects; carnitine-acyl-carnitine deficiency; carnitine deficiency; creatine deficiency syndromes; co-enzyme Q10 deficiency; complex I deficiency; complex II deficiency; complex III deficiency; complex IV deficiency; complex V deficiency; COX deficiency; chronic progressive external ophthalmoplegia syndrome; CPT I deficiency; CPT II deficiency; glutaric aciduria type II; Kearns-Sayre syndrome; lactic acidosis; long-chain acyl-CoA dehydrogenase deficiency; Leigh disease or syndrome; Leigh Syndrome French Canadian variant; lethal infantile cardiomyopathy; Luft disease; medium-chain acyl-CoA dehydrogenase deficiency; myoclonic epilepsy and ragged-red fiber syndrome; mitochondrial cytopathy; mitochondrial recessive ataxia syndrome; mitochondrial DNA depletion syndrome; myoneurogastrointestinal disorder and encephalopathy; Pearson syndrome; pyruvate dehydrogenase deficiency; pyruvate carboxylase deficiency; POLG mutations; medium/short-chain 3-hydroxyacyl-CoA dehydrogenase deficiency; and very long-chain acyl-CoA dehydrogenase deficiency; peroxisomal disorders; methylmalonic acidemia; mevalonate kinase deficiency; age-dependent decline in cognitive function and muscle strength; muscle structure disorders; and cognitive impairment associated with neurodegenerative and neuropsychiatric disorders.

31. The method according to claim 30, wherein the neurodegenerative disease is selected from Parkinson's disease, Alzheimer's disease, amyotrophic lateral sclerosis, Huntington's disease, ischemia, stroke, dementia with Lewy bodies, multiple system atrophy, progressive supranuclear palsy, corticobasal degeneration, frontotemporal dementia; and Parkinson's disease related to mutations in α-synuclein, parkin, PINK1, GBA, and LRRK2, and autosomal recessive juvenile Parkinson's disease where parkin is mutated.

32. The method according to claim 30, wherein the neurodegenerative disease is selected from Leigh syndrome or disease, X-linked Leigh's disease, Leigh Syndrome French Canadian Variant, and/or the symptoms associated with Leigh's disease.

33. The method according to claims 27 to 29, wherein the cancer is selected from breast, ovarian, prostate, lung, kidney, gastric, colon, testicular, head and neck, pancreas, brain, melanoma, bone, liver, soft tissue, cancers of tissue organs, cancers of the blood cells, CML, AML, mantle cell lymphoma, neuroblastoma, melanoma, soft tissue sarcoma, liposarcoma, fibroblastic sarcoma, leiomyosarcoma, hepatocellular carcinoma, osteosarcoma, oesophageal cancer, leukaemia, lymphoma, multiple myeloma, metastatic carcinoma, osteosarcoma, chondosarcoma, Ewing's sarcoma, nasopharyngeal carcinoma, colorectal cancer, colorectal cancer, non-small cell lung carcinoma, cancer where apoptotic pathways are dysregulated, and cancer where proteins of the BCL-2 family are mutated, or over or under expressed.

34. The method according to claim 29, wherein the fibrosis is selected from fibrosis or a fibrotic disorder associated with the accumulation of extracellular matrix constituents that occurs following trauma, inflammation, tissue repair, immunological reactions, cellular hyperplasia, and neoplasia.

35. The method according to claim 34, wherein the fibrosis is selected from fibrosis, a fibrotic disorder associated with major organ diseases, fibroproliferative disorders, and scarring associated with trauma.

36. The method according to claim 35, wherein the fibrosis is selected from fibrosis or a fibrotic disorder associated with interstitial lung disease, liver cirrhosis, non-alcoholic fatty liver disease, non-alcoholic fatty liver disease, and non-alcoholic steatohepatitis, kidney disease, acute kidney injury, chronic kidney disease, delayed kidney graft function, heart or vascular disease, diseases of the eye, systemic and local scleroderma, keloids, hypertrophic scars, atherosclerosis, restenosis, Dupuytren's contracture, surgical complications, chemotherapeutics drug-induced fibrosis, radiation-induced fibrosis, accidental injury and burns, retroperitoneal fibrosis, and peritoneal fibrosis/peritoneal scarring.

37. The method according to claim 36, wherein the fibrosis associated with interstitial lung disease is selected from sarcoidosis, silicosis, drug reactions, infections, collagen vascular diseases, rheumatoid arthritis, systemic sclerosis, scleroderma, pulmonary fibrosis, idiopathic pulmonary fibrosis, usual interstitial pneumonitis, interstitial lung disease, cryptogenic fibrosing alveolitis, bronchiolitis obliterans, and bronchiectasis.

38. The method according to claim 36, wherein the kidney disease is acute kidney injury or chronic kidney disease.

39. A pharmaceutical composition comprising a compound of formula (I) as defined in claim 1, a tautomer thereof, or a pharmaceutically acceptable salt of said compound or tautomer, together with one or more pharmaceutically acceptable excipients.

40. A compound selected from formulae (II) and (III): 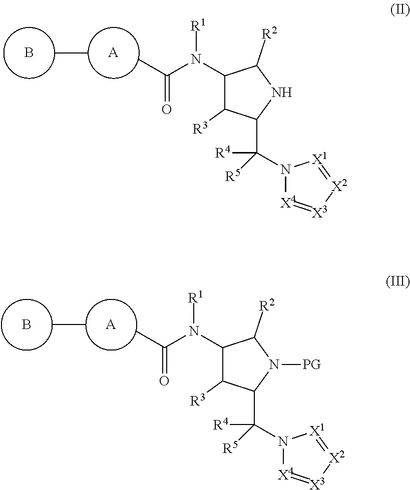
wherein PG is a protecting group and ring A, ring B, X¹, X², X³, X⁴, R¹, R², R³, R⁴ and R⁵ are as defined for the compound of formula (I) claim 1, a tautomer thereof, or a salt of said compound or tautomer; and wherein the protecting group is preferably selected from tert-butyloxycarbonyl, benzyloxycarbonyl, p-methoxybenzyl carbonyl, 9-fluorenylmethyloxycarbonyl, acetyl, benzoyl, benzyl, carbamate, p-methoxybenzyl, 3,4-dimethoxybenzyl, p-methoxyphenyl, tosyl, trichloroethoxycarbonyl, 4-nitrobenzenesulfonyl and 2-nitrophenylsulfenyl.
